(19) United States
(12) Patent Application Publication
(10) Pub. No.: US 2022/0259305 A1
(43) Pub. Date: Aug. 18, 2022

(54) METHODS FOR TREATMENT OF REFRACTORY GENERALIZED MYASTHENIA GRAVIS WITH ECULIZUMAB

(71) Applicant: Alexion Pharmaceuticals, Inc., Boston, MA (US)

(72) Inventors: Jing Jing Wang, Woodbridge, MA (US); Warren Wasiewski, Lancaster, PA (US); Fanny O'Brien, Norwell, MA (US); Kenji Fujita, Milburn, NJ (US)

(73) Assignee: Alexion Pharmaceuticals, Inc., Boston, MA (US)

(21) Appl. No.: 17/630,601

(22) PCT Filed: Aug. 4, 2020
(86) PCT No.: PCT/US2020/044894

§ 371 Date: Jan. 27, 2022

Related U.S. Application Data
(60) Provisional application No. 62/882,892, filed on Aug. 5, 2019.
(60) Provisional application No. 62/890,429, filed on Aug. 22, 2019.
(60) Provisional application No. 62/915,550, filed on Oct. 15, 2019.
(60) Provisional application No. 62/944,670, filed on Dec. 6, 2019.
(60) Provisional application No. 62/966,967, filed on Jan. 28, 2020.
(60) Provisional application No. 62/981,901, filed on Feb. 26, 2020.

(51) Int. Cl. C07K 16/28; A61P 21/04

(52) U.S. Cl. CPC C07K 16/28; A61K 2039/545; A61P 21/04

(57) ABSTRACT

The disclosure provides methods of treating refractory myasthenia gravis (MG) in a subject in need thereof by administering to the subject a substance that specifically binds complement component 5 (C5). In certain embodiments, the substance that specifically binds C5 is a binding protein, such as an anti-C5 antibody. In certain embodiments, the patient achieves and maintains the status of improved or MM according to the MGFA Post-Intervention Status.

### RELATED APPLICATIONS

This application claims the benefit of priority to U.S. Provisional Patent Application No. 62/882,892, filed Aug. 5, 2019, U.S. Provisional Patent Application No. 62/890,429, filed Aug. 22, 2019, U.S. Provisional Patent Application No. 62/915,550, filed Oct. 15, 2019, U.S. Provisional Patent Application No. 62/944,670, filed Dec. 6, 2019, U.S. Provisional Patent Application No. 62/966,967, filed Jan. 28, 2020, and U.S. Provisional Patent Application No. 62/981,901, filed Feb. 26, 2020, the entire contents of which are incorporated herein by reference for all purposes.

### REFERENCE TO SEQUENCE LISTING SUBMITTED ELECTRONICALLY

The content of the electronically submitted sequence listing in ASCII text file (Name: 706898_AX9_006PC_ST25_Sequence_Listing.txt; Size: 56 KB; and Date of Creation: Jul. 22, 2020) is incorporated herein by reference in its entirety.

### BACKGROUND

Myasthenia Gravis (MG) is a rare, debilitating, acquired autoimmune neurologic disorder of the neuromuscular junction (NMJ) caused by the failure of neuromuscular transmission, which results from the binding of auto-antibodies (Abs) to proteins involved in signaling at the NMJ. These proteins include the nicotine acetylcholine receptors (AChRs) or, less frequently, a muscle-specific tyrosine kinase (MuSK) involved in AChR clustering.

MG has a prevalence of 14-20 per 100,000 in the U.S., affecting roughly 60,000 Americans. It affects males and females in equal ratio, although the incidence in females peaks in the 3rd decade as compared to males in whom the peak age at onset is in the 6th or 7th decade. Mortality from MG is approximately 4%, mostly due to respiratory failure.

Myasthenia gravis is clinically characterized by weakness and fatigability of voluntary skeletal muscles. MG may initially present with ocular muscle weakness affecting eye and eyelid movement, referred to as ocular MG (oMG). Ten percent of subjects have disease limited to ocular muscles. Ninety percent of subjects have generalized MG, with muscle weakness involving neck, head, spine, bulbar, respiratory, or limb muscles. Bulbar weakness refers to muscles controlled by nerves originating from the bulb-like part of the brainstem and manifests as difficulty in talking, chewing, swallowing, and control of the head. MG may cause life-threatening respiratory failure, referred to as myasthenic crisis. About 15% to 20% of subjects will experience a myasthenic crisis during the course of their disease, 75% within 2 years of diagnosis, requiring hospitalization and ventilatory support.

While there is no cure for MG, there are a variety of therapies that reduce muscle weakness and improve neuromuscular function. Current available treatments for myasthenia gravis aim to modulate neuromuscular transmission, inhibit the production or effects of pathogenic antibodies, or inhibit inflammatory cytokines. There is currently no specific treatment that targets the underlying pathophysiology of NMJ injury specifically: anti-AChR antibody-AChR interactions resulting in complement activation via the classical pathway and inflammation, with the resultant destruction of the NMJ. There is no specific treatment that corrects the autoimmune defect in MG. With immunosuppressive therapies (ISTs) the current standard of care, which usually combines cholinesterase inhibitors, corticosteroids and immunosuppressive drugs (most commonly azathioprine [AZA], cyclosporine, and mycophenolate mofetil [MMF]), the majority of subjects with MG have their disease reasonably well controlled.

However, a proportion of 10-15% of refractory subjects do not respond adequately to ISTs, or cannot tolerate ISTs, and those who require repeated treatments with plasma exchange (PE) and/or intravenous immunoglobulin (IVIg) to maintain clinical stability. These patients are considered to have refractory MG. Although there is no generally recognized standard definition of ‘refractory’ disease in gMG, criteria for refractory disease that have been used include failure to respond to conventional treatments such as immunosuppressive therapies (ISTs), inability to reduce IST use without clinical relapse, intolerable adverse reactions to conventional treatments, requirement for large doses of potentially harmful agents such as ISTs, presence of comorbidities that contraindicate conventional treatments, requirement for repeated short-term rescue therapy (e.g. intravenous immunoglobulin and plasma exchange), and recurrent myasthenic crises.

A study in two US health plan databases reported 4-fold and 4.7-fold increases in rates of myasthenic crisis/exacerbation, respectively, in refractory versus non-refractory cases. A UK study reported increased healthcare resource use in refractory versus non-refractory MG. Therefore, refractory MG can have a severe impact on the health-related quality of life (QOL) and increase the socioeconomic burden of the disease. It is important, therefore, to consider therapeutic needs in treatment-refractory MG.

The Myasthenia Gravis Foundation of America (MGFA) post-intervention status can be used to evaluate changes in a patient's condition following treatment, including improvement, worsening or no change of clinical manifestations from pretreatment. Patients are considered to have achieved minimal manifestations (MM) if they have no symptoms indicating functional limitations. Those experiencing no symptoms of MG for at least 1 year, apart from isolated ocular muscle weakness, while receiving therapy other than cholinesterase inhibitors, are considered to have achieved pharmacologic remission (PR). There is a consensus within the medical community that achievement of an MGFA post-intervention status of MM or better should be the goal of treatment for patients with MG. To achieve this goal, an alternative therapy is needed.

### SUMMARY

This disclosure provides a method of treating refractory generalized myasthenia gravis in a patient in need thereof comprising administering a therapeutically effective amount of an anti-C5 antibody or an antigen binding fragment thereof to the patient; wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability, and wherein the patient is treated for at least 52 weeks and achieves a Myasthenia Gravis Foundation of America (MGFA) post-intervention status of Improved or Minimal Manifestations (MM) after at least 4 weeks of treatment.

In one embodiment, the anti-C5 antibody is eculizumab. In another embodiment, eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter. In some embodiments, the method further comprises performing plasmapheresis on the patient and administering eculizumab at a dose of between 300 mg and 1200 mg to the patient within 4 hours of completion of plasmapheresis. In some embodiments, the method further comprises performing plasmapheresis on the patient and administering eculizumab at a dose of between 600 mg and 900 mg to the patient within 90 minutes of completion of plasmapheresis. In some embodiments, the method further comprises performing plasmapheresis on the patient and administering eculizumab at a dose of 600 mg to the patient within 1 hour of completion of plasmapheresis.

In some embodiments, the therapeutically effective amount of the anti-C5 antibody or antigen binding fragment thereof for treating refractory generalized myasthenia gravis is based on the weight of the subject. In another embodiment, the anti-C5 antibody is ravulizumab. In another embodiment, ravulizumab, or an antigen binding fragment thereof is administered to a patient weighing ≥40 and <60 kg: once on Day 1 of the administration cycle at a loading dose of 2400 mg; and on Day 15 of the administration cycle and every eight weeks thereafter at a maintenance dose of 3000 mg. In another embodiment, ravulizumab, or an antigen binding fragment thereof is administered to a patient weighing ≥60 and <100 kg: once on Day 1 of the administration cycle at a loading dose of 2700 mg; and on Day 15 of the administration cycle and every eight weeks thereafter at a maintenance dose of 3300 mg. In another embodiment, ravulizumab, or antigen binding fragment thereof, is administered to a patient weighing ≥100 kg: once on Day 1 of the administration cycle at a loading dose of 3000 mg; and on Day 15 of the administration cycle and every eight weeks thereafter at a maintenance dose of 3600 mg.

In some embodiments, the patient achieves a MGFA post-intervention status of Improved or MM after 4 weeks of treatment. In some embodiments, the patient achieves a MGFA post-intervention status of Improved or MM after 12 weeks of treatment. In some embodiments, the patient achieves a MGFA post-intervention status of Improved or MM after 26 weeks of treatment. In some embodiments, the patient achieves a MGFA post-intervention status of Improved or MM after 52 weeks of treatment. In some embodiments, the patient achieves a MGFA post-intervention status of Improved or MM after 66 weeks of treatment. In some embodiments, the patient achieves a MGFA post-intervention status of Improved or MM after 78 weeks of treatment. In some embodiments, the patient achieves a MGFA post-intervention status of Improved or MM after 104 weeks of treatment. In some embodiments, the patient achieves a MGFA post-intervention status of Improved or MM after 130 weeks of treatment. In some embodiments, the patient achieves a MGFA post-intervention status of Improved or MM after 156 weeks of treatment. In some embodiments, the patient achieves a MGFA post-intervention status of Improved. In some embodiments the patient achieves a MGFA post-intervention status of MM.

In some embodiments, the patient experiences a clinically meaningful improvement (reduction) in a measurement of generalized myasthenia gravis severity after 26 weeks of treatment selected from the group consisting of Myasthenia Gravis Activities of Daily Living (MG-ADL) score, quantitative Myasthenia Gravis (QMG), score and Myasthenia Gravis Composite (MGC) score. In some embodiments, the clinically meaningful improvement the patient experiences is an at least a 3 point reduction in the patient's MG-ADL score after 26 weeks of treatment. In some embodiments, the clinically meaningful improvement the patient experiences is an at least a 4 point reduction in the patient's QMG score after 26 weeks of treatment. In some embodiments, the clinically meaningful improvement the patient experiences is an at least a 6 point reduction in the patient's MGC score after 26 weeks of treatment. In some embodiments, the patient experiences a clinically meaningful improvement (reduction) in quality of life as measured by Myasthenia Gravis Quality of Life (MG-QOL-15) score after 26 weeks of treatment. In some embodiments, the clinically meaningful improvement the patient experiences is an at least a 6 point reduction in the patient's MG-QOL-15 score after 26 weeks of treatment.

In some embodiments, the patient experiences a clinically meaningful improvement (reduction) in neuro-fatigue as measured by Neuro-QOL Fatigue score after 26 weeks of treatment. In some embodiments, the clinically meaningful improvement the patient experiences is an at least an 8 point reduction in the patient's Neuro-QOL score after 26 weeks of treatment. In some embodiments, the patient experiences a clinically meaningful improvement (increase) in health status as measured by EQ-5D health status score after 26 weeks of treatment.

This disclosure further provides a method of treating refractory generalized myasthenia gravis in a patient in need thereof comprising administering an anti-C5 antibody or an antigen binding fragment thereof to the patient; wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; and wherein the patient is treated for at least 52 weeks and achieves a Myasthenia Gravis Foundation of America (MGFA) post-intervention status of Improved or Minimal Manifestations (MM) after at least 4 weeks of treatment; and wherein the patient has a clinically meaningful improvement (reduction) in at least two measurements of generalized myasthenia gravis severity selected from the group consisting of MG-ADL QMG, MGC, MG-QOL, and Neuro-QOL.

The disclosure further provides a method of treating refractory generalized myasthenia gravis in a patient in need thereof comprising administering an anti-C5 antibody or antigen binding fragment thereof to the patient; wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; wherein the patient is treated for at least 52 weeks and achieves a Myasthenia Gravis Foundation of America (MGFA) post-intervention status of Improved or Minimal Manifestations (MM) after at least 4 weeks of treatment; and wherein the patient has a clinically meaningful improvement (reduction) in five measurements of generalized myasthenia gravis severity, wherein the five measurements of generalized myasthenia gravis severity are a reduction in MG-ADL of at least 3 points, a reduction of QMG of at least 4 points, a reduction in MGC of at least 6 points, a reduction in MG-QOL of at least 6 points, and a reduction in Neuro-QOL of at least 8 points.

In some embodiments, the patient has a clinically meaningful improvement (reduction) in five measurements of generalized myasthenia gravis, wherein the five measurements of generalized myasthenia gravis severity are a reduction in MG-ADL of at least 4 points, a reduction of QMG of at least 5 points, a reduction in MGC of at least 10 points, a reduction in MG-QOL of at least 11 points, and a reduction in Neuro-QOL of at least 16 points.

The disclosure also provides a method of maintaining a Myasthenia Gravis Foundation of America (MGFA) post-intervention status of Improved or Minimal Manifestations (MM) in a patient with refractory generalized myasthenia gravis in need thereof comprising administering a therapeutically effective amount of an anti-C5 antibody or an antigen binding fragment thereof to the patient; wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; wherein the patient had achieved the Improved or MM status.

In some embodiments, the improved or MM status is maintained for at least 4, 12, 26, 52, 66, 78, 104, 130 or 156 weeks. In some embodiments, the patient starts the maintenance with the MM status. In other embodiments, the MM status is maintained for at least 4, 12, 26, 52, 66, 78, 104, 130 or 156 weeks.

The following embodiments, apply to any of the disclosures and embodiments described in the Summary above. In some embodiments, eculizumab is administered by intravenous infusion. In some embodiments, eculizumab is administered subcutaneously.

In some embodiments, the eculizumab comprises a heavy chain amino acid sequence according to SEQ ID NO: 10 and a light chain amino acid sequence according to SEQ ID NO: 11. In some embodiments, ravulizumab comprises a heavy chain amino acid sequence according to SEQ ID NO: 14 and a light chain amino acid sequence according to SEQ ID NO: 11.

In some embodiments, the patient has failed treatment over one year or more with two or more ISTs in sequence or in combination. In some embodiments, the patient has failed at least one IST and requires chronic plasma exchange or IVIg to control symptoms.

In some embodiments, the therapeutically effective amount of the anti-C5 antibody or antigen binding fragment thereof is maintained at a concentration of between 50-100 μg/mL in the patient's serum.

In some embodiments, the patient experiences a reduction in the administration of one or more IST following at least 26 weeks of treatment. In another embodiment, the patient experiences a reduction in IST dosing following at least 26 weeks of treatment. In some embodiments, the patient experiences a reduction in one or more IST dosing and a discontinuation in one or more IST following at least 26 of treatment.

In some embodiments, the anti-C5 antibody or an antigen binding fragment thereof is selected from the group consisting of eculizumab, ravulizumab, BNJ421, 7086 antibody, 8110 antibody, 305LO5, SKY59 and REGN3918. In some embodiments, the patient switches from receiving one anti-C5 antibody or antigen binding fragment thereof to a different anti-C5 antibody or antigen binding fragment thereof during the course of treatment. In other embodiments, the different anti-C5 antibodies may be administered during separate treatment periods.

This disclosure also provides a use for treating refractory generalized myasthenia gravis in a patient in need thereof. In some embodiments the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; and the patient is treated for at least 52 weeks and achieves a Myasthenia Gravis Foundation of America (MGFA) post-intervention status of Improved or Minimal Manifestations (MM) after at least 4 weeks of treatment.

In some embodiments, eculizumab is used for administration at a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.

In some embodiments, the use of eculizumab further comprises performing plasmapheresis on the patient and administering eculizumab at a dose of between 300 mg and 1200 mg to the patient within 4 hours of completion of plasmapheresis. In some embodiments, the use of eculizumab further comprises performing plasmapheresis on the patient and administering eculizumab at a dose of between 600 mg and 900 mg to the patient within 90 minutes of completion of plasmapheresis. In some embodiments, the use of eculizumab further comprises performing plasmapheresis on the patient and administering eculizumab at a dose of 600 mg to the patient within 1 hour of completion of plasmapheresis. In some embodiments, the use of eculizumab is at a therapeutically effective amount, wherein the therapeutically effective amount is based on the weight of the subject.

In some embodiments, eculizumab is for use in achieving a MGFA post-intervention status of Improved or MM after 4 weeks of treatment in a patient. In some embodiments, eculizumab is for use in achieving a MGFA post-intervention status of Improved or MM after 12 weeks of treatment in a patient. In some embodiments, eculizumab is for use in achieving a MGFA post-intervention status of Improved or MM after 26 weeks of treatment in a patient. In some embodiments, eculizumab is for use in achieving a MGFA post-intervention status of Improved or MM after 52 weeks of treatment in a patient. In some embodiments, eculizumab is for use in achieving a MGFA post-intervention status of Improved or MM after 66 weeks of treatment in a patient. In some embodiments, eculizumab is for use in achieving a MGFA post-intervention status of Improved or MM after 78 weeks of treatment in a patient. In some embodiments, eculizumab is for use in achieving a MGFA post-intervention status of Improved or MM after 104 weeks of treatment in a patient. In some embodiments, eculizumab is for use in achieving a MGFA post-intervention status of Improved or MM after 130 weeks of treatment in a patient. In some embodiments, eculizumab is for use in achieving a MGFA post-intervention status of Improved or MM after 156 weeks of treatment in a patient. In some embodiments, eculizumab is for use in achieving a MGFA post-intervention status of Improved in a patient. In some embodiments, eculizumab is for use in achieving a MGFA post-intervention status of MM in a patient.

In some embodiments, the patient experiences a clinically meaningful improvement (reduction) in a measurement of generalized myasthenia gravis severity after 26 weeks of treatment selected from the group consisting of Myasthenia Gravis Activities of Daily Living (MG-ADL) score, quantitative Myasthenia Gravis (QMG), score and Myasthenia Gravis Composite (MGC) score. In some embodiments, the clinically meaningful improvement the patient experiences is an at least a 3 point reduction in the patient's MG-ADL score after 26 weeks of treatment. In some embodiments, the clinically meaningful improvement the patient experiences is an at least a 4 point reduction in the patient's QMG score after 26 weeks of treatment. In some embodiments, the clinically meaningful improvement the patient experiences is an at least a 6 point reduction in the patient's MGC score after 26 weeks of treatment. In some embodiments, the patient experiences a clinically meaningful improvement (reduction) in quality of life as measured by Myasthenia Gravis Quality of Life (MG-QOL-15) score after 26 weeks of treatment. In some embodiments, the clinically meaningful improvement the patient experiences is an at least a 6 point reduction in the patient's MG-QOL-15 score after 26 weeks of treatment. In some embodiments, wherein the patient experiences a clinically meaningful improvement (reduction) in neuro-fatigue as measured by Neuro-QOL Fatigue score after 26 weeks of treatment. In some embodiments, the clinically meaningful improvement the patient experiences is an at least an 8 point reduction in the patient's Neuro-QOL score after 26 weeks of treatment. In some embodiments, the patient experiences a clinically meaningful improvement (increase) in health status as measured by EQ-5D health status score after 26 weeks of treatment.

This disclosure also provides a use of eculizumab for treating refractory generalized myasthenia gravis in a patient in need thereof comprising administering eculizumab to the patient; wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; wherein the patient is treated for at least 52 weeks and achieves a Myasthenia Gravis Foundation of America (MGFA) post-intervention status of Improved or Minimal Manifestations (MM) after at least 4 weeks of treatment; and wherein the patient has a clinically meaningful improvement (reduction) in at least two measurements of generalized myasthenia gravis severity selected from the group consisting of MG-ADL, QMG, MGC, MG-QOL, and Neuro-QOL.

In some embodiments, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter. In some embodiments, eculizumab is for use at a therapeutically effective amount, wherein the therapeutically effective amount is based on the weight of the subject.

This disclosure also provides eculizumab for use in treating refractory generalized myasthenia gravis in a patient in need thereof comprising administering eculizumab to the patient; wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; wherein the patient is treated for at least 52 weeks and achieves a Myasthenia Gravis Foundation of America (MGFA) post-intervention status of Improved or Minimal Manifestations (MM) after at least 4 weeks of treatment; and wherein the patient has a clinically meaningful improvement (reduction) in five measurements of generalized myasthenia gravis severity, wherein the five measurements of generalized myasthenia gravis severity are a reduction in MG-ADL of at least 3 points, a reduction of QMG of at least 4 points, a reduction in MGC of at least 6 points, a reduction in MG-QOL of at least 6 points, and a reduction in Neuro-QOL of at least 8 points.

In some embodiments, eculizumab is for use at a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter. In some embodiments, eculizumab is for use at a therapeutically effective amount, wherein the therapeutically effective amount is based on the weight of the subject. In some embodiments, the patient has a clinically meaningful improvement (reduction) in five measurements of generalized myasthenia gravis, wherein the five measurements of generalized myasthenia gravis severity are a reduction in MG-ADL of at least 4 points, a reduction of QMG of at least 5 points, a reduction in MGC of at least 10 points, a reduction in MG-QOL of at least 11 points, and a reduction in Neuro-QOL of at least 16 points.

This disclosure also provides eculizumab for the use in maintaining a Myasthenia Gravis Foundation of America (MGFA) post-intervention status of Improved or Minimal Manifestations (MM) in a patient with refractory generalized myasthenia gravis in need thereof comprising administering a therapeutically effective amount of eculizumab to the patient; wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; and wherein the patient had achieved the Improved or MM status. In some embodiments, eculizumab is for use at a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter. In some embodiments, the use of eculizumab further comprises performing plasmapheresis on the patient and administering eculizumab at a dose of between 300 mg and 1200 mg to the patient within 4 hours of completion of plasmapheresis. In some embodiments, the use of eculizumab further comprises performing plasmapheresis on the patient and administering eculizumab at a dose of between 600 mg and 900 mg to the patient within 90 minutes of completion of plasmapheresis. In some embodiments, the use of eculizumab further comprises performing plasmapheresis on the patient and administering eculizumab at a dose of 600 mg to the patient within 1 hour of completion of plasmapheresis. In some embodiments, eculizumab is for use at a therapeutically effective amount, wherein the therapeutically effective amount is based on the weight of the subject.

In some embodiments, use of eculizumab results in Improved or MM status is maintained for at least 4, 12, 26, 52, 66, 78, 104, 130 or 156 weeks. In some embodiments, the patient starts the maintenance with the MM status. In some embodiments, the MM status is maintained for at least 4, 12, 26, 52, 66, 78, 104, 130 or 156 weeks. In some embodiments eculizumab is for use by administration by intravenous infusion. In some embodiments eculizumab is for use by subcutaneous administration.

In some embodiments, the eculizumab comprises a heavy chain amino acid sequence according to SEQ ID NO: 10 and a light chain amino acid sequence according to SEQ ID NO: 11.

In some embodiments, eculizumab is for use in a patient that has failed treatment over one year or more with two or more ISTs in sequence or in combination. In some embodiments, eculizumab is for use in a patient that has failed at least one IST and requires chronic plasma exchange or IVIg to control symptoms.

In some embodiments, the therapeutically effective amount of eculizumab is maintained at a concentration of between 50-100 μg/mL in the patient's serum.

In some embodiments, the patient experiences a reduction in the administration of one or more IST following at least 26 weeks of treatment. In some embodiments, the patient experiences a reduction in IST dosing following at least 26 weeks of treatment.

In some embodiments, the patient experiences a reduction in IST dosing and a discontinuation in one or more IST following at least 26 of treatment. In some embodiments, the patient switches from receiving one anti-C5 antibody or antigen binding fragment thereof to eculizumab during the course of treatment.

### BRIEF DESCRIPTION OF THE DRAWINGS

FIG. 1 is a schematic of the overall design of the clinical trial disclosed herein.

FIGS. 2A and 2B are schematics of the EUROQOL (EQ-5D) survey of health status questionnaire used in the clinical trial disclosed herein.

FIG. 3 is a schematic of the N. meningitidis vaccination schedule used in the clinical trial disclosed herein.

FIG. 4 is a schematic of the dosing schedule used in the clinical trial disclosed herein.

FIG. 5 is a schematic of the dosing, clinical evaluation and safety follow-up schedule, used in the clinical trial disclosed herein.

FIG. 6 is a schematic of the dosing schedule used in the clinical trial also including the extension period disclosed herein.

FIG. 7 is a graphical depiction of the changes from baseline in MG-ADL values obtained for placebo and eculizumab groups over the initial 26 weeks of the trial.

FIG. 8 is a graphical depiction of the changes from baseline in QMG values obtained for placebo and eculizumab groups over the initial 26 weeks of the trial.

FIG. 9 is a graphical depiction of the changes from baseline in MGC values obtained for placebo and eculizumab groups over the initial 26 weeks of the trial.

FIG. 10 is a graphical depiction of the changes from baseline in MG-QOL 15 values obtained for placebo and eculizumab groups over the initial 26 weeks of the trial.

FIG. 11 is a graphical depiction of the numbers of patients in both the placebo and eculizumab treated groups achieving between a 5 and 10 point reduction in QMG score over the initial 26 weeks of the trial.

FIG. 12 is a schematic of the REGAIN study design.

FIG. 13 is a graphical depiction of responder analyses (MG-ADL and QMG) illustrating the proportion of patients with improvement in total score and no rescue therapy at week 26 from baseline.

FIG. 14 is a graphical depiction of the proportion of patients with a ≥3, ≥5, or ≥8-point reduction in MG-ADL total score and no rescue therapy over time from baseline to week 26.

FIG. 15 is a graphical depiction of the proportion of patients with ≥5, ≥7, or ≥10-point reduction in QMG total score and no rescue over time from baseline to week 26.

FIG. 16 is a graphical depiction of dual responders (assessed by MG-ADL and QMG total scores) with no rescue therapy at week 26.

FIG. 17 is a graphical depiction of the proportion of patients with at least 3-point improvement in MG-ADL total score and ≥5-point improvement in QMG total score and no rescue therapy assessed over time from baseline to week 26.

FIG. 18 is a graphical depiction of the percentage of patients who simultaneously met increasingly stringent criteria based on MG-ADL and QMG. The bottom row describes a threshold for both scales above the MCID (minimal clinically meaningful difference: 3 for MG-ADL and 5 for QMG). Higher bars represent increasing thresholds. The right-most panel displays odds ratios for meeting each threshold for eculizumab vs. placebo treated patients.

FIG. 19 is a graphical depiction of the change from baseline in MG-ADL total score (LS Mean and 95% CI) by treatment arm over time from ECU-MG-302 baseline to week 52 in study ECU-MG-302 using a repeated-measures model.

FIG. 20 is a graphical depiction of the change from baseline in MG-ADL total score (Mean and 95% CI) by treatment arm over time from ECU-MG-301 baseline to week 52 in study ECU-MG-302.

FIG. 21 is a graphical depiction of the study design of the REGAIN and open-label study.

FIG. 22 is a graphical depiction of the patient disposition in REGAIN and the open-label study.

FIG. 23 is a graphical depiction of the proportions of patients who entered the open-label study who achieved a Myasthenia Gravis Foundation of America post-intervention status of improved or minimal manifestations.

FIG. 24 is a graphical depiction of the clinical response in REGAIN and the open-label extension study, versus REGAIN baseline. ECU, eculizumab; MG-ADL, Myasthenia Gravis Activities of Daily Living; PLC, placebo; QMG, Quantitative Myasthenia Gravis test.

FIG. 25 is a graphical depiction of the proportions of patients who entered the open-label study who achieved a status of ‘minimal symptom expression’ defined as an MG-ADL total score of 0-1 (FIG. 25A), or MG-QOL15 total score of 0-3 (FIG. 25B).

FIG. 26 is a graphical depiction of the percent change from baseline in MG-ADL total score in the response analysis set of the REGAIN and the open-label study.

FIG. 27 is a graphical depiction of the percent change from baseline in QMG total score in the response analysis set of the REGAIN and the open-label study.

FIG. 28 is a graphical depiction of changes in MG-ADL mean total score (FIG. 28A) and QMG mean total score (FIG. 28B) from REGAIN baseline to OLE week 130. Patient numbers were not the same for each assessment.

FIG. 29 is a graphical depiction of changes in MG-ADL least squares (LS) mean total score (FIG. 29A) and QMG LS mean total score (FIG. 29B) from REGAIN baseline to OLE week 130. Patient numbers were not the same for each assessment.

FIG. 30 is a graphical depiction of linear regression models showing correlations between MG-ADL and QMG total scores for changes from eculizumab baseline to last OLE assessment (FIG. 30A) and at last OLE assessment (FIG. 30B).

FIG. 31 is a graphical depiction of changes in MG-ADL mean domain scores from REGAIN baseline to OLE week 130 for ocular (FIG. 31A), bulbar (FIG. 31B), respiratory (FIG. 31C), and limb domains (FIG. 31D). Patient numbers were not the same for each assessment.

FIG. 32 is a graphical depiction of changes in MG-ADL LS mean domain scores from open-label baseline to week 130 for ocular (FIG. 32A), bulbar (FIG. 32B), respiratory (FIG. 32C), and limb domains (FIG. 32D). Patient numbers were not the same for each assessment.

FIG. 33 is a graphical depiction of changes in QMG mean domain scores from REGAIN baseline to OLE week 130 for ocular (FIG. 33A), bulbar (FIG. 33B), respiratory (FIG. 33C), and gross motor domains (FIG. 33D). Patient numbers were not the same for each assessment.

FIG. 34 is a graphical depiction of changes in in QMG LS mean domain scores from open-label baseline to week 130 for ocular (FIG. 34A), bulbar (FIG. 34B), respiratory (FIG. 34C), and gross motor domains (FIG. 34D). Patient numbers were not the same for each assessment.

### DETAILED DESCRIPTION

The disclosure provides methods of treating myasthenia gravis (MG) in subjects or patients in need thereof by administering an antibody that specifically binds complement component 5 (C5). In certain embodiments, the antibody that specifically binds C5 reduces the rate at which C5 is cleaved, in vivo, into C5a and C5b. In other embodiments, the antibody that specifically binds C5, binds to one or both of the C5a and/or C5b fragments. In any of these embodiments, the antibody that specifically binds C5 blocks the complement cascade at C5, thereby reducing the release of proinflammatory mediators such as C5a and the formation of a C5b-9 Membrane Attack Complex (MAC).

In certain embodiments, the antibody that specifically binds C5 is eculizumab. In more specific embodiments, eculizumab is an antibody or a fragment thereof.

Eculizumab (h5G1.1-mAb) is a humanized monoclonal antibody (mAb) that was derived from the murine anti-human C5 antibody m5G1.1. Eculizumab specifically binds the terminal complement protein C5, thereby inhibiting its cleavage to C5a and C5b during complement activation. This strategic blockade of the complement cascade at C5 prevents the release of proinflammatory mediators and the formation of the Membrane Attack Complex or cytolytic pore, while preserving the early components of complement activation that are essential for the opsonization of microorganisms and clearance of immune complexes.

In certain embodiments, the antibody that specifically binds C5 is ravulizumab. Ravulizumab was engineered from eculizumab, a humanized monoclonal antibody that specifically binds with high affinity to the human terminal complement component (C5), inhibiting C5 enzymatic cleavage and thereby preventing the generation of the proinflammatory/prothrombotic complement activation products, C5a, and the cytolytic and proinflammatory/prothrombotic membrane attack complex, C5b-9, which are responsible for the antibody-mediated destruction of the neuromuscular junction (NMJ), loss of acetylcholine receptors, and failure of neuromuscular transmission associated with generalized myasthenia gravis (gMG). Eculizumab is approved for the treatment of gMG, paroxysmal nocturnal hemoglobinuria, neuromyelitis optica spectrum disorders (NMOSD) and/or atypical hemolytic uremic syndrome in many countries worldwide, including the USA, and countries in the European Union under the trade name Soliris®. Ravulizumab is approved for the treatment of paroxysmal nocturnal hemoglobinuria and/or atypical hemolytic uremic syndrome in several countries worldwide, including the USA, the European Union and Japan under the trade name Ultomiris®.

C5 binding proteins are described in U.S. Pat. No. 6,355,245, which is hereby incorporated herein by reference in its entirety. In certain embodiments, the anti-C5 antibody is a monoclonal antibody having a hybrid IgG2/4 isotype, such as that described in U.S. Pat. No. 9,732,149, which is hereby incorporated herein by reference in its entirety. In other embodiments, the anti-C5 antibodies are effective in reducing the cell-lysing ability of complement present in human blood. This property of the antibodies can be determined by methods well known in the art such as, for example, by the chicken erythrocyte hemolysis method described in U.S. Pat. No. 6,355,245.

In certain embodiments, anti-C5 antibodies bind to C5 or fragments thereof, e.g., C5a or C5b. In other embodiments, the anti-C5 antibodies recognize and bind epitopes on either the alpha chain or the beta chain of purified human complement component C5 and are capable of blocking the conversion of C5 into C5a and C5b by C5 convertase. See Wurzner et al., Complement. Inflamm. 8(5-6): 328-40 (1991).

In other embodiments, the anti-C5 antibodies recognize and bind epitopes within the alpha chain of purified human complement component C5. In this embodiment, the antibodies are capable of blocking the conversion of C5 into C5a and C5b by C5 convertase. In one example of this embodiment, the antibodies can provide this blockade at substantially the same concentrations needed to block hemolytic activity.

In some embodiments, the antibodies specifically bind to an amino-terminal region within the alpha chain, however, they do not specifically bind to free C5a. In certain embodiments, the C5 antibody is able to substantially inhibit complement hemolytic activity and to substantially inhibit the conversion of C5 to produce C5a. In some embodiments, the C5 antibodies provide these functions when used at a molar ratio of antibody to antigen (C5) of 3:1 or less.

As used herein, the term “antibodies” refers to immunoglobulins produced in vivo, as well as those produced in vitro by a hybridoma, and antigen binding fragments (e.g., Fab′ preparations) of such immunoglobulins, as well as to recombinantly expressed antibodies or antigen binding proteins, including immunoglobulins, chimeric immunoglobulins, “humanized” immunoglobulins, antigen binding fragments of such immunoglobulins, single chain antibodies, and other recombinant proteins containing antigen binding domains derived from immunoglobulins such as DVD-Ig and CODV-Ig. See U.S. Pat. Nos. 7,161,181 and 9,181,349. “Specificity” refers to the ability of a binding protein to selectively recognize and bind an antigen at a particular location or structure, known as an epitope, often found on the surface of the antigen.

The term “specifically binds,” means that a binding protein or fragment thereof forms a complex with an antigen that is relatively stable under physiologic conditions. Specific binding can be characterized by a dissociation constant of at least about 1×10⁻⁶ M or smaller. In other embodiments, the dissociation constant is at least about 1×10⁻⁷ M, 1×10⁻⁸ M, 1×10⁻⁹ M, or 1×10⁻¹⁰ M. Methods for determining whether two molecules specifically bind are well known in the art and include, for example, equilibrium dialysis, surface plasmon resonance, and the like.

The anti-C5 antibodies described herein bind to complement component C5 (e.g., human C5) and inhibit the cleavage of C5 into fragments C5a and C5b. Anti-C5 antibodies (or VH/VL domains derived therefrom) suitable for use in the invention can be generated using methods known in the art.

An exemplary anti-C5 antibody is eculizumab comprising heavy and light chains having the sequences shown in SEQ ID NOs: 10 and 11, respectively, or antigen binding fragments and variants thereof. Eculizumab is a humanized monoclonal antibody that is a terminal complement inhibitor.

In other embodiments, the antibody comprises the heavy and light chain complementarity determining regions (CDRs) or variable regions of eculizumab.

Accordingly, in one embodiment, the antibody comprises the CDR1, CDR2, and CDR3 domains of the VH region of eculizumab having the sequence set forth in SEQ ID NO: 7, and the CDR1, CDR2, and CDR3 domains of the VL region of eculizumab having the sequence set forth in SEQ ID NO: 8. In another embodiment, the antibody comprises heavy chain CDR1, CDR2, and CDR3 domains having the sequences set forth in SEQ ID NOs: 1, 2, and 3, respectively, and light chain CDR1, CDR2, and CDR3 domains having the sequences set forth in SEQ ID NOs: 4, 5, and 6, respectively. In another embodiment, the antibody comprises VH and VL regions having the amino acid sequences set forth in SEQ ID NO: 7 and SEQ ID NO: 8, respectively.

Empirical data indicate that serum eculizumab concentrations greater than 50 μg/mL and closer to at least 100 μg/mL are required to significantly reduce free C5 concentrations. Specifically, free C5 concentration was reduced significantly with increasing concentrations of eculizumab beginning at >50 μg/mL and was at near zero levels with eculizumab concentrations above 100 μg/ml. Thus, in various embodiments, the method comprises administering a therapeutically effective amount of eculizumab to the subject, wherein the therapeutically effective amount of eculizumab is maintained at a concentration of at least 50 μg/mL of eculizumab in serum of the subject. In another embodiment, the method comprises administering a therapeutically effective amount of eculizumab to the subject, wherein the therapeutically effective amount of eculizumab is maintained at a concentration of at least 60 μg/mL of eculizumab in serum of the subject. In one embodiment, the method comprises administering a therapeutically effective amount of eculizumab to the subject, wherein the therapeutically effective amount of eculizumab is maintained at a concentration of at least 70 μg/mL of eculizumab in serum of the subject. In another embodiment, the method comprises administering a therapeutically effective amount of eculizumab to the subject, wherein the therapeutically effective amount of eculizumab is maintained at a concentration of at least 80 μg/mL of eculizumab in serum of the subject. In another embodiment, the method comprises administering a therapeutically effective amount of eculizumab to the subject, wherein the therapeutically effective amount of eculizumab is maintained at a concentration of at least 90 μg/mL of eculizumab in serum of the subject. In another embodiment, the method comprises administering a therapeutically effective amount of eculizumab to the subject, wherein the therapeutically effective amount of eculizumab is maintained at a concentration of at least 100 μg/mL of eculizumab in serum of the subject.

In various embodiments, eculizumab is administered in a multiphase dosing regimen. For example, the multiphase dosing regimen comprises a first phase and a second phase in various embodiments. In certain embodiments, the first phase is an induction phase and comprises administration of eculizumab at between 900 mg once a week to the subject for between 1-10 weeks. The induction phase is concluded by administering the first maintenance phase dose of 1200 mg one week after the last 900 mg dose.

In other embodiments, the second phase is a maintenance phase and comprises administration of eculizumab at between 1000 and 1400 mg once every two weeks to the subject for 2 weeks, 4 weeks, 6 weeks, 8 weeks, 12, weeks, 26 weeks, or as long as myasthenia gravis persists. In other embodiments, the maintenance phase comprises administration of eculizumab at between 1000 and 1400 mg once every two weeks to the subject for 2 months, 4 months, 6 months, 8 months, 12 months, 2 years, three years, 4 years, 5 years, or for the remaining lifetime of the patient. In other embodiments, the maintenance phase comprises administration of eculizumab at about 1200 mg twice a month (biweekly) once the induction phase is complete.

In another embodiment, the method comprises administering a therapeutically effective amount of eculizumab or an eculizumab variant to the subject, wherein the therapeutically effective amount of eculizumab or eculizumab variant is maintained at a concentration of between 50-100 μg/mL, between 60-100 μg/mL, between 70-100 μg/mL, between 80-100 μg/mL, or between 90-100 μg/mL of eculizumab in serum of the subject.

Another exemplary anti-C5 antibody is antibody BNJ421 comprising heavy and light chains having the sequences shown in SEQ ID NOs: 20 and 11, respectively, or antigen binding fragments and variants thereof. BNJ421 (also known as ALXN1211) is described in International Publication No. WO 2015/134894 A1 and U.S. Pat. No. 9,079,949, the teachings or which are hereby incorporated by reference.

In other embodiments, the antibody comprises the heavy and light chain CDRs or variable regions of BNJ421. Accordingly, in one embodiment, the antibody comprises the CDR1, CDR2, and CDR3 domains of the VH region of BNJ421 having the sequence set forth in SEQ ID NO: 12, and the CDR1, CDR2, and CDR3 domains of the VL region of BNJ421 having the sequence set forth in SEQ ID NO: 8. In another embodiment, the antibody comprises heavy chain CDR1, CDR2, and CDR3 domains having the sequences set forth in SEQ ID NOs: 19, 18, and 3, respectively, and light chain CDR1, CDR2, and CDR3 domains having the sequences set forth in SEQ ID NOs: 4, 5, and 6, respectively. In another embodiment, the antibody comprises VH and VL regions having the amino acid sequences set forth in SEQ ID NO: 12 and SEQ ID NO: 8, respectively. In another embodiment, the antibody may comprise the heavy chain constant region of BNJ421 having the amino acid sequence set forth in SEQ ID NO: 9.

Another exemplary anti-C5 antibody is an eculizumab variant, known as antibody BNJ441, also known as ravulizumab, and engineered to have a longer half-life (T½) in humans comprising heavy and light chains having the sequences shown in SEQ ID NOs: 14 and 11, respectively, or antigen binding fragments and variants thereof. BNJ441 (also known as ALXN1210) is described in International Publication No. WO 2015/134894 A1 and U.S. Pat. No. 9,079,949, the teachings or which are hereby incorporated by reference. BNJ441 (ravulizumab) is a humanized monoclonal antibody that is structurally related to eculizumab (SOLIRIS®). BNJ441 selectively binds to human complement protein C5, inhibiting its cleavage to C5a and C5b during complement activation. This inhibition prevents the release of the proinflammatory mediator C5a and the formation of the cytolytic pore-forming membrane attack complex C5b-9 while preserving the proximal or early components of complement activation (e.g., C3 and C3b) essential for the opsonization of microorganisms and clearance of immune complexes.

In other embodiments, the antibody comprises the heavy and light chain CDRs or variable regions of BNJ441 (also known as ravulizumab, and Ultomiris®). Accordingly, in one embodiment, the antibody comprises the CDR1, CDR2, and CDR3 domains of the VH region of BNJ441 having the sequence set forth in SEQ ID NO: 12, and the CDR1, CDR2, and CDR3 domains of the VL region of BNJ441 having the sequence set forth in SEQ ID NO: 8. In another embodiment, the antibody comprises heavy chain CDR1, CDR2, and CDR3 domains having the sequences set forth in SEQ ID NOs: 19, 18, and 3, respectively, and light chain CDR1, CDR2, and CDR3 domains having the sequences set forth in SEQ ID NOs: 4, 5, and 6, respectively. In another embodiment, the antibody comprises VH and VL regions having the amino acid sequences set forth in SEQ ID NO: 12 and SEQ ID NO: 8, respectively. In another embodiment, the antibody may comprise the heavy chain constant region of BNJ441 having the amino acid sequence set forth in SEQ ID NO: 13.

In certain embodiments ravulizumab is administered once on Day 1 of the administration cycle, once on Day 15 of the administration cycle, and every eight weeks thereafter. In one embodiment, the anti-C5 antibody, or antigen binding fragment thereof, is administered every eight weeks after the administration cycle for an extension period up to two years (e.g., at a dose of 3000 mg, 3300 mg, or 3600 mg).

In another embodiment, ravulizumab is administered for one or more administration cycles. In one embodiment, the administration cycle is 26 weeks. In another embodiment, the treatment comprises at least 1, 2, 3, 4, 5, 6, 7, 8, 9, 10, or 11 cycles. In another embodiment, the treatment is continued for the lifetime of the human patient.

In another embodiment, a method of treating a human patient is provided, the method comprising administering to the patient during an administration cycle an effective amount of ravulizumab, or antigen binding fragment thereof, wherein ravulizumab is administered: once on Day 1 of the administration cycle at a dose of: 2400 mg to a patient weighing ≥40 to <60 kg, 2700 mg to a patient weighing ≥60 to <100 kg, or 3000 mg to a patient weighing ≥100 kg; and on Day 15 of the administration cycle and every eight weeks thereafter at a dose of 3000 mg to a patient weighing ≥40 to <60 kg, 3300 mg to a patient weighing ≥60 to <100 kg, or 3600 mg to a patient weighing ≥100 kg.

In certain embodiments, the dose of anti-C5 antibodies, or antigen binding fragment thereof, is based on the weight of the patient. For example, in one embodiment, about 2400 mg, about 2700 mg, about 3000 mg, about 3300 mg, and/or about 3600 mg of ravulizumab, or antigen binding fragment thereof, is administered to a patient based on their weight. In one embodiment, 2400 mg or 3000 mg of ravulizumab, or antigen binding fragment thereof, is administered to a patient weighing ≥40 to <60 kg. In another embodiment, 2700 mg or 3300 mg of ravulizumab, or antigen binding fragment thereof, is administered to a patient weighing 2 60 to <100 kg. In another embodiment, 3000 mg or 3600 mg of ravulizumab, or antigen binding fragment thereof, is administered to a patient weighing ≥100 kg. In certain embodiments, dosage regimens are adjusted to provide the optimum desired response (e.g., an effective response).

Another exemplary anti-C5 antibody is the 7086 antibody described in U.S. Pat. Nos. 8,241,628 and 8,883,158. In one embodiment, the antibody may comprise the heavy and light chain CDRs or variable regions of the 7086 antibody. See U.S. Pat. Nos. 8,241,628 and 8,883,158. In another embodiment, the antibody, or a fragment thereof, may comprise heavy chain CDR1, CDR2, and CDR3 domains having the sequences set forth in SEQ ID NOs: 21, 22, and 23, respectively, and light chain CDR1, CDR2, and CDR3 domains having the sequences set forth in SEQ ID NOs: 24, 25, and 26, respectively. In another embodiment, the antibody or fragment thereof may comprise the VH region of the 7086 antibody having the sequence set forth in SEQ ID NO: 27, and the VL region of the 7086 antibody having the sequence set forth in SEQ ID NO: 28.

Another exemplary anti-C5 antibody is the 8110 antibody also described in U.S. Pat. Nos. 8,241,628 and 8,883,158. In one embodiment, the antibody may comprise the heavy and light chain CDRs or variable regions of the 8110 antibody. The antibody, or fragment thereof may comprise heavy chain CDR1, CDR2, and CDR3 domains having the sequences set forth in SEQ ID NOs: 29, 30, and 31, respectively, and light chain CDR1, CDR2, and CDR3 domains having the sequences set forth in SEQ ID NOs: 32, 33, and 34, respectively. In another embodiment, the antibody may comprise the VH region of the 8110 antibody having the sequence set forth in SEQ ID NO: 35, and the VL region of the 8110 antibody having the sequence set forth in SEQ ID NO: 36.

In another embodiment, an exemplary anti C5 antibody is the 305LO5 antibody described in US2016/0176954A1. The anti-C5 antibody can comprise, for example, the heavy and light chain CDRs or variable regions of the 305LO5 antibody. The anti C5 antibody can comprise, for example, heavy chain CDR1, CDR2 and CDR3 domains having the sequences set forth in SEQ ID NOs: 37, 38 and 39, respectively, and light chain CDR1, CDR2 and CDR3 domains having the sequences set forth in SEQ ID NOs: 40, 41 and 42, respectively. In another embodiment, the antibody comprises the VH region of the 305LO5 antibody having the sequence set forth in SEQ ID NO: 43, and the VL region of the 305LO5 antibody having the sequence set forth in SEQ ID NO: 44.

Another exemplary anti-C5 antibody is the SKY59 antibody (Fukuzawa, T. et al., Sci. Rep., 7:1080, 2017). The anti-C5 antibody can comprise, for example, the heavy and light chain CDRs or variable regions of the SKY59 antibody. The anti-C5 antibody can comprise, for example, a heavy chain comprising SEQ ID NO:45 and a light chain comprising SEQ ID NO:46.

Another exemplary anti-C5 antibody is the REGN3918 antibody (also known as H4H12166PP) described in US20170355757. The anti-C5 antibody can comprise, for example, a heavy chain variable region comprising SEQ ID NO:47 and a light chain variable region comprising SEQ ID NO:48, or a heavy chain comprising SEQ ID NO:49 and a light chain comprising SEQ ID NO:50.

Another exemplary anti-C5 antibody comprises a heavy chain variable region amino acid sequence according to SEQ ID NO: 51 and a light chain variable region amino acid sequence according to SEQ ID NO: 52.

In another embodiment, the antibody competes for binding with, and/or binds to the same epitope on C5 as, the above-mentioned antibodies (e.g., eculizumab, ravulizumab, 7086 antibody, 8110 antibody, 305LO5 antibody, SKY59 antibody, or REGN3918 antibody). The anti-C5 antibody can have, for example, at least about 90% variable region amino acid sequence identity with the above-mentioned antibodies (e.g., at least about 90%, 91%, 92%, 93%, 94%, 95%, 96%, 97%, 98% or 99% variable region identity).

An anti-C5 antibody described herein can, in some embodiments, comprise a variant human Fc constant region that binds to human neonatal Fc receptor (FcRn) with greater affinity than that of the native human Fc constant region from which the variant human Fc constant region was derived. The Fc constant region can comprise, for example, one or more (e.g., two, three, four, five, six, seven, or eight or more) amino acid substitutions relative to the native human Fc constant region from which the variant human Fc constant region was derived. The substitutions, for example, can increase the binding affinity of an IgG antibody containing the variant Fc constant region to FcRn at pH 6.0, while maintaining the pH dependence of the interaction. Methods for testing whether one or more substitutions in the Fc constant region of an antibody increase the affinity of the Fc constant region for FcRn at pH 6.0 are known in the art and exemplified in the working examples (PCT/US2015/019225 and U.S. Pat. No. 9,079,949 the disclosures of each of which are incorporated herein by reference in their entirety).

Substitutions that enhance the binding affinity of an antibody Fc constant region for FcRn are known in the art and include, e.g., (1) the M252Y/S254T/T256E triple substitution (Dall'Acqua, W. et al., J. Biol. Chem., 281: 23514 24, 2006); (2) the M428L or T250Q/M428L substitutions (Hinton, P. et al., J. Biol. Chem., 279:6213 6, 2004; Hinton, P. et al., J. Immunol., 176:346 56, 2006); and (3) the N434A or T307/E380A/N434A substitutions (Petkova, S. et al., Int. Immunol., 18:1759 69, 2006). Additional substitution pairings, e.g., P257I/Q311I, P257I/N434H, and D376V/N434H, have also been described (Datta-Mannan, A. et al., J. Biol. Chem., 282:1709 17, 2007). The entire teachings of each of the cited references are hereby incorporated by reference.

In some embodiments, the variant constant region has a substitution at EU amino acid residue 255 for valine. In some embodiments, the variant constant region has a substitution at EU amino acid residue 309 for asparagine. In some embodiments, the variant constant region has a substitution at EU amino acid residue 312 for isoleucine. In some embodiments, the variant constant region has a substitution at EU amino acid residue 386.

In some embodiments, the variant Fc constant region comprises no more than 30 (e.g., no more than 29, 28, 27, 26, 25, 24, 23, 22, 21, 20, 19, 18, 17, 16, 15, 14, 13, 12, 11, 10, nine, eight, seven, six, five, four, three or two) amino acid substitutions, insertions or deletions relative to the native constant region from which it was derived. In some embodiments, the variant Fc constant region comprises one or more amino acid substitutions selected from the group consisting of: M252Y, S254T, T256E, N434S, M428L, V259I, T250I and V308F. In some embodiments, the variant human Fc constant region comprises a methionine at position 428 and an asparagine at position 434, each in EU numbering. In some embodiments, the variant Fc constant region comprises a 428L/434S double substitution as described in, e.g., U.S. Pat. No. 8,088,376 the disclosure of which is incorporated herein by reference in its entirety.

In some embodiments the precise location of these mutations may be shifted from the native human Fc constant region position due to antibody engineering. The 428L/434S double substitution when used in a IgG2/4 chimeric Fc, for example, may correspond to 429L and 435S as in the M429L and N435S variants found in ravulizumab and described in U.S. Pat. No. 9,079,949 the disclosure of which is incorporated herein by reference in its entirety.

In some embodiments, the variant constant region comprises a substitution at amino acid position 237, 238, 239, 248, 250, 252, 254, 255, 256, 257, 258, 265, 270, 286, 289, 297, 298, 303, 305, 307, 308, 309, 311, 312, 314, 315, 317, 325, 332, 334, 360, 376, 380, 382, 384, 385, 386, 387, 389, 424, 428, 433, 434 or 436 (EU numbering) relative to the native human Fc constant region. In some embodiments, the substitution is selected from the group consisting of: methionine for glycine at position 237; alanine for proline at position 238; lysine for serine at position 239; isoleucine for lysine at position 248; alanine, phenylalanine, isoleucine, methionine, glutamine, serine, valine, tryptophan or tyrosine for threonine at position 250; phenylalanine, tryptophan or tyrosine for methionine at position 252; threonine for serine at position 254; glutamic acid for arginine at position 255; aspartic acid, glutamic acid or glutamine for threonine at position 256; alanine, glycine, isoleucine, leucine, methionine, asparagine, serine, threonine or valine for proline at position 257; histidine for glutamic acid at position 258; alanine for aspartic acid at position 265; phenylalanine for aspartic acid at position 270; alanine or glutamic acid for asparagine at position 286; histidine for threonine at position 289; alanine for asparagine at position 297; glycine for serine at position 298; alanine for valine at position 303; alanine for valine at position 305; alanine, aspartic acid, phenylalanine, glycine, histidine, isoleucine, lysine, leucine, methionine, asparagine, proline, glutamine, arginine, serine, valine, tryptophan or tyrosine for threonine at position 307; alanine, phenylalanine, isoleucine, leucine, methionine, proline, glutamine or threonine for valine at position 308; alanine, aspartic acid, glutamic acid, proline or arginine for leucine or valine at position 309; alanine, histidine or isoleucine for glutamine at position 311; alanine or histidine for aspartic acid at position 312; lysine or arginine for leucine at position 314, alanine or histidine for asparagine at position 315; alanine for lysine at position 317; glycine for asparagine at position 325; valine for isoleucine at position 332; leucine for lysine at position 334: histidine for lysine at position 360; alanine for aspartic acid at position 376; alanine for glutamic acid at position 380; alanine for glutamic acid at position 382; alanine for asparagine or serine at position 384; aspartic acid or histidine for glycine at position 385; proline for glutamine at position 386; glutamic acid for proline at position 387: alanine or serine for asparagine at position 389; alanine for serine at position 424; alanine, aspartic acid, phenylalanine, glycine, histidine, isoleucine, lysine, leucine, asparagine, proline, glutamine, serine, threonine, valine, tryptophan or tyrosine for methionine at position 428; lysine for histidine at position 433; alanine, phenylalanine, histidine, serine, tryptophan or tyrosine for asparagine at position 434; and histidine for tyrosine or phenylalanine at position 436, all in EU numbering.

In one embodiment, the antibody binds to C5 at pH 7.4 and 25° C. (and, otherwise, under physiologic conditions) with an affinity dissociation constant (KD) that is at least 0.1 (e.g., at least 0.15, 0.175, 0.2, 0.25, 0.275, 0.3, 0.325, 0.35, 0.375, 0.4, 0.425, 0.45, 0.475, 0.5, 0.525, 0.55, 0.575, 0.6, 0.625, 0.65, 0.675, 0.7, 0.725, 0.75, 0.775, 0.8, 0.825, 0.85, 0.875, 0.9, 0.925, 0.95 or 0.975) nM. In some embodiments, the KD of the anti-C5 antibody, or antigen binding fragment thereof, is no greater than 1 (e.g., no greater than 0.9, 0.8, 0.7, 0.6, 0.5, 0.4, 0.3 or 0.2) nM.

In other embodiments, the [(KD of the antibody for C5 at pH 6.0 at 25° C.)/(KD of the antibody for C5 at pH 7.4 at 25° C.)] is greater than 21 (e.g., greater than 22, 23, 24, 25, 26, 27, 28, 29, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 350, 400, 450, 500, 600, 700, 800, 900, 1000, 1500, 2000, 2500, 3000, 3500, 4000, 4500, 5000, 5500, 6000, 6500, 7000, 7500 or 8000).

In various embodiments, eculizumab, an eculizumab variant such as BNJ441 (ravulizumab), or other anti-C5 antibody is administered to the subject once a month, once every two months, or once every three months depending on the dose. In another embodiment, the eculizumab, eculizumab variant such as BNJ441, or other anti-C5 antibody is administered once every two weeks, once a week, twice a week, or three times a week. In other embodiments, eculizumab, eculizumab variant such as BNJ441, or other anti-C5 antibody is administered once a week, once every two weeks, once every three weeks, once every four weeks, once every five weeks, once every six weeks, or once every eight weeks depending on the needs of the patient. In certain embodiments, eculizumab, eculizumab variant such as BNJ441, or other anti-C5 antibody in administered intravenously (IV) or subcutaneously (SubQ).

In other embodiments, a patient switches from receiving one C5 inhibitor to a different C5 inhibitor during the course of treatment. Different anti-C5 antibodies may be administered during separate treatment periods. For example, in one embodiment, a method of treating a human patient having a complement-associated disorder (e.g., generalized myasthenia gravis (gMG)) who is being treated with eculizumab is provided, the method comprising discontinuing treatment with eculizumab and switching the patient to treatment with an alternative complement inhibitor. For example, in one embodiment, the patient is treated with eculizumab during a treatment period (e.g., for 26 weeks), followed by treatment with another anti-C5 antibody (e.g., ravulizumab) during an extension period. In one embodiment, eculizumab is administered to the patient at a dose of 900 mg on Days 1, 8, 15, and 22 of the administration cycle during an induction phase, followed by a maintenance dose of 1200 mg of eculizumab on Day 19 of the administration cycle and every two weeks thereafter (e.g., for a total of 26 weeks), followed by treatment with ravulizumab for an extension period of up to two years.

Also, provided herein are pharmaceutical compositions comprising an anti-C5 antibody or antigen binding fragment thereof with a pharmaceutically acceptable excipient for treating MG. In one embodiment, the composition comprises an antibody comprising the CDR1, CDR2, and CDR3 domains of the VH region of eculizumab having the sequence set forth in SEQ ID NO: 7, and the CDR1, CDR2, and CDR3 domains of the VL region of eculizumab having the sequence set forth in SEQ ID NO: 8. In another embodiment, the antibody comprises heavy chain CDR1, CDR2, and CDR3 domains having the sequences set forth in SEQ ID NOs: 1, 2, and 3, respectively, and light chain CDR1, CDR2, and CDR3 domains having the sequences set forth in SEQ ID NOs: 4, 5, and 6, respectively. In another embodiment, the antibody comprises VH and VL regions having the amino acid sequences set forth in SEQ ID NO: 7 and SEQ ID NO: 8, respectively.

In some embodiments, the antibody comprises the heavy and light chain CDRs or variable regions of BNJ441. In one embodiment, the antibody comprises the CDR1, CDR2, and CDR3 domains of the VH region of BNJ441 having the sequence set forth in SEQ ID NO: 12, and the CDR1, CDR2, and CDR3 domains of the VL region of BNJ441 having the sequence set forth in SEQ ID NO: 8. In another embodiment, the antibody comprises heavy chain CDR1, CDR2, and CDR3 domains having the sequences set forth in SEQ ID NOs: 19, 18, and 3, respectively, and light chain CDR1, CDR2, and CDR3 domains having the sequences set forth in SEQ ID NOs: 4, 5, and 6, respectively. In another embodiment, the antibody comprises VH and VL regions having the amino acid sequences set forth in SEQ ID NO: 12 and SEQ ID NO: 8, respectively.

In some embodiments, the antibody comprises the heavy and light chain CDRs or variable regions of BNJ421. In one embodiment, the antibody comprises the CDR1, CDR2, and CDR3 domains of the VH region of BNJ421 having the sequence set forth in SEQ ID NO: 12, and the CDR1, CDR2, and CDR3 domains of the VL region of BNJ421 having the sequence set forth in SEQ ID NO: 8. In another embodiment, the antibody comprises heavy chain CDR1, CDR2, and CDR3 domains having the sequences set forth in SEQ ID NOs: 19, 18, and 3, respectively, and light chain CDR1, CDR2, and CDR3 domains having the sequences set forth in SEQ ID NOs: 4, 5, and 6, respectively. In another embodiment, the antibody comprises VH and VL regions having the amino acid sequences set forth in SEQ ID NO: 12 and SEQ ID NO: 8, respectively.

### 1. Methods of Treating Myasthenia Gravis

The disclosure provides methods of treating subjects suffering from myasthenia gravis (MG) by administering an antibody that specifically binds C5. In other embodiments, the subject is a mammalian subject.

As used herein, the term “subject” and “patient” are interchangeable. In certain embodiments, subjects and/or patients are mammals. According to certain embodiments, primates include humans. Thus, in certain embodiments, the subjects or patients suffering from MG described herein are humans.

As used herein, the term “bulbar signs” or “bulbar symptoms” refers to symptoms associated with bulbar weakness. These bulbar signs can include, but are not limited to, symptoms such as difficulty with speech (dysarthria), difficulty chewing, difficulty swallowing (dysphagia), and control of the head. In some embodiments, a bulbar sign is the onset of dysarthria. In some embodiments, the bulbar sign is difficulty swallowing. In some embodiments, the bulbar sign is difficulty chewing. In some embodiments, the bulbar sign is the onset of dysarthria, difficulty chewing, difficulty swallowing, and/or any combination thereof.

In certain embodiments, MG includes refractory generalized myasthenia gravis. In some embodiments, refractory generalized myasthenia gravis is characterized as including subjects or patients positive for auto-antibodies binding to nicotinic acetylcholine receptor (anti-AChR) who continue to show marked generalized weakness or bulbar signs and symptoms of myasthenia gravis while receiving current standard of care for myasthenia gravis such as cholinesterase inhibitor therapy and immunosuppressant therapy (IST) or who require chronic plasma exchange or chronic IVIg to maintain clinical stability. In other embodiments, refractory generalized myasthenia gravis is characterized as including subjects or patients who continue to show marked generalized weakness or bulbar signs and symptoms of myasthenia gravis while receiving current standard of care for myasthenia gravis such as cholinesterase inhibitor therapy and immunosuppressant therapy (IST) or who require chronic plasma exchange or chronic IVIg to maintain clinical stability.

In other embodiments, MG includes refractory generalized myasthenia gravis. In some embodiments, refractory generalized myasthenia gravis is characterized as including subjects or patients positive for auto-antibodies binding to nicotinic acetylcholine receptor (anti-AChR) who continue to show marked generalized weakness or bulbar signs and symptoms of myasthenia gravis while receiving cholinesterase inhibitor therapy and immunosuppressant therapy (IST) and who require chronic plasma exchange or chronic IVIg to maintain clinical stability. In other embodiments, refractory generalized myasthenia gravis is characterized as including subjects or patients who continue to show marked generalized weakness or bulbar signs and symptoms of myasthenia gravis while receiving cholinesterase inhibitor therapy and immunosuppressant therapy (IST) and who require chronic plasma exchange or chronic IVIg to maintain clinical stability.

As used herein, the phrase “requires chronic plasma exchange” to maintain clinical stability refers to the use of plasma exchange therapy on a patient on a regular basis for the management of muscle weakness at least every 3 months over the last 12 months.

As used herein, the phrase “requires chronic IVIg” to maintain clinical stability refers to the use of IVIg therapy on a patient on a regular basis for the management of muscle weakness at least every 3 months over the last 12 months.

In certain embodiments, treatment of MG includes the amelioration or improvement of one or more symptoms associated with MG. Symptoms associated with MG include muscle weakness and fatigability. Muscles primarily affected by MG include muscles that control eye and eyelid movement, facial expressions, chewing, talking, swallowing, breathing, neck movements, and limb movements.

In other embodiments, treatment of MG includes the improvement of a clinical marker for MG progression. These markers include MG activity of daily living profile (MG-ADL), quantitative Myasthenia Gravis (QMG) score for disease severity, Myasthenia Gravis composite (MGC), negative inspiratory force (NIF), forced vital capacity, MGFA post-intervention status, and other quality of life measurements. In certain embodiments, MG-ADL is the primary score for measuring improvement of MG.

The MG-ADL is an 8-point questionnaire that focuses on relevant symptoms and functional performance of activities of daily living (ADL) in MG subjects (see Table 1). The 8 items of the MG-ADL were derived from symptom-based components of the original 13-item QMG to assess disability secondary to ocular (2 items), bulbar (3 items), respiratory (1 item), and gross motor or limb (2 items) impairment related to effects from MG. In this functional status instrument, each response is graded 0 (normal) to 3 (most severe). The range of total MG-ADL score is 0-24. A clinically meaningful improvement in a patient's MG-ADL would be a 3 point or greater reduction in score after 26 weeks of treatment.

The current QMG scoring system consists of 13 items: ocular (2 items), facial (1 item), bulbar (2 items), gross motor (6 items), axial (1 item), and respiratory (1 item), each graded 0 to 3, with 3 being the most severe (see Table 2). The range of total QMG score is 0-39. The QMG scoring system is considered to be an objective evaluation of therapy for MG and is based on quantitative testing of sentinel muscle groups. The MGFA task force has recommended that the QMG score be used in prospective studies of therapy for MG. A clinically meaningful improvement in a patient's QMG would be a 5 point or greater reduction in score after 26 weeks of treatment.

TABLE 1
MG ACTIVITY OF DAILY-LIVING (MG-ADL) PROFILE

| | | | | | Score (0, |
|---|---|---|---|---|---|
| Items | Grade 0 | Grade 1 | Grade 2 | Grade 3 | 1, 2, 3) |
| 1. Talking | Normal | Intermittent | Constant | Difficult to | |
| | | slurring or | slurring | understand | |
| | | nasal | or nasal, | speech | |
| | | speech | but can be | | |
| | | | understood | | |
| 2. Chewing | Normal | Fatigue | Fatigue | Gastric | |
| | | with | with | Tube | |
| | | solid food | soft food | | |
| 3. Swallowing | Normal | Rare | Frequent | Gastric | |
| | | episode | choking | Tube | |
| | | of choking | neces- | | |
| | | | sitating | | |
| | | | changes in | | |
| | | | diet | | |
| 4. Breathing | Normal | Shortness | Sliminess | Ventilator | |
| | | of breath | of breath | depen- | |
| | | with | at rest | dence | |
| | | exertion | | | |
| 5. Impairment | None | Extra | Rest | Cannot do | |
| of ability | | effort, | periods | one | |
| to brush | | but no rest | needed | of these | |
| teeth or | | periods | | functions | |
| comb hair | | needed | | | |
| 6. Impairment | None | Mild, | Moderate, | Severe, | |
| of ability | | sometimes | always | requires | |
| to arise from | | uses | uses arms | assistance | |
| a chair | | arms | | | |
| 7. Double | None | Occurs, | Daily, but | Constant | |
| vision | | but | not | | |
| | | not daily | constant | | |
| 8. Eyelid drop | None | Occurs, | Daily, but | Constant | |
| | | but | not | | |
| | | not daily | constant | | |

TABLE 2
QUANTITATIVE MG (QMG) SCORE FOR DISEASE SEVERITY
QUANTITATIVE MYASTHENIA GRAVIS TESTING FORM
Patient Name: ___________ Patient #: ___ __________Date: _______
MR#: ______ DOB: _____ Sex: ___ Ht. (in): ______ Wt. (kg) ____
Evaluator: ___ Handedness: ___ Leggedness: ___ Time of Exam: _____
Anticholinesterase Medication: ________________________________
Comments: ________________________________________________

| TEST | | | | | |
|---|---|---|---|---|---|
| ITEMS | | | | | |
| WEAK- | | | | | |
| NESS | NONE | MILD | MODERATE | SEVERE | SCORE |
| GRADE | 0 | 1 | 2 | 3 | |
| Double | 60 | 11-59 | 1-10 | Spontaneous | |
| vision | | | | | |
| (lateral | | | | | |
| gaze) Sec. | | | | | |
| Ptosis | 60 | 11-59 | 1-10 | Spontaneous | |
| (upward | | | | | |
| gaze) Sec. | | | | | |
| Facial | Normal | Complete, | Complete, | Incomplete | |
| Muscles | lid | weak, some | without: | | |
| | closure | resistance | resistance | | |
| Swallowing | Normal | Minimal | Severe | Cannot | |
| 4 oz. Water | | coughing | coughing | swallow | |
| (1/2 cup) | | or | Choking: | (test | |
| | | throat | or | not | |
| | | clearing | nasal | attempted) | |
| | | | regurgitation | | |
| Speech | None | Dysarthria | Dysarthria | Dysarthria | |
| following | at #50 | at | at | at | |
| counting | | #30-49 | # 10-29 | #9 | |
| aloud from | | | | | |
| 1-50 (onset | | | | | |
| of dysarthria) | | | | | |
| Right arm | 240 | 90-239 | 10-89 | 0-9 | |
| outstretched | | | | | |
| (90°, sitting) | | | | | |
| Sec. | | | | | |
| Left arm | 240 | 90-239 | 10-89 | 0-9 | |
| outstretched | | | | | |
| (90°, sitting) | | | | | |
| Sec. | | | | | |
| Forced vital | ≥80% | 65-79% | 50-64% | <50% | |
| capacity | | | | | |
| Rt hand grip: | ≥45 | 15-44 | 5-14 | 0-4 | |
| male | | | | | |
| (Kg) : female | ≥30 | 10-29 | 5-9 | 0-4 | |
| Left hand | ≥35 | 15-34 | 5-14 | 0-4 | |
| grip: male | | | | | |
| (Kg) : female | ≥25 | 10-24 | 5-9 | 0-4 | |
| Head, lifted | 120 | 30-119 | 1-29 | 0 | |
| (45%, | | | | | |
| supine) Sec. | | | | | |
| Right leg, | 100 | 31-99 | 1-30 | 0 | |
| outstretched | | | | | |
| (45-50%, | | | | | |
| supine) Sec. | | | | | |
| Left leg, | 100 | 31-99 | 1-30 | 0 | |
| outstretched | | | | | |
| (45-50%, | | | | | |
| supine) Sec. | | | | | |

TOTAL MG SCORE: __________

The MGC is a validated assessment tool for measuring clinical status of subjects with MG (16). The MGC assesses 10 important functional areas most frequently affected by MG and the scales are weighted for clinical significance that incorporates subject-reported outcomes. See Table 3. MGC is administered at Screening, Day 1, Weeks 1-4, 8, 12, 16, 20, and 26 or ET (Visits 1-6, 8, 10, 12, 14, and 17 or ET). A clinically meaningful improvement in a patient's MGC would be a 3 point or greater reduction in score after 26 weeks of treatment.

TABLE 3
MG COMPOSITE SCALE

| | | | | | | | | |
|---|---|---|---|---|---|---|---|---|
| Ptosis, upward gaze (PE) | >45 seconds | 0 | 11-45 seconds | 1 | 1-10 seconds | 2 | Immediate | [symbol] |
| Double vision on lateral | >45 seconds | 0 | 11-45 seconds | 1 | 1-10 seconds | 2 | Immediate | [symbol] |
| gaze, left or right (PE) | | | | | | | | |
| Eye closure (PE) | Normal | 0 | Mild weakness | 0 | Moderate | 1 | Severe | 2 |
| | | | (can be forced | | weakness (can | | weakness | |
| | | | open with effort) | | be forced open | | (unable to | |
| | | | | | easily) | | keep eyes | |
| | | | | | | | closed) | |
| Talking (Pt) | Normal | 0 | Intermittent | 2 | Constant slurring | 4 | Difficult | [symbol] |
| | | | slurring or nasal | | or nasal but can | | to understand | |
| | | | speech | | be understood | | | |
| Chewing (Pt) | Normal | 0 | Fatigue with | 2 | Fatigue with | 4 | Gastric tube | [symbol] |
| | | | solid food | | soft food | | | |
| Swallowing (Pt) | Normal | 0 | Rare trouble or | 2 | Frequent trouble | 5 | Gastric tube | [symbol] |
| | | | choking | | (change in diet) | | | |
| Breathing | Normal | 0 | SOB with exertion | 2 | SOB at rest | 4 | Ventilator | [symbol] |
| Neck Flex/Ext | Normal | 0 | Mild | 1 | Moderate | 3 | Severe | 4 |
| (weakest PE) | | | | | (~50% weak +/− | | | |
| | | | | | 15%) | | | |
| Shoulder Abd (PE | Normal | 0 | Mild | 2 | Moderate | 4 | Severe | 5 |
| | | | | | (~50% weak +/− | | | |
| | | | | | 15%) | | | |
| Hip flexion | Normal | 0 | Mild | 2 | Moderate | 4 | Severe | 5 |
| | | | | | (~50% weak +/− | | | |
| | | | | | 15%) | | | |
| | | 0 | | [symbol] | | [symbol] | | [symbol] |

[symbol]

indicates data missing or illegible when filed

The 15-item Myasthenia Gravis Qualify of Life 15 scale (MG-QOL 15) is a health-related quality of life evaluative instrument specific to subjects with MG. See Table 4. MG-QOL15 was designed to provide information about subjects' perception of impairment and disability and the degree to which disease manifestations are tolerated and to be easy to administer and interpret. The range of total scores is from 0 to 60. Higher scores translate into a greater extent of a patient's dissatisfaction with MG related dysfunction. The MG-QOL 15 is completed by the subject. Higher scores indicate greater extent of and dissatisfaction with MG-related dysfunction. A clinically meaningful improvement in a patient's MG-QOL 15 would be a decrease in score after 26 weeks of treatment.

TABLE 4
MYASTHENIA GRAVIS QUALIFY OF LIFE
15 SCALE (MG-QOL, 15)

| Statement: How true in | Not | A little | | Quite | Very |
|---|---|---|---|---|---|
| past 4 weeks? | at all | bit | Somewhat | a bit | Much |
| Frustrated by condition | 0 | 1 | 2 | 3 | 4 |
| Trouble using my eyes | 0 | 1 | 2 | 3 | 4 |
| Trouble eating | 0 | 1 | 2 | 3 | 4 |
| Condition limits social life | 0 | 1 | 2 | 3 | 4 |
| Condition limits hobbies/fun | 0 | 1 | 2 | 3 | 4 |
| Trouble meeting family's needs | 0 | 1 | 2 | 3 | 4 |
| Need to plan around condition | 0 | 1 | 2 | 3 | 4 |
| Occupational skills/job | 0 | 1 | 2 | 3 | 4 |
| negatively affected | | | | | |
| Difficulty speaking | 0 | 1 | 2 | 3 | 4 |
| Trouble driving | 0 | 1 | 2 | 3 | 4 |
| Depressed about condition | 0 | 1 | 2 | 3 | 4 |
| Trouble walking | 0 | 1 | 2 | 3 | 4 |
| Trouble getting around in | 0 | 1 | 2 | 3 | 4 |
| public places | | | | | |
| Feel overwhelmed by condition | 0 | 1 | 2 | 3 | 4 |
| Trouble performing personal | 0 | 1 | 2 | 3 | 4 |
| grooming | | | | | |

The Neuro-QOL Fatigue is a reliable and validated brief 19-item survey of fatigue completed by the subject. Higher scores indicate greater fatigue and greater impact of MG on activities (see Table 5). A clinically meaningful improvement in a patient's Neuro-QOL Fatigue score would be reflected in a decrease in score after 26 weeks of treatment.

TABLE 5
NEURO-QOL FATIQUE
Please respond to each question or statement by marking one box per row.

| | In the past 7 days . . . | Never | Rarely | Sometimes | Often | Always |
|---|---|---|---|---|---|---|
| NQFTG13 | I felt exhausted | □ | □ | □ | □ | □ |
| | | 1 | 2 | 3 | 4 | 5 |
| NQFTG11 | I felt that I had no energy | □ | □ | □ | □ | □ |
| | | 1 | 2 | 3 | 4 | 5 |
| NQFTG15 | I felt fatigued | □ | □ | □ | □ | □ |
| | | 1 | 2 | 3 | 4 | 5 |
| NQFTG06 | I was too tired to do my | □ | □ | □ | □ | □ |
| | household chores | 1 | 2 | 3 | 4 | 5 |
| NQFTG07 | I was too tired to leave | □ | □ | □ | □ | □ |
| | the house | 1 | 2 | 3 | 4 | 5 |
| NQFTG10 | I was frustrated by being | □ | □ | □ | □ | □ |
| | too tired to do the things | 1 | 2 | 3 | 4 | 5 |
| | I wanted to do | | | | | |
| NQFTG14 | I felt tired | □ | □ | □ | □ | □ |
| | | 1 | 2 | 3 | 4 | 5 |
| NQFTG02 | I had to limit my social | □ | □ | □ | □ | □ |
| | activity because I was | 1 | 2 | 3 | 4 | 5 |
| | tired | | | | | |
| NQFTG01 | I need help doing my | □ | □ | □ | □ | □ |
| | usual activites because | 1 | 2 | 3 | 4 | 5 |
| | of my fatigue | | | | | |
| NQFTG03 | I needed to sleep during | □ | □ | □ | □ | □ |
| | the day | 1 | 2 | 3 | 4 | 5 |
| NQFTG04 | I had trouble starting | □ | □ | □ | □ | □ |
| | things because I was | 1 | 2 | 3 | 4 | 5 |
| | too tired | | | | | |
| NQFTG05 | I had trouble finishing | □ | □ | □ | □ | □ |
| | things because I was | 1 | 2 | 3 | 4 | 5 |
| | too tired | | | | | |
| NQFTG08 | I was too tired to take a | □ | □ | □ | □ | □ |
| | short walk | 1 | 2 | 3 | 4 | 5 |
| NQFTG09 | I was too tired to eat | □ | □ | □ | □ | □ |
| | | 1 | 2 | 3 | 4 | 5 |
| NQFTG12 | I was so tired that I | □ | □ | □ | □ | □ |
| | needed to rest during | 1 | 2 | 3 | 4 | 5 |
| | the day | | | | | |
| NQFTG16 | I felt weak all over | □ | □ | □ | □ | □ |
| | | 1 | 2 | 3 | 4 | 5 |
| NQFTG17 | I needed help doing my | □ | □ | □ | □ | □ |
| | usual activites because | 1 | 2 | 3 | 4 | 5 |
| | of weakness | | | | | |
| NQFTG18 | I had to limit my social | □ | □ | □ | □ | □ |
| | acitivity because I was | 1 | 2 | 3 | 4 | 5 |
| | physically weak | | | | | |
| NQFTG20 | I had to force myself to | □ | □ | □ | □ | □ |
| | get up and do things | 1 | 2 | 3 | 4 | 5 |
| | because I was physically | | | | | |
| | too weak | | | | | |

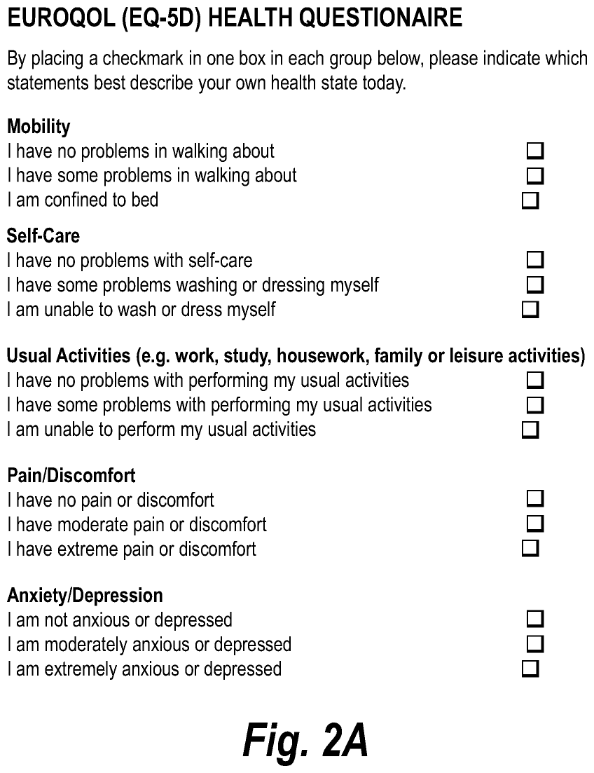

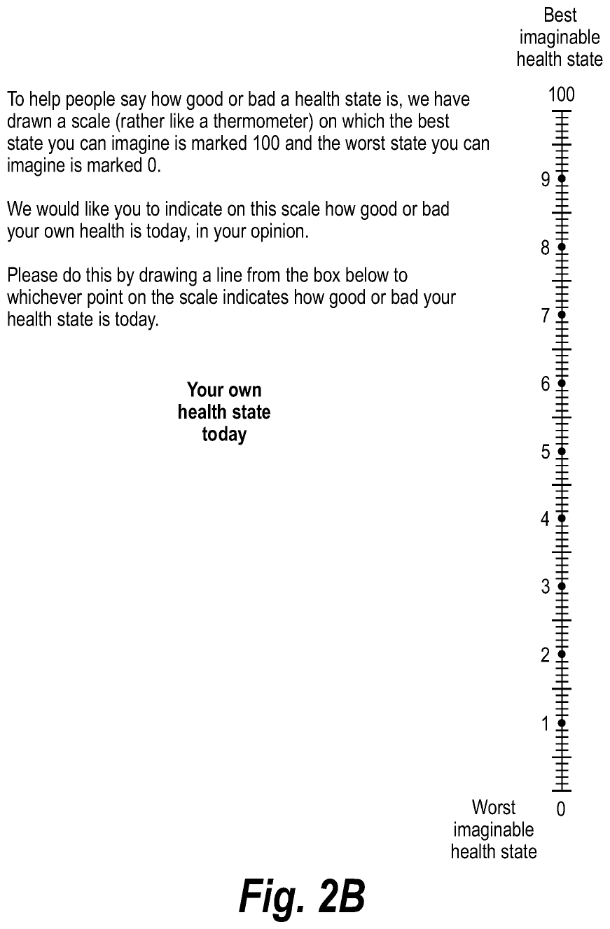

The EUROQOL (EQ-5D) is a reliable and validated survey of health status in 5 areas: mobility, self-care, usual activities, pain/discomfort, and anxiety/depression, completed by the subject. Each area has 3 levels: level 1 (no problems), level 2 (some problems), and level 3 (extreme problems) (see FIGS. 2A and 2B). The EQ VAS records the subject's self-rated health on a vertical, 20 cm visual analogue scale where the endpoints are labeled “Best imaginable health state, marked as 100” and “Worst imaginable health state, marked as 0.” The EQ-5D is administered at Day 1, Weeks 4, 8, 12, 16, 20, and 26 or ET (Visits 2, 6, 8, 10, 12, 14, and 17 or ET). A clinically meaningful improvement in a patient's EQ-5D would be reflected as an increase in score after 26 weeks of treatment.

Subjects with increasingly severe MG can suffer from potentially fatal respiratory complications including profound respiratory muscle weakness. Respiratory function is monitored closely for evidence of respiratory failure in MG subjects and ventilator support is recommended in the event of consistent declines in serial measurements of Forced Vital Capacity (FVC) or Negative Inspiratory Force (NIF), loss of upper airway integrity (difficulty handling oral secretions, swallowing, or speaking) or in the setting of emerging respiratory failure. FVC as one of the test items in QMG is performed when QMG is performed. NIF was performed using the NIF Meter.

The MG clinical state is assessed using the MGFA Post-Intervention Status (see Table 6). Change in status categories of Improved, Unchanged, Worse, Exacerbation and Died of MG as well as the Minimal Manifestation (MM) can be assessed. Patients are considered to have achieved MM if they have no symptoms indicating functional limitations.

TABLE 6
MGFA POST-INTERVENTION STATUS

| | |
|---|---|
| Complete stable remission | The patient has had no symptoms or |
| (CSR) | signs of MG for at least 1 year and has |
| | received no therapy for MG during that |
| | time. There is no weakness of any |
| | muscle on careful examination by |
| | someone skilled in the evaluation of neuro- |
| | muscular disease. Isolated weakness |
| | of eyelid closure is accepted. |
| Pharmacological remission | The same criteria as for CSR, except |
| (PR) | that the patient continues to take some |
| | form of therapy for MG. Patients taking |
| | cholinestarase inhibitors are excluded |
| | from this category beacuse their use |
| | suggests the presence of weakness. |
| Minimal manifestations | The patient has no symptoms of |
| (MM) | functional limitations from MG, but |
| | has some weakness on examination |
| | of some muscles. This class |
| | recognizes that some patients who |
| | otherwise meet the definition of |
| | CSR or PR do have weakness that is |
| | only detectable by careful examination. |
| MM-0 | The patient has received no MG |
| | treatment for at least 1 year. |
| MM-1 | The patient continues to receive some |
| | form of immunosuppression, but |
| | no cholinesterase inhibitors or other |
| | symptomatic therapy. |
| MM-2 | The patient has received only low-dose |
| | cholinesterase inhibitors (<120 mg |
| | pyridostigmine/day) for at least 1 year. |
| MM-3 | The patient has received cholinesterase |
| | inhibitors or other symptomatic |
| | therapy and some form of immuno- |
| | suppression during the past year. |
| Change in Status | |
| Improved (I) | A substantial decrease in pretreatment |
| | clinical manifestations or a sustained |
| | substantial reduction in MG medications |
| | as defined in the protocol, in prospective |
| | studies, this should be defined |
| | as a specific decrease in QMG score. |
| Unchanged (II) | No substantial change in pretreatment |
| | clinical manifestations or reduction in |
| | MG medications as defined in the |
| | protocol. In prospective studies, this |
| | should be defined in terms of a |
| | maximum change in QMG score. |
| Worse (W) | A substantial increase in pretreatment |
| | clinical manifestations or a substantial |
| | increase in MG medications as defined in |
| | the protocol. In prospective studies, |
| | this should be defined as a specific |
| | increase in QMG score. |
| Exacerbation (E) | Patients who have fulfilled criteria of |
| | CSR, PR, or MM, but subsequently |
| | developed clinical findings greater than |
| | permitted by these criteria. |
| Died of MG (D of MG) | Patients who died of MG, of complications |
| | of MG therapy, or within 30 days after |
| | thymectomy. List the cause (see |
| | Morbidity and Mortality data). |

In some embodiments, the patient is treated and achieves the status of Improved or MM according to the MGFA Post-Intervention Status. In some embodiments, the patient achieves a status of improved or MM after being treated for 26 or 52 weeks. In some embodiments, the patient achieves a status of improved or MM after being treated for 4, 12, 66, 78, 104, 130, 156, or more weeks. In some embodiments, the patient is administered eculizumab in any manner as described herein. In some embodiments, the patient maintains an Improved or MM status by continuing treatment with eculizumab for 4, 12, 26, 52, 66, 78, 104, 130, 156, or more weeks. In some embodiments, the treatment comprises administering eculizumab using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.

In some embodiments, the patient with MG or refractory MG achieves an Improved or MM score and seeks to maintain this score. In some embodiments, the patient is administered eculizumab in any manner as described herein. In some embodiments, the patient maintains an Improved or MM status by continuing treatment with eculizumab for 4, 12, 26, 52, 66, 78, 104, 130, 156, or more weeks. In some embodiments, the treatment comprises administering eculizumab using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter. In some embodiments, patients maintain an Improved status when they maintain a status of Improved or MM for a certain period.

According to certain embodiments, patients administered eculizumab show a reduced MG-ADL. In certain embodiments, the subjects have an initial MG-ADL score of greater than 6 points. In other embodiments, the subjects have an initial MG-ADL score greater than 0, 1, 2, 3, 4, 5, 6, 7, 8, 9, 10, 11, 12, 13, 14, 15, 16, 17, 18, 19, 20, 21, 22, or 23 points. In certain embodiments, after a course of treatment with eculizumab, the MG-ADL score of the subject has been reduced to less than 6 points. In other embodiments, the MG-ADL score has been reduced at least 1 point, at least 2 points, at least 3 points, at least 4 points, at least 5 points, at least 6 points, at least 7 points, at least 8 points, at least 9 points, at least 10 points, at least 11 points, at least 12 points, at least 13 points, at least 14 points, at least 15 points, at least 16 points, at least 17 points, at least 18 points, at least 19 points, at least 20 points, at least 21 points, at least 22 points, at least 23 points, or at least 24 points after treatment with eculizumab. In certain embodiments, the MG-ADL score of the patient is reduced by at least 1 point after a course of treatment with eculizumab. In other embodiments, the MG-ADL of the patient is reduced by 1, 2, 3, 4, 5, 6, 7, 8, 9, 10, 11, 12, 13, 14, 15, 16, 17, 18, 19, 20, 21, 22, 23, or 24 points after a course of treatment with eculizumab.

According to certain embodiments, the course of treatment with eculizumab lasts for 26 weeks. According to other embodiments, the course of treatment lasts for 26-52, 26-78, 26-104, 26-130, 26-156, 26-182, 26-208 weeks, or more. In other embodiments, the course of treatment lasts for greater than 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156, or 182 weeks. According to other embodiments, the course of treatment lasts for greater than 1, 2, 3, 4, 5, 10, 15, 20, 25, 30, 35, 40, 45, 50, 55, 60, 65, 70, 75, 80, or more years. In certain embodiments, the course of treatment lasts for the remainder of the subject's life.

According to certain embodiments, during the course of treatment, one or more symptoms or scores associated with MG improves during the course of treatment and is maintained at the improved level throughout treatment. For example, MG-ADL can improve after 26 weeks of treatment with a therapeutic antibody that specifically binds C5 and then remain at the improved level for the duration of the treatment, which is 52 weeks of treatment with a therapeutic antibody that specifically binds C5. One example of a therapeutic antibody that binds C5 is eculizumab.

In certain embodiments, the first sign of improvement occurs by 26 weeks of treatment with a therapeutic antibody that specifically binds C5. According to other embodiments, the first sign of improvement occurs between weeks 1-26, 26-52, 52-78, 78-104, 104-130, 130-156, 156-182, or 182-208 of treatment with a therapeutic antibody that specifically binds C5. In other embodiments, the first sign of improvement occurs at week 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156, or 182.

According to certain embodiments, the first sign of improvement is maintained for a number of weeks during treatment with a binding protein that specifically binds C5, such as eculizumab or an eculizumab variant such as BNJ441. According to certain embodiments, this number of weeks is at least 26. According to other embodiments, this number of weeks is 1-26, 26-52, 52-78, 78-104, 104-130, 130-156, 156-182, or 182-208. In other embodiments, this number of weeks is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156, or 182.

According to certain embodiments, eculizumab or other anti-C5 antibodies such as BNJ441, BNJ421, 7086, and 8110 can be administered to a subject suffering from MG at between 600 and 6000 mg. According to other embodiments, the induction dose of eculizumab or other anti-C5 antibodies such as BNJ441, BNJ421, 7086, and 8110 is between 900 and 1500 mg, 900 and 1200 mg, 900 mg, or 1200 mg. According to other embodiments, the maintenance dose of eculizumab or other anti-C5 antibodies such as BNJ441, BNJ 421, 7086, and 8110 is about 600, 700, 800, 900, 1000, 1100, 1200, 1300, 1400, 1500, 1600, 1700, 1800, 1900, 2000, 2500, 3000, 4000, 5000, or 6000 mg.

These doses can be administered once a month, once every two weeks, once a week, twice a week, or daily. According to certain embodiments, the dose is administered once every two weeks or once a week. According to other embodiments, eculizumab is administered to a subject suffering from MG in a multiphase dosing regimen. According to certain embodiments, the multiphase dosing regimen has 2, 3, 4, 6, 7, 8, 9, 10, or more phases. In certain embodiments, each phase provides a higher dose than the phase before it.

In certain embodiments, the eculizumab multiphase dosing regimen has two phases. The first phase is an induction phase. This phase provides a dose of 600, 900, 1200, 1500, or 1800 mg per week. In certain embodiments, this phase lasts for 2, 3, 4, 5, 6, 7, 8, 9, or 10 weeks. In other embodiments, this phase lasts between 2 and 6 weeks. In other embodiments, the phase lasts for 5 weeks. According to certain embodiments, the dose given any week is higher than the previous week. In other embodiments, the dose remains the same for a number of weeks and is then increased. In some embodiments, the dose remains the same for the first 1, 2, 3, 4, 5, 6, 7, 8, or 9 weeks and is then increased. In other embodiments, the dose remains the same for the first 4 weeks. According to some embodiments, the eculizumab dose is administered at between 600 and 1200 mg, 800 and 1500 mg, 900 and 1200 mg, 900 and 1100 mg, 900 and 1000 mg, 800 and 1000 mg, 800 and 1100 mg, or 800 and 1200 mg for a number of weeks and is then increased. In one embodiment, the eculizumab dose is administered at about 900 mg on day 1 and is followed by doses of 900 mg on day 7, 900 mg on day 14, 900 mg on day 21, and then is increased to 1200 mg for the fifth dose on day 28, and then 1200 mg is administered every 14±2 days thereafter.

In one particular embodiment, the eculizumab induction phase dosing regimen comprises five administered doses on the following schedule:

900 mg on day 1; 900 mg on day 7 (week 1), 900 mg on day 14 (week 2), 900 mg on day 21 (week 3), and 1200 mg on day 28 (week 4), and then 1200 mg is administered every 14 ±2 days thereafter. The actual days between each dose may vary during the induction by 1 or 2 days to accommodate unexpected events in the patients' schedule.

According to this embodiment, the second phase of eculizumab dosing is the maintenance phase. The maintenance phase of eculizumab dosing can last for between 6 weeks and the life of the subject. According to other embodiments, the maintenance phase lasts for 26-52, 26-78, 26-104, 26-130, 26-156, 26-182, 26-208 weeks, or more. In other embodiments, the maintenance phase lasts for greater than 26, 27, 28, 29, 30, 31, 32, 33, 34, 35, 36, 37, 38, 39, 40, 41, 42, 43, 44, 45, 46, 47, 48, 49, 50, 51, 52, 78, 104, 130, 156, or 182 weeks. According to other embodiments, the maintenance phase lasts for greater than 1, 2, 3, 4, 5, 10, 15, 20, 25, 30, 35, 40, 45, 50, 55, 60, 65, 70, 75, 80 years, or more years. In certain embodiments, the maintenance phase lasts for the remainder of the subject's life.

In certain embodiments, the eculizumab multiphase dosing regimen includes a third phase. This third phase is used when an MG patient must undergo a rescue procedure to maintain clinical stability and includes administering plasma exchange and/or dosing with IVIg. In this phase after plasma is exchanged a dose of eculizumab is administered to replace the drug lost in plasma exchange. According to certain embodiments, this post-rescue eculizumab dose is between 300 and 1200 mg, 400 and 1500 mg, 500 and 1000 mg, 400 and 800 mg, or 500 and 700 mg. According to certain embodiments, this post-rescue eculizumab dose is about 600 mg. In another embodiment, in this post-rescue or third phase a 600 mg eculizumab dose is administered within 1 hour after completion of plasmapheresis. In another embodiment, in the third phase a 600 mg dose is administered within 2 hours after completion of plasmapheresis. In another embodiment, in the third phase a 600 mg dose is administered within 3 hours after completion of plasmapheresis. In another embodiment, in the third phase a 600 mg dose is administered within 4 hours after completion of plasmapheresis. In another embodiment, in the third phase a 600 mg dose is administered within 5 hours after completion of plasmapheresis. In another embodiment, in the third phase a 600 mg dose is administered within 6 hours after completion of plasmapheresis.

In certain embodiments, the ravulizumab multiphase dosing regimen includes a third phase. This third phase is used when an MG patient must undergo a rescue procedure to maintain clinical stability and includes administering plasma exchange/plasmapheresis (PE/PP) and/or dosing with IVIg. In this phase after plasma is exchanged a dose of ravulizumab is administered to replace the drug lost in plasma exchange/plasmapheresis. According to certain embodiments, supplemental study drug (or placebo) dosing is required if PE/PP or IVIg rescue therapy is provided on nondosing days. In another embodiment, if PE/PP or IVIg infusion is provided on a dosing day, it must occur prior to study drug administration. According to certain embodiments, if PE/PP or IVIg is administered on nonscheduled dosing visits, patients receiving PE/PP will be administered a supplemental dose 4 hours after the PE/PP session is completed. In another embodiment, patients receiving IVIg will be administered a supplemental dose 4 hours after the last continuous session(s) of IVIg is completed. In certain embodiments, supplemental dose amounts may or may not vary depending on PE/PP or IVIg. In other embodiments, if PE/PP or IVIg is administered on scheduled dosing visits, regular dosing will be followed 60 minutes after the completion of PE/PP or IVIg. In certain embodiments, no gap is required between a supplemental dose and the regular scheduled dose.

In some embodiments, a loading dose of the anti-C5 antibody is administered intravenously at 1000 mg for patients between 40 kg and 100 kg (inclusive) or a loading dose of 1500 mg for patients >100 kg on week 1 day 1. In some embodiments, the anti-C5 antibody is administered on week 1, day 2, at a subcutaneous dose of 340 mg. In some embodiments, the anti-C5 antibody is administered on week 2, week 3, and week 4 at a subcutaneous dose of 340 mg. In some embodiments, the anti-C5 antibody is administered on week 5 at a subcutaneous dose of 680 mg for patients between 40 kg and 100 kg (inclusive) or a subcutaneous dose of 1020 mg for patients >100 kg, followed by a subcutaneous dose of 680 mg for patients between 40 kg and 100 kg (inclusive) or a subcutaneous dose of 1020 mg for patients >100 kg every 4 weeks. In some embodiments, the anti-c5 antibody is administered for 24 weeks. In some embodiments, the anti-C5 antibody used with this dosing regimen is crovalimab (SKY59/RG6107).

In one embodiment, the anti-C5 dosing regimen comprises doses on the following schedule:

administration intravenously at 1000 mg for patients between 40 kg and 100 kg (inclusive) or a loading dose of 1500 mg for patients >100 kg on day 1; administration subcutaneously at a dose of 340 mg on day 2 (week 1); administration subcutaneously at a dose of 340 mg on week 2, administration subcutaneously at a dose of 340 mg on week 3, administration subcutaneously at a dose of 340 mg on week 4, administration subcutaneously at a dose of 680 mg for patients between 40 kg and 100 kg (inclusive) or 1020 mg for patients >100 kg on week 5, and then administration subcutaneously at a dose of 680 mg for patients between 40 kg and 100 kg (inclusive) or 1020 mg for patients >100 kg every 4 weeks thereafter. The actual days between each dose may vary by 1 or 2 days to accommodate unexpected events in the patients' schedule.

In some embodiments, a loading dose of the anti-C5 antibody is administered intravenously at a dose of 30 mg/kg, followed by a once weekly dose of 800 mg administered subcutaneously. In some embodiments, the anti-C5 antibody used with this dosing regimen is pozelimab.

### 2. Pharmaceutical Compositions

Pharmaceutical compositions comprising eculizumab, either alone or in combination with prophylactic agents, therapeutic agents, and/or pharmaceutically acceptable carriers are provided. The pharmaceutical compositions comprising eculizumab provided herein are for use in, but not limited to, diagnosing, detecting, or monitoring a disorder, in preventing, treating, managing, or ameliorating a disorder or one or more symptoms thereof, and/or in research. The formulation of pharmaceutical compositions, either alone or in combination with prophylactic agents, therapeutic agents, and/or pharmaceutically acceptable carriers, is known to one skilled in the art.

An exemplary, non-limiting range for a therapeutically or prophylactically effective amount of eculizumab or other anti-C5 antibodies such as BNJ441 (ravulizumab), BNJ 421, 7086, and 8110 provided herein is 600-5000 mg, for example, 900-2000 mg. It is to be noted that dosage values may vary with the type and severity of the condition to be alleviated. It is to be further understood that for any particular subject, specific dosage regimens may be adjusted over time according to the individual need and the professional judgment of the person administering or supervising the administration of the compositions, and that dosage ranges set forth herein are exemplary only and are not intended to limit the scope or practice of the claimed methods.

Also provided are pharmaceutical compositions comprising other anti-C5 antibodies, either alone or in combination with prophylactic agents, therapeutic agents, and/or pharmaceutically acceptable carriers. In some embodiments, a non-limiting range for a therapeutically or prophylactically effective amount of crovalimab is 1000 mg for patients between 40 kg and 100 kg (inclusive) or 1500 mg for patients >100 kg. In some embodiments, a non-limiting range for a therapeutically or prophylactically effective amount of crovalimab is 340 mg. In some embodiments, a non-limiting range for a therapeutically or prophylactically effective amount of crovalimab is 680 mg for patients between 40 kg and 100 kg (inclusive) or 1020 mg for patients >100 kg. In some embodiments, a non-limiting range for a therapeutically or prophylactically effective amount of pozelimab is 30 mg/kg. In some embodiments, a non-limiting range for a therapeutically or prophylactically effective amount of pozelimab is 800 mg.

### 3. Combination Therapy

An anti-C5 antibody provided herein also can also be administered with one or more additional medicaments or therapeutic agents useful in the treatment of MG. For example, the additional agent can be a therapeutic agent art-recognized as being useful to treat myasthenia gravis or condition being treated by the antibody provided herein. The combination can also include more than one additional agent, e.g., two or three additional agents.

The binding agent in various embodiments is administered with an agent that is a protein, a peptide, a carbohydrate, a drug, a small molecule, or a genetic material (e.g., DNA or RNA). In various embodiments, the agent is one or more cholinesterase inhibitors, one or more corticosteroids, and/or one or more immunosuppressive drugs (most commonly azathioprine [AZA], cyclosporine, and/or mycophenolate mofetil [MMF]).

Without limiting the disclosure, a number of embodiments of the disclosure are described below for purpose of illustration.

### 4. Exemplary Sequences

Table 7 below contains sequences of anti-complement protein C5 specific humanized antibodies that can be used in treating refractory MG. The antibody was an anti-C5 antibody such as eculizumab having three heavy chain complementarity determining regions (CDRs) as set forth in Table 7 using the Kabat definitions of CDRs as heavy chain CDR1 in SEQ ID NO: 1, heavy chain CDR2 in SEQ ID NO: 2, and heavy chain CDR3 in SEQ ID NO: 3. The eculizumab light chain CDRs are set forth below as light chain CDR1 in SEQ ID NO: 4, light chain CDR2 in SEQ ID NO: 5 and light chain CDR3 in SEQ ID NO: 6. The heavy chain variable region of eculizumab is set forth in SEQ ID NO: 7 and the light chain variable region of eculizumab is set forth in SEQ ID NO: 8. The complete heavy chain of eculizumab is set forth below as SEQ ID NO: 10 and the light chain is set forth below as SEQ ID NO: 11

The antibody may be an eculizumab variant known as BNJ441, also known as ravulizumab, and having selected mutations in the CDR regions combined with mutations in the Fc region to increase the T½ of the antibody in the patient. The BNJ441 antibody has heavy chain variable region as set forth in SEQ ID NO: 12 and the light chain variable region of BNJ441 is set forth in SEQ ID NO: 8. The complete heavy chain of BNJ441 is set forth below as SEQ ID NO: 14 and the light chain is set forth below as SEQ ID NO: 11

The antibody may be an anti-C5 antibody unrelated to eculizumab such as the 7086 antibody and having three heavy chain complementarity determining regions (CDRs) as set forth in Table 7 using the Kabat definitions of CDRs as heavy chain CDR1 in SEQ ID NO: 21, heavy chain CDR2 in SEQ ID NO: 22, and heavy chain CDR3 in SEQ ID NO: 23. The 7086 antibody light chain CDRs are set forth below as light chain CDR1 in SEQ ID NO: 24, light chain CDR2 in SEQ ID NO: 25 and light chain CDR3 in SEQ ID NO: 26. The heavy chain variable region of 7086 is set forth in SEQ ID NO: 27 and the light chain variable region of 7086 is set forth in SEQ ID NO: 28.

The antibody may be an anti-C5 antibody unrelated to eculizumab such as the 8110 antibody and having three heavy chain complementarity determining regions (CDRs) as set forth in Table 7 using the Kabat definitions of CDRs as heavy chain CDR1 in SEQ ID NO: 29, heavy chain CDR2 in SEQ ID NO: 30, and heavy chain CDR3 in SEQ ID NO: 31. The 7086 antibody light chain CDRs are set forth below as light chain CDR1 in SEQ ID NO: 32, light chain CDR2 in SEQ ID NO: 33 and light chain CDR3 in SEQ ID NO: 34. The heavy chain variable region of 8110 is set forth in SEQ ID NO: 35 and the light chain variable region of 8110 is set forth in SEQ ID NO: 36.

The anti C5 antibody can comprise, for example, the heavy and light chain CDRs or variable regions of the 305LO5 antibody. The anti C5 antibody can comprise, for example, heavy chain CDR1, CDR2 and CDR3 domains having the sequences set forth in SEQ ID NOs: 37, 38 and 39, respectively, and light chain CDR1, CDR2 and CDR3 domains having the sequences set forth in SEQ ID NOs: 40, 41 and 42, respectively. In another embodiment, the antibody comprises the VH region of the 305LO5 antibody having the sequence set forth in SEQ ID NO: 43, and the VL region of the 305LO5 antibody having the sequence set forth in SEQ ID NO: 44.

The anti-C5 antibody can comprise, for example, the heavy and light chain CDRs or variable regions of the SKY59 antibody. The anti C5 antibody can comprise, for example, a heavy chain comprising SEQ ID NO:45 and a light chain comprising SEQ ID NO:46.

The anti-C5 antibody can comprise, for example, the REGN3918 antibody (also known as H4H12166PP). The anti C5 antibody can comprise, for example, a heavy chain variable region comprising SEQ ID NO:47 and a light chain variable region comprising SEQ ID NO:48, or a heavy chain comprising SEQ ID NO:49 and a light chain comprising SEQ ID NO:50.

The antibody may also be an anti-C5 antibody or antigen binding fragment thereof comprising a heavy chain variable region amino acid sequence according to SEQ ID NO: 51 and a light chain variable region amino acid sequence according to SEQ ID NO: 52.

TABLE 7
EXEMPLARY SEQUENCE SUMMARY

SEQ ID NO: 1
GYIFSNYWIQ
SEQ ID NO: 2
EILPGSGSTEYTENFKD
SEQ ID NO: 3
YFFGSSPNWYFDV
SEQ ID NO: 4
GASENIYGALN
SEQ ID NO: 5
GATNLAD
SEQ ID NO: 6
QNVLNTPLT
SEQ ID NO: 7
QVQLVQSGAEVKKPGASVKVSCKASGYIFSNYWIQWVRQAPGQGLEWMGEILPGSGSTEYTEN
FKDRVTMTRDTSTSTVYMELSSLRSEDTAVYYCARYFFGSSPNWYFDVWGQGTLVTVSS
SEQ ID NO: 8
DIQMTQSPSSLSASVGDRVTITCGASENIYGALNWYQQKPGKAPKLLIYGATNLADGVPSRFS
GSGSGTDFTLTISSLQPEDFATYYCQNVLNTPLTFGQGTKVEIK
SEQ ID NO: 9
ASTKGPSVFPLAPCSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPPVAGPSVFLFPPKP
KDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVMHEALHNHY
TQKSLSLSLGK
SEQ ID NO: 10
QVQLVQSGAEVKKPGASVKVSCKASGYIFSNYWIQWVRQAPGQGLEWMGEILPGSGSTEYTEN
FKDRVTMTRDTSTSTVYMELSSLRSEDTAVYYCARYFFGSSPNWYFDVWGQGTLVTVSSASTK
GPSVFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKS
LSLSLGK
SEQ ID NO: 11
DIQMTQSPSSLSASVGDRVTITCGASENIYGALNWYQQKPGKAPKLLIYGATNLADGVPSRFS
GSGSGTDFTLTISSLQPEDFATYYCQNVLNTPLTFGQGTKVEIKRTVAAPSVFIFPPSDEQLK
SGTASVVCLLNNFYPREAKVQWKVDNALQSGNSQESVTEQDSKDSTYSLSSTLTLSKADYEKH
KVYACEVTHQGLSSPVTKSFNRGEC
SEQ ID NO: 12
QVQLVQSGAEVKKPGASVKVSCKASGHIFSNYWIQWVRQAPGQGLEWMGEILPGSGHTEYTEN
FKDRVTMTRDTSTSTVYMELSSLRSEDTAVYYCARYFFGSSPNWYFDVWGQGTLVTVSS
SEQ ID NO: 13
ASTKGPSVFPLAPCSRSTSESTAALGCLVKDYFPEPVTVSWNSGALTSGVHTFPAVLQSSGLY
SLSSVVTVPSSNFGTQTYTCNVDHKPSNTKVDKTVERKCCVECPPCPAPPVAGPSVFLFPPKP
KDTLMISRTPEVTCVVVDVSQEDPEVQFNWYVDGVEVHNAKTKPREEQFNSTYRVVSVLTVLH
QDWLNGKEYKCKVSNKGLPSSIEKTISKAKGQPREPQVYTLPPSQEEMTKNQVSLTCLVKGFY
PSDIAVEWESNGQPENNYKTTPPVLDSDGSFFLYSRLTVDKSRWQEGNVFSCSVLHEALHSHY
TQKSLSLSLGK
SEQ ID NO: 14
QVQLVQSGAEVKKPGASVKVSCKASGHIFSNYWIQWVRQAPGQGLEWMGEILPGSGHTEYTEN
FKDRVTMTRDTSTSTVYMELSSLRSEDTAVYYCARYFFGSSPNWYFDVWGQGTLVTVSSASTK
GPSVFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVLHEALHSHYTQKS
LSLSLGK
SEQ ID NO: 15
ASTKGPSVFPLAPCSRSTSESTAALGCLVKDYFPEPVTVSWNSGALTSGVHTFPAVLQSSGLY
SLSSVVTVTSSNFGTQTYTCNVDHKPSNTKVDKTVERKCCVECPPCPAPPVAGPSVFLFPPKP
KDTLYITREPEVTCVVVDVSHEDPEVQFNWYVDGMEVHNAKTKPREEQFNSTFRVVSVLTVVH
QDWLNGKEYKCKVSNKGLPAPIEKTISKTKGQPREPQVYTLPPSREEMTKNQVSLTCLVKGFY
PSDIAVEWESNGQPENNYKTTPPMLDSDGSFFLYSKLTVDKSRWQQGNVFSCSVMHEALHNHY
TQKSLSLSPGK
SEQ ID NO: 16
QVQLVQSGAEVKKPGASVKVSCKASGYIFSNYWIQWVRQAPGQGLEWMGEILPGSGSTEYTEN
FKDRVTMTRDTSTSTVYMELSSLRSEDTAVYYCARYFFGSSPNWYFDVWGQGTLVTVSSASTK
GPSVFPLAPCSRSTSESTAALGCLVKDYFPEPVTVSWNSGALTSGVHTFPAVLQSSGLYSLSS
VVTVTSSNFGTQTYTCNVDHKPSNTKVDKTVERKCCVECPPCPAPPVAGPSVFLFPPKPKDTL
YITREPEVTCVVVDVSHEDPEVQFNWYVDGMEVHNAKTKPREEQFNSTFRVVSVLTVVHQDWL
NGKEYKCKVSNKGLPAPIEKTISKTKGQPREPQVYTLPPSREEMTKNQVSLTCLVKGFYPSDI
AVEWESNGQPENNYKTTPPMLDSDGSFFLYSKLTVDKSRWQQGNVFSCSVMHEALHNHYTQKS
LSLSPGK
SEQ ID NO: 17
GASENIYHALN
SEQ ID NO: 18
EILPGSGHTEYTENFKD
SEQ ID NO: 19
GHIFSNYWIQ
SEQ ID NO: 20
QVQLVQSGAEVKKPGASVKVSCKASGHIFSNYWIQWVRQAPGQGLEWMGEILPGSGHTEYTEN
FKDRVTMTRDTSTSTVYMELSSLRSEDTAVYYCARYFFGSSPNWYFDVWGQGTLVTVSSASTK
GPSVFPLAPCSRSTSESTAALGCLVKDYFPEPVTVSWNSGALTSGVHTFPAVLQSSGLYSLSS
VVTVPSSNFGTQTYTCNVDHKPSNTKVDKTVERKCCVECPPCPAPPVAGPSVFLFPPKPKDTL
MISRTPEVTCVVVDVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVSLTCLVKGFYPSDI
AVEWESNGQPENNYKTTPPVLDSDGSFFLYSRLTVDKSRWQEGNVFSCSVMHEALHNHYTQKS
LSLSLGK
SEQ ID NO: 21
SYAIS
SEQ ID NO: 22
GIGPFFGTANYAQKFQG
SEQ ID NO: 23
DTPYFDY
SEQ ID NO: 24
SGDSIPNYYVY
SEQ ID NO: 25
DDSNRPS
SEQ ID NO: 26
QSFDSSLNAEV
SEQ ID NO: 27
QVQLVQSGQEVKKPGSSVKVSCKASGGTFSSYAISVWRQAPGQGLEWMGGIGPFFGTANYAQK
FQGRVTITADESTSTAYMELSSLRSEDTAVYYCARDTPYFDYWGQGTLVTVSS
SEQ ID NO: 28
DIELTQPPSVSVAPGQTARISCSGDSIPNYYVYWYQQKPGQAPVLVIYDDSNRPSGIPERFSG
SNSGNTATLTISGTQAEDEADYYCQSFDSSLNAEVFGGGTKLTVL
SEQ ID NO: 29
NYIS
SEQ ID NO: 30
IIDPDDSYTEYSPSFQG
SEQ ID NO: 31
YEYGGFDI
SEQ ID NO: 32
SGDNIGNSYVH
SEQ ID NO: 33
KDNDRPS
SEQ ID NO: 34
GTYDIESYV
SEQ ID NO: 35
EVQLVQSGAEVKKPGESLKISCKGSGYSFTNYISWVRQMPGKGLEWMGIIDPDDSYTEYSPSF
QGQVTISADKSISTAYLQWSSLKASDTAMYYCARYEYGGFDIWGQGTLVTVSS
SEQ ID NO: 36
SYELTQPPSVSVAPGQTARISCSGDNIGNSYVHWYQQKPGQAPVLVIYKDNDRPSGIPERFSG
SNSGNTATLTISGTQAEDEADYYCGTYDIESYVFGGGTKLTVL
SEQ ID NO: 37
SSYYVA
SEQ ID NO: 38
AIYTGSGATYKASWAKG
SEQ ID NO: 39
DGGYDYPTHAMHY
SEQ ID NO: 40
QASQNIGSSLA
SEQ ID NO: 41
GASKTHS
SEQ ID NO: 42
QSTKVGSSYGNH
SEQ ID NO: 43
QVQLVESGGGLVQPGGSLRLSCAASGFTSHSSYYVAWVRQAPGKGLEWVGAIYTGSGATYKAS
WAKGRFTISKDTSKNQVVLTMTNMDPVDTATYYCASDGGYDYPTHAMHYWGQGTLVTVSS
SEQ ID NO: 44
DVVMTQSPSSLSASVGDRVTITCQASQNIGSSLAWYQQKPGQAPRLLIYGASKTHSGVPSRFS
GSGSGTDFTLTISSLQPEDVATYYCQSTKVGSSYGNHFGGGTKVEIK
SEQ ID NO: 45
QVQLVESGGGLVQPGRSLRLSCAASGFTVHSSYYMAWVRQAPGKGLEWVGAIFTGSGAEYKAE
WAKGRVTISKDTSKNQVVLTMTNMDPVDTATYYCASDAGYDYPTHAMHYWGQGTLVTVSSAST
KGPSVFPLAPSSKSTSGGTAALGCLVKDYFPEPVTVSWNSGALTSGVHTFPAVLQSSGLYSLS
SVVTVPSSSLGTQTYICNVNHKPSNTKVDKKVEPKSCDKTHTCPPCPAPELRRGPKVFLFPPK
PKDTLMISRTPEVTCVVVDVSHEDPEVKFNWYVDGVEVHNAKTKPREEQYNSTYRVVSVLTVL
HQDWLNGKEYKCKVSNKGLPSSIEKTISKAKGQPREPQVYTLPPSREEMTKNQVSLTCLVKGF
YPSDIAVEWESNGQPENNYKTTPPVLDSDGSFFLYSKLTVDKSRWQQGNVFSCSVLHEALHAH
YTRKELSLSP
SEQ ID NO: 46
DIQMTQSPSSLSASVGDRVTITCRASQGISSSLAWYQQKPGKAPKLLIYGASETESGVPSRFS
GSGSGTDFTLTISSLQPEDFATYYCQNTKVGSSYGNTFGGGTKVEIKRTVAAPSVFIFPPSDE
QLKSGTASVVCLLNNFYPREAKVQWKVDNALQSGNSQESVTEQDSKDSTYSLSSTLTLSKADY
EKHKVYACEVTHQGLSSPVTKSFNRGEC
SEQ ID NO: 47
QVQLQESGPGLVKPSETLSLTCTVSGDSVSSSYWTWIRQPPGKGLEWIGYIYYSGSSNYNPSLK [symbol]
ATISVDTSKNQFSLKLSSVTAADTAVYYCAREGNVDTTMIFDYWGQGTLVTVSS
SEQ ID NO: 48
AIQMTQSPSSLSASVGDRVTITCRASQGIRNDLGWYQQKPGKAPKLLIYAASSLQSGVPSRFAG [symbol]
SGTDFTLTISSLQPEDFATYYCLQDFNYPWTFGQGTKVEIK
SEQ ID NO: 49
QVQLQESGPGLVKPSETLSLTCTVSGDSVSSSYWTWIRQPPGKGLEWIGYIYYSGSSNYNPSLK [symbol]
ATISVDTSKNQFSLKLSSVTAADTAVYYCAREGNVDTTMIFDYWGQGTLVTVSSASTKGPSVFP [symbol]
PCSRSTSESTAALGCLVKDYFPEPVTVSWNSGALTSGVHTFPAVLQSSGLYSLSSVVTVPSSSL [symbol]
KTYTCNVDHKPSNTKVDKRVESKYGPPCPPCPAPEFLGGPSVFLFPPKPKDTLMISRTPEVTCV [symbol]
DVSQEDPEVQFNWYVDGVEVHNAKTKPREEQFNSTYRVVSVLTVLHQDWLNGKEYKCKVSNKGL [symbol]
SIEKTISKAKGQPREPQVYTLPPSQEEMTKNQVSLTCLVKGFYPSDIAVEWESNGQPENNYKTT [symbol]
VLDSDGSFFLYSRLTVDKSRWQEGNVFSCSVMHEALHNHYTQKSLSLSLGK
SEQ ID NO: 50
AIQMTQSPSSLSASVGDRVTITCRASQGIRNDLGWYQQKPGKAPKLLIYAASSLQSGVPSRFAG [symbol]
SGTDFTLTISSLQPEDFATYYCLQDFNYPWTFGQGTKVEIKRTVAAPSVFIFPPSDEQLKSGTA [symbol]
VCLLNNFYPREAKVQWKVDNALQSGNSQESVTEQDSKDSTYSLSSTLTLSKADYEKHKVYACEV [symbol]
QGLSSPVTKSFNRGEC
SEQ ID NO: 51
QVQLVESGGGLVQPGRSLRLSCAASGFTVHSSYYMAWVRQAPGKGLEWVGAIFTGSGAEYKAE
WAKGRVTISKDTSKNQVVLTMTNMDPVDTATYYCASDAGYDYPTHAMHYWGQGTLVTVSS
SEQ ID NO: 52
DIQMTQSPSSLSASVGDRVTITCRASQGISSSLAWYQQKPGKAPKLLIYGASETESGVPSRFS
GSGSGTDFTLTISSLQPEDFATYYCQNTKVGSSYGNTFGGGTKVEIK [symbol]
indicates data missing or illegible when filed

### EXAMPLES

### Example 1: REGAIN Study Comprising ECU-MG-301 26 Week (301) Trial

The primary objective of this trial was to assess the efficacy of eculizumab as compared with placebo in the treatment of refractory gMG based on the improvement in the MG-specific Activities of Daily Living profile (MG-ADL).

The secondary objectives of this trial included the following:
Characterize the overall safety and tolerability of eculizumab as compared with placebo in gMG subjects
Assess the efficacy of eculizumab as compared with placebo by additional efficacy measures including:
   Quantitative MG (QMG) Score for Disease Severity
   Myasthenia Gravis Composite (MGC)
   Improvement in primary symptoms that are most clinically meaningful to the subjects
   MG-ADL subcategories for bulbar, respiratory, limb and ocular
Characterize the effect of eculizumab as compared with placebo on Quality of life measures
Describe the PK and PD of eculizumab in gMG subjects.

### 1. Investigational Plan

### 1.1. Overall Trial Design and Plan

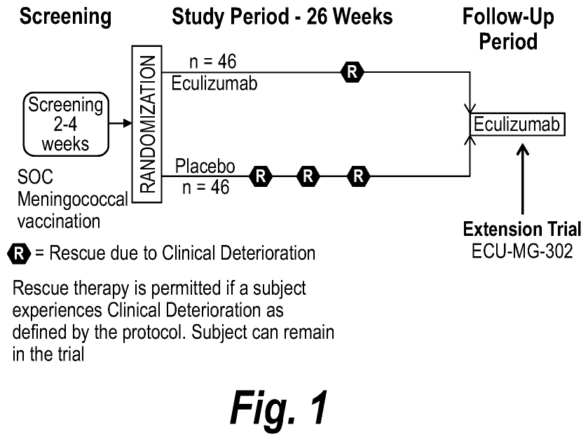

Described herein is a randomized, double-blind, parallel-group, placebo-controlled, multicenter (˜100 sites in North America, South America, Europe, Asian Pacific) approximately two year trial that evaluated the safety and efficacy of eculizumab for the treatment in subjects with refractory gMG. Approximately 92 eligible subjects were randomized on Day 1 on a 1:1 ratio to one of two treatment arms (1) eculizumab infusion or (2) placebo infusion. Subjects continued to receive stable dose/type of immunosuppressive therapy (IST), but no new ISTs and no increase in IST dosage were permitted during the trial. There were 3 periods in this study: Screening Period, Study Period, and Follow-up Period (for subjects who withdrew from this trial or who did not enter the extension trial). See FIG. 1. The overall trial duration for an individual subject was estimated to take up to 38 weeks including enrollment and Follow-up. Subjects were provided the opportunity to participate in an extension trial (separate protocol) to receive eculizumab after completion of this trial. A schedule of assessments for the screening, study and follow-up period is provided in Table 8.

### 1.1.1. Screening Period (2-4 Weeks)

At the screening visit, after obtaining the informed consent from the subject, the subject was screened for trial eligibility through medical history review, demographic data, and laboratory assessments. The medical history review includes confirmation of MG diagnosis as defined in the inclusion criteria of this protocol, history of previous treatment/therapies for MG, e.g., thymectomy, IST including corticosteroids, IVIg and plasma exchange, history of MG exacerbation or crisis including the duration of each exacerbation/crisis, the medication taken at the time of each exacerbation/crisis and the treatment for each exacerbation/crisis.

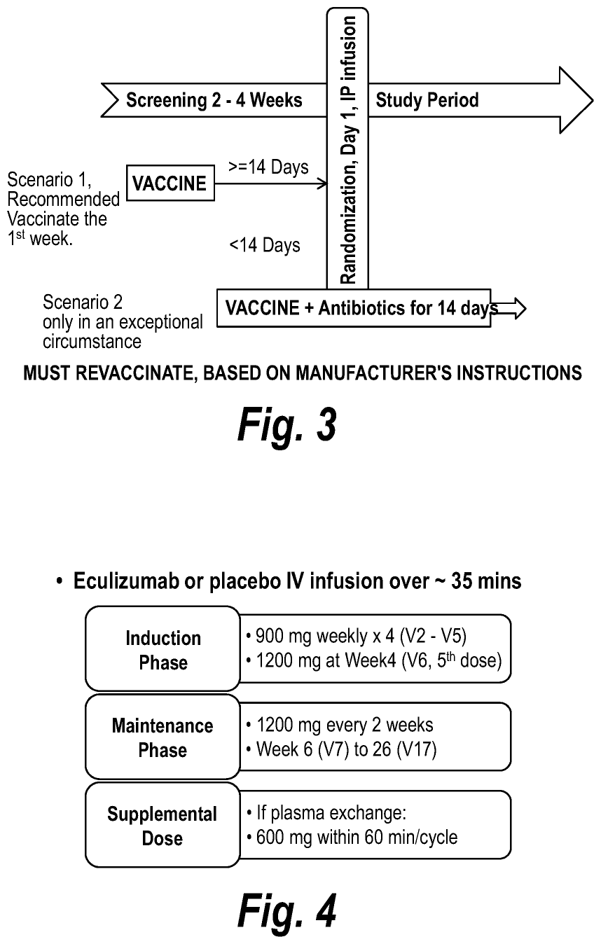

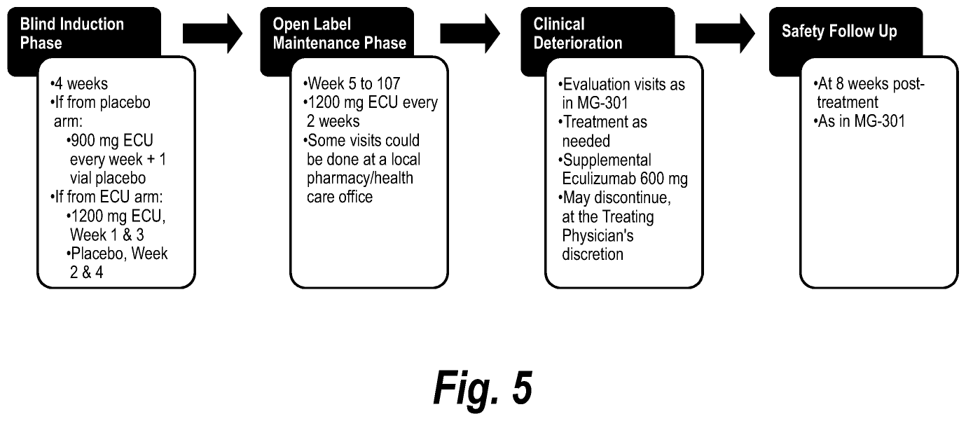

If all inclusion criteria and none of the exclusion criteria were met, subjects were vaccinated against N. meningitidis, if not already vaccinated within the time period of active coverage specified by the vaccine manufacturer or vaccinate according to current medical/country guidelines. Subjects were vaccinated at least 14 days prior to receiving the first dose of study medication were vaccinated and received treatment with appropriate antibiotics until 14 days after the vaccination. See FIG. 3.

Use of cholinesterase inhibitor and supportive IST were allowed during the trial under certain restrictions (see Concomitant Medications, below). The washout period for IVIg was 4 weeks prior to randomization. The washout period for PE was also 4 weeks prior to randomization. If a subject experienced an MG Crisis during the Screening Period, the sponsor was notified. Following discussion with the sponsor, a decision was made about whether the subject may continue in the trial, be withdrawn and possibly, re-screened at a later date.

TABLE 8
TRIAL DESIGN AND SCHEDULE OF ASSESSMENTS (STUDY PERIOD)

| Period/Phase | Screening | Induction | | | | | Maintenance | | | |
|---|---|---|---|---|---|---|---|---|---|---|
| Trial Visit | 1 | 2 | 3 | 4 | 5 | 6 | 7 | 8 | 9 | 10 |
| Trial Weeks | 2-4 Weeks | D1 | W1 | W2 | W3 | W4 | W6 | W8 | W10 | W12 |
| Informed Consent | X | | | | | | | | | |
| Medical History | X | | | | | | | | | |
| MG History ¹ | X | | | | | | | | | |
| MGFA Clinical | X | | | | | | | | | |
| Classification | | | | | | | | | | |
| Weight | X | | | | | | | | | |
| Height | X | | | | | | | | | |
| Vital Signs | X | X | X | X | X | X | X | X | X | X |
| Physical Exam | X | | | | | | | | | |
| 12-Lead ECG | X | | | | | | | | | |
| Concomitant | X | X | X | X | X | X | X | X | X | X |
| Medication | | | | | | | | | | |
| MG Therapy Status | X | X | | | | | | | | |
| Adverse Event | | X | X | X | X | X | X | X | X | X |
| MG-QOL 15 | X | X | | | | X | | X | | X |
| Neuro-QOL Fatigue | | X | | | | X | | X | | X |
| EQ-5D | | X | | | | X | | X | | X |
| MG-ADL ² | X | X | X | X | X | X | | X | | X |
| QMG ³ | X | X | X | X | X | X | | X | | X |
| NIF ³ | X | X | X | X | X | X | | X | | X |
| MGC ³ | X | X | X | X | X | X | | X | | X |
| MGFA PIS ⁴ | | | | | | X | | | | X |
| C-SSRS | | X | | | | | | | | X |
| AChR Ab | X | | | | | | | | | X |
| Clinical Lab Tests ⁵ | X | X | | | | X | | X | | X |
| Pregnancy Test ⁶ | X | X | | | | | | | | |
| PK/PD, Free C5 ⁷ | | B/P | T/P | | | T/P | | T/P | | T/P |
| HAHA ⁷ | | B | | | | X | | | | X |
| Medically Indicated | | | | | | | | | | |
| Tests | | | | | | | | | | |
| N. meningitidis | X | | | | | | | | | |
| Vaccination ⁸ | | | | | | | | | | |
| Patient Safety | | X | X | X | X | X | X | X | X | X |
| Information Card | | | | | | | | | | |
| Randomization ⁹ | | X | | | | | | | | |
| IP Infusion ¹⁰ | | X | X | X | X | X | X | X | X | X |
| | Maintenance | | | | | | | | | Post- |
| Period/Phase | | | | | | | 17/ | | ++ | Treatment |
| Trial Visit | 11 | 12 | 13 | 14 | 15 | 16 | ET* | \hat{ }Clinical | UNS | Follow-up |
| Trial Weeks | W14 | W16 | W18 | W20 | W22 | W24 | W26 | Deterioration | Visit | +W8 |
| Informed Consent | | | | | | | | | | |
| Medical History | | | | | | | | | | |
| MG History ¹ | | | | | | | | | | |
| MGFA Clinical | | | | | | | | | | |
| Classification | | | | | | | | | | |
| Weight | | | | | | | X | | | |
| Height | | | | | | | | | | |
| Vital Signs | X | X | X | X | X | X | X | X | | X |
| Physical Exam | | | | | | | | | | |
| 12-Lead ECG | | | | | | | X | | | |
| Concomitant | X | X | X | X | X | X | X | X | | X |
| Medication | | | | | | | | | | |
| MG Therapy Status | | | | | | | X | | | |
| Adverse Event | X | X | X | X | X | X | X | X | | X |
| MG-QOL 15 | | X | | X | | | X | | | X |
| Neuro-QOL Fatigue | | X | | X | | | X | | | |
| EQ-5D | | X | | X | | | X | | | |
| MG-ADL ² | | X | | X | | | X | X | | X |
| QMG ³ | | X | | X | | | X | X | | X |
| NIF ³ | | X | | X | | | X | X | | X |
| MGC ³ | | X | | X | | | X | X | | X |
| MGFA PIS ⁴ | | | | | | | X | | | X |
| C-SSRS | | | | | | | X | | | |
| AChR Ab | | | | | | | X | X | | |
| Clinical Lab Tests ⁵ | | X | | X | | | X | X | | |
| Pregnancy Test ⁶ | | | | | | | X | | | |
| PK/PD, Free C5⁷ | | | | T/P | | | T/P | X | | |
| HAHA ⁷ | | | | | | | X | | | |
| Medically Indicated | | | | | | | | X | | |
| Tests | | | | | | | | | | |
| N. meningitidis | | | | | | | | | | |
| Vaccination ⁸ | | | | | | | | | | |
| Patient Safety | X | X | X | X | X | X | X | | | |
| Information Card | | | | | | | | | | |
| Randomization ⁹ | | | | | | | | | | |
| IP Infusion ¹⁰ | X | X | X | X | X | X | X | X ¹⁰ | | |

Abbreviations:
AChR Ab = Acetylcholine receptor antibody; B = Baseline sample; C5 = Complement protein 5; C-SSRS = Columbia-Suicide Severity Rating Scale; ECG = Electrocardiogram; ICU = Intensive Care Unit; HAHA = human anti-human antibody; ; IP = investigational product; MG = Myasthenia Gravis; MGC = MG Composite Score; MG-ADL = MG Activity of Daily Living (MG-ADL) Profile; MGFA = Myasthenia Gravis Foundation of America; MGFA PIS = MGFA Post-Intervention Status; NIF = negative inspiratory force; P = peak sample; PK/PD = Pharmacokinetics/Pharmacodynamics QMG = Quantitiative MG (QMG) Score for Disease Severity; QOL = Quality of Life; T = trough sampl

### 1.1.2. Randomization

All subjects who were vaccinated, and continued to meet the MG-ADL entry criteria, i.e., MG-ADL total score ≥6 at Randomization (Day 1), and were cleared for randomization by their respective Principal Investigator (PI), were randomized on Day 1 on a 1:1 basis to the Eculizumab Arm or the Placebo Arm. The randomization stratification is based on the assessment of clinical classification by the Myasthenia Gravis Foundation of America (MGFA) (see Table 9) performed at the Screening Visit according to the following 4 groupings:
a. MGFA Class IIa and IIIa
b. MGFA Class IVa
c. MGFA Class IIb and IIIb, and
d. MGFA Class IVb

### 1.1.3. Study Period (26 Weeks)

Subjects received IP, either eculizumab or placebo, according to the randomization and the regimen described in the Investigational Product and Administration, described below. The treatment duration for each subject was 26-weeks. Subjects were informed of potential signs and symptoms of MG crisis and were instructed to contact the Investigator as soon as possible at onset of symptom. Every effort was made for the subject reporting Clinical Deterioration to be evaluated as soon as possible and within 48 hours of notification of the Investigator of the symptom onset. At the evaluation visit, the Investigator or his/her designee performed the assessments as specified by this protocol. The Investigator determined whether or not the subject met the definition of Clinical Deterioration as defined by this protocol and treated the subject accordingly.

TABLE 9
MGFA CLINICAL CLASSIFICATION

| Class | Clinical signs |
|---|---|
| I | Any ocular muscle weakness. |
| | May have weakness of eye closure. |
| | All other muscle strength is normal. |
| II | Mild weakness affecting other than ocular muscles. |
| | May also have ocular muscle weakness of any severity. |
| IIa | Predominantly affecting limb or axial muscles or both. |
| | May also have lesser involvement of oropharyngeal muscles. |
| IIb | Predominantly affecting oropharyngeal or respiratory |
| | muscles or both. May also have lesser or equal |
| | involvement of limb or axial muscles or both. |
| III | Moderate weakness affecting other than ocular muscles. |
| | May also have ocular muscle weakness of any severity. |
| IIIa | Predominantly affecting limb or axial muscles or both. |
| | May also have lesser involvement of oropharyngeal muscles. |
| IIIb | Predominantly affecting oropharyngeal or respiratory |
| | muscles or both. May also have lesser or equal |
| | involvement of limb or axial muscles or both. |
| IV | Severe weakness affecting other than ocular muscles. |
| | May also have ocular muscle weakness of any severity, |
| IVa | Predominantly affecting limb and/or axial muscles. |
| | May also have lesser involvement of oropharyngeal muscles. |
| IVb | Predominantly affecting oropharyngeal or respiratory |
| | muscles or both. May also have lesser or equal |
| | involvement of limb or axial muscles or both. |
| V | Defined by intubation, with or without mechanical |
| | ventilation, except when employed during routine |
| | postoperative management The use of a feeding |
| | tube without intubation places the patient in class IVb. |

After completing the 26-week Study Period, subjects were provided an opportunity to enter an extension trial (separate protocol) to receive open-label eculizumab. The visit interval between this trial and the extension trial was 2 weeks from the last of IP administration (Visit 17) so there was no interruption in IP dosing. Subjects that entered the extension trial underwent a blinded eculizumab induction phase similar to the induction in this trial in order to maintain the blinded treatment assignment of this trial. If a subject withdrew from this trial at any time after receiving any amount of IP or did not wish to enter the extension trial after completion of this trial, the subject was required to complete the Follow-up Visit for safety measures.

### 1.1.4. Follow-Up Period (8 Weeks Post-Treatment)

If a subject withdrew or is discontinued from this trial at any time after receiving any amount of IP or did not wish to enter the extension trial after completion of this trial, the subject was required to complete the Follow-up Visit for safety measures 8 weeks after the last IP dose administration. If a subject is discontinued due to an AE, the event was followed until it was resolved or in the opinion of the PI was medically stable.

### 1.2. Standard Protocol Definitions

Abbreviations and definitions for the study and follow-up period are provided in Table 10.

TABLE 10
LIST OF ABBREVIATIONS AND DEFINITIONS OF TERMS

| Abbreviation or Specialist Term | Explanation |
|---|---|
| Ab | Antibody |
| AChR | Acetylcholine receptor |
| AE | Adverse event |
| aHUS | Atypical hemolytic uremic syndrome |
| ANCOVA | Analysis of covariance |
| AZA | Azathioprine |
| BP | Blood Pressure |
| C5 | Complement protein 5 |
| CMAX | Maximal concentration |
| CMIN | Minimal concentration |
| eCRF | Electronic Case Report Form |
| C-SSRS | Columbia-Suicide Severity |
| | Rating Scale |
| ECG | Electrocardiogram |
| EDC | Electronic Data Capture |
| EIU | Exposure in-utero |
| EOI | Event of Interest |
| EOS | End of Study |
| EQ-5D | EuroQoL |
| ET | Early Termination |
| EU | European Union |
| FAS | Full Analysis Set |
| FVC | Forced Vital Capacity |
| GCP | Good Clinical Practices |
| gMG | Generalized Myasthenia Gravis |
| HAHA | Human Anti-human Antibody |
| HCG | human chorionic gonadotropin |
| HR | Heart Rate |
| IB | Investigator Brochure |
| ICF | Informed Consent Form |
| ICH | International Conference |
| | on Harmonization |
| ICU | Intensive Care Unit |
| IEC | Independent Ethics Committee |
| IVIg | Intravenous Immunoglobulin G |
| IP | Investigational Product |
| IRB | Institutional Review Board |
| IST | Immunosuppressant Therapy |
| IV | Intravenous |
| IVIg | Intravenous immunoglobulin |
| IXRS | Interactive voice or web |
| | response system |
| mAb | Monoclonal Antibody |
| MedDRA | Medical Dictionary for |
| | Regulatory Activities |
| MG | Myasthenia Gravis |
| MG-ADL | MG activity of daily living profile |
| MGC | Myasthenia Gravis Composite |
| MGFA | Myasthenia Gravis Foundation |
| | of America |
| MM | Minimal manifestation |
| MMF | Mycophenolate Mofetil |
| MMT | Manual Muscle Test |
| MTX | Methotrexate |
| MuSK | Muscle-specific tyrosine kinase |
| NIF | Negative inspiratory force |
| NMJ | Neuromuscular junction |
| oMG | Ocular Myasthenia Gravis |
| PD | Pharmacodynamics |
| PE | Plasmapheresis or Plasma Exchange |
| PI | Principal Investigator |
| PIS | Post-Intervention Status |
| PK | Pharmacokinetics |
| PNH | Paroxysmal Nocturnal Hemglobinuria |
| PP | Per-Protocol Population |
| QOL | Quality of Life |
| QMG | Quantitative Myasthenia Gravis |
| RR | Respiration Rate |
| RSI | Reference Safety Information |
| SAE | Serious Adverse Event |
| SAP | Statistical Analysis Plan |
| SFEMG | single-fiber electromyography |
| SOC | System Organ Class |
| TEAE | Treatment Emergent Adverse Events |
| TESAE | Treatment Emergent SAE |
| US | United States |
| VAS | Visual Analog Scale |
| WHODmg | World Health Organization |
| | Drug Dictionary |

### 1.2.1. Clinical Deterioration

For this protocol. Clinical Deterioration was defined as follows:
Subjects who experienced an MG Crisis, which was defined as weakness from MG that was severe enough to necessitate intubation or to delay extubation following surgery. The respiratory failure was due to weakness of respiratory muscles. Severe bulbar (oropharyngeal) muscle weakness often accompanied the respiratory muscle weakness, or was the predominant feature in some subjects; or,
Significant symptomatic worsening to a score of 3 or a 2-point worsening on any one of the individual MG-ADL items other than double vision or eyelid droop; or,
Subjects for whom the treating physician believes that the subject's health was in jeopardy if rescue therapy was not given (e.g., emergency situations).

### 1.2.2. Clinical Evaluation

The Clinical Evaluators were study staff that have been trained and certified in administering the MG-ADL, QMG and MGC. The Clinical Evaluator may have been a neurologist, physical therapist or other study team member delegated by the PI. Clinical Evaluator training and certification for this protocol took place either at the Investigator's meeting or via the sponsor's designated on-line training portal.1.2.3.

Responsibilities for MG Assessments

Responsibilities for MG assessments are listed in Table 11. Throughout the trial, MG assessments were performed at approximately the same time of day by a properly trained evaluator, preferably the same evaluator.

TABLE 11
MG ASSESSMENTS (RESPONSIBILITIES)

| Assessment | Evaluator |
|---|---|
| MG-ADL | Clinical Evaluator |
| QMG including FVC | Clinical Evaluator |
| NIF | Clinical Evaluator |
| MGC | Clinical Evaluator |
| MGC (MMT Components) | PI or Neurologist |
| MGFA-PIS | PI or Neurologist |
| MGFA Classification | PI or Neurologist |

Abbreviations: FVC = forced vital capacity;
MG-ADL = Myasthenia Gravis Activity of Daily Living Profile;
MGC = Myasthenia Gravis Composite;
MGFA = Myasthenia Gravis Foundation of America;
MGFA-PIS = Myasthenia Gravis Foundation of America Post Intervention Status;
MMT = manual muscle test;
NIF = negative inspiratory force;
PI = Principal Investigator;
QMG = Quantitative MG

### 1.4. Trial Visit Procedures

### 1.4.1. Screening Visit

(Days −28 to −14 Prior to Baseline [Visit 2/Day 1])

After obtaining a signed informed consent form, the following tests and evaluations were performed within 2-4 weeks prior to randomization at the Baseline Visit (Visit 2/Day 1) to determine subject eligibility for participation in this trial:
Review of inclusion and exclusion criteria; Registered the subject in the IXRS system to get the subject identification number in the study and trigger drug shipment if necessary; Recorded medical history and demographics; Record MGFA Clinical Classification
Record MG history:
   a. Confirmed MG diagnosis as defined by the protocol inclusion criterion #2
   b. Recorded the initial MG clinical presentation (i.e., oMG or gMG). If the initial clinical presentation was oMG, recorded the time (date) to onset of gMG
   c. Recorded the maximum MGFA classification since diagnosis, if available
   d. Recorded whether the subject ever required ventilatory support since the diagnosis
   e. Recorded the number of hospitalizations, including number of ICU stays (days) and any ventilatory support associated with the hospitalization within the last 2 years prior to screening
   f. Recorded number and duration of all previous MG exacerbations or recorded number and duration of all previous MG exacerbations or crisis, the medication/therapy taken at the time of each exacerbation or crisis, and medication/therapy use for treatment of each exacerbation or crisis, if applicable.
Recorded MG Therapy Status (see Table 12)
Measured body weight and height
Measured vital signs, including assessments of systolic and diastolic blood pressure (BP), temperature, respiration rate (RR) and heart rate (HR)
Completed physical examination including assessments of the following organ/body systems: skin, head, ears, eyes, nose, throat, neck, lymph nodes, chest, heart, abdomen, extremities, musculoskeletal, and general neurologic examination.
Performed a 12-Lead ECG
Recorded concomitant medications, including prior IST, IVIg, and/or PE for MG from the time of diagnosis up to screening and all other concomitant medications within 30 days prior to the Screening Visit.
Administered MG-ADL by a properly trained evaluator. The recall period was the preceding 7 days.
Administered clinical assessments QMG, NIF, and MGC; these were performed at approximately the same time of day by a properly trained evaluator. If the subject is taking a cholinesterase inhibitor, the dose must be withheld for at least 10 hours prior to the QMG and MGC tests.
Administered MG-QOL15 questionnaire to evaluate quality of life.
Obtained blood sample for AChR Abs test.
Obtained blood samples for laboratory tests (chemistry and hematology) (see Table 8)
Obtained pregnancy test (serum) for all women of childbearing potential. Note: if the subject was taking/using contraceptive medication/device, recorded the medication or device on the appropriate electronic case report form (eCRF) pages (concomitant medication or procedure).
If all inclusion criteria and none of the exclusion criteria were met, subjects were vaccinated against N. meningitides, if not already vaccinated within the time period of active coverage specified by the vaccine manufacturer or according to current medical/country guidelines. Subjects were vaccinated at least 14 days prior to receiving the first dose of study medication or were vaccinated and received treatment with appropriate antibiotics until 14 days after the vaccination.
If a subject experienced an MG Crisis during the Screening Period, the sponsor was notified. Following discussion with the sponsor, a decision was made about whether the subject may continue in the trial, be withdrawn and possibly, re-screened at a later date.

### 1.4.2. Study Period

Visit intervals during Induction Phase (Visits 2, 3, 4, 5 and 6) were weekly (every 7 f 2 days after the last visit). Visit intervals during the Maintenance Phase (Visits 7-17) were every 2 weeks (every 14 days±2 days since the last visit). Subjects who fail to return for a scheduled visit were contacted by the site study staffs to determine the reason for missing the appointment. Subjects were strongly encouraged to return to the investigational site for evaluation if Clinical Deterioration or an AE was suspected to have occurred. In the exceptional circumstance where a subject could not or did not come to the study site for examination, then the subject was instructed to see his or her local neurologist or physician. In this event, the investigational site obtained relevant medical records as documentation from the local physician's examination, and entered relevant data in the eCRF as appropriate.

TABLE 12
MGFA MG THERAPY STATUS

| | |
|---|---|
| NT | No therapy |
| SPT | Status postthymectomy (record type of resection) |
| CH | Cholinesterase inhibitors |
| PR | Prednisone |
| IM | Immunosuppression therapy other than prednisone (define) |
| PE(a) | Plasma exchange therapy, acute |
| | (for exacerbations or preoperatively) |
| PE(c) | Plasma exchange therapy, chronic (used on a regular basis) |
| IG(a) | IVIg therapy, acute (for exacerbations or preoperatively) |
| IG(c) | IVIg therapy, chronic (used on a regular basis) |
| OT | Other forms of therapy (define) |

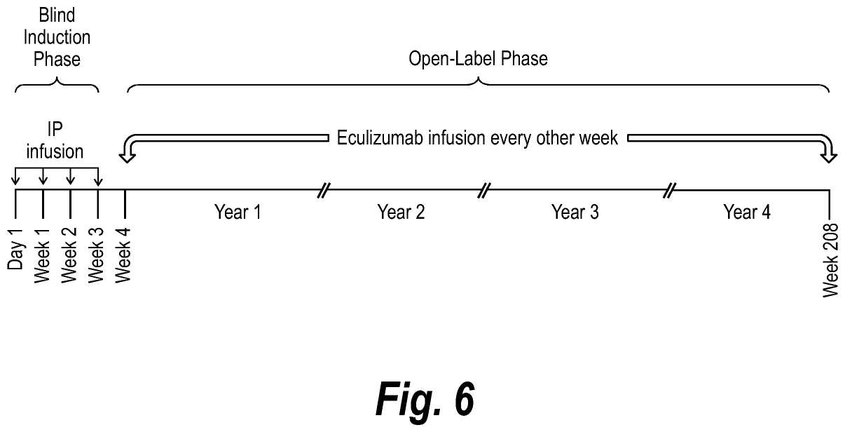

As it was vital to obtain information on any subject's missing visit to assure the missing appointment was not due to a clinical deterioration or an AE, every effort was made to undertake protocol-specified follow-up procedures (see Table 8). Follow-up due diligence documentation consisted of 3 phone calls followed by 1 registered letter to the subject's last known address. The study period is summarized in Table 8 and FIG. 6.

### 1.4.2.1. Induction Phase (Baseline [Visit 2/Day 1] Until Visit 6 [Week 4])

### 1.4.2.1.1. Baseline (Visit 2/Day 1)

Once all of the eligibility criteria were confirmed by the P1, the subject was randomized on Day 1. The following tests and procedures were completed at the Baseline Visit (Visit 2/Day 1):
Measured vital signs, including assessments of systolic and diastolic BP, temperature, RR and HR
Recorded MG Therapy Status (see Table 12)
Recorded any new medications or changes to concomitant medications
Evaluated and record AEs since the previous visit
Administered questionnaires to evaluate quality of life (MG-QOL 15, Neuro-QOL Fatigue, and EuroQoL [EQ-5D])
Administered MG-ADL by a properly trained evaluator, preferably the same evaluator, throughout the trial. The recall period was the preceding 7 days. If the number of days since the last visit was <7, the recall period was since the last visit.
Administered clinical assessments QMG, NIF, and MGC; these were performed at approximately the same time of day by a properly trained evaluator, preferably the same evaluator, throughout the trial. If the subject was taking a cholinesterase inhibitor, the dose was withheld for at least 10 hours prior to the QMG and MGC tests.
Performed Columbia-Suicide Severity Rating Scale (C-SSRS)
Obtained blood samples for clinical laboratory tests (chemistry and hematology)
Obtained pregnancy test (serum) for all women of childbearing potential.
Collected baseline blood samples for PK, PD, free C5, and HAHA assays 5-90 minutes before the infusion of IP.
Instructed the subject on the signs and symptoms of N. meningitis. Provided the Patient Safety Information Card describing the IP and emergency contact information to the subject prior to the first dose of IP.
Randomized the subject using the IXRS.
Administered the IP infusion over approximately 35 minutes according to the regimen described in Section 4.5, and observed subjects for 1 hour after the end of the IP infusion.
Collected peak blood samples for PK, PD, and free C5 assays at least 60 minutes after completion of the IP infusion.

### 1.4.2.1.2. Visits 3-5 (Weeks 1-3)

The following tests and procedures are completed:
Measured vital signs, including assessments of systolic and diastolic BP, temperature, RR, and HR
Recorded any new medications or changes to concomitant medications
Evaluated and record any new AEs or changes in AEs since the previous visit.
Administered MG-ADL by a properly trained evaluator, preferably the same evaluator, throughout the trial. The recall period was the preceding 7 days. If the number of days since the last visit was <7, the recall period was since the last visit.
Administered clinical assessments QMG, NIF, and MGC; these were performed at approximately the same time of day by a properly trained evaluator, preferably the same evaluator, throughout the trial. If the subject was taking a cholinesterase inhibitor, the dose was withheld for at least 10 hours prior to the QMG and MGC tests.
At Visit 3 (Week 1) only, collected trough (before IP infusion) blood samples for PK, PD, and free C5 assays. Trough blood samples were taken 5-90 minutes before the IP infusion.
Ensured that the subject has the Patient Safety Information Card that describes the IP and emergency contact information.
Obtained study drug kit assignation through the IXRS.
Administered the IP infusion over approximately 35 minutes according to the regimen described in Section 4.5, and observed subjects for 1 hour after the end of the IP infusion.
At Visit 3 (Week 1) only, collected peak (after IP infusion) blood samples for PK, PD, and free C5 assays. Peak blood samples were taken at least 60 minutes after the completion of the IP infusion.

### 1.4.2.1.3. Visit 6 (Week 4)

The following tests and procedures are completed at this visit:
Measured vital signs, including assessments of systolic and diastolic BP, temperature, RR, and HR
Recorded any new medications or changes to concomitant medications
Evaluated and record any new AEs or changes in AEs since the previous visit
Administered questionnaires to evaluate quality of life (MG-QOL 15, Neuro-QOL Fatigue, and EQ-5D)
Administered MG-ADL by a properly trained evaluator, preferably the same evaluator, throughout the trial. The recall period was the preceding 7 days. If the number of days since the last visit was <7, the recall period was since the last visit.
Administered clinical assessments QMG, NIF, and MGC; these were performed at approximately the same time of day by an appropriately trained evaluator, preferably the same evaluator, throughout the trial. If the subject was taking a cholinesterase inhibitor, the dose was withheld for at least 10 hours prior to the QMG and MGC tests.
Assessed change from baseline in the MGFA Post-Intervention Status (see Table 6).
Collected blood samples for clinical laboratory tests (chemistry and hematology).
Collected trough blood samples for PK, PD, free C5, and HAHA assays 5-90 minutes before the infusion of IP.
Ensured that the subject has the Patient Safety Information Card that describes the IP and emergency contact information.
Obtained study drug kit assignation through the IXRS.
Administered the IP infusion over approximately 35 minutes according to the regimen described in Section 4.5, and observed subjects for 1 hour after the end of the IP infusion.
Collected peak blood samples for PK, PD, and free C5 assays at least 60 minutes after completion of the IP infusion.

### 1.4.2.2. Maintenance Phase (Visit 7 [Week 6] Until End of Study Period Visit 17 [Week 26] or Early Termination of Visit)

During the Maintenance Phase, subjects returned for infusions of IP every 2 weeks (14±2 days), according to the regimen described in Section 4.5. The following tests and procedures were completed at every visit beginning at Visit 7 (Week 6) and continued until the End of Study (EOS), Visit 17 (Week 26) or at Early Termination (ET):
Measured vital signs, including assessments of systolic and diastolic BP, temperature, RR, and HR
Recorded any new medications or changes to concomitant medications
Evaluated and record any new AEs or changes in AEs since the previous visit.
Ensured that the subject has the Patient Safety Information Card that describes the IP
and emergency contact information.
Administered the IP and observed subjects for 1 hour after the end of the IP infusion. IP was administered after completion of other tests and procedures, excluding the peak blood sampling for PK/PD and free C5 assay.

At Visit 8 (Week 8), Visit 10 (Week 12), Visit 12 (Week 16), Visit 14 (Week 20), and until the EOS, Visit 17 (Week 26) or at ET, the following procedures were also completed, in addition to the 5 preceding procedures listed for the maintenance phase:
Administered questionnaires to evaluate quality of life (MG-QOL 15, Neuro-QOL Fatigue, and EQ-5D)
Administered MG-ADL by a properly trained evaluator, preferable the same evaluator, throughout the trial. The recall period was the preceding 7 days.
Administered clinical assessments QMG, NIF, and MGC; these were performed at approximately the same time of day by a properly trained evaluator, preferably the same evaluator, throughout the trial. If the subject was taking a cholinesterase inhibitor, the dose was withheld for at least 10 hours prior to the QMG and MGC tests.
Performed C-SSRS only at Visit 10 (Week 12) and Visit 17 (Week 26)/ET. The blood sample for the HAHA assay was collected 5-90 minutes before the infusion of IP.
Obtained blood sample for clinical laboratory tests (chemistry and hematology).
Obtained blood sample for the AChR Abs test and HAHA assay only at Visit 10 (Week 12) and Visit 17 (Week 26)/ET.
Collected trough blood samples for PK, PD, and free C5 assays 5-90 minutes before the infusion of IP only at Visits 8, 10, 14 and 17/ET (Weeks 8, 12, 20, and 26).
Collected peak blood samples for PK, PD, and free C5 assays at least 60 minutes after completion of the IP infusion only at Visits 8, 10, 14 and 17/ET (Weeks 8, 12, 20, and 26).
Measured body weight only at Visit 17 (Week 26)/ET.
Performed a 12-Lead ECG only at Visit 17 (Week 26)/ET.
Recorded MG Therapy Status (see Table 12) only at Visit 17 (Week 26)/ET.
Obtained pregnancy test must for all women of childbearing potential at Visit 17 (Week 26)/ET.
Assessed change from baseline in the MGFA Post-Intervention Status only at Visit 10 (Week 12) and Visit 17 (Week 26)/ET.

### 1.4.2.3. Visits for MG Crisis or Clinical Deterioration

The evaluation visit for an MG crisis or Clinical Deterioration was performed as soon as possible, within 48 hours of notification of the Investigator of the symptom onset. Additional evaluation visits were scheduled at the discretion of the investigator. The following tests and procedures were completed at this visit:
Measured vital signs, including assessments of systolic and diastolic BP, temperature, RR, and HR
Recorded any new medications or changes to concomitant medications, including all treatments for MG
Evaluated and recorded any new AEs or changes in AEs since the previous visit
Administered MG-ADL by a properly trained evaluator, preferably the same evaluator, throughout the trial. The recall period was the preceding 7 days or since the last visit whichever occurred earlier.
Administered clinical assessments QMG, NIF, and MGC; these were performed at approximately the same time of day by a properly trained evaluator, preferably the same evaluator, throughout the trial.
Collected blood sample for the AChR Abs test
Collected blood samples for clinical laboratory tests (chemistry and hematology)
If medically indicated for evaluation of Clinical Deterioration, additional tests were performed at the discretion of the Investigator.
PK, PD sampling at or during crisis or deterioration Visit:
   Collected one blood sample for PK, PD, and free C5 assays if no IP was administered.
   If IP was administered at the MG Crisis evaluation visit or at the visit for Clinical Deterioration, according to the protocol schedule, collected two blood samples, trough and peak, at [1] 5-90 minutes before the IP infusion and [2] at least 60 minutes after completion of the IP infusion.
   If the subject received PE at the time of a crisis or Clinical Deterioration, a supplemental dose of IP will be administered. Collected three blood samples for PK, PD, and free C5 at [1] 5-90 minutes before PE, [2] 60 minutes after PE and before IP infusion, and [3] at least 60 minutes after completion of the IP infusion.
IP administration:
   Subject continued IP administration in accordance with protocol specified IP administration schedule.
   If the crisis or Clinical Deterioration Visit coincided with a regular visit per protocol, subject received the regular scheduled IP administration per protocol schedule.
   If subjects underwent PE, a supplemental dose (2 vials IP) was administered within 60 minutes after each PE session. If the subject was scheduled to receive the protocol-scheduled dose on the day of a PE session, then the scheduled dose was administered within 60 minutes after the end of the PE.

### 1.4.2.4 Unscheduled Visit

Additional (Unscheduled) visits outside the specified visits were permitted at the discretion of the Investigator. Procedures, tests, and assessments were performed at the discretion of the Investigator. If an Unscheduled Visit was performed, any tests, procedures, or assessments performed at the Unscheduled Visits were recorded on the eCRFs.

### 1.4.3. Safety Follow-Up Period (Post-Treatment+Week 4) Safety Follow-Up Period (Post-Treatment+Week 8)

If a subject withdrew from the trial at any time during the Study Period after receiving any amount of IP (eculizumab or placebo) or did not wish to enter the extension trial after completion of this trial, a follow up visit for safety assessment was required at 4 weeks after the last dose of IP. The following tests and procedures were completed at the safety follow-up visit:
Measured vital signs, including assessments of systolic and diastolic BP, temperature, RR, and HR
Recorded any new medications or changes to concomitant medications
Evaluated and record any new AEs or changes in AEs since the previous visit.
Administered MG-QOL15
Administered MG-ADL by a properly trained evaluator, preferably the same evaluator, throughout the trial. The recall period was the preceding 7 days.
Administered clinical assessments QMG, NIF, and MGC; these were performed at approximately the same time of day by a properly trained evaluator, preferably the same evaluator, throughout the trial. If the subject was taking a cholinesterase inhibitor, the dose was withheld for at least 10 hours prior to the QMG and MGC tests.
Assessed change from baseline in MGFA Post-Intervention Status (see Table 6.

If a subject discontinued due to an AE, the AE was followed until it was resolved or, in the opinion of the PI, was determined medically stable.

### 1.5. Number of Subjects

126 subjects with refractory gMG were randomized in a 1:1 (eculizumab: placebo) ratio at approximately 100 centers. Randomization was across centers and was stratified based on MGFA clinical classifications (Class a vs. Class b and Classes II and II vs. Class IV) (see Table 8).

### 1.6. Treatment Assignment

126 subjects with refractory gMG are randomized, 63 subjects to eculizumab and 63 subjects to placebo. All patients remained on assigned double-blind treatment until the EOS/ET visit. Randomized subjects who discontinued after initiation of study treatment were not replaced. Assignment was performed through the IXRS at each visit.

### 2. Selection and Withdrawal of Subjects

### 2.1. Subject Inclusion Criteria

1. Male or female subjects 218 years old
2. Diagnosis of MG must be made by the following tests:
   Positive serologic test for anti-AChR Abs as confirmed at screening, and
   One of the following:
   a. History of abnormal neuromuscular transmission test demonstrated by single-fiber electromyography (SFEMG) or repetitive nerve stimulation, or
   b. History of positive anticholinesterase test, e.g., edrophonium chloride test, or c. Subject has demonstrated improvement in MG signs on oral cholinesterase inhibitors, as assessed by the treating physician.
3. MGFA Clinical Classification Class II to IV at screening.
4. MG-ADL total score must be ≥6 at screening and Randomization (Day 1)
5. Subjects who have
   a. Failed treatment over one year or more with 2 or more ISTs* (either in combination or as mono-therapy), i.e., continue to have impairment ADLs (persistent weakness, experience crisis, or unable to tolerate IST) despite ISTs. Or,
   b. Failed at least one IST and require chronic plasma exchange or IVIg to control symptoms, i.e., subjects who require PE or IVIg on a regular basis for the management of muscle weakness at least every 3 months over last 12 months.
      Immunosuppressant's include, but are not limited to, corticosteroids AZA, MMF, methotrexate (MTX), cyclosporine, tacrolimus, or cyclophosphamide.
6. If subjects who entered the study were receiving AZA they must have been on AZA for ≥6 months and have been on a stable dose for ≥2 months prior to screening.
7. If subjects who entered the study were receiving other ISTs, i.e., MMF, MTX, cyclosporine, tacrolimus, or cyclophosphamide, they must have been on the IST for ≥3 months and have been on a stable dose for 21 month prior to screening.
8. If subjects who entered the study were receiving oral corticosteroids, they must have been on a stable dose for ≥4 weeks (28 days) prior to screening.
9. If subjects who entered the study were receiving a cholinesterase inhibitor they must have been on a stable dose for ≥2 weeks prior to screening.
10. Female subjects of child-bearing potential must have had a negative pregnancy test (serum human chorionic gonadotropin [HCG]). All subjects must have practiced an effective, reliable and medically approved contraceptive regimen during the study and for up to 5 months following discontinuation of treatment.
11. Subject must have been given written informed consent.
12. Subject must have been able and willing to comply with study procedures.

### 2.2. Subject Exclusion Criteria

1. History of thymoma or other neoplasms of thymus.
2. History of thymectomy within 12 months prior to screening.
3. Weakness only affecting ocular or peri-ocular muscles (MGFA Class 1).
4. MG crisis at screening (MGFA Class V)
5. Pregnancy or lactation.
6. Any systemic bacterial or other infection, which is clinically significant in the opinion of the Investigator and has not been treated with appropriate antibiotics
7. Unresolved meningococcal infection.
8. Use of IVIg within 4 weeks prior to Randomization (Day 1).
9. Use of PE within 4 weeks prior to Randomization (Day 1).
10. Use of rituximab within 6 months prior to screening.
11. Participation in any other investigational drug trial or exposure to other investigational agent, device, or procedures within 30 days prior to screening.
12. Subjects who have received previous treatment with eculizumab.
13. Hypersensitivity to murine proteins or to one of the excipients of eculizumab.
14. Any medical condition that, in the opinion of the Investigator, might have interfered with the subject's participation in the study, posed any added risk for the subject, or confounded the assessment of the subjects.

### 2.3. Subject Withdrawal Criteria

2.3.1. Withdrawal of Subjects from the Trial

Subjects were allowed to withdraw consent at any time. Every effort was made to ensure subjects were willing to comply with trial participation prior to conducting the screening procedures and the subjects were fully informed of the restrictions related to the change of concomitant medications during the trial. Investigators may have chosen to discontinue a subject's treatment because of AEs, as well as conditions or illnesses that preclude compliance with the protocol from the standpoint of the subject's safety or well-being. The study staff notified the Sponsor and their site monitor of all trial withdrawals as soon as possible.

Reproduction and development studies with eculizumab have not been performed; therefore, eculizumab should not be administered to pregnant women. At the time of the last follow-up visit, all subjects of childbearing potential continued to use adequate contraception for up to 5 months following discontinuation of eculizumab treatment. If a subject became pregnant, the IP was immediately discontinued and the Sponsor was notified. Each pregnancy was followed to term and the Sponsor notified regarding the outcome.

### 2.3.2. Handling of Withdrawals

When a subject withdrew or was withdrawn from the trial, the Investigator recorded the withdrawal reason(s). Whenever possible, all subjects who prematurely withdrew from the trial underwent all assessments at the ET visit for safety as per the Schedule of Assessments (Table 8). A follow-up visit for safety assessment was required at 8 weeks after the last dose of IP administration (Table 8).

If a subject discontinued due to an AE, the event was followed until it was resolved or in the opinion of the PI the subject is determined to be medically stable. Every effort was made to undertake protocol-specified safety follow-up procedures.

Subjects who failed to return for final assessments were contacted by the site study staffs in an attempt to have them comply with the protocol. As it was vital to obtain follow-up data on any subject withdrawn because of an AE or SAE, follow-up due diligence documentation consisted of 3 phone calls followed by 1 registered letter to the subject's last known address. In any case, every effort was made to undertake protocol-specified safety follow-up procedures.

### 2.3.3. Sponsor's Termination of Trial

Alexion Pharmaceuticals, Inc. or a regulatory authority may have discontinued the trial at any time for any reason including, for example, clinical or administrative reasons.

### 2.3.4. End of Trial Definition

The end of trial is defined as the last visit completed by the last patient.

### 3. Treatment of Subjects

### 3.1. Description of Investigational Product

Eculizumab (600 mg, 900 mg or 1200 mg) or matching placebo was administered intravenously over approximately 35 minutes according to the regimen shown in Table 13.

TABLE 13
TRIAL DOSE REGIMEN

| | | | | Equivalent |
|---|---|---|---|---|
| | Frequency of Investigational | | # of | Eculizumab |
| Dose Period | Product Administration | Visits | Vials | Dose |
| Induction | Weekly (every 7 ± 2 days) | 2-5 | 3 | 900 mg |
| Phase | | 6 | 4 | 1200 mg |
| Maintenance | Every 2 weeks (14 ± 2 days) | 7-17 | 4 | 1200 mg |
| Phase | from the fifth dose onward | | | |
| Supplement | If PE is given due to a Clinical | | 2 | 600 mg |
| Doses* | Deterioration, administer within | | | |
| | 60 minutes after the end of each | | | |
| | PE session as described below*. | | | |

Induction Phase

Eculizumab or placebo: 3 vials of IP (equivalent to 900 mg of eculizumab) weekly for 4 weeks (every 7 days±2 days) followed by 4 vials of IP (equivalent to 1200 mg of eculizumab) one week later for the fifth dose (Visit 6/Week 4).

Maintenance Phase

Eculizumab or placebo: 4 vials of IP (equivalent to 1200 mg of eculizumab) every 2 weeks (14 days±2 days)

Supplemental Doses

If PE was administered due to a Clinical Deterioration (as defined by this protocol), supplemental IP (2 vials, equivalent to 600 mg of eculizumab or matching placebo) was administered within 60 minutes after the end of each PE session. If PE was administered on a day of regularly scheduled IP administration, subjects received the regularly scheduled number of vials (3 vials on Visits 2-4; 4 vials on all other visits) within 60 minutes after each PE session.

### 3.2. Concomitant Medications

### 3.2.1. Allowed Medications

### 3.2.1.1. Palliative and Supportive Care

Palliative and supportive care was permitted during the course of the trial for underlying conditions.

The following medications were allowed under certain circumstances and restrictions.

### 3.2.1.2. Cholinesterase Inhibitors

For subjects who entered the trial receiving a cholinesterase inhibitor for at least two weeks prior to screening, the dose and schedule of their cholinesterase inhibitor was maintained stable throughout the entire Study Period, unless there was compelling medical need. Increases in cholinesterase therapy that were required as a result of inter-current illness or other medical cause of deterioration were permitted but dosing was returned to dosing levels at trial entry as soon as feasible and the trial sponsor was notified of the change.
Cholinesterase inhibitor treatment was withheld for at least 10 hours prior to QMG and MGC tests.
If a decrease in cholinesterase inhibitor was considered based on clinical evaluation, sponsor approval was obtained prior to the change in dose in order for the subject to remain on study. Dose increase as a result of inter-current illness or other medical cause was permitted, but dose was returned to dose level at trial entry as soon as feasible and the trial sponsor was notified.

### 3.2.1.3. Immunosuppressive Agents

The following immunosuppressive agents were allowed during the trial: corticosteroid, AZA, MMF, MTX, tacrolimus, cyclosporine, or cyclophosphamide. The immunosuppressive agent(s) and its appropriate dose level to be used for an individual subject was at the discretion of the treating physician.
Corticosteroid—for subjects who entered the trial receiving oral corticosteroid, e.g., prednisone, the dose/schedule must have been stable for four weeks prior to trial and may not be changed during the entire double-blind Study Period. If a decrease or taper in steroid dose was considered during the Study Period based on clinical evaluation, sponsor approval was obtained prior to the change in order for the subject to remain on trial. If the dose level subsequently was increased, the dose level increase was not be above the dose level reported at the baseline (at the start of randomized treatment).
High-dose steroid was reserved for subjects that experience Clinical Deterioration as defined by this protocol. Every effort was made to notify the Sponsor within 24 hours of administration if a subject required a rescue therapy for Clinical Deterioration.
AZA, MMF, MTX, tacrolimus, cyclosporine or cyclophosphamide—for subjects who entered the trial receiving above mentioned immunosuppressive agents, the dose regimen of the immunosuppressive agent was be changed during the entire double-blind Study Period. If a change in the dose regimen was considered due to known toxicity or side effects associated with the given immunosuppressive agent, sponsor approval was obtained prior to the dose change in order for the subject to remain on the trial. A different immunosuppressive agent was not added or substituted during the 26-week double-blind Study Period.

### 3.2.1.4. Plasma Exchange/Plasmapheresis (PE)/IVIg

Use of PE or IVIg was allowed for subjects who experience a Clinical Deterioration as defined by this protocol. The rescue therapy used for a particular subject was at the discretion of the treating physician. Every effort should was to notify the Sponsor within 24 hours should a subject require a rescue therapy.

If PE was administered as a rescue therapy, supplemental IP (2 vials) were administered within 60 minutes after the end of each PE session. Routine (per protocol schedule) IP administration was continued per the specified dose-administration schedule for the subject. If the subject was scheduled to receive the protocol-scheduled dose on the day of a PE session, then the scheduled dose was administered within 60 minutes after the end of the PE session.

### 3.2.2. Disallowed Medications

The following concurrent medications were prohibited during the trial:
Use of rituximab

### 3.3. Treatment Compliance

The infusion of IP into subjects was under the supervision of the PT/Sub-Investigator or their designee, to ensure that the subject received the appropriate dose at the appropriate time-points during the trial.

Subjects who failed to return for a scheduled visit within the accepted intervals were contacted by the site study staffs to determine the reason for missing the appointment. Instructions for handling of missing visits are provided in Section 1.4.2.

### 3.4. Randomization and Blinding

### 3.4.1. Randomization

Subjects were randomized on Day 1 after the Investigator verified that they are eligible. Subjects were randomized in a 1:1 ratio of eculizumab infusion to placebo infusion. The randomization will be across centers using an IXRS. The randomization stratification will be based on MGFA clinical classification assessed at the Screening Visit according to the following 4 groupings:
a. MGFA Class IIa and IIIa
b. MGFA Class IVa
c. MGFA Class IIb and IIIb, and
d. MGFA Class IVb
   The MGFA clinical classifications are described in Table 9.

### 3.4.2. Blinding and Unblinding

All trial subjects, investigational site personnel, sponsor staff, sponsor designees, and all staff directly associated with the conduct of the trial were blinded to the subject treatment assignments. The double blind is maintained by using identical IP kits and labels for eculizumab and placebo. The placebo had an identical appearance to that of eculizumab. The random code was maintained by Almac Clinical Services. There is no antidote to reverse the effects of eculizumab.

Therefore, unblinding would not be helpful in the planning of patient treatment for a given event. Unblinding was only considered for the safety of the subject. If unblinding was deemed necessary by the Investigator, the Investigator could have unblinded the patient's treatment allocation using IXRS. The Investigator must have noted the date, time and reason for unblinding.

The Investigator should have informed the Medical Monitor that the patient was unblinded, however they are not required to reveal to the Medical Monitor the patients' treatment allocation.

When an AE was an unexpected related serious AE, the blind was broken by the Sponsor only for that specific subject. The blind was maintained for persons responsible for the ongoing conduct of the study (such as the management, monitors, investigators, etc.) and those responsible for data analysis and interpretation of results at the conclusion of the study, such as biometrics personnel. Unblinded information was only accessible to those who need to be involved in the safety reporting to Health Authorities, Ethics Committees and/or IRBs.

Investigators received only blinded information unless unblinded information is judged necessary for safety reasons.

### 4. Investigational Product Materials and Management

### 4.1. Investigational Product

Each vial of IP contains eculizumab 300 mg or matching placebo for IV administration.

### 4.2. Investigational Product Packaging and Labeling

The active IP, eculizumab is manufactured and supplied by Alexion in single 30 mL vials as a solution concentration of 10 mg/ml. The comparator product is manufactured by Alexion Pharmaceuticals, Inc., as a matching sterile, clear, colorless solution with the same buffer components but without active ingredient, in an identical 30 ml vial. See Table 14.

All study medication was prepared in vials, packaged in kits, labeled in an identical manner.

IP vials were individually packaged into a kit. Both vials and kits were labeled according to the protocol and local regulatory requirements. Each kit had a label describing the contents and a place for the pharmacist to record the subject number and initials.

Study medication was shipped and released to each participating trial center upon receipt of all required essential documents based upon federal, state, and local regulations (Table 14).

TABLE 14
INVESTIGATIONAL PRODUCT

| | Investigational | |
|---|---|---|
| Product Name: | Eculizumab | Placebo |
| Dosage Form: | Concentrate for solution for infusion | Solution for infusion |
| Unit Dose: | 300 mg | 0 mg |
| Route of Administration: | Intravenous Infusion | Intravenous Infusion |
| Physical Description: | 30 mL vial | 30 mL vial |
| Manufacturer: | Alexion Pharmaceuticals, Inc. | Alexion Pharmaceuticals, Inc. |

### 4.3. Investigational Product Storage

IP was released to the site upon receipt of all required essential documents based upon federal, state, and local regulations. See Table 14.

Upon arrival at the center, the IP was promptly removed from the shipping cooler and stored in refrigerated conditions at 2 to 8° C. The pharmacist immediately recorded the reception of the IP and notified the distributor if vials were damaged and/or if temperature excursions occurred during transportation. IP was stored in a secure, limited-access storage area and temperature was monitored daily.

Diluted solutions of IP were stored at 2 to 8° C. (36-46° F.) for up to 24 hours prior to administration. If the IP is prepared more than 4 hours in advance of a subject's visit, the diluted material was stored at 2 to 8° C. The solution was allowed to warm to room temperature prior to administration. The material was not heated (e.g., by using a microwave or other heat source) other than by ambient air temperature.

### 4.4. Investigational Product Preparation

Infusions of IP were prepared using aseptic technique. Each vial of IP contained 300 mg of active ingredient in 30 mL of product solution or matching placebo.

The required amount of IP was withdrawn from the vials. The recommended dose was transferred to an infusion bag. The IP was diluted to a final concentration of 5 mg/mL by addition to the infusion bag of the appropriate amount (equal volume) of 0.9% Sodium Chloride Injection, USP; 0.45% Sodium Chloride Injection, USP; 5% Dextrose in Water Injection, USP; or Ringer's Injection, USP. The final volume of a 5 mg/mL diluted IP solution is 120 mL for 600 mg doses (2 vials), 180 mL for 900 mg doses (3 vials) and 240 mL for 1200 mg doses (4 vials) as shown in Table 15.

TABLE 15
INVESTIGATIONAL PRODUCT RECONSTRUCTION

| | | Volume | Total Volume of |
|---|---|---|---|
| Investigational Product | Volume of IP | of Diluent ^{a} | Administration |
| 600 mg (2 vials) | 60 mL | 60 mL | 120 |
| 900 mg (3 vials) | 90 mL | 90 mL | 180 |
| 1200 mg (4 vials) | 120 mL | 120 mL | 240 |

^{a} Choose one of the following diluents: a. 0.9% sodium chloride; b. 0.45% sodium chloride; c. 5% dextrose in water; d. Ringer's injection

The infusion bag containing the diluted IP solution was gently inverted to ensure thorough mixing of the product and diluents. Any unused portion left in a vial was discarded, as the product contains no preservatives. The diluted solution was allowed to warm to room temperature by exposure to ambient air prior to administration.

### 4.5. Administration

Do not Administer as an IV Push or Bolus Injection

IP was only administered via IV infusion and was diluted to a final concentration of 5 mg/mL prior to administration. Prior to administration, if the diluted solution was refrigerated, it was allowed to warm to room temperature by exposure to ambient air. The diluted solution was not heated in a microwave or with any heat source other than ambient air temperature. Parenteral drug products were inspected visually for particulate matter and discoloration prior to administration.

The diluted IP was intravenously administered over 35 minutes (range 25 to 45 minutes). It was not necessary to protect the infusion bags from light while IP was being administered to the subject. At the site's discretion, the diluted IP was administered via gravity feed, a syringe-type pump, or an infusion pump. The subjects were monitored for 1 hour following infusion.

If an AE occurred during the administration of the IP, the infusion was slowed or stopped at the discretion of the Investigator, depending upon the nature and severity of the event. The overall time of infusion did not exceed 2 hours. The AE must be captured in the subject's source document and CRF.

### 5. Assessment of Efficacy

Duration of treatment commenced with the first infusion of IP (eculizumab or placebo). The 26-week Study Period defined the time period for assessment of the study endpoints (specified in Table 8, the schedule of assessments). Efficacy was assessed comparing eculizumab outcomes to placebo outcomes. Statistical analyses of the efficacy endpoints are summarized below and described in more detail in the statistical analysis plan. For all scales noted below except the EQ Visual Analog Scale (VAS) and Myasthenia Gravis Foundation of America (MGFA) Post-Intervention Status (PIS) the higher the score the greater the impairment.

### 5.1. MG Activities of Daily Living Profile (MG-ADL)

The MG-ADL is an 8-point questionnaire that focuses on relevant symptoms and functional performance of activities of daily living (ADL) in MG subjects (see Table 1). The 8 items of the MG-ADL were derived from symptom-based components of the original 13-item QMG to assess disability secondary to ocular (2 items), bulbar (3 items), respiratory (1 item), and gross motor or limb (2 items) impairment related to effects from MG. In this functional status instrument, each response is graded 0 (normal) to 3 (most severe). The range of total MG-ADL score is 0-24. A clinically meaningful improvement in a patient's MG-ADL would be a 3 point or greater reduction in score after 26 weeks of treatment. The recall period for MG-ADL is the preceding 7 days. MG-ADL was performed at Screening, Day 1, Weeks 1-4, 8, 10, 12, 16, 20, and 26 or ET (Visits 2-6, 8, 10, 12, 14, and 17, or ET) by a properly trained evaluator, preferably the same evaluator throughout the study.

### 5.2. QMG Scoring System

The current QMG scoring system consists of 13 items: ocular (2 items), facial (1 item), bulbar (2 items), gross motor (6 items), axial (1 item) and respiratory (1 item); each graded 0 to 3, with 3 being the most severe (see Table 2). The range of total QMG score is 0-39. The QMG scoring system is considered to be an objective evaluation of therapy for MG and is based on quantitative testing of sentinel muscle groups. The MGFA task force has recommended that the QMG score be used in prospective studies of therapy for MG(15). A clinically meaningful improvement in a patient's QMG would be a 4 point or greater reduction in score after 26 weeks of treatment. The QMG was administered at Screening, Day 1, Weeks 1-4, 8, 12, 16, 20, and 26 or ET (Visits 1-6, 8, 10, 12, 14, and 17 or ET).

5.3. MGC Score

The MGC is a validated assessment tool for measuring clinical status of subjects with MG (16). The MGC assesses 10 important functional areas most frequently affected by MG and the scales are weighted for clinical significance that incorporate subject-reported outcomes (see Table 3). A clinically meaningful improvement in a patient's MGC would be a 3 point or greater reduction in score after 26 weeks of treatment. MGC was administered at Screening, Day 1, Weeks 1-4, 8, 12, 16, 20, and 26 or ET (Visits 1-6, 8, 10, 12, 14, and 17 or ET).

### 5.4. Quality of Life Assessments

### 5.4.1. MG-QOL 15

The 15-item Myasthenia Gravis Qualify of Life scale (MG-QOL 15) (see FIG. 1) is a health-related quality of life evaluative instrument specific to subjects with MG. MG-QOL15 was designed to provide information about subjects' perception of impairment and disability and the degree to which disease manifestations are tolerated and to be easy to administer and interpret (17). The MG-QOL 15 is completed by the subject. Higher scores indicate greater extent of and dissatisfaction with MG-related dysfunction. A clinically meaningful improvement in a patient's MG-QOL 15 would be an increase in score after 26 weeks of treatment. The MG-QOL 15 was administered at Screening, Day 1, Weeks 4, 8, 12, 16, 20, and 26 or ET (Visits 1-2, 6, 8, 10, 12, 14, and 17 or ET).

### 5.4.2. Neuro-QOL Fatigue

The Neuro-QOL Fatigue is a reliable and validated brief 19-item survey of fatigue, completed by the subject (18). Higher scores indicate greater fatigue and greater impact of MG on activities (see Table 5). A clinically meaningful improvement in a patient's Neuro-QOL Fatigue score would be reflected in a decrease in score after 26 weeks of treatment. The Neuro-QOL Fatigue was administered at Day 1, Weeks 4, 8, 12, 16, 20, and 26 or ET (Visits 2, 6, 8, 10, 12, 14, and 17 or ET).

### 5.4.3. EUROQOL (EQ-SD)

The EUROQOL (EQ-5D) is a reliable and validated survey of health status in 5 areas: mobility, self-care, usual activities, pain/discomfort, and anxiety/depression, completed by the subject (19). Each area has 3 levels: level 1 (no problems), level 2 (some problems), and level 3 (extreme problems) (see FIGS. 2A and 2B). The EQ VAS records the subject's self-rated health on a vertical, 20 cm visual analogue scale where the endpoints are labeled “Best imaginable health state, marked as 100” and “Worst imaginable health state, marked as 0.” A clinically meaningful improvement in a patient's EQ-5D would be reflected as an increase in score after 26 weeks of treatment. The EQ-5D was administered at Day 1, Weeks 4, 8, 12, 16, 20, and 26 or ET (Visits 2, 6, 8, 10, 12, 14, and 17 or ET).

### 5.5. Other Efficacy Assessments

### 5.5.1. Negative Inspiratory Force NIF and Forced Vital Capacity

Subjects with increasingly severe MG can suffer from potentially fatal respiratory complications including profound respiratory muscle weakness. Respiratory function is monitored closely for evidence of respiratory failure in MG subjects and ventilator support is recommended in the event of consistent declines in serial measurements of Forced Vital Capacity (FVC) or Negative Inspiratory Force (NIF), loss of upper airway integrity (difficulty handling oral secretions, swallowing, or speaking) or in the setting of emerging respiratory failure. FVC as one of the test items in QMG is performed when QMG is performed. NIF was to be performed using the NIF Meter. It was measured at Screening, Day 1, Weeks 1-4, 8, 12, 16, 20, and 26 or ET (Visits 1-6, 8, 10, 12, 14, and 17 or ET).

### 5.5.2. MGFA Post-Intervention Status

The MG clinical state is assessed using the MGFA Post-Intervention Status. See Table 6. Change in status categories of Improved, Unchanged, Worse, Exacerbation and Died of MG as well as the Minimal manifestation (MM) was assessed and recorded at Weeks 4, 12 and 26 or ET (Visits 6, 10 and 17 or ET) by the PI or the same neurologist skilled in the evaluation of MG subjects throughout the trial. The sub-scores of MM, i.e., MM-0, MM-1, and MM-3, will not be used in this protocol.

### 6.2. Determination of Sample Size

The study design was a randomized, double blind, placebo-controlled design. Subjects were randomly assigned 1:1 to eculizumab or placebo. The randomization stratification variable was based on MG clinical classification by the Myasthenia Gravis Foundation of America (MGFA) according to the following 4 groupings (Class IIa and IIIa, Class IVa, Class IIb and IIIb and Class IVb).

The sample size and power calculation assumptions are as follows:
1:1 randomization (eculizumab: placebo)
Power 90% for both the primary and the first secondary endpoints
Two-sided 5% level of significance
Drop-out rate 15%
Mean changes from baseline for MG-ADL was assumed to be 4 for eculizumab and 1.5 for placebo with a standard deviation of 3.25, mean changes in QMG total score of 7 for eculizumab and 3 for placebo with a standard deviation of 6, and mean ranked differences between the treatment groups is assumed to be 3 with a standard deviation of 4 for both endpoints. Sample size calculations based on t-test.

With these assumptions, a sample size of approximately 92 subjects (46 eculizumab and 46 placebos) provides 90% power to detect a treatment difference at 26 weeks.

### 6.3. Analyses Sets

Analyses were produced for the double-blind Study Period in order to compare the eculizumab group with placebo group. The analyses include efficacy, safety, and PK/PD analyses.

### 6.3.1. Full Analysis Set

The full analysis set (FAS) was the population on which primary, secondary, and tertiary efficacy analyses is performed and consists of all subjects who are randomized to IP and who have received at least 1 dose of IP (eculizumab or placebo treatment) and have at least one efficacy assessment post IP infusion. Subjects were compared for efficacy according to the treatment they were randomized to receive, irrespective of the treatment they actually received.

### 6.3.2. Per-Protocol Set

The Per-Protocol (PP) Set is a subset of the Full Analysis Set (FAS) population, excluding subjects with major protocol deviations. The possible categories of major protocol deviations are defined in the statistical analysis plan. The per-protocol population included all subjects who:
Had no major protocol deviations or inclusion/exclusion criteria deviations that might potentially affect efficacy,
Subjects who took at least 80% of the required doses and remained enrolled in the trial for 26 weeks or subjects who took at least 80% of the required doses up to the time of being discontinued for Clinical Deterioration (e.g., MG crisis/exacerbation).
   The PP population will be fully described in the statistical analysis plan, and subjects identified prior to database lock. Efficacy analyses will also be performed on the PP data set.

### 6.7. Efficacy Analyses

Note: During the Study Period, Baseline is defined as the last available assessment prior to treatment for all subjects, regardless of treatment group.

### 6.7.1. Primary Efficacy Endpoint

The primary efficacy endpoint was change from baseline in the MG-ADL total score at Week 26 of the Study Period. The primary efficacy analysis was conducted on the available 26 week data from the Study Period for all subjects. The trial was considered to have met its primary efficacy objective if a statistically significant difference (p≤0.05) between the eculizumab treatment group and placebo group was observed for change from baseline in the MG-ADL total score at Week 26. Confidence intervals and p-values are presented. For the primary analysis concerning the change from baseline in the MG-ADL total score at Week 26, treatment groups were compared using a worst-rank score analysis (i.e., analysis of covariance [ANCOVA] analysis with ranks) with effects for treatment. The baseline MG-ADL total score and the randomization stratification variables are also covariates in the model. In this analysis, the actual changes from baseline are ranked from highest (best improvement in MG-ADL score) to lowest (least improvement/most worsening in MG-ADL score) across all subjects who did not need rescue therapy. Then, any subject who needed rescue therapy is given lower ranks. These lower ranks are based on the time to rescue therapy from the start of investigational product (Day 1). The subject with the shortest time to rescue therapy would get the absolute lowest rank in the analysis and the subject with the longest time to rescue therapy would get a rank that is one lower than the lowest ranked subject without rescue therapy. Last observation carried-forward is used for missing changes from baseline at Week 26 for patients with missing Week 26 who did not require rescue therapy.

A sensitivity analysis for the actual change from baseline in the MG-ADL total score at Week 26 was also performed. Treatment groups are compared using ANCOVA analysis using the actual change from baseline in the MG-ADL total score at Week 26 with effects for treatment. The baseline MG-ADL total score and the randomization stratification variable were also covariates in the model. Last observation carried-forward is used for missing changes from baseline at Week 26.

A sensitivity analysis for the actual change from baseline in the MG-ADL total score at Week 26 was also performed. In the sensitivity analysis, treatment groups were compared using repeated measures model with effects for treatment and visits. The baseline MG-ADL total score, the randomization stratification variable, and an indicator for the IST treatment status of the subject were also covariates in the model. Subjects have an IST treatment status variable defined based on the IST treatments the subject receives.

In addition, summaries of changes from baseline in the MG-ADL total score at Week 26 were produced by treatment group for subjects who have failed ISTs.
Subjects who have failed treatment over one year or more with 2 or more ISTs in sequence or in combination.
Subjects who have failed at least one IST and require chronic plasma exchange or IVIg to control symptoms.

### 6.7.2. Secondary Efficacy Analysis

Unless otherwise specified, the secondary efficacy analyses use the available 26-week data from the Study Period. Hypothesis testing comparing eculizumab treatment with placebo treatment for the secondary efficacy analyses were performed using a closed testing procedure with the following rank order:
1. Change from baseline in QMG total score at Week 26
2. Proportion of subjects with at least a 3-point reduction in the MG-ADL total score from baseline to Week 26 and with no rescue therapy
3. Proportion of subjects with at least a 5-point reduction in the QMG total score from baseline to Week 26 and with no rescue therapy
4. Change from baseline in the MGC score at Week 26
5. Change from baseline in MG-QOL15 at Week 26

The hypothesis testing will proceed from highest rank (#1) Change from baseline in QMG total score at Week 26 to (#5) Change from baseline in MG-QOL-15, and if statistical significance is not achieved at an endpoint (p≤0.05), then endpoints of lower rank were not considered to be statistically significant. Confidence intervals and p-values were presented for all secondary efficacy endpoints for descriptive purposes, regardless of the outcome of the closed testing procedure.

The secondary endpoints that involve changes from baseline were analyzed using a worst-case ranked analysis of covariance (ANCOVA) like that described for the primary efficacy endpoints as the primary analysis for the particular secondary endpoint. The ranked ANCOVA will have effects for treatment, the baseline for the particular endpoint, and the randomization stratification variable.

A sensitivity analysis for the change from baseline in QMG at Week 26 is analyzed using repeated measures model with effects for treatment, visits, and baseline QMG score in order to compare treatment groups. The randomization stratification variable was also a covariate in the model. A sensitivity analysis for the actual change from baseline in QMG score at Week 26 was performed. Treatment groups were compared using ANCOVA analysis using the actual change from baseline in the QMG score at Week 26 with effects for treatment. The baseline QMG score and the randomization stratification variable are also covariates in the model. Last observation carried-forward will be used for missing changes from baseline at Week 26.

A sensitivity analysis for the change from baseline in MGC at Week 26 was analyzed using repeated measures model with effects for treatment, visits, and baseline MGC score in order to compare treatment groups. The randomization stratification variable is also a covariate in the model.

A sensitivity analysis for the change from baseline in MG-QOL-15 at Week 26 was analyzed using repeated measures model with effects for treatment, visits, and baseline MG-QOL-15 score in order to compare treatment groups. The randomization stratification variable was also a covariate in the model.

The proportion of subjects with at least a 3 point reduction in the MG-ADL total score from baseline to Week 26 with no rescue therapy are analyzed by the Cochran-Mantel-Haenszel test stratified by randomization stratification variable in order to compare eculizumab versus placebo.

The proportion of subjects with at least a 5 point reduction in the QMG total score from baseline to Week 26 with no rescue therapy was analyzed by the Cochran-Mantel-Haenszel test stratified by randomization stratification variable in order to compare eculizumab versus placebo.

Additional sensitivity analyses were performed in order to assess the impact of IST treatment status on the various secondary endpoints. A sensitivity analysis for the change from baseline in the secondary endpoints (i.e., QMG, MGC, and MG-QOL-15) at Week 26 are analyzed using repeated measures model with effects for treatment, visits, and baseline score in order to compare treatment groups. The randomization stratification variable and an indicator for the IST treatment status of the subject are also covariates in the model. Subjects will have an IST treatment status variable defined based on the IST treatments the subject receives.

In addition, summaries of changes from baseline in QMG, MGC, and MG-QOL-15 at Week 26 are produced by treatment group for subjects who have failed ISTs.
Subjects who have failed treatment over one year or more with 2 or more ISTs in sequence or in combination
Subjects who have failed at least one IST and require chronic plasma exchange or IVIg to control symptoms.

### 6.7.3. Tertiary Efficacy Endpoints

The tertiary efficacy analyses for the Study Period included the following:
1. Time to response as measured by the reduction in the MG-ADL total score (3-point reduction from baseline)
2. Change from baseline in Neuro-QOL Fatigue at Week 26
3. Change from baseline in EQ-5D at 26 weeks
4. Change from baseline in NIF at Week 26 in subjects with abnormal NIF at baseline
5. Change from baseline in FVC at Week 26 in subjects with abnormal FVC at baseline
6. Change from baseline in the MG-ADL individual items and change from baseline in the MG-ADL sub-categories for the bulbar (items 1, 2 and 3), respiratory (item 4), limb (items 5 and 6) and ocular (items 7 and 8) at Week 26 in subjects with an abnormal baseline score for the particular item or sub-category
7. Change from baseline in the MGFA Post-Intervention Status at Week 26.

For the time to response on the MG-ADL total score (3-point reduction in MG-ADL from baseline), treatment groups were compared using Cox PH regression with robust variance estimation. The randomization stratification variable was also a covariate in the model. Inference was based on the Wald test of the log hazard ratio.

Quality of life is summarized as appropriate to the quality of life instrument and treatment group comparisons is performed as specified in the statistical analysis plan (SAP).

The tertiary endpoints that involved changes from baseline are analyzed using a worst-case ranked ANCOVA like that described for the primary efficacy endpoints as the primary analysis for the particular tertiary endpoint. The ranked ANCOVA has effects for treatment, the baseline for the particular endpoint, and the randomization stratification variable.

A sensitivity analysis for the change from baseline in NIF at Week 26 for subjects with abnormal NIF at baseline was analyzed using repeated measures model with effects for treatment, visits, and baseline NIF in order to compare treatment groups. The randomization stratification variables were also covariates in the model.

A sensitivity analysis for the change from baseline in FVC was analyzed using repeated measures model with effects for treatment, visits, and baseline FVC in order to compare treatment groups. The randomization stratification variable was also a covariate in the model.

A sensitivity analysis for the change from baseline in the MG-ADL individual items and sub-categories at Week 26 in subjects that are abnormal at baseline were analyzed using repeated measures model with effects for treatment, visits, and baseline MG-ADL individual item and sub-categories, as applicable for the analysis, in order to compare treatment groups. The randomization stratification variable is also a covariate in the model. In addition, for all full analysis set (FAS) and all PP subjects, a sensitivity analysis for the change from baseline in the MG-ADL individual items and sub-categories at Week 26 are performed using repeated measures model with effects for treatment, visits, and baseline MG-ADL individual item or sub-categories score, as applicable for the analysis, in order to compare treatment groups. The randomization stratification variable is also a covariate in the model. Finally, similar sensitivity analyses and/or summaries were produced (depending on the number of subjects) in the subset of subjects who were normal at baseline and became abnormal after baseline in the MG-ADL individual items and sub-categories.

A summary of subjects going from normal to abnormal for NIF and FVC are presented. A summary of subjects going from normal to abnormal for a particular MG-ADL individual items and sub-categories was produced.

### 6.11. Other Statistical Issues

### 6.11.1. Significance Levels

For all analyses, the eculizumab treated group was compared to the placebo group and all hypothesis testing is two-sided and performed at the 0.05 level of significance, unless otherwise specified. Estimates of treatment effect on efficacy parameters are accompanied by two-sided 95% confidence intervals for the effect size.

6.11.2. Missing or invalid Data

For efficacy and safety analyses, missing post-baseline efficacy and safety data were not imputed unless indicated in the described analysis in the SAP.

### 6.11.3. Interim Analysis

There is no interim analysis planned for this trial.

### Example 2: ECU-MG-302 Extension (302) Trial

An extension trial is described herein that was run to evaluate the long-term safety of eculizumab in subjects with refractory gMG. Other secondary objectives included:
Evaluation of the long-term efficacy as measured by MG-ADL
Evaluation of long-term efficacy by additional efficacy measures including:
   QMG, MGC,
   MG-ADL individual items and subcategories
   Quality of life
Description of the PK and PD of eculizumab in refractory gMG patients.

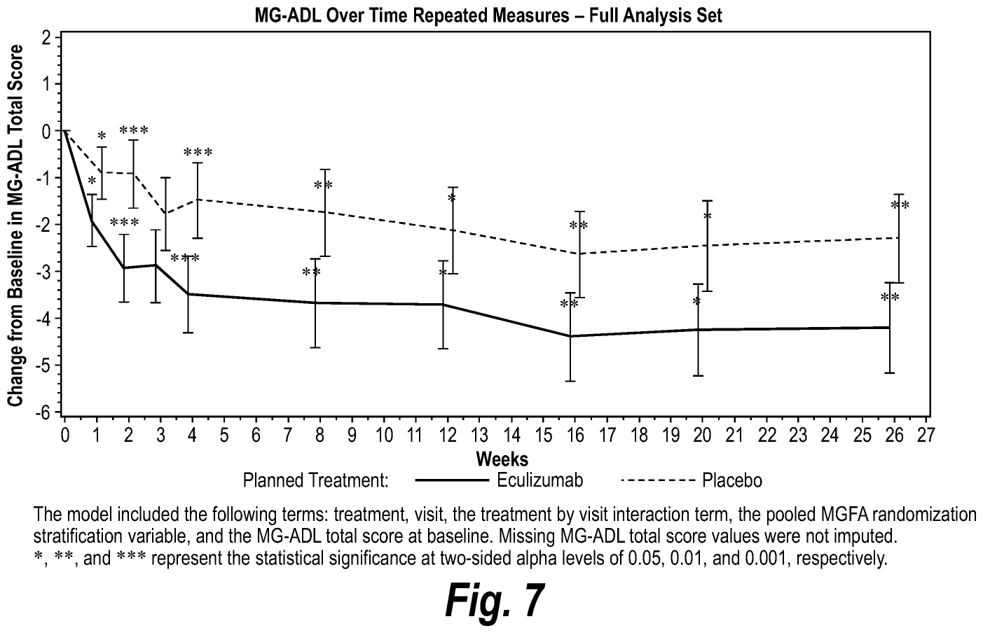

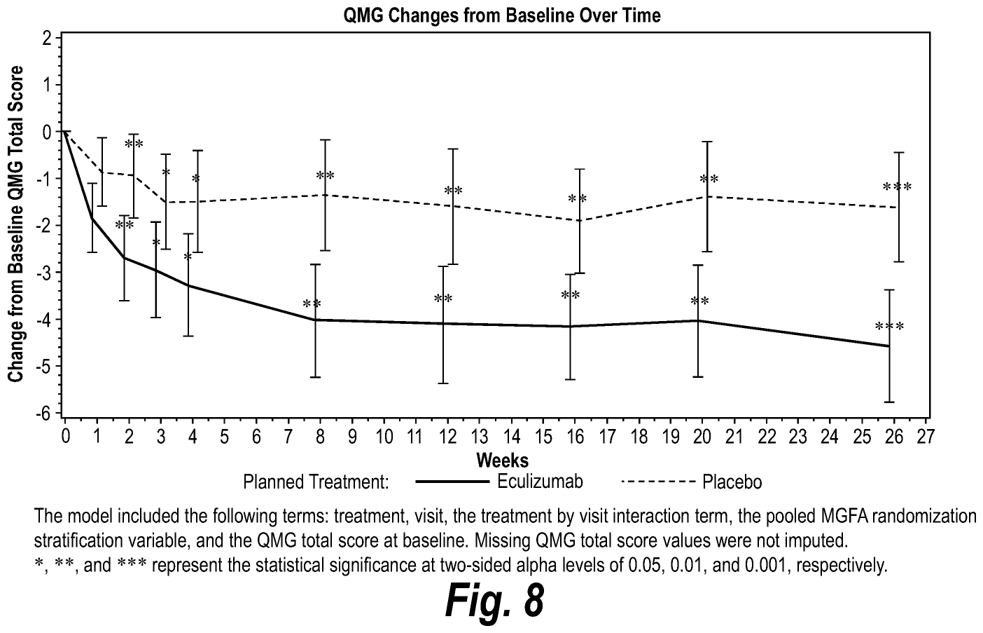

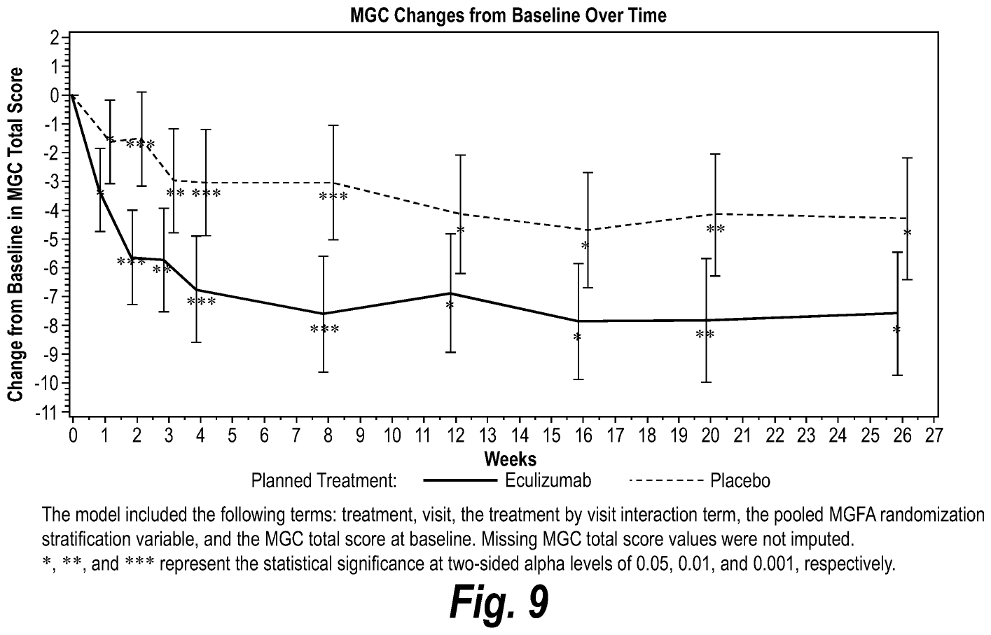

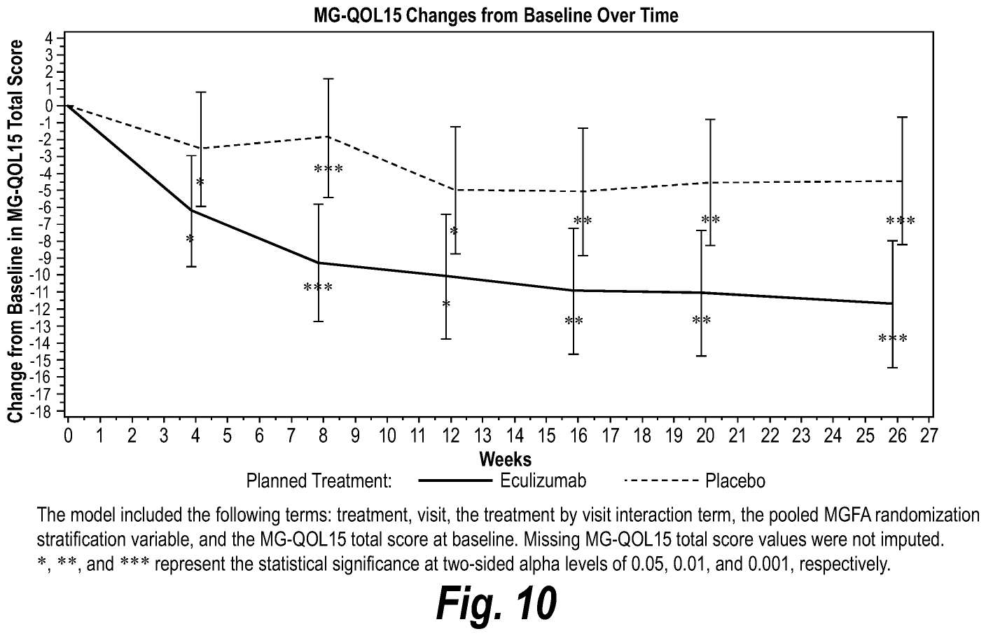

The extension trial lasted for 4 years (FPFV to LPLV). The first visit occurred within 2 weeks of Visit 17 (Week 26) in the trial described above. To maintain the blind of the previous trial, all subjects underwent a blind induction phase, followed by an open label maintenance phase. This is summarized in FIGS. 6 and 7. “Home infusion” at selected visits was performed with permission of the primary investigator in accordance with regulations. Assessments, Treatment, Concomitant/Prohibited medications were performed as in the study described above.

The inclusion criteria for the extension trial was completion of the previous trial. Exclusion criteria were withdrawing from the previous trial and pregnancy or intention to get pregnant. IST treatment could be changed at the treating physician's discretion but rituximab was prohibited.

Efficacy was measured by MG-ADL, QMG, MGC, NIF, FVC, QOL, G-QOL15, Neuro-QOL Fatigue, EQ-5D and MGFA Post-Intervention Status.

### Example 3: Results from REGAIN Study Comprising ECU-MG-301 26 Week (301) Trial and ECU-MG-302 Extension (302) Trial

The REGAIN study is a randomized, double-blind, placebo-controlled, multicenter trial evaluating the safety and efficacy of eculizumab in patients with refractory gMG. The study enrolled and treated 125 adult patients across North America, South America, Europe, and Asia. Patients had a confirmed MG diagnosis with positive serologic test for anti-AChR antibodies. All patients had previously failed treatment with at least two immunosuppressive agents or failed treatment with at least one immunosuppressive agent and required chronic plasma exchange or IVIg, and had an MG-ADL total score ≥6 at study entry.

As discussed above the patients were initially randomized according to MGFA Clinical Classification shown in Table 9 into the following four groups:
MGFA IIa/IIIa
MGFA IIb/IIIB
MGFA IVa
MGFA IVb

The breakdown of the MGFA classification at screening was as follows: Class IIa 25 total patients; Class IIb 22 total patients; Class IIIa 36 total patients; Class IIIb 30 total patients; Class IVa 6 total patients; and Class IVb 6 total patients.

The patients were assigned to the placebo group as follows: Class IIa 15 (23.8%) total patients; Class IIb 14 (22.2%) total patients; Class IIIa 16 (25.4%) total patients; Class IIIb 13 (20.6%) total patients; Class IVa 2 (3.2%) total patients; and Class IVb 3 (4.8%) total patients.

The patients were assigned to the eculizumab group were as follows: Class IIa 10 (16.1%) total patients; Class IIb 8 (12.9%) total patients; Class IIIa 20 (32.3%) total patients; Class IIIb 17 (27.4%) total patients; Class IVa 4 (6.5%) total patients; and Class IVb 3 (4.8%) total patients.

The disposition of patients completing the 301 trial and entering the 302 trial is shown below in Table 16.

TABLE 16
PATIENT DISPOSITION IN THE 301 AND 302 TRIALS

| | Placebo | Eculizumab | Total |
|---|---|---|---|
| Status | n (%) | n (%) | n (%) |
| Randomized | 63 (100.0) | 63 (100.0) | 126 (100.0) |
| Treated | 63 (100.0) | 62 (98.4) | 125 (99.2) |
| Completed the Study | 61 (96.8) | 57 (90.5) | 118 (93.7) |
| Discontinued | 2 (3.2) | 6 (9.5) | 8 (6.3) |
| Adverse Event | 0 (0.0) | 4 (6.3) | 4 (3.2) |
| Death | 0 (0.0) | 0 (0.0) | 0 (0.0) |
| Withdrawal by | 2 (3.2) | 1 (1.6) | 3 (2.4) |
| Subject | | | |
| Other | 0 (0.0) | 1 (1.6) | 1 (0.8) |
| Enrolled in Open- | 61 (96.8) | 56 (88.9) | 117 (92.9) |
| Label | | | |
| Extension Study | | | |

Therefore 96.8% of the placebo patients and 88.9% of the eculizumab patients proceeded into the extension trial.

The demographics of the 301 trial participants were as is shown below in Table 17.

TABLE 17
DEMOGRAPHICS OF 301 CLINICAL TRIAL PARTICIPANTS

| | | Placebo | Eculizumab | Total |
|---|---|---|---|---|
| Variable | Statistic | (N = 63) | (N = 62) | (N = 125) |
| Age at First IP | n | 63 | 62 | 125 |
| Dose (years) (1) | Mean | 46.9 (17.98) | 47.5 (15.66) | 47.2 (16.80) |
| | (SD) | | | |
| | Median | 48.0 | 44.5 | 46.0 |
| | Min, Max | 19.79 | 19.74 | 19.79 |
| Sex | | | | |
| Male | n (%) | 22 (34.9) | 21 (33.9) | 43 (34.4) |
| Female | n (%) | 41 (65.1) | 41 (66.1) | 82 (65.6) |
| Race | | | | |
| Asian | n (%) | 16 (25.4) | 3 (4.8) | 19 (15.2) |
| Black or | n (%) | 3 (4.8) | 0 (0.0) | 3 (2.4) |
| African American | | | | |
| White | n (%) | 42 (66.7) | 53 (85.5) | 95 (76.0) |
| Other | n (%) | 2 (3.2) | 6 (9.7) | 8 (6.4) |
| Is the subject of | | | | |
| Japanese descent? | | | | |
| Yes | n (%) | 9 (14.3) | 3 (4.8) | 12 (9.6) |
| No | n (%) | 54 (85.7) | 59 (95.2) | 113 (90.4) |

The protocol defines clinical deterioration as a subject who has one of the following:

1. MG Crisis

2. Significant symptomatic worsening, defined as worsening on any one of the MG-ADL individual items excluding ocular (i.e., talking, chewing, swallowing, breathing, upper and lower extremity weakness):
To Grade 3, or
2-point worsening in MG-ADL

3. The treating physician believes that the subject's health is in jeopardy if rescue therapy is not administered.

Rescue therapy is defined in the protocol as follows: Use of PE or IVIg will be allowed for subjects who experience a Clinical Deterioration as defined by this protocol. The rescue therapy used for a particular subject is at the discretion of the treating physician

If PE is administered as a rescue therapy, supplemental IP (2 vials) are administered within 60 minutes after the end of each PE session. Routine (per protocol schedule) IP administration is continued per the specified dose-administration schedule for the subject. If the subject is scheduled to receive the protocol-scheduled dose on the day of a PE session, then the scheduled dose is administered within 60 minutes after the end of the PE session.

The total numbers of patients who experienced clinical deterioration during the protocol were as is shown below in Table 18.

TABLE 18
CLINICAL DETERIORATION DURING THE 301

| | | Placebo | Eculizumab |
|---|---|---|---|
| Variable | Statistic | (N = 63) | (N = 62) |
| Total Number of Subjects Reporting Clinical Deterioration | n (%) | 15 (23.8) | 6 (9.7) |
| Total Number of Subjects Experiencing Clinical Deterioration Per Protocol | n (%) | 11 (17.5) | 6 (9.7) |
| Criteria | | | |
| Total Number of Subjects Experiencing the Following Events: | | | |
| MG Crisis | n (%) | 0 (0.0) | 1 (1.6) |
| Significant symptomatic worsen | n (%) | 9 (14.3) | 4 (6.5) |
| Subjects health is in jeopardy | n (%) | 3 (4.8) | 2 (3.2) |
| Other | n (%) | 4 (6.3) | 0 (0.0) |
| Total Number of Clinical Deterioration Events: | n | 27 | 13 |
| MG Crisis | n | 0 | 1 |
| Significant symptomatic worsening | n | 14 | 4 |
| Subjects health is in jeopardy | n | 7 | 8 |
| Other | n | 6 | 0 |

The clinical deteriorations requiring rescue therapy are shown in Table 19 below:

TABLE 19
CLINICAL DETERIORATION REQUIRING RESCUE THERAPIES
DURING THE 301

| | | Placebo | Eculizumab |
|---|---|---|---|
| Variable | Statistic | (N = 63) | (N = 62) |
| Total Number of Subjects Requiring Rescue Therapy: | n (%) | 12 (19.0) | 6 (97) |
| Total Number of Subjects Requiring High Dose Corticosteroids | n (%) | 5 (7.9) | 0 (0.0) |
| Total Number of Subjects Requiring Plasmapheresis/Plasma Exchange | n (%) | 4 (6.3) | 3 (4.8) |
| Total Number of Subjects Requiring IVIg | n (%) | 6 (9.5) | 4 (6.5) |
| Total Number of Subjects Requiring Other Rescue Therapy | n (%) | 2 (3.2) | 1 (1.6) |
| Total Number of Clinical Deterioration Events Requiring Rescue Therapy: | n | 24 | 13 |
| Total Number of Clinical Deterioration Events Requiring High Dose Corticosteriods | n | 8 | 0 |
| Total Number of Clinical Deterioration Events Requiring Plasmapheresis/Plasma | n | 10 | 4 |
| Exchange | | | |
| Total Number of Clinical Deterioration Events Requiring IVIg | n | 13 | 10 |
| Total Number of Clinical Deterioration Events Requiring Other Rescue Therapy | n | 2 | 2 |

The primary and secondary endpoints as described above were used as shown below:
Primary Endpoint:
   Change from baseline in MG-ADL Total Score at Week 26
Secondary Endpoints (hierarchal):
   Change from baseline in QMG Total Score at Week 26
   Proportion of subjects with ≥3-point reduction in MG-ADL Total Score from baseline to Week 26 and without rescue therapy
   Proportion of subjects with ≥5-point reduction in QMG Total Score from baseline to Week 26 and without rescue therapy
   Change from baseline in the Myasthenia Gravis Composite (MGC) Total Score at Week 26
   Change from baseline in MG-QoL15 at Week 26

One primary endpoint in the MG-ADL score at week 26. The score ranges from 0-24 and contains 3 bulbar items, 1 respiratory item, 2 gross motor or limb items, and 2 ocular items. A clinically meaningful improvement in MG-ADL is defined as a 3 points or greater reduction. See Table 1.

The results from the patients who finished the entire protocol are shown in Table 20. Therefore, as shown in Table 22 the median value for the eculizumab group showed a −4 reduction in MG-ADL. This result demonstrates eculizumab produced a clinically meaningful improvement in MG patients as measured by their MG-ADL score.

TABLE 20
MG-ADL WORST RANK ANALYSIS: SAP3 PER PROTOCOL SET

| | | | | Difference in LS | |
|---|---|---|---|---|---|
| | | Placebo | Eculizumab | Means and 95% | |
| Variable | Statistic | (N = 56) | (N = 54) | Cl | p-value |
| Worst Ranked Change from Baseline | Ranked Score LS Mean (SEM) | 61.3 (4.10) | 48.4 (4.20) | −12.8 | 0.0305 |
| | 95% Cl for LS Mean | (53.15, 69.39) | (40.11, 56.74) | (−24.46, −1.24) | |
| Baseline MG-ADL Total Score for patients not | n | 48 | 49 | | |
| needing rescue therapy or dropping out of the study | | | | | |
| | Mean (SD) | 9.8 (2.70) | 10.1 (3.07) | | |
| | Median | 9.0 | 10.0 | | |
| | Min, Max | 6, 18 | 6, 18 | | |
| Week 26 MG-ADL Total Score (LOCF) for patients | n | 48 | 49 | | |
| not needing rescue therapy or dropping out of the | | | | | |
| study | | | | | |
| | Mean (SD) | 7.0 (3.37) | 5.5 (4.04) | | |
| | Median | 6.0 | 5.0 | | |
| | Min, Max | 2, 16 | 0, 15 | | |
| Change from Baseline to Week 26 in MG-ADL Total | n | 48 | 49 | | |
| Score for patients not needing rescue therapy or | | | | | |
| dropping out of the study | | | | | |
| | Mean (SD) | −2.8 (3.05) | −4.7 (4.35) | | |
| | Median | −2.0 | −4.0 | | |
| | Min, Max | −8, 7 | −15, 4 | | |

The data were analyzed in multiple ways for statistical purposes as shown in Tables 20, 21, 22, and 23, but in each case the eculizumab group produced clinically meaningful improvement in MG-ADL and the placebo group failed to produce clinically meaningful improvement in MG-ADL. See Tables 20-23.

TABLE 21
MG-ADL WORST RANK ANALYSIS: SAP3 FULL ANALYSIS SET

| | | Placebo | Eculizumab | Difference in LS | |
|---|---|---|---|---|---|
| Variable | Statistic | (N = 63) | (N = 62) | Means and 95% Cl | p-value |
| Worst Ranked Change from Baseline | Ranked Score LS Mean (SEM) | 68.3 (4.49) | 56.6 (4.53) | −11.7 | 0.0698 |
| | 95% Cl for LS Mean | (59.43, 77.20) | (47.66, 65.61) | (−24.33, 0.96) | |
| Baseline MG-ADL Total Score for patients not needing | n | 51 | 52 | | |
| rescue therapy or dropping out of the study | | | | | |
| | Mean (SD) | 9.9 (2.64) | 10.1 (3.00) | | |
| | Median | 9.0 | 10.0 | | |
| | Min, Max | 5, 18 | 5, 18 | | |
| Week 26 MG-ADL Total Score (LOCF) for patients not | n | 51 | 52 | | |
| needing rescue therapy or dropping out of the study | | | | | |
| | Mean (SD) | 7.0 (3.36) | 5.4 (4.05) | | |
| | Median | 6.0 | 5.0 | | |
| | Min, Max | 2, 16 | 0, 15 | | |
| Change from Baseline to Week 26 in MG-ADL Total | n | 51 | 52 | | |
| Score for patients not needing rescue therapy or dropping | | | | | |
| out of the study | | | | | |
| | Mean (SD) | −2.8 (3.07) | −4.7 (4.32) | | |
| | Median | −2.0 | −4.5 | | |
| | Min, Max | −8, 7 | −15, 4 | | |

Refractory gMG is an ultra-rare segment of MG—a debilitating, complement-mediated neuromuscular disease—in which patients have largely exhausted conventional therapy and continue to suffer profound muscle weakness throughout the body, resulting in slurred speech, impaired swallowing and choking, double vision, upper and lower extremity weakness, disabling fatigue, shortness of breath due to respiratory muscle weakness, and episodes of respiratory failure. In the study, the primary efficacy endpoint of change from baseline in Myasthenia Gravis-Activities of Daily Living Profile (MG-ADL) total score, a patient-reported assessment, at week 26, did not reach statistical significance (p=0.0698) as measured by a worst-rank analysis.

TABLE 22
MG-ADL ANCOVA ACTUAL CHANGES FULL ANALYSIS SET

| | | Placebo | Eculizumab | Difference in LS | |
|---|---|---|---|---|---|
| Variable | Statistic | (N = 63) | (N = 62) | Means and 95% Cl | p-value |
| Change from Baseline | LS Mean (SEM) | −2.6 (0.48) | −4.0 (0.48) | −1.4 | 0.0390 |
| | 95% Cl for LS Mean | (−3.52, −1.63) | (−4.96, −3.04) | (−2.77, −0.07) | |
| Baseline MG-ADL Total Score | n | 63 | 62 | | |
| | Mean (SD) | 9.9 (2.58) | 10.5 (3.06) | | |
| | Median | 9.0 | 10.0 | | |
| | Min, Max | 5, 18 | 5, 18 | | |
| Week 26 MG-ADL Total Score (LOCF) | n | 63 | 62 | | |
| | Mean (SD) | 7.4 (3.50) | 6.4 (4.76) | | |
| | Median | 7.0 | 6.0 | | |
| | Min, Max | 0, 16 | 0, 17 | | |
| Change from Baseline | n | 63 | 62 | | |
| | Mean (SD) | −2.4 (3.32) | −4.1 (4.48) | | |
| | Median | −2.0 | −4.0 | | |
| | Min, Max | −8, 7 | −15, 4 | | |

TABLE 23
MG-ADL ANCOVA ACTUAL CHANGES PER PROTOCOL SET

| | | | | Difference in | |
|---|---|---|---|---|---|
| | | | | LS | |
| | | Placebo | Eculizumab | Means and | |
| Variable | Statistic | (N = 56) | (N = 54) | 95% Cl | p-value |
| Change from Baseline | LS Mean (SEM) | −2.6 (0.48) | −4.3 (0.49) | −1.7 | 0.0153 |
| | 95% Cl for LS Mean | (−3.54, −1.63) | (−5.25, −3.30) | (−3.05, −0.33) | |
| Baseline MG-ADL Total Score | n | 56 | 54 | | |
| | Mean (SD) | 9.9 (2.63) | 10.3 (3.04) | | |
| | Median | 9.0 | 10.0 | | |
| | Min, Max | 5, 18 | 5, 18 | | |
| Week 26 MG-ADL Total Score (LOCF) | n | 56 | 54 | | |
| | Mean (SD) | 7.4 (3.39) | 6.0 (4.36) | | |
| | Median | 7.0 | 6.0 | | |
| | Min, Max | 2, 16 | 0, 17 | | |
| Change from Baseline | fi | 56 | 54 | | |
| | Mean (SD) | −2.4 (3.16) | −4.3 (4.47) | | |
| | Median | −2.0 | −4.0 | | |
| | Min, Max | −8, 7 | −15, 4 | | |

Next the QMG scores were evaluated for all study participants. The current QMG scoring system consists of 13 items: ocular (2 items), facial (1 item), bulbar (2 items), gross motor (6 items), axial (1 item) and respiratory (1 item); each graded 0 to 3, with 3 being the most severe (see Table 2). The range of total QMG score is 0-39. The QMG scoring system is considered to be an objective evaluation of therapy for MG and is based on quantitative testing of sentinel muscle groups. The MGFA task force has recommended that the QMG score be used in prospective studies of therapy for MG. A clinically meaningful improvement in a patient's QMG would be a 5 point or greater reduction in score after 26 weeks of treatment.

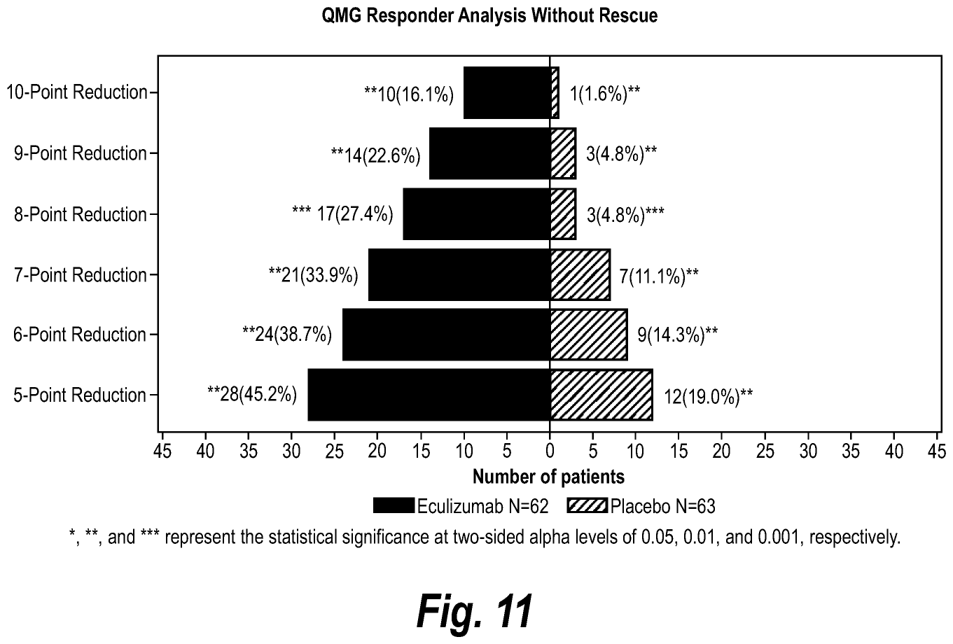

The QMG score for the full data set was −5 in the eculizumab treated group and therefore resulted in a clinically significant improvement for all patients not needing rescue or dropping out of the study. See Table 24 below and FIG. 11 for the results.

TABLE 24
QMG WORST RANK ANALYSIS: SAP3 FULL ANALYSIS SET

| | | Placebo | Eculizumab | Difference in LS | |
|---|---|---|---|---|---|
| Variable | Statistic | (N = 63) | (N = 62) | Means and 95% Cl | p-value |
| Worst Ranked Change from Baseline | Ranked Score LS Mean (SEM) | 70.7 (4.46) | 54.7 (4.50) | −16.0 | 0.0129 |
| | 95% Cl for LS Mean | (61.85, 79.51) | (45.82, 63.64) | (−28.48, −3.43) | |
| Baseline QMG Total Score for patients not needing | n | 51 | 52 | | |
| rescue therapy or dropping out of the study | | | | | |
| | Mean (SD) | 16.4 (5.76) | 17.1 (4.96) | | |
| | Median | 15.0 | 17.0 | | |
| | Min, Max | 8, 34 | 6, 31 | | |
| Week 26 QMG Total Score (LOCF) for patients not | n | 51 | 52 | | |
| needing rescue therapy or dropping out of the study | | | | | |
| | Mean (SD) | 14.1 (5.40) | 11.7 (5.83) | | |
| | Median | 13.0 | 12.0 | | |
| | Min, Max | 5, 32 | 1, 27 | | |
| Change from Baseline to Week 26 in QMG Total Score | n | 51 | 52 | | |
| for patients not needing rescue therapy or dropping | | | | | |
| out of the study | | | | | |
| | Mean (SD) | −2.4 (3.70) | −5.4 (4.80) | | |
| | Median | −3.0 | −5.0 | | |
| | Min, Max | −11, 8 | −16, 2 | | |

Next, the MGC score was evaluated for all study participants over time. The MGC is a validated assessment tool for measuring clinical status of subjects with MG (16). The MGC assesses 10 important functional areas most frequently affected by MG and the scales are weighted for clinical significance that incorporate subject-reported outcomes (see Table 3). MGC will be administered at Screening, Day 1, Weeks 1-4, 8, 12, 16, 20, and 26 or ET (Visits 1-6, 8, 10, 12, 14, and 17 or ET). Total scores range from 0-50. A clinically meaningful improvement in a patient's MGC would be a 3 point or greater reduction in score after 26 weeks of treatment.

TABLE 25
MG COMPOSITE WORST RANK ANALYSIS: SAP3 FULL ANALYSIS SET

| | | Placebo | Eculizumab | Difference in LS | |
|---|---|---|---|---|---|
| Variable | Statistic | (N = 63) | (N = 62) | Means and 95% Cl | p-value |
| Worst Ranked Change from Baseline | Ranked Score LS Mean (SEM) | 67.7 (4.47) | 57.3 (4.52) | −10.5 | 0.1026 |
| | 95% Cl for LS Mean | (58.89, 76.57) | (48.32, 66.21) | (−23.07, 2.13) | |
| Baseline MGC Total Score for patients not needing | n | 51 | 52 | | |
| rescue therapy or dropping out of the study | | | | | |
| | Mean (SD) | 19.0 (6.19) | 19.4 (5.97) | | |
| | Median | 19.0 | 20.0 | | |
| | Min, Max | 7, 40 | 7, 35 | | |
| Week 26 MGC Total Score (LOCF) for patients not | n | 51 | 52 | | |
| needing rescue therapy or dropping out of the study | | | | | |
| | Mean (SD) | 13.0 (6.96) | 10.3 (7.00) | | |
| | Median | 12.0 | 9.5 | | |
| | MM, Max | 3, 37 | 0, 28 | | |
| Change from Baseline to Week 26 in MGC Total | | 51 | 52 | | |
| Score for patients not needing rescue therapy or | | | | | |
| dropping out of the study | | | | | |
| | Mean (SD) | −6.0 (6.19) | −9.2 (8.08) | | |
| | Median | −6.0 | −10.0 | | |
| | Min, Max | −21, 13 | −24, 17 | | |

The MGC score for the full data set was (−10) in the eculizumab treated group and therefore resulted in a clinically significant improvement for all patients not needing rescue or dropping out of the study. See Table 25 above for the results.

The 15-item Myasthenia Gravis Qualify of Life scale (MG-QOL 15) is a health-related quality of life evaluative instrument specific to subjects with MG. See Table 4. MG-QOL15 was designed to provide information about subjects' perception of impairment and disability and the degree to which disease manifestations are tolerated and to be easy to administer and interpret. The MG-QOL 15 is completed by the subject. Total scores range from 0 to 60 and higher scores indicate greater extent of and dissatisfaction with MG-related dysfunction. A clinically meaningful improvement in a patient's MG-QOL would be a decrease in score after 26 weeks of treatment.

The MG-QOL15 median score for the full data set was (−11.5) in the eculizumab treated group and therefore resulted in a clinically significant improvement for all patients not needing rescue or dropping out of the study. See Table 26 below for the results.

TABLE 26
MG-QOL15 WORST RANK ANALYSIS FULL ANALYSIS SET

| | | | | Difference in LS | |
|---|---|---|---|---|---|
| | | Placebo | Eculizumab | Means and 95% | |
| Variable | Statistic | (N = 63) | (N = 62) | Cl | p-value |
| Worst Ranked Change from Baseline | Ranked Score LS Mean | 69.7 (4.51) | 55.5 (4.55) | −14.3 | 0.0281 |
| | (SEM) | | | | |
| | 95% Cl for LS Mean | (60.79, 78.66) | (46.43, 64.47) | (−26.98, −1.56) | |
| Baseline MG-QOL15 Total Score for patients | n | 51 | 52 | | |
| not needing rescue therapy or dropping out of | | | | | |
| the study | | | | | |
| | Mean (SD) | 30.2 (13.10) | 31.5 (11.82) | | |
| | Median | 30.0 | 32.0 | | |
| | Min, Max | 6, 60 | 6, 59 | | |
| Week 26 MG-QOL15 Total Score (LOCF) for | n | 51 | 52 | | |
| patients not needing rescue therapy or dropping | | | | | |
| out of the study | | | | | |
| | Mean (SD) | 23.7 (13.38) | 18.0 (14.37) | | |
| | Median | 20.0 | 16.0 | | |
| | Min, Max | 3, 58 | 0, 59 | | |
| Change from Baseline to Week 26 in MG- | n | 51 | 52 | | |
| QOL15 Total Score for patients not needing | | | | | |
| rescue therapy or dropping out of the study | | | | | |
| | Mean (SD) | −6.5 (9.40) | −13.5 (14.07) | | |
| | Median | −6.0 | −11.5 | | |
| | Min, Max | −30, 16 | −44, 19 | | |

The Neuro-QOL Fatigue is a reliable and validated brief 19-item survey of fatigue completed by the subject. Higher scores indicate greater fatigue and greater impact of MG on activities (see Table 5). A clinically meaningful improvement in a patient's Neuro-QOL Fatigue score would be reflected in a decrease in score after 26 weeks of treatment.

As shown in Table 27 below, the eculizumab treated group realized a clinically meaningful improvement (reduction) in their Neuro-QOL Fatigue score after 26 weeks of treatment.

TABLE 27
NEURO FATIGUE QOL WORST RANK ANALYSIS FULL ANALYSIS SET

| | | | | Difference in LS | |
|---|---|---|---|---|---|
| | | Placebo | Eculizumab | Means and 95% | |
| Variable | Statistic | (N = 63) | (N = 62) | Cl | p-value |
| Worst Ranked Change from Baseline | Ranked Score LS Mean | 74.1 (6.26) | 58.5 (6.06) | −15.6 | 0.0145 |
| | (SEM) | | | | |
| | 95% Cl for LS Mean | (61.73, 86.53) | (46.49, 70.48) | (−28.13, −3.15) | |
| Baseline Neuro-QOL Fatigue Total Score for | n | 51 | 52 | | |
| patients not needing rescue therapy or dropping | | | | | |
| out of the study | | | | | |
| | Mean (SD) | 61.7 (15.36) | 61.8 (13.57) | | |
| | Median | 65.0 | 62.0 | | |
| | Min, Max | 29, 88 | 36, 92 | | |
| Week 26 Neuro-QOL Fatigue Total Score | n | 49 | 51 | | |
| (LOCF) for patients not needing rescue therapy | | | | | |
| or dropping out of the study | | | | | |
| | Mean (SD) | 52.6 (18.66) | 43.6 (19.44) | | |
| | Median | 55.0 | 38.0 | | |
| | Min, Max | 21, 85 | 19, 95 | | |
| Change from Baseline to Week 26 in Neuro-QOL | n | 49 | 51 | | |
| Fatigue Total Score for patients not needing | | | | | |
| rescue therapy or dropping out of the study | | | | | |
| | Mean (SD) | −9.1 (14.58) | −18.2 (19.60) | | |
| | Median | −8.0 | −16.0 | | |
| | Min, Max | −51, 20 | −69, 30 | | |

### Discussion of the Significance of the REGAIN Study

The first prospectively defined secondary efficacy endpoint of change from baseline in Quantitative Myasthenia Gravis (QMG) total score, a physician-administered assessment of MG clinical severity, with eculizumab treatment compared to placebo at week 26, achieved a p-value of 0.0129 as measured by a worst-rank analysis. In addition, the second and third prospectively defined secondary efficacy endpoints of responder status in MG-ADL and QMG achieved p-values of <0.05: the proportion of patients with at least a 3-point reduction in MG-ADL total score and no rescue therapy from baseline to week 26 with eculizumab treatment, compared to placebo, achieved a p-value of 0.0229, and the proportion of patients with at least a 5-point reduction in QMG total score and no rescue therapy from baseline to week 26 with eculizumab treatment compared to placebo achieved a p-value of 0.0018.

It is encouraging that the REGAIN study achieved clinically meaningful improvements in MG-ADL and QMG measures in patients treated with eculizumab compared with placebo. The magnitude of effect on QMG observed in this large, prospective registration trial is unprecedented in more than 30 years of clinical investigation of refractory MG patients. There is an urgent need in the MG community for a therapy with the potential to dramatically improve the lives of patients with refractory gMG, who continue to experience profound complement-mediated muscle weakness that makes it difficult or impossible to perform simple daily activities, including walking, talking, swallowing, and even breathing normally.

Pre-specified sensitivity analyses were prospectively defined to validate results for the primary and first secondary endpoints. Three of the four prospectively defined MG-ADL sensitivity analyses achieved p-values <0.05, including the sensitivity analysis around the primary endpoint for change from baseline in MG-ADL using repeated measures, which showed a mean change with eculizumab treatment at week 26 of −4.2 versus a mean change with placebo at week 26 of −2.3 and achieved a p-value of 0.0058. Additionally, all four prospectively defined QMG sensitivity analyses achieved p-values <0.05, including the sensitivity analysis around the first secondary endpoint for change from baseline in QMG using repeated measures, which showed a mean change with eculizumab treatment at week 26 of −4.6 versus a mean change with placebo at week 26 of −1.6 and achieved a p-value of 0.0006.

The findings from this study underscore the pivotal role of complement inhibition in addressing the underlying pathophysiology of refractory gMG. Importantly, the totality of data including the first three secondary endpoints and a series of prospectively defined sensitivity analyses, shows early and sustained substantial improvements over 26 weeks for patients treated with eculizumab compared to placebo

### Example 4: Analysis of QMG and MG-ADL Dual Responder Refractory Generalized Myasthenia Gravis (gMG) Patients from ECU-MG-301 26 Week (301) REGAIN Trial

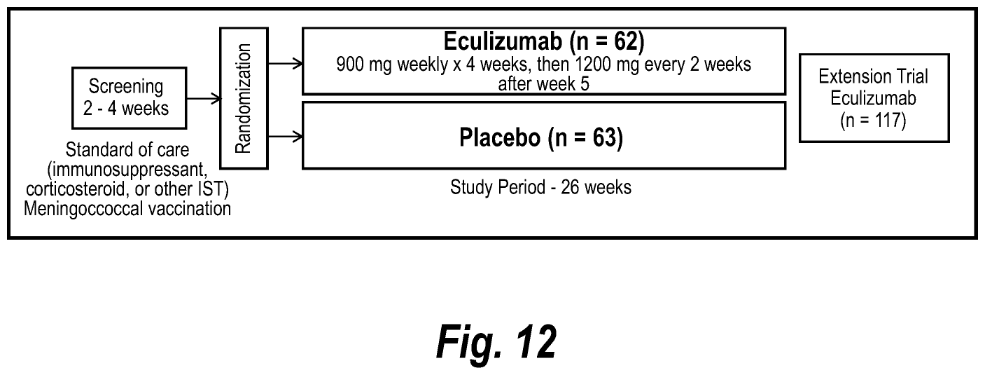

The objective of the example was to assess the time course of response in patients who demonstrated a clinically meaningful response to eculizumab and the proportion of patients who had clinically meaningful relevant responses on both the MG-ADL and the QMG during the ECU-MG-301 REGAIN Trial (see Example 1 for protocol). Patients with refractory gMG continued to receive stable doses of ISTs (including corticosteroids) throughout the study; patients were randomized to receive blinded eculizumab 900 mg weekly for 4 weeks, 1200 mg on the fifth week, and then 1200 mg every 2 weeks thereafter (n=62) or blinded placebo (n=63) (FIG. 12).

The Myasthenia Gravis Activities of Daily Living (MG-ADL) is a physician-directed, patient-reported measure of symptom severity related to MG-specific ADLs (Muppidi, Ann. N.Y. Acad. Sci. 1274: 114-19 (2012)), and the Quantitative Myasthenia Gravis (QMG) tool is a clinician-reported measure of muscle strength (Barohn et al., Ann. N.Y. Acad. Sci. 841: 769-72 (1998)). Pre-specified responder analyses included the proportion of patients who responded with ≥3-point improvement in MG-ADL total score with no rescue; and the proportion of patients with ≥5-point improvement in QMG total score with no rescue. In an ad hoc dual responder analysis, response was defined as an improvement of ≥3 points from baseline in the MG-ADL total score and improvement of ≥5 points from baseline in the QMG total score, with no rescue therapy. In addition to the prespecified responder thresholds (i.e., 3-point improvement for MG-ADL and ≥5-point improvement for QMG), thresholds of ≥4, 5, 6, 7, and 8 for MG-ADL and ≥6, 7, 8, 9, and 10 for QMG were also examined. P values from a Cochran-Mantel-Haenszel (CMH) test were provided for the more stringent criteria to aid interpretation.

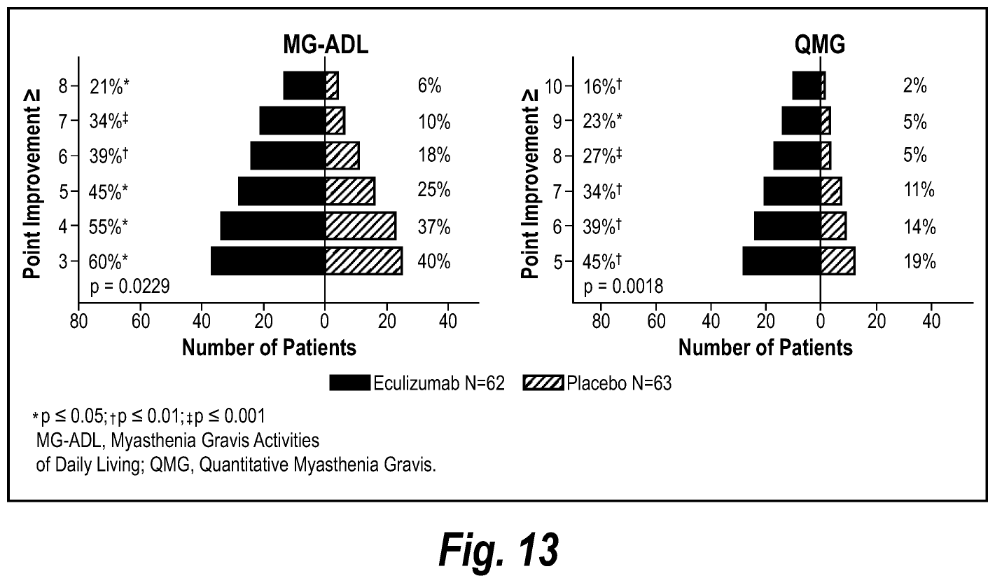

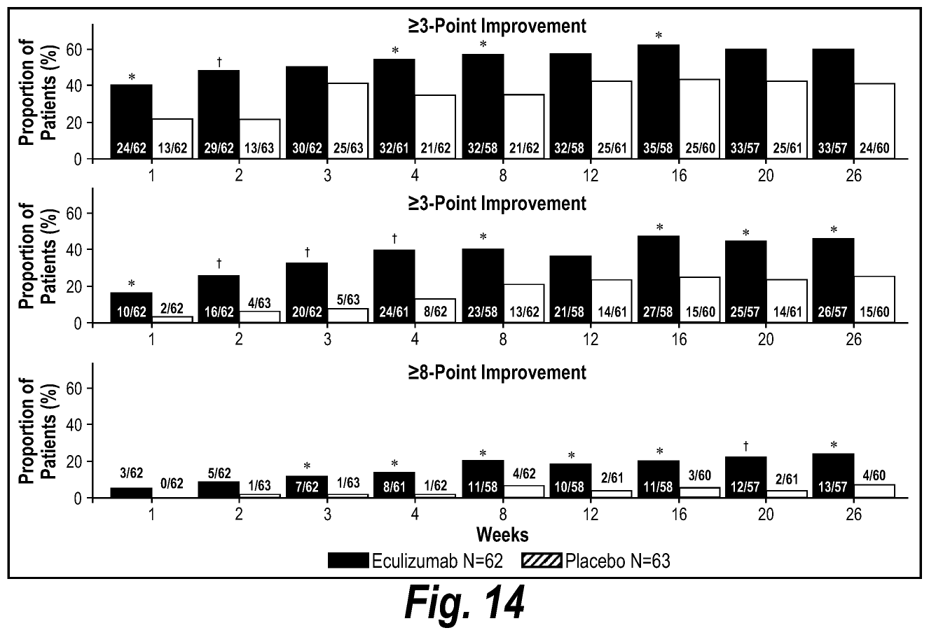

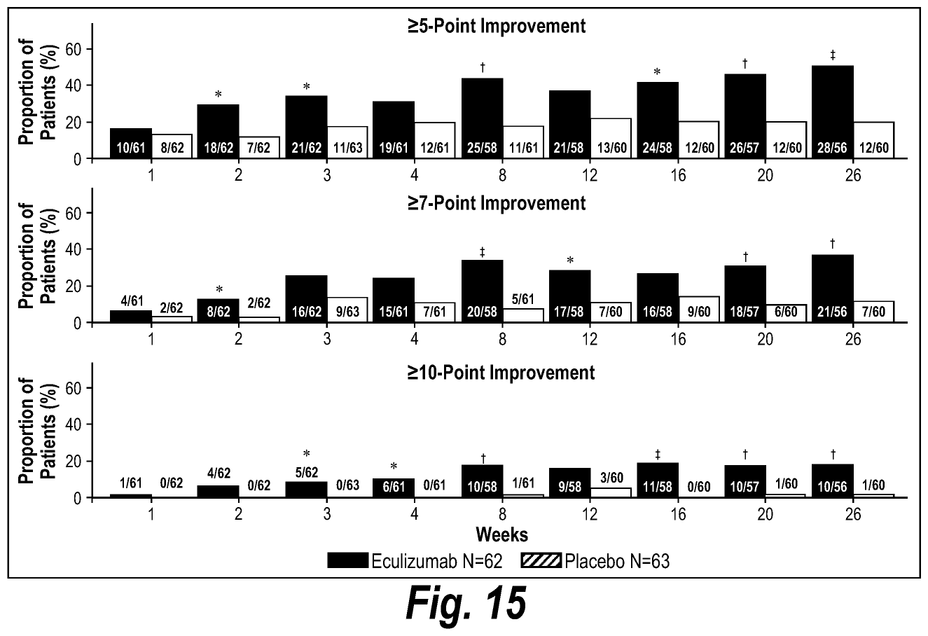

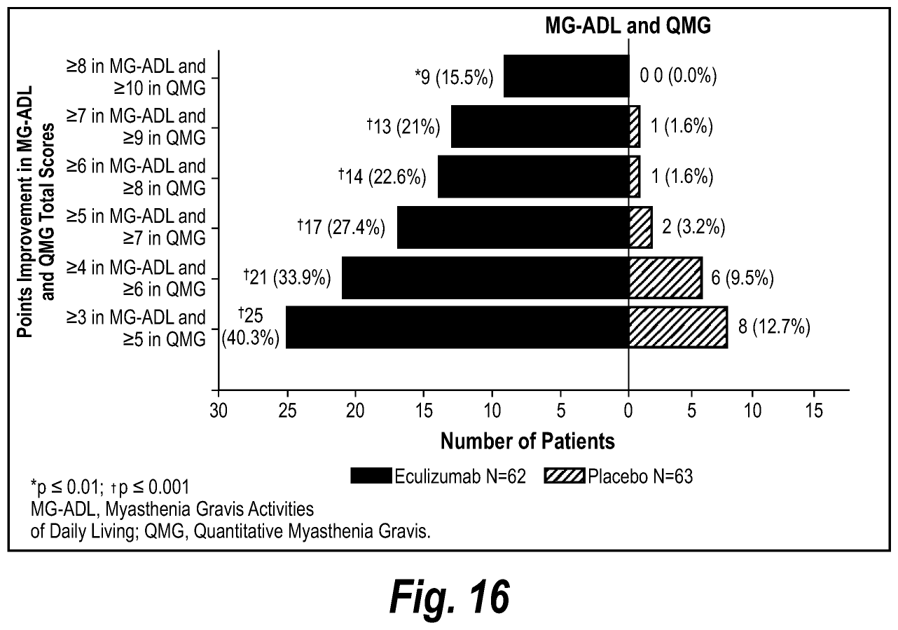

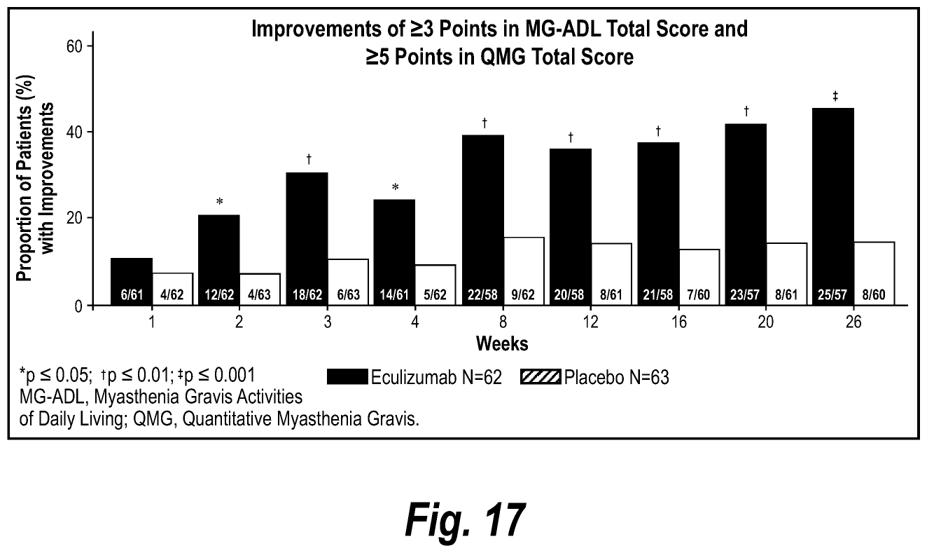

More patients receiving eculizumab than those who received placebo experienced clinically meaningful responses as defined above, and also clinically meaningful relevant responses based on the more stringent thresholds for both MG-ADL and QMG total scores (FIG. 13). MG-ADL responder analyses conducted at each assessment date over the 26-week study are shown in FIG. 14 (the proportion of patients with a ≥3, 5, or 8 point change in the MG-ADL). QMG responder analyses conducted at each assessment date over the 26-week study are shown in FIG. 15 (the proportion of patients with a greater than 5, 7, or 10 point change in the QMG). There was a substantial overlap of patients who achieved a clinically meaningful response in both the MG-ADL total score and QMG total score (FIG. 16). For each of the categorical thresholds of response, a >3-fold increase was seen in the proportion of improved patients in the eculizumab group compared with those in the placebo group (FIG. 16). More patients receiving eculizumab versus placebo achieved clinically meaningful responses at week 26 in both the MG-ADL and QMG scores (week 26: eculizumab 40% vs placebo 13%; nominal P<0.001) (FIG. 16). The benefit of eculizumab treatment was apparent within the first 2 weeks (week 2: eculizumab 19% vs placebo 6% were dual responders; nominal P=0.0297) and was sustained through week 26 (all P≤0.05) (FIG. 17).

Three times as many patients with refractory gMG who were treated with eculizumab experienced clinically meaningful improvements in both muscle strength and ADLs compared with the placebo group by week 26. An increased proportion of individual assessment responders (on both the MG-ADL and the QMG) as well as dual responders occurred in the eculizumab-treated patient group compared with the placebo group which was observed early and generally maintained over the course of the study.

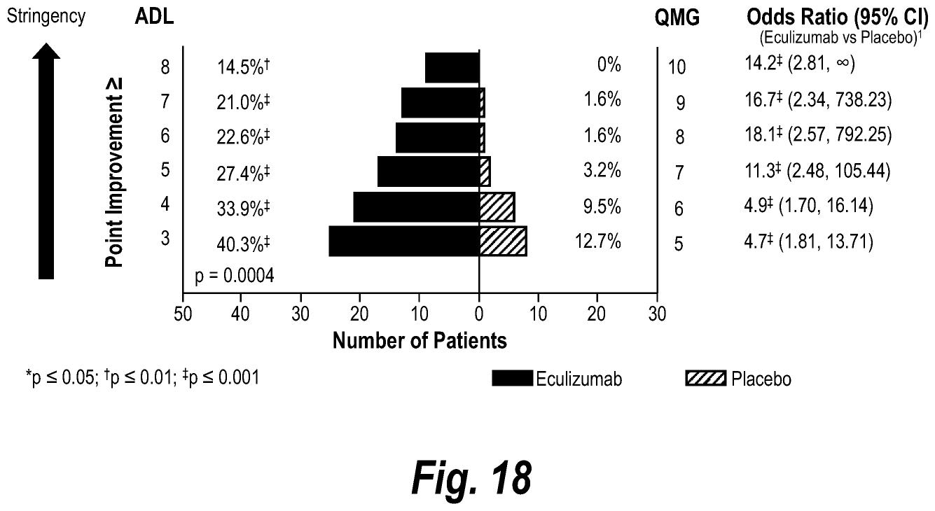

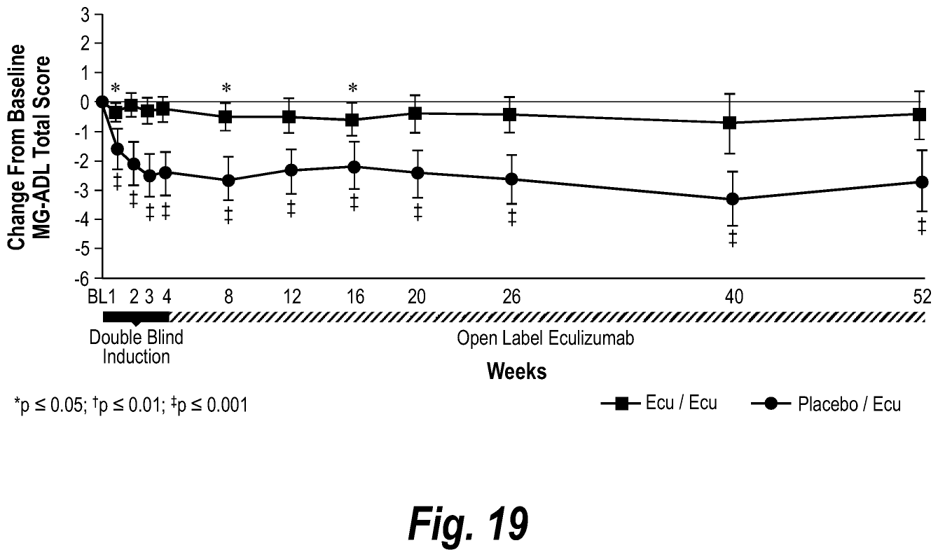

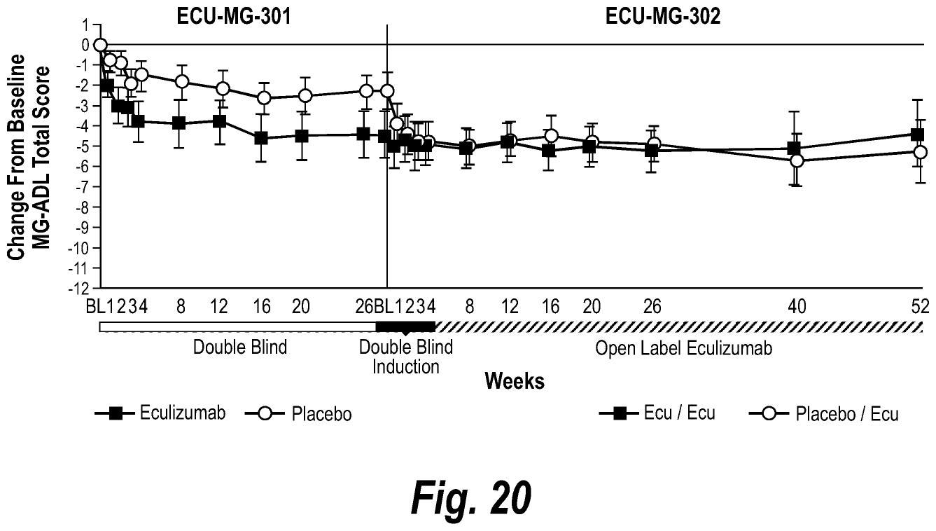

As shown in FIG. 18, the response rate was substantially higher in eculizumab-treated patients (40.3%) than in placebo-treated patients (12.7%), showing a clinically significant response by both patient- and physician-assessed outcome measures in patients treated with eculizumab. With increasingly stringent response criteria, the superiority of response to eculizumab over placebo becomes more pronounced, with odds ratios exceeding 10.

### Example 5: Efficacy of Eculizumab is Maintained Beyond 26 Weeks in Patients with AChR+ Refractory Generalized Myasthenia Gravis (gMG) Enrolled in ECU-MG-302 (302) REGAIN Extension Trial

Patients who completed REGAIN were allowed to continue into an open-label extension study known as ECU-MG-302 (see Example 2 for protocol). In contrast with Study ECU-MG-301, in which patients were required to maintain stable MG therapy throughout the 26-week study period, adjustment of background immunosuppressant therapy (IST), including corticosteroids and acetylcholinesterase inhibitors (AChI), was permitted in Study ECU-MG-302. Investigators could change dosing of an existing IST/AChI, discontinue an existing IST/AChI, or add a new IST/AChI.

Each patient enrolled in the extension trial underwent an initial 4-week blinded induction before receiving open-label eculizumab maintenance treatment (1200 mg every 2 weeks for up to 4 years). MG-ADL, QMG, MGC, and MG-QOL15 scores and safety were assessed. Patients entering the ECU-MG-302 open-label study had baseline MG-ADL total scores of 5.8 (standard deviation, 4.27) for the eculizumab/eculizumab group, and 7.6 (standard deviation 3.63) for the placebo/eculizumab group.

The MG-ADL total score in eculizumab/eculizumab patients (n=55) was unchanged (0.2 point reduction; standard deviation 1.77) from open-label baseline through week 4 of the blind induction phase. The MG-ADL total score in this group of patients remained unchanged (standard deviation 3.97) from open-label baseline through week 52 of the ECU-MG-302 open-label-study. These patients had a 4.5 point mean reduction (standard deviation 3.96) in MG-ADL total score from REGAIN baseline measurements. A total of 35 eculizumab/eculizumab patients completed 130 weeks of the ECU-MG-302 open-label study and were measured for MG-ADL score. The MG-ADL score in this group of patients was slightly improved (0.7 point reduction; standard deviation 4.19) from open-label baseline.

In the placebo/eculizumab patients (n=61), rapid improvement in MG-ADL total score from open-label baseline was demonstrated with a change from ECU-MG-302 baseline in MG-ADL total score observed as early as week 1 (1.6 point reduction [−2.28, −0.89]; p<0.0001). The majority of the overall treatment effect was achieved by week 4 (2.4 point reduction [−3.19, −1.71]; p<0.000l) during the blind induction phase, and was sustained through week 52 (2.7 point reduction [−3.73, −1.63]; p<0.0001). This group of patients demonstrated a 5.0 point mean reduction (standard deviation 3.39) in MG-ADL total score from REGAIN baseline measurements. A total of 36 placebo/eculizumab patients completed 130 weeks of the ECU-MG-302 open-label study and were measured for MG-ADL score. The MG-ADL score in this group of patients was improved (3.9 point reduction; standard deviation, 3.68) from open-label baseline.

Changes in QMG, MGC, and MG-QOL 15 total scores followed a pattern similar to that of the MG-ADL (QMG: −4.6; P<0.0001; MGC: −5.1; P<0.0001; and MG-QOL15: −5.7; P=0.005 at week 52). Similar patterns of response were seen on the respiratory, bulbar, limb, and ocular MG-ADL domains. The safety profile of eculizumab remained unchanged throughout the open-label extension study and was consistent with the known profile.

Overall, 65 (55.6%) patients reported a change in their IST usage during the study. Greater proportions of patients had dose reductions or stopped ≥1 IST than those who had dose increases or started ≥1 IST (Table 28). 55 (47.0%) patients decreased their daily dose of 1 IST and 2 (1.7%) patients decreased the daily dose of >1 IST; 29 (24.8%) patients increased their daily dose of 1 IST, and none increased their dose of >1 IST. 19 (16.2%) patients stopped an existing IST; 5 (4.3%) patients started a new IST. The most common reason for change in IST therapy was improvement in MG symptoms, with 42 (35.9%) patients reporting improvement in MG symptoms as the reason for changing IST therapy. In comparison, 21 (17.9%) patients reported worsening of MG symptoms as the primary reason for changing IST therapy. Side-effects/intolerance to an IST was reported as the reason for change in IST therapy in 13 (11.1%) patients.

TABLE 28
SUMMARY OF CHANGES IN IMMUNOSUPPRESSANT THERAPY STATUS-EXTENSION SAFETY SET

| | Placebo/Eculizumab | | Eculizumab/Eculizumab | | All Patients | |
|---|---|---|---|---|---|---|
| | (N = 61) | | (N = 56) | | (N = 117) | |
| | Change IST | Patients, | Change IST | Patients, | Change IST | Patients, |
| Parameter | Events, n | n (%) | Events, n | n (%) | Events, n | n (%) |
| IST Change Events and Patients | 148 | 36 (59.0) | 157 | 29 (51.8) | 305 | 65 (55.6) |
| with IST Changes | | | | | | |

Changes Made in IST Status

| | | | | | | |
|---|---|---|---|---|---|---|
| Start of New IST | 2 | 2 (3.3) | 5 | 3 (5.4) | 7 | 5 (4.3) |
| Stop of an Existing IST | 9 | 7 (11.5) | 13 | 12 (21.4) | 22 | 19 (16.2) |
| Increase the Daily dose of one IST | 33 | 16 (26.2) | 37 | 13 (23.2) | 70 | 29 (24.8) |
| Decrease the Daily dose of one IST | 102 | 30 (49.2) | 102 | 25 (44.6) | 204 | 55 (47.0) |
| Increased the Daily dose of more | 0 | 0 (0.0) | 0 | 0 (0.0) | 0 | 0 (0.0) |
| than one IST | | | | | | |
| Decreased the Daily dose of more | 2 | 2 (3.3) | 0 | 0 (0.0) | 2 | 2 (1.7) |
| than one IST | | | | | | |

Primary reason for change in IST Status

| | | | | | | |
|---|---|---|---|---|---|---|
| MG symptoms improved | 88 | 26 (42.6) | 70 | 16 (28.6) | 158 | 42 (35.9) |
| MG symptoms worsened | 22 | 11 (18.0) | 19 | 10 (17.9) | 41 | 21 (17.9) |
| Side effects-intolerant to existing IST | 12 | 6 (9.8) | 15 | 7 (12.5) | 27 | 13 (11.1) |
| New indication other than MG for | 0 | 0 (0.0) | 1 | 1 (1.8) | 1 | 1 (0.9) |
| IST usage | | | | | | |
| Other | 26 | 11 (18.0) | 51 | 12 (21.4) | 77 | 23 (19.7) |

Abbreviations: IST = immunosuppressant therapy;
MG = myasthenia gravis

Overall, the extension study demonstrated that patients who received eculizumab in Study ECU-MG-301 sustained their improvements through the Study ECU-MG-302. For patients who received placebo in Study ECU-MG-301, an improvement occurred rapidly after starting eculizumab treatment and was maintained through the Study ECU-MG-302, similar to the effect observed in eculizumab-treated patients in Study ECU-MG-301.

### Example 6: Achievement of Minimal Manifestations in Eculizumab-Treated Patients with Acetylcholine Receptor Antibody-Positive Refractory Myasthenia Gravis Enrolled in ECU-MG-302 (302) REGAIN Extension Trial

This example provides a detailed evaluation of patients' response to eculizumab treatment during REGAIN (ECU-MG-301 REGAIN; see Example 1 for protocol) and up to week 130 of an open-label extension (OLE) study ECU-MG-302; see Example 2 for protocol. The 6-month, phase 3, randomized, placebo-controlled REGAIN study demonstrated the efficacy and safety of eculizumab in patients with anti-AChR antibody-positive (AChR+) refractory gMG. An interim analysis of results of the open-label extension of REGAIN found that the benefits of eculizumab for this patient population were maintained through 3 years of treatment. At the last assessment before Dec. 31, 2017 (the data cut-off date for the interim analysis), 74.1% of patients had an MGFA post-intervention status of improved, and 56.0% were considered to have achieved MM or PR. Here, we report a detailed evaluation of patients' response to eculizumab treatment during REGAIN and up to week 130 of the open-label study using MGFA post-intervention status.

Patients who completed REGAIN (ClinicalTrials.gov identifier: NCT01997229) were eligible for inclusion in an open-label study (ClinicalTrials.gov identifier: NCT02301624) and were required to enroll within 2 weeks of completing REGAIN. Patients were eligible for inclusion in REGAIN if they had confirmed gMG, AChR+serology, a Myasthenia Gravis Activities of Daily Living (MG-ADL) total score of at least 6, and had received two or more ISTs, or at least one IST with intravenous immunoglobulin or plasma exchange treatment at least four times in 12 months without symptom control. Patients with ocular MG (MGFA class I) or myasthenic crisis at screening (MGFA class V) were excluded from the trial. Full eligibility criteria have been published previously. All participants were required to have been vaccinated against Neisseria meningitidis at least 2 weeks before starting study treatment; individuals who were not vaccinated at the appropriate time received prophylactic antibiotics until 2 weeks after vaccination. During the open-label study, patients were revaccinated according to local guidelines. During REGAIN, patients who previously received ISTs were required to maintain their pre-study dose and schedule. During the open-label study, modifications to IST dose and schedule were permitted at the discretion of the investigator; however, changes were not required by the protocol.

All patients provided written, informed consent. Independent ethics committees or institutional review boards provided written approval for the study protocols and all amendments. The studies were performed in accordance with the ethical standard laid down in the 1964 Declaration of Helsinki and are registered with www.clinicaltrials.gov.

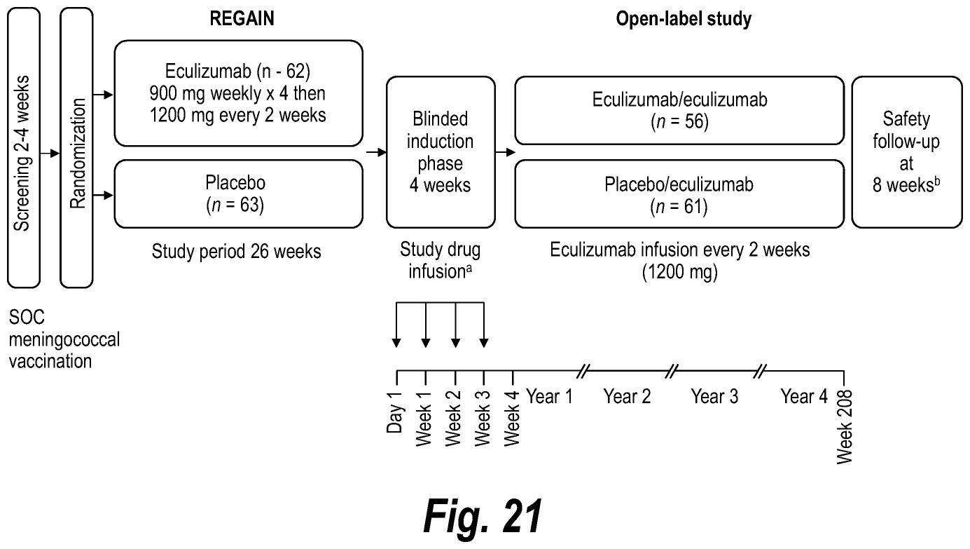

In REGAIN, patients randomized to receive eculizumab were given an induction dose of 900 mg on day 1 and at weeks 1, 2 and 3, followed by a maintenance dose of 1200 mg at week 4 and every 2 weeks thereafter (FIG. 21). Placebo was administered on the same schedule. Patients who received eculizumab during REGAIN continued to receive it during the open-label study (eculizumab/eculizumab arm) and those who received placebo during REGAIN started eculizumab treatment upon entering the open-label study (placebo/eculizumab arm). To preserve the blinded nature of REGAIN, patients who continued into the open-label study underwent a 4-week blinded induction phase (FIG. 21). During this phase, patients in the eculizumab/eculizumab group received eculizumab 1200 mg on day 1 and at week 2, and placebo at weeks 1 and 3. Patients in the placebo/eculizumab group received eculizumab 900 mg on day 1 and at weeks 1, 2 and 3. All patients were then given eculizumab 1200 mg open-label at week 4 and every 2 weeks thereafter.

The objectives of REGAIN and the open-label study were to assess the efficacy of eculizumab, as measured by change in MG-ADL total score from baseline, and to evaluate its safety. This secondary analysis assessed MGFA post-intervention status and safety data during REGAIN and the open-label study for patients who continued into the open-label study.

MGFA post-intervention status following administration of eculizumab or placebo during REGAIN, including achievement of MM, was assessed at weeks 4, 12 and 26 of REGAIN and weeks 26, 40, 52 and 130 of the open-label study. MGFA post-intervention status was reported as improved if a patient's pretreatment clinical manifestations were substantially decreased, worse if they were substantially increased, or unchanged if they were not substantially changed compared with REGAIN baseline. In patients with improved status, MM was achieved if they had no symptoms indicating functional limitations from MG but had some weakness on examination of some muscles. Subcategories of MM relating to treatment status were not assessed. Patients were evaluated for PR at open-label study weeks 26, 40, 52 and 130. PR was achieved if patients had no signs or symptoms of MG for at least 1 year and, upon examination, had no weakness of any muscle, other than isolated weakness of eyelid closure.

Adverse events were recorded and coded by preferred term using the Medical Dictionary for Regulatory Activities Version 20.1. MG exacerbations, use of rescue therapy and study discontinuations because of adverse events were also recorded.

Common odds ratios for achievement of improved status or MM at REGAIN week 26 for patients who received eculizumab compared with those who received placebo were calculated using an ordinal regression model.

Results

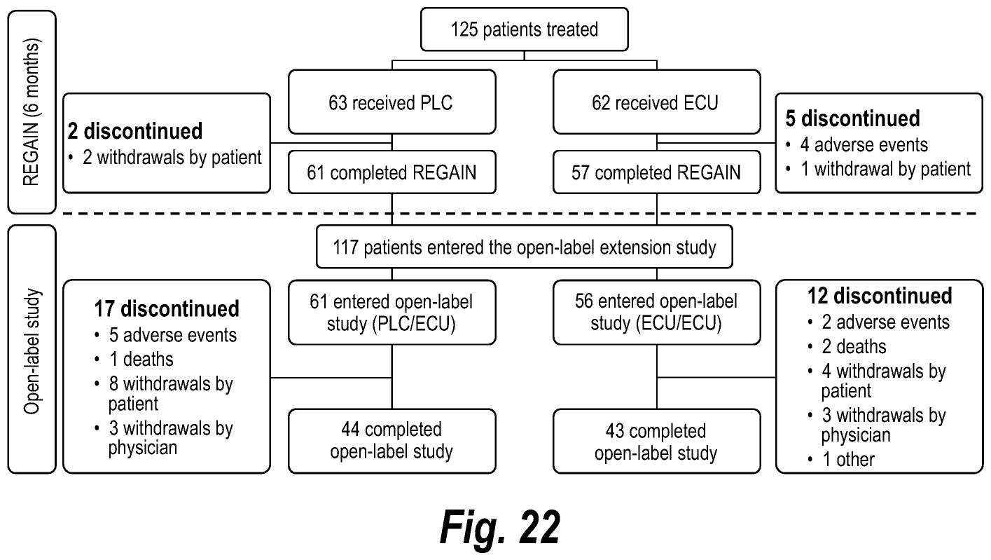

A total of 117 patients who completed REGAIN continued into the open-label study (eculizumab/eculizumab, 56; placebo/eculizumab, 61. FIG. 22) and were included in the efficacy and safety analysis. One patient from the eculizumab/eculizumab group withdrew from the open-label study during the blind induction phase, prior to entering the maintenance phase. Patient demographics and characteristics, published previously, were similar for the eculizumab/eculizumab and placebo/eculizumab groups, with the exception that there was a greater proportion of Asian patients in the placebo/eculizumab group.

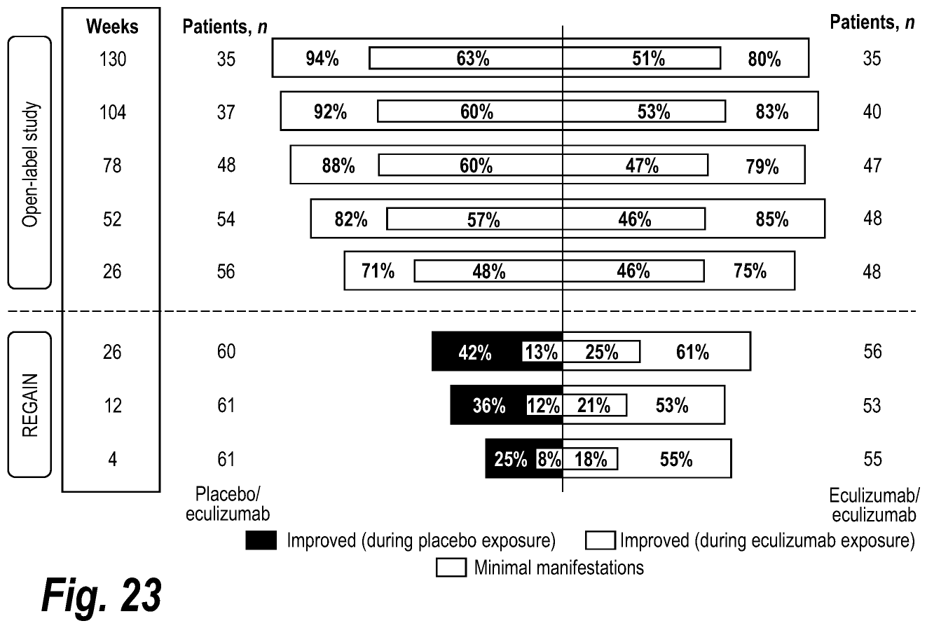

During REGAIN, at all time points assessed, a higher proportion of patients who received eculizumab than of those who were given placebo achieved improved status (Table 29, FIG. 23). At week 4, 54.5% of eculizumab-treated patients (30/55) achieved a status of improved compared with 24.6% of placebo-treated patients (15/61). At week 26, 60.7% of eculizumab-treated patients (34/56) had achieved a status of improved, compared with 41.7% of placebo-treated patients (25/60). One patient who received eculizumab had a status of worse at week 26 compared with five individuals who received placebo. By week 130 of the open-label study, most patients in both groups had achieved improved status: 80.0% of participants in the eculizumab/eculizumab group (28/35) and 94.3% of those in the placebo/eculizumab group (33/35).

During REGAIN, the proportion of patients receiving eculizumab who achieved MM increased from 18.2% (10/55) at week 4 to 25.0% (14/56) at week 26 (Table 29, FIG. 23). MM status was achieved by a smaller proportion of the placebo group (8.2% [5/61] at week 4 and 13.3% [8/60] at week 26) than of the eculizumab group. Eculizumab-treated patients were significantly more likely to achieve a status of improved or MM at REGAIN week 26 than those given placebo (common odds ratio, 2.2; 95% confidence interval, 1.1-4.4; P=0.0233). By week 130 of the open-label study, the proportion of patients achieving MM had increased to 51.4% (18/35) in the eculizumab/eculizumab group and to 62.9% (22/35) in the placebo/eculizumab group.

Patients in the eculizumab/eculizumab and placebo/eculizumab groups received eculizumab for different periods of time during REGAIN and its open-label extension; of all patients who received eculizumab for 26 weeks (eculizumab/eculizumab group to week 26 of REGAIN and placebo/eculizumab group to week 26 of the open-label study), most (66.1%; 74/112) achieved improved status and over one-third (36.6%; 41/112) achieved MM status (Table 30). Almost one-third of participants (32.1%; 36/112) had a status of unchanged and two patients had a status of worse after 26 weeks' eculizumab therapy (Table 30). The proportions of patients who achieved improved or MM status increased with continued eculizumab treatment: 88.0% (66/75) of those who received eculizumab for 130 weeks achieved improved status and 57.3% (43/75) achieved MM status (Table 30). In addition, two patients achieved PR after 130 weeks of eculizumab therapy.

There were no differences in mean age (46.5 versus 47.4 years; P=0.7919) or mean disease duration (9.3 versus 10.3 years; P=0.5334) between eculizumab-treated patients who achieved MM to open-label study week 130 (n=76) and those who did not (n=37). these groups

Safety

Safety data have previously been published for REGAIN and the interim analysis of the open-label study. Across these two studies, the most common adverse events with eculizumab for patients included in this analysis were headache and nasopharyngitis, which were experienced by 44.4% and 38.5% of patients, respectively (Table 31). Serious adverse events of worsening of MG and MG crisis occurred in 15.4% and 3.4% of patients, respectively. MG exacerbations were experienced by 29.1% of patients, and 25.6% used rescue therapy.

TABLE 29
CHANGE IN MYASTHENIA GRAVIS FOUNDATION OF AMERICA POST-INTERVENTION STATUS
FROM REGAIN BASELINE DURING REGAIN AND ITS OPEN-LABEL EXTENSION BY TREATMENT GROUP

| | | Placebo/eculizumab N = 61 | | | | Eculizumab/eculizumab N = 56 | | | |
|---|---|---|---|---|---|---|---|---|---|
| | | | Minimal | | | | Minimal | | |
| | | Improved | manifestations | Unchanged | Worse | Improved | manifestations | Unchanged | Worse |
| Study | Visit | n/N (%) | n/N (%) | n/N (%) | n/N (%) | n/N (%) | n/N (%) | n/N (%) | n/N (%) |
| REGAIN | Week 4 | 15/61 (24.6) | 5/61 (8.2) | 41/61 (67.2) | 5/61 (8.2) | 30/55 (54.5) | 10/55 (18.2) | 25/55 (45.5) | 0/55 (0.0) |
| | Week 12 | 22/61 (36.1) | 7/61 (11.5) | 35/61 (57.4) | 4/61 (6.6) | 28/53 (52.8) | 11/53 (20.8) | 24/53 (45.3) | 1/53 (1.9) |
| | Week 26 | 25/60 (41.7) | 8/60 (13.3) | 30/60 (50.0) | 5/60 (8.3) | 34/56 (60.7) | 14/56 (25.0) | 21/56 (37.5) | 1/56 (1.8) |
| Open-label | Week 26 | 40/56 (71.4) | 27/56 (48.2) | 15/56 (26.8) | 1/56 (1.8) | 36/48 (75.0) | 22/48 (45.8) | 12/48 (25.0) | 0/48 (0.0) |
| study | Week 40 | 46/54 (85.2) | 34/54 (63.0) | 7/54 (13.0) | 1/54 (1.9) | 37/49 (75.5) | 21/49 (42.9) | 10/49 (20.4) | 2/49 (4.1) |
| | Week 52 | 44/54 (81.5) | 31/54 (57.4) | 10/54 (18.5) | 0/54 (0.0) | 41/48 (85.4) | 22/48 (45.8) | 6/48 (12.5) | 1/48 (2.1) |
| | Week 78 | 42/48 (87.5) | 29/48 (60.4) | 5/48 (10.4) | 1/48 (2.1) | 37/47 (78.7) | 22/47 (46.8) | 9/47 (19.1) | 1/47 (2.1) |
| | Week 104 | 34/37 (91.9) | 23/37 (59.5) | 2/37 (5.4) | 1/37 (2.7) | 33/40 (82.5) | 21/40 (52.5) | 6/40 (15.0) | 1/40 (2.5) |
| | Week 130 | 33/35 (94.3) | 22/35 (62.9) | 2/35 (5.7) | 0/35 (0.0) | 28/35 (80.0) | 18/35 (51.4) | 5/35 (14.3) | 2/35 (5.7) |

TABLE 30
CHANGE IN MYASTHENIA GRAVIS FOUNDATION OF
AMERICA POST-INTERVENTION STATUS FROM REGAIN
BASELINE BY ECULIZUMAB TREATMENT DURATION

| Duration of | | Minimal | | |
|---|---|---|---|---|
| eculizumab | Improved | manifestations | Unchanged | Worse |
| treatment | n/N (%) | n/N (%) | n/N (%) | n/N (%) |
| 26 weeks | 74/112 (66.1) | 41/112 (36.6) | 36/112 (32.1) | 2/112 (1.8) |
| 52 weeks | 80/102 (78.4) | 53/102 (52.0) | 22/102 (21.6) | 0/102 (0.0) |
| 78 weeks | 83/96 (86.5) | 51/96 (53.1) | 11/96 (11.5) | 2/96 (2.1) |
| 104 weeks | 71/84 (84.5) | 44/84 (52.4) | 11/84 (13.1) | 2/84 (2.4) |
| 130 weeks | 66/75 (88.0) | 43/75 (57.3) | 8/75 (10.7) | 1/75 (1.3) |

TABLE 31
SAFETY OUTCOMES FOR THE STUDY POPULATION DURING REGAIN
AND THE OPEN-LABEL STUDY

| | REGAIN | | REGAIN | | REGAIN and open-label study | |
|---|---|---|---|---|---|---|
| | Placebo | | Eculizumab | | Eculizumab | |
| | (N = 61, 30.9 PY^{a}) | | (N = 56, 28.2 PY^{a}) | | (N = 117, 305.1 PY^{a}) | |
| | Patients with | Event rate | Patients with | Event rate | Patients with | Event rate |
| Outcome | event, n (%) | (events/100 PY^{a}) | event, n (%) | (events/100 PY^{a}) | event, n (%) | (events/100 PY^{a}) |
| Exacerbation | 13 (21.3) | 77.7 | 4 (7.1) | 35.5 | 34 (29.1) | 23.9 |
| Rescue therapy use | 10 (16.4) | 68.0 | 4 (7.1) | 35.5 | 30 (25.6) | 22.0 |

Most common adverse events^{b,c} (>15% of all patients)

| | | | | | | |
|---|---|---|---|---|---|---|
| Headache | 12 (19.7) | 90.6 | 10 (17.9) | 88.7 | 52 (44.4) | 32.8 |
| Nasopharyngitis | 10 (16.4) | 42.1 | 9 (16.1) | 46.1 | 45 (38.5) | 32.1 |
| Diarrhea | 8 (13.1) | 29.1 | 8 (14.3) | 35.5 | 33 (28.2) | 17.4 |
| Upper respiratory | 12 (19.7) | 45.3 | 9 (16.1) | 46.1 | 31 (26.5) | 25.2 |
| tract infection | | | | | | |
| Nausea | 9 (14.8) | 84.1 | 7 (12.5) | 35.5 | 27 (23.1) | 12.1 |
| Myasthenia | 9 (14.8) | 58.3 | 4 (7.1) | 14.2 | 30 (25.6) | 17.4 |
| gravis^{d} | | | | | | |
| Arthralgia | 5 (8.2) | 29.1 | 1 (1.8) | 3.5 | 24 (20.5) | 10.5 |
| Pain in extremity | 2 (3.3) | 6.5 | 4 (7.1) | 14.2 | 20 (17.1) | 8.5 |
| Urinary tract | 5 (8.2) | 22.7 | 3 (5.4) | 14.2 | 20 (17.1) | 12.5 |
| infection | | | | | | |

Most common myasthenia gravis- and infection-related serious adverse events^{b,c} (≥2 patients)

| | | | | | | |
|---|---|---|---|---|---|---|
| Myasthenia gravis^{d} | 6 (9.8) | 45.3 | 3 (5.4) | 10.6 | 18 (15.4) | 10.5 |
| Pyrexia | 0 (0.0) | 0.0 | 2 (3.6) | 7.1 | 5 (4.3) | 1.6 |
| Myasthenia gravis | 0 (0.0) | 0.0 | 0 (0.0) | 0.0 | 4 (3.4) | 1.3 |
| crisis | | | | | | |
| Acute respiratory | 0 (0.0) | 0.0 | 0 (0.0) | 0.0 | 3 (2.6) | 1.0 |
| failure | | | | | | |
| Gastroenteritis | 1 (1.6) | 9.7 | 0 (0.0) | 0.0 | 3 (2.6) | 1.0 |
| Pneumonia | 0 (0.0) | 0.0 | 0 (0.0) | 0.0 | 4 (3.4) | 1.6 |
| Sepsis | 0 (0.0) | 0.0 | 0 (0.0) | 0.0 | 3 (2.6) | 1.0 |
| Bronchitis | 0 (0.0) | 0.0 | 0 (0.0) | 0.0 | 2 (1.7) | 1.0 |
| Urinary tract | 0 (0.0) | 0.0 | 0 (0.0) | 0.0 | 2 (1.7) | 1.0 |
| infection | | | | | | |
| Influenza | 0 (0.0) | 0.0 | 0 (0.0) | 0.0 | 2 (1.7) | 0.7 |
| Upper respiratory | 2 (3.3) | 6.5 | 0 (0.0) | 0.0 | 2 (1.7) | 0.7 |
| tract infection | | | | | | |
| Pneumonia | 0 (0.0) | 0.0 | 0 (0.0) | 0.0 | 2 (1.7) | 0.7 |
| aspiration | | | | | | |
| Localized infection | 0 (0.0) | 0.0 | 0 (0.0) | 0.0 | 2 (1.7) | 0.7 |

^{a}PY is the sum of all years for all patients and the observed event rate is the number of events per PY multiplied by 100. B. Medical Dictionary for Regulatory Activities preferred term.
^{c}If a patient had more than one adverse event for a particular preferred term, that patient is counted only once for that preferred term.
^{d}Worsening (increased frequency and/or intensity) of a preexisting condition, including myasthenia gravis, is considered to be an adverse event.
PY, patient year.

This analysis found that patients with AChR+refractory gMG treated with eculizumab experienced rapid improvements in their clinical condition based on MGFA post-intervention status. Over 50% of patients achieved a status of improved within 4 weeks of their first dose of eculizumab in REGAIN and one-third of these patients also achieved MM. By REGAIN week 26, significantly higher proportions of eculizumab-treated patients than placebo-treated patients achieved an MGFA post-intervention status of improved or MM.

Long-term eculizumab treatment was associated with further increases in the proportions of patients who achieved a status of improved or MM. After 130 weeks of eculizumab therapy, almost 90% of all patients had attained a status of improved and nearly 60% had achieved MM. These findings suggest that for some patients with AChR+refractory gMG, long-term treatment with eculizumab was required for optimal disease control. Furthermore, two patients achieved PR after 130 weeks of eculizumab treatment, reflecting long-term maintenance of symptom relief.

The long-term safety profile of eculizumab was consistent with its known profile from over 10 years of clinical use in other indications, and no new safety signals were observed during the open-label study.

The main limitation of this analysis was the open-label design of the extension study, which could yield unconscious bias in reporting. Because over 90% of patients who enrolled in REGAIN continued into the open-label study, selection bias in the open-label study population was unlikely.

In conclusion, the results of this example confirmed the rapid and sustained clinical response to eculizumab that was observed during REGAIN and the open-label study. Over 50% of patients who were considered to have refractory disease achieved the consensus treatment goal of MM or better after 1 year of eculizumab treatment. These findings further supported the long-term effectiveness of eculizumab for use in patients with AChR+refractory gMG.

### Example 7: Response to Eculizumab in Patients with Myasthenia Gravis Recently Treated with Chronic Intravenous Immunoglobulin (IVIg) Enrolled in ECU-MG-301 REGAIN and ECU-MG-302 Extension Trial

IVIg is a proven short-term therapy for MG exacerbations/crises, and is also considered as a maintenance therapy in treatment-refractory MG inadequately controlled with standard IST. The longevity of its effects is limited, however, and there are some tolerability issues associated with chronic IVIg therapy. In this example, the response of patients recently treated with IVIg to eculizumab enrolled in the ECU-MG-301 REGAIN Trial (see Example 1 for protocol) and the Open-Label Extension (OLE) study (ECU-MG-302; see Example 2 for protocol) was analyzed.

The response to eculizumab was evaluated over a period of up to 18 months in patients receiving chronic intravenous immunoglobulin (IVIg) before participating in REGAIN. This subgroup comprised patients who had received IVIg at least four times in 1 year, with at least one IVIg treatment cycle during the 6 months before the first REGAIN study dose. Patients were excluded from REGAIN if they had received IVIg in the 4 weeks before randomization, and concomitant IVIg use was only permitted during REGAIN/OLE as rescue therapy. Response to eculizumab versus placebo was assessed using four validated, disease-specific measures. Incidence of exacerbations and safety endpoints were recorded.

Specifically, response was assessed using MG-ADL, QMG, MG Composite scale (MGC) and 15-item MG Quality of Life Questionnaire (MG-QOL15). A clinical response was defined as a ≥3-point improvement in MG-ADL total score or a ≥5-point improvement in QMG total score at the evaluation time point compared with REGAIN baseline. The incidence of exacerbations (defined as an MG crisis, significant symptomatic worsening or requirement for rescue therapy) was evaluated throughout REGAIN/OLE, and during the year before REGAIN enrollment. Safety endpoints were assessed throughout REGAIN/OLE.

Changes from REGAIN baseline in MG-ADL, QMG, MGC and MG-QOL15 mean total scores were based on t-tests. Data are presented as the mean change from REGAIN baseline; mean differences between treatment groups are presented as 95% confidence intervals (CIs). The responder analyses measured the proportion of patients with clinically meaningful improvements from REGAIN baseline. Exact (Clopper-Pearson) 95% CIs for the responder proportions are presented. For exacerbations, model-based event rates per 100 patient-years were calculated following previously published methodology. All statistical analyses were performed using SAS version 9.4.

A total number of 18 patients were included (eculizumab, n=9; placebo, n=9) in this analysis. One eculizumab-treated patient withdrew from REGAIN; 17 patients continued into the OLE (eculizumab/eculizumab, n=8; placebo/eculizumab, n=9). Baseline demographics and characteristics, including mean baseline scores for MG-ADL, QMG, MGC and MG-QOL15, were generally similar for both subgroup treatment arms and the overall REGAIN population. The duration between last recorded IVIg dose end date and first REGAIN study dose was 4-16 weeks (n=17; one patient missing accurate end-date data). In the year before entering REGAIN, 9/18 patients experienced at least one exacerbation (eculizumab: six events in four patients; placebo: 19 events in five patients); there was also one patient with MG crisis in the eculizumab group. Compared with the overall REGAIN population, this subgroup experienced a higher rate of exacerbations in the year before REGAIN start (150.0 vs 102.4 exacerbations/100 patient-years).

At REGAIN week 26, eculizumab-treated patients had numerically larger improvements from baseline in MG-ADL and QMG mean total scores than placebo-treated patients (Table 32). Improvements from baseline in MG-ADL, QMG, MGC and MG-QOL15 mean total scores with eculizumab during REGAIN were sustained in the eculizumab/eculizumab group during the OLE. Patients receiving open-label eculizumab after placebo during REGAIN experienced rapid improvements in assessment scores.

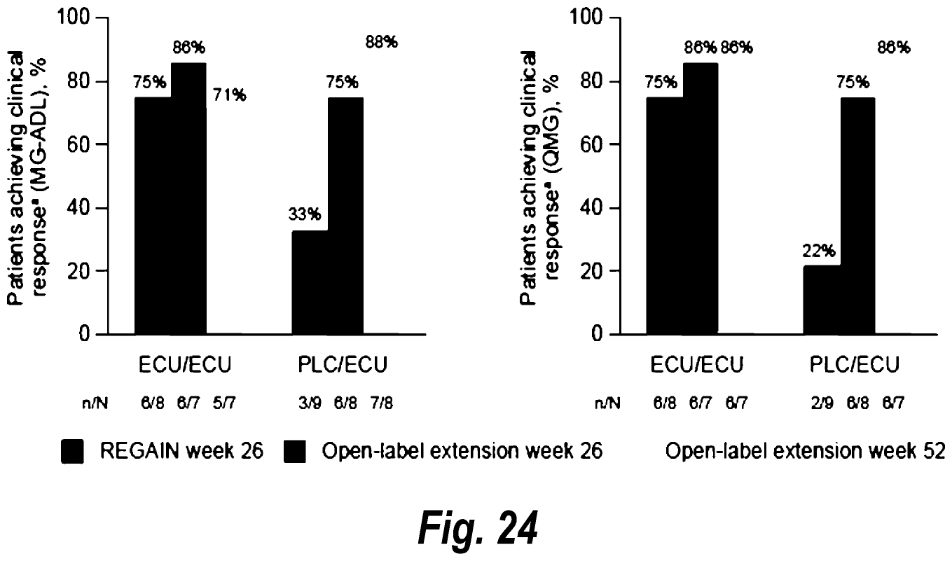

A clinical response (MG-ADL or QMG) was achieved by most subgroup patients receiving eculizumab during REGAIN and the OLE (FIG. 24). In contrast, only a third or fewer subgroup patients achieved a clinical response while receiving placebo in REGAIN (FIG. 24). Most of these patients subsequently achieved a clinical response (MG-ADL or QMG) after receiving eculizumab during the OLE.

TABLE 32
CHANGE FROM REGAIN BASELINE IN MG-ADL, QMG, MGC, AND MG-QOL15 MEAN
TOTAL SCORES TO REGAIN WEEK 26 AND OPEN-LABEL EXTENSION WEEKS 26 AND 52

| | | | | Open-label extension | | Open-label extension | | Open-label extension | |
|---|---|---|---|---|---|---|---|---|---|
| | | REGAIN week 26^{a} | | week 4^{b} | | week 26^{b} | | week 52^{b} | |
| | REGAIN | | Mean change | | Mean change | | Mean change | | Mean change |
| | treatment | | from REGAIN | | from REGAIN | | from REGAIN | | from REGAIN |
| Assessment | group | n | baseline (95% CI) | n | baseline (95% CI) | n | baseline (95% CI) | n | baseline (95% CI) |
| MG-ADL | Eculizumab | 9 | −5.3 | 8 | −4.6 | 7 | −5.1 | 7 | −4.7 |
| | | | (−8.4 to −2.3) | | (−8.0 to −1.3) | | (−7.6 to −2.7) | | (−7.2 to −2.2) |
| | Placebo | 9 | −2.1 | 8 | −6.6 | 8 | −5.4 | 8 | −5.9 |
| | | | (−4.3 to 0.0) | | (−10.2 to −3.1) | | (−8.0 to −2.8) | | (−9.5 to −2.3) |
| QMG | Eculizumab | 9 | −4.1 | 7 | −5.3 | 7 | −3.9 | 7 | −6.3 |
| | | | (−8.8 to −0.6) | | (−9.3 to −1.2) | | (−8.9 to 1.2) | | (−9.1 to −3.5) |
| | Placebo | 9 | −1.3 | 7 | −8.3 | 8 | −6.6 | 7 | −7.7 |
| | | | (−4.0 to 1.4) | | (−12.9 to −3.7) | | (−11.7 to −1.5) | | (−11.1 to −4.3) |
| MGC | Eculizumab | 9 | −9.7 | 7 | −9.4 | 7 | −10.3 | 7 | −11.3 |
| | | | (−14.1 to −5.2) | | (−16.6 to −2.2) | | (−14.2 to −6.4) | | (−16.2 to −6.3) |
| | Placebo | 9 | −3.9 | 7 | −14.0 | 8 | −8.6 | 8 | −9.0 |
| | | | (−8.2 to 0.4) | | (−19.8 to −8.2) | | (−15.4 to −1.8) | | (−17.5 to −0.5) |
| MG-QOL15 | Eculizumab | 9 | −9.9 | 8 | −13.1 | 6 | −15.5 | 7 | −14.7 |
| | | | (−21.5 to 1.8) | | (−25.2 to −1.1) | | (−32.2 to 1.2) | | (−23.3 to −6.1) |
| | Placebo | 9 | −4.2 | 8 | −14.6 | 8 | −13.6 | 8 | −16.5 |
| | | | (−10.8 to 2.4) | | (−26.5 to −2.8) | | (−22.4 to −4.9) | | (−27.8 to −5.2) |

^{a}Last observation carried forward for one patient who discontinued before REGAIN week 26.
^{b}Data calculated for patients remaining in the study at this assessment. CI, confidence interval; MG-ADL, Myasthenia Gravis Activities of Daily Living; MGC, Myasthenia Gravis Composite scale; Mg-QOL15, 15-item Myasthenia Gravis Quality of Life Questionaire; QMG, Quantitative Myasthenia Gravis test.

During REGAIN, 4/18 patients experienced exacerbations; one eculizumab-treated and three placebo-treated patients experienced 7 and 12 exacerbations, respectively. During the OLE, 6/17 patients experienced exacerbations; all six were in the eculizumab/eculizumab group. Most exacerbations occurring in eculizumab-treated patients during REGAIN (7/7 exacerbations) and the OLE (9/15 exacerbations) were experienced by one patient. The exacerbation rate was reduced by 68.6% from 150.0 exacerbations/100 patient-years in the year before REGAIN (n=18) to 47.0 exacerbations/100 patient-years during the OLE (n=17; p=0.1531). The exacerbation rate in the OLE population (n=17) also compared favorably with the REGAIN placebo group (n=9; 240.9 exacerbations per 100 patient-years; 80.4% reduction, p=0.059).

The safety profile of eculizumab in the chronic IVIg subgroup was consistent with that in all eculizumab-treated patients in REGAIN. The most common adverse events with eculizumab in this subgroup were headache (REGAIN, 22.2%; OLE, 52.9%; versus REGAIN placebo, 11.1%) and upper respiratory tract infection (33.3%, 47.1% and 33.3%, respectively). One patient in this subgroup analysis died during the OLE. This death was attributed to end-stage liver disease; the patient had cryptogenic liver cirrhosis and a history of fatty liver.

The baseline patient characteristics were similar to the REGAIN population; the IVIg subgroup had previously demonstrated poorly controlled disease by virtue of the need for chronic IVIg and the high exacerbation rate in the year before the start of the study.

During the 26-week REGAIN study and the OLE, eculizumab was associated with sustained improvements from baseline in all assessment scores in this subgroup. As in the overall OLE population, subgroup patients who received placebo in REGAIN experienced a rapid and sustained clinical response with eculizumab. During REGAIN, MG exacerbations were more frequent in subgroup patients receiving placebo versus eculizumab, consistent with active disease in this cohort. The safety profile in this group was similar to that in the overall population.

During the OLE, there were 15 exacerbations in the eculizumab/eculizumab group, but none in patients receiving placebo in REGAIN and then eculizumab (placebo/eculizumab). This discrepancy is largely attributable to one patient, who was the only eculizumab-treated patient in the subgroup to experience any exacerbations during REGAIN and who contributed over half of all exacerbations reported in the OLE. The exacerbation rate during eculizumab treatment in the OLE was reduced considerably from that in the year before REGAIN start and that in the REGAIN placebo group.

These data demonstrated that, for patients who had previously received chronic IVIg, eculizumab resulted in rapid improvements in MG signs and symptoms across four validated measures of disease severity, which were maintained over 18 months. There were also fewer MG exacerbations with eculizumab compared with placebo during REGAIN.

### Example 8: ‘Minimal Symptom Expression’ in Patients with Acetylcholine Receptor Antibody-Positive Refractory Myasthenia Gravis Treated with Eculizumab Enrolled in ECU-MG-301 REGAIN and ECU-MG-302 Extension Trial

In this example, the response of AChR+refractory gMG patients to eculizumab enrolled in the ECU-MG-301 REGAIN Trial (see Example 1 for protocol) and the Open-Label Extension (OLE) study (ECU-MG-302; see Example 2 for protocol) was analyzed. The objective of REGAIN and the open-label extension study was to assess the tolerability of eculizumab and its efficacy, as measured by change in MG-ADL total score from each study's baseline. This sub-analysis evaluated the achievement of ‘minimal symptom expression’ in both studies, defined as achievement of an MG-ADL total score of 0-1 (range 0-24) or an MG-QOL15 total score of 0-3 (range 0-60). In contrast to current definitions of minimal symptoms, which rely on physical evaluation by a clinician, this analysis of ‘minimal symptom expression’ is based exclusively on patients' assessments of their symptoms.

In a validation study for the MG-QOL15, patients in remission had a mean MG-QOL15 total score of 3.3 (standard deviation, 4.4), with a range of 0-15. Remission was defined as an MG composite score of 0 and a score of 0 on either the MG-ADL or the MG manual muscle test, with the exception that an eye closure score of 1 (mild weakness) was permitted. The proportions of patients achieving ‘minimal symptom expression’ were calculated for the eculizumab and placebo treatment groups at week 26 of REGAIN and up to week 130 of the open-label extension (a total of 156 weeks of eculizumab treatment for the eculizumab/eculizumab group and 130 weeks of eculizumab treatment for the placebo/eculizumab group).

Adverse events were recorded and coded by preferred term using the Medical Dictionary for Regulatory Activities version 20.1. MG exacerbations, rescue therapy use and discontinuations because of adverse events were also recorded.

The significance of differences between groups was evaluated by calculating p values based on Fisher's exact test.

Results

Data are reported from the REGAIN study and its open-label extension for up to a maximum total of 156 weeks of eculizumab treatment. Of the 118 patients who completed REGAIN, 117 patients continued into the open-label study (eculizumab/eculizumab n=56, placebo/eculizumab n=61; FIG. 22) and were included in the efficacy and safety analyses. One patient from the eculizumab/eculizumab group withdrew from the open-label study during the blind induction phase, prior to entering the maintenance phase. Patient demographics and characteristics were similar for the eculizumab/eculizumab and placebo/eculizumab groups, with the exception that there was a greater proportion of Asian patients in the placebo/eculizumab group (Table 33).

TABLE 33
DEMOGRAPHICS AND CHARACTERISTICS AT REGAIN
BASELINE OF PATIENTS WHO CONTINUED FROM REGAIN
INTO THE OPEN-LABEL EXTENSION STUDY

| | Eculizumab/ | Placebo/ | All |
|---|---|---|---|
| | eculizumab | eculizumab | patients |
| Variable | n = 56 | n = 61 | N = 117 |
| Age, years,^{a} mean (SD) | 46.8 (15.6) | 47.0 (17.8) | 46.9 (16.7) |
| Sex, n (%) | | | |
| Male | 18 (32.1) | 20 (32.8) | 38 (32.5) |
| Female | 38 (67.9) | 41 (67.2) | 79 (67.5) |
| Race, n (%) | | | |
| Asian | 3 (5.4) | 16 (26.2) | 19 (16.2) |
| Black or African-American | 0 (0.0) | 2 (3.3) | 2 (1.7) |
| White | 47 (83.9) | 41 (67.2) | 88 (75.2) |
| Other/multiple/unknown | 6 (10.7) | 2 (3.3) | 8 (6.8) |
| Duration of MG,^{b} years, mean | 10.2 (7.9) | 9.2 (8.6) | 9.7 (8.2) |
| (SD) | | | |
| Baseline MG-ADL total score, | 10.3 (3,0) | 9.9 (2.6) | 10.1 (2.8) |
| mean (SD) | | | |
| Baseline MG-QOL15 total score, | 32.5 (12.0) | 30.8 (12.9) | 31.6 (12.5) |
| mean (SD) | | | |

^{a}At first dose in REGAIN
^{b}Time from MG diagnosis to date of first dose in REGAIN
MG myasthenia gravis, MG-ADL myasthenia gravis activities of daily living questionnaire, MG-QOL15 15-item myasthenia gravis quality of life questionnaire, SD standard deviation

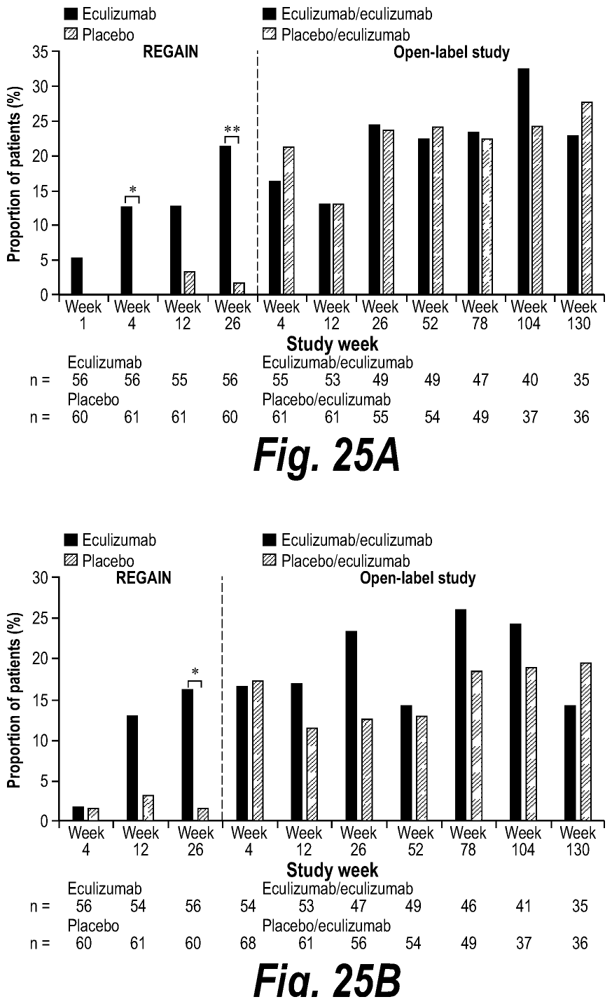

At week 26 of REGAIN, a significantly higher proportion of patients receiving eculizumab achieved ‘minimal symptom expression’ than of those receiving placebo according to MG ADL score (21.4% and 1.7%, respectively; difference 19.8%; 95% confidence interval [CI] 8.5, 31.0; p=0.0007; *p<0.01; **p<0.001 vs placebo; FIG. 25a) and MG-QOL 15 score (16.1% and 1.7%, respectively; difference 14.4%; 95% C14.3, 24.6; p=0.0069; FIG. 25b).

During the open-label extension, the proportion of patients in the eculizumab/eculizumab group with ‘minimal symptom expression’ was maintained for 2.5 years, between REGAIN week 26 and open-label week 130 (MG-ADL: 21.4% and 22.9%, respectively; MG-QOL15: 16.1% and 14.3%, respectively). In the placebo/eculizumab group, the proportion of patients with ‘minimal symptom expression’ increased to levels similar to those in the eculizumab/eculizumab group within 4 weeks of starting open-label eculizumab therapy, between REGAIN week 26 and open-label week 4 (MG-ADL: 1.7% and 21.3%, respectively; MG-QOL15: 1.7% and 17.2%, respectively). This increase was sustained to open-label week 130 (MG-ADL: 27.8%; MG-QOL15: 19.4%).

At week 130 of the open-label extension, ‘minimal symptom expression’ was achieved by similar proportions of patients in the eculizumab/eculizumab and placebo/eculizumab groups as assessed by MG-ADL score (22.9% and 27.8%, respectively; difference −4.9%; 95% CI −25.1, 15.3; p=0.7861; FIG. 25a). The proportions of patients achieving ‘minimal symptom expression’ at week 130 based on MG-QOL15 score were also similar in the two groups: 14.3% in the eculizumab/eculizumab group and 19.4% in the placebo/eculizumab group (difference −5.2%; 95% CI −22.5, 12.2; p=0.7531; FIG. 25b). Overall, 25.4% of eculizumab-treated patients experienced ‘minimal symptom expression’ according to MG-ADL and 16.9% according to MG-QOL15 at this time point.

There was no significant difference in mean age at first eculizumab dose between eculizumab-treated patients who achieved ‘minimal symptom expression’ according to MG-ADL during REGAIN and the open-label study (up to week 130) and those who did not (47.4 vs 47.0 years; p=0.8847). Mean disease duration at first eculizumab dose was shorter for patients who achieved ‘minimal symptom expression’ by open-label week 130 than for those who did not (8.27 [range 1.6-27.0] vs 11.16 [range 1.7-34.4] years; p=0.0474). For achievement of ‘minimal symptom expression’ according to MG-QOL15 up to open-label week 130, there were no significant differences in mean age (44.6 vs 48.4 years; p=0.2611) or mean disease duration at first eculizumab dose (8.73 [range 1.6-24.6] vs 10.51 [range 1.7-34.4] years; p=0.2091).

The mean MG-ADL total score for the open-label study population decreased from 10.1 (standard deviation [SD] 2.80; n=117) at REGAIN baseline to 3.9 (SD 3.08; n=71) at open-label week 130. The mean MG-QOL15 total score also reduced between these time points, from 31.6 (SD 12.48) to 15.3 (SD 12.15).

In conclusion, this analysis found that, at the end of REGAIN, a significantly greater proportion of patients with AChR+refractory gMG treated with eculizumab experienced ‘minimal symptom expression’ than of those receiving placebo according to an MG-ADL total score of 0-1 or an MG-QOL15 total score of 0-3. The proportions of patients experiencing ‘minimal symptom expression’ were maintained through 2.5 years of open-label eculizumab therapy in the extension study.

It is notable that, among a group of patients with refractory gMG with a mean MG-ADL total score of 10.1 at the start of REGAIN, approximately a quarter reported ‘minimal symptom expression’ defined as an MG-ADL total score of 0-1 through week 130 of the open-label study, by which time point the mean MG-ADL total score had reduced by more than half to 3.9. This reflects patient-reported improvements in disease burden in excess of the two-point reduction in MG-ADL total score that is considered to be a clinically meaningful improvement, to a level that has previously been described as disease remission.

In addition, ‘minimal symptom expression’, defined as an MG-QOL15 total score of 0-3, was achieved by one-sixth of these patients, and the mean MG-QOL15 total score halved between the start of REGAIN (31.6) and week 130 of the open-label study (15.3). The smaller proportion achieving ‘minimal symptom expression’ according to MG-ADL versus MG-QOL15 (one-quarter vs one-sixth) may be due to the conservative MG-QOL15 total score range (0-3) used in the definition of ‘minimal symptom expression’ in this analysis.

The long-term safety profile of eculizumab was consistent with its known profile from over 10 years of clinical use in other indications, and no new safety signals were observed during the open-label study.

### Example 9: Long-Term Efficacy of Eculizumab in Refractory Generalized Myasthenia Gravis Patients: Responder Analyses of Patients Enrolled in ECU-MG-301 REGAIN and ECU-MG-302 Extension Trial

In this example, long-term efficacy of eculizumab in responder patients enrolled in the ECU-MG-301 REGAIN Trial (see Example 1 for protocol) and the Open-Label Extension (OLE) study (ECU-MG-302; see Example 2 for protocol) was analyzed by MG-ADL and QMG score. The objective of REGAIN and the open-label extension study was to assess the tolerability of eculizumab and its efficacy, as measured by change in MG-ADL total score from each study's baseline. This sub-analysis evaluated responder data for patients enrolled in the REGAIN trial and its OLE, using MG-ADL and QMG scores. The outcomes examined included time to response and baseline/demographic factors that may predict the likelihood of response. Responder analysis baseline for eculizumab/eculizumab patients was the REGAIN baseline assessment. Responder analysis baseline for placebo/eculizumab patients was the OLE baseline assessment.

For all response assessments, patients only had to achieve a response once during the evaluation timeframe, rather than for a sustained period. An MG-ADL response was defined as an achievement of at least a three-point (i.e., ≥3-point) reduction from baseline, whereas a QMG response was defined as at least a five-point (i.e., ≥5-point) reduction from baseline. An “early responder” was defined as a response achieved on or before the Week 12 assessment of the REGAIN study. A “late responder” was defined as a response achieved on or after the Week 12 assessment of the REGAIN study. A “non-responder” was defined as no response on eculizumab up to the end of the OLE study.

Results

A total of 98 patients were evaluated in this sub-analysis, all of whom received eculizumab. Patients who received placebo during REGAIN and had an MG-ADL total score of greater than 6 at eculizumab initiation were excluded from this analysis.

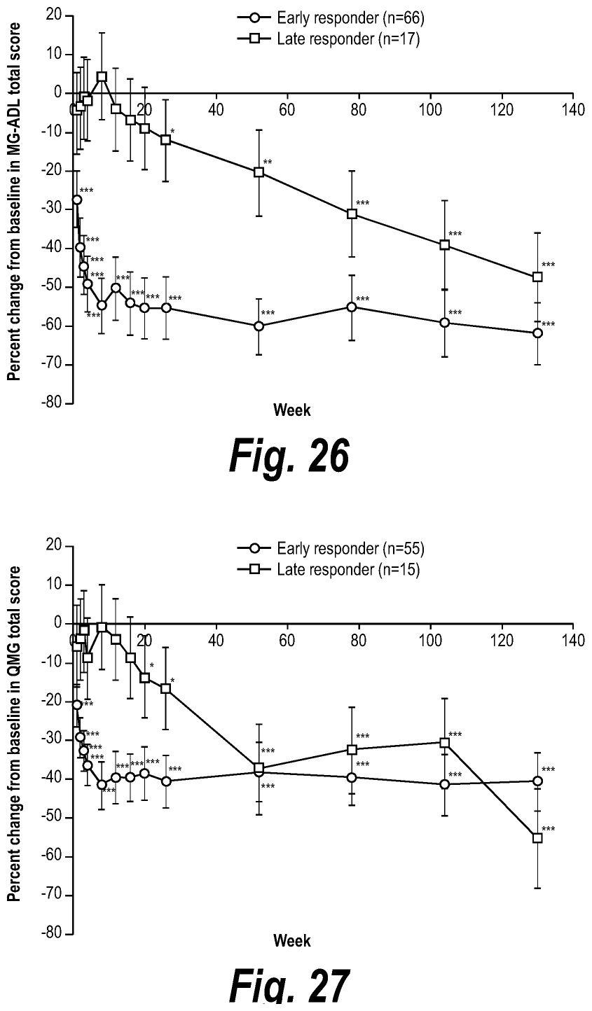

An MG-ADL response was achieved by 66 patients (67.3%) at some point by Week 12 (Table 34; FIG. 26). A further 17 patients (17.3%) achieved an MG-ADL response after Week 12. Of these, 11 patients (11.2%) achieved a response after Week 26 (Table 34; FIG. 26). A total of patients (15.3%) did not achieve an MG-ADL response during the REGAIN or OLE studies (Table 34). Baseline characteristics by MG-ADL responder category is shown in Table 35.

A QMG response was achieved by 55 patients (56.1%) at some point by Week 12 (Table 35; FIG. 27). A further 15 patients (15.3%) achieved a QMG response after Week 12. Of these, 9 patients (9.2%) achieved a response after Week 26 (Table 36; FIG. 27). A total of 28 patients (28.6%) did not achieve a QMG response during the REGAIN or OLE studies (Table 36). Baseline characteristics by QMG responder category is shown in Table 37.

The majority of patients showed a clinically meaningful response in MG-ADL and QMG scores within the first 12 weeks of receiving eculizumab. Among these “early responders,” 30 patients (45.5%) were classified as MG-ADL responders and 20 patients (36.4%) were classified as QMG responders, at all timepoints at and following the timepoint at which the response was initially observed. The majority of MG-ADL early responders (59/66; 89.4%) and QMG early responders (43/55; 78.1%) were classified as responders at >50% of timepoints at and following the first response timepoint. After the first response, an MG-ADL response was recorded at 85.1% (941/1106) of the assessments, and a QMG response was recorded at 72.2% (642/889) of the assessments. 56.1% (37/66) of MG-ADL early responders had changes to their corticosteroid therapy. Of these, 83.8% (31/37) reduced their dose or stopped treatment. 52.7% (29/55) of QMG early responders had changes to their corticosteroid therapy. Of these, 89.7% (26/29) reduced their dose or stopped treatment.

In contrast to “early responders,” 15-17% of patients showed a clinically meaningful response at a later timepoint. Of these “late responders,” 6 patients (35.3%) were classified as MG-ADL responders and 1 patient (6.7%) was classified as a QMG responder, at all timepoints at and following the timepoint at which the response was initially observed. 12 out of 17 patients (70.6%) were classified as MG-ADL responders, and 9 out of 15 patients (60.0%) were classified as QMG responders at >50% of timepoints at and following the first response timepoint. After the first response, an MG-ADL response was recorded at 58.5% (76/130) of assessments, and a QMG response was recorded at 57.2% (83/145) of assessments. 35.3% (6/17) of MG-ADL late responders had changes to their corticosteroid therapy. Of these, 66.7% (4/6) decreased treatment. 53.3% (8/15) of QMG late responders had changes to their corticosteroid therapy. Of these, 62.5% (5/8) decreased or stopped treatment.

These findings suggest that although most patients with refractory generalized MG will achieve clinical response (assessed by MG-ADL or QMG scores) by Week 12 of eculizumab treatment, additional first responses can be observed with longer-term treatment.

TABLE 34
NUMBER OF PATIENTS ACHIEVING FIRST MG-ADL
RESPONSE BY EACH ASSESSMENT VISIT

| | Patients achieving | Cumulative number |
|---|---|---|
| | first response | of patients achieving |
| Visit | (N = 98), n (%) | a response (N = 98) (%) |
| Week 1 | 32 (32.7) | 32 (32.7) |
| Week 2 | 14 (14.3) | 46 (46.9) |
| Week 3 | 5 (5.1) | 51 (52.0) |
| Week 4 | 5 (5.1) | 56 (57.1) |
| Week 8 | 7 (7.1) | 63 (64.3) |
| Week 12 | 3 (3.1) | 66 (67.3) |
| Week 16 | 2 (2.0) | 68 (69.4) |
| Week 20 | 2 (2.0) | 70 (71.4) |
| Week 26 | 2 (2.0) | 72 (73.5) |
| After Week 26 | 11 (11.2) | 83 (84.7) |
| Never achieved | 15 (15.3) | — |
| responder level | | |

MG-ADL, myasthenia gravis activities of daily living

TABLE 35
BASELINE CHARACTERISTICS BY MG-
ADL RESPONDER CATEGORIES

| | Early responders | Late responders | Non-responders |
|---|---|---|---|
| Variable | (N = 66) | (N = 17) | (N = 15) |
| Age at first | 44.8 (16.25) | 49.5 (17.10) | 52.3 (14.02) |
| eculizumab dose; | | | |
| years, mean (SD) | | | |
| Male, n (%) | 19 (28.8) | 5 (29.4) | 5 (33.3) |
| Duration of MG; | 9.93 (7.68) | 9.96 (9.50) | 14.15 (10.82) |
| years, mean (SD) | | | |
| Baseline MG-ADL | 10.5 (3.00) | 9.1 (2.71) | 8.5 (2.17) |
| score, mean (SD) | | | |
| MGFA class at | | | |
| screening, n (%) | | | |
| IIa | 14 (21.2) | 4 (23.5) | 3 (20.0) |
| IIb | 7 (10.6) | 4 (23.5) | 1 (6.7) |
| IIIa | 18 (27.3) | 6 (35.3) | 4 (26.7) |
| IIIb | 16 (24.2) | 2 (11.8) | 7 (46.7) |
| IVa | 6 (9.1) | 0 (0) | 0 (0) |
| IVb | 5 (7.6) | 1 (5.9) | 0 (0) |
| ISTs before | | | |
| REGAIN, n (%) | | | |
| 2 | 28 (42.4) | 9 (52.9) | 3 (20.0) |
| 3 | 25 (37.9) | 5 (29.4) | 7 (46.7) |
| ≥4 | 11 (16.7) | 3 (17.6) | 5 (33.3) |
| MG crisis before | 11 (16.7) | 3 (17.6) | 3 (20.0) |
| REGAIN, n (%) | | | |

IST, immunosuppressive therapy;
MG, myasthenia gravis;
MG-ADL, myasthenia gravis-activities of daily living;
MGFA. Myasthenia Gravis Foundation of America;
SD, standard deviation; all comparisons were non-significant based on two-sample t test for continuous variables and Fisher's exact test for categotical variables

TABLE 36
NUMBER OF PATIENTS ACHIEVING FIRST QMG
RESPONSE BY EACH ASSESSMENT VISIT

| | Patients achieving | Cumulative number |
|---|---|---|
| | first response | of patients achieving |
| Visit | (N = 98), n (%) | a response (N = 98) (%) |
| Week 1 | 19 (19.4) | 19 (19.4) |
| Week 2 | 13 (13.3) | 32 (32.7) |
| Week 3 | 10 (10.2) | 42 (42.9) |
| Week 4 | 4 (4.1) | 46 (46.9) |
| Week 8 | 6 (6.1) | 52 (53.1) |
| Week 12 | 3 (3.1) | 55 (56.1) |
| Week 16 | 2 (2.0) | 57 (58.2) |
| Week 20 | 4 (4.1) | 61 (62.2) |
| Week 26 | 0 (0) | 61 (62.2) |
| After Week 26 | 9 (9.2) | 70 (71.4) |
| Never achieved | 28 (28.6) | — |
| responder level | | |

QMG, quantitative myasthenia gravis

TABLE 37
BASELINE CHARACTERISTICS BY
QMG RESPONDER CATEGORIES

| | Early responders | Late responders | Non-responders |
|---|---|---|---|
| Variable | (N = 55) | (N = 15) | (N = 28) |
| Age at first | 42.6 (15.17) | 49.1 (18.70) | 53.7 (14.60) |
| eculizumab dose; | | | |
| years, mean (SD) | | | |
| Male, n (%) | 14 (25.5) | 6 (40.0) | 9 (32.1) |
| Duration of MG; | 10.46^{†} (7.83) | 5.46 (2.70) | 13.58 (10.68) |
| years, mean (SD) | | | |
| Baseline QMG | 18.6^{‡} (5.24) | 15.1 (4.12) | 14.5 (5.32) |
| score, mean (SD) | | | |
| MGFA class at | | | |
| screening, n (%) | | | |
| IIa | 13 (23.6) | 1 (6.7) | 7 (25.0) |
| IIb | 7 (12.7) | 2 (13.3) | 3 (10.7) |
| IIIa | 14 (25.5) | 4 (26.7) | 10 (35.7) |
| IIIb | 12 (21.8) | 5 (33.3) | 8 (28.6) |
| IVa | 5 (9.1) | 1 (6.7) | 0 (0) |
| IVb | 4 (7.3) | 2 (13.3) | 0 (0) |
| ISTs before | | | |
| REGAIN, n (%) | | | |
| 2 | 25 (45.5) | 6 (40.0) | 9 (32.1) |
| 3 | 19 (34.5) | 5 (33.3) | 13 (46.4) |
| ≥4 | 10 (18.2) | 3 (20.0) | 6 (21.4) |
| MG crisis before | 7 (12.7) | 2 (13.3) | 8 (28.6) |
| REGAIN, n (%) | | | |

IST, immunosuppressive therapy;
MG, myasthenia gravis;
MGFA. Myasthenia Gravis Foundation of America;
QMG, quantitative myasthenia gravis;
SD, standard deviation
^{†}p = 0.0002 for early vs late responders based on two-sample t test
^{‡}p = 0.0223 for early vs late responders based on two-sample t test

### Example 10: MG-ADL and QMG Score Correlation and Muscle Group Domain Analyses of Patients Enrolled in ECU-MG-301 REGAIN and ECU-MG-302 Extension Trial

In this example, response of patients enrolled in the ECU-MG-301 REGAIN Trial (see Example 1 for protocol) and the Open-Label Extension (OLE) study (ECU-MG-302; see Example 2 for protocol) to eculizumab was analyzed by MG-ADL and QMG domain scores for individual muscle groups. The objective of this sub-analysis was to evaluate the MG-ADL and QMG domain scores for each muscle group in patients during REGAIN and its OLE to determine whether eculizumab is clinically beneficial across all muscle groups in patients with refractory AChR+gMG.

The MG-ADL is a patient-reported, 8-item questionnaire that reports on the functional impact of muscle weakness on activities of daily living in patients with gMG. It comprises four domains, representing ocular (two items), bulbar (three items), respiratory (one item), and limb (two items) muscle groups, which assess visual, oral, breathing, and limb motor abilities, respectively. Each item is scored from 0 to 3, with a maximum total score of 24. The QMG is an objective, physician-reported, 13-item measure of muscle strength that comprises four domains, representing ocular (three ocular and facial muscle items), bulbar (two swallowing and speech items), respiratory (one forced vital capacity item), and gross motor (seven limb and axial motor items) muscle groups. Each item is scored from 0 to 3, with a maximum total score of 39.

The scores for all domains of the MG-ADL and QMG were recorded throughout REGAIN and its OLE. Assessments were performed weekly from week 1 to week 3 and then at weeks 4, 8, 12, 16, 20, 26, 40, and 52 in year 1, then every 6 months afterward and at each patient's end-of-study visit. Patients with an abnormal score (≥0) at REGAIN baseline for a domain of either measure were included in the analysis of that domain for its respective measure. All OLE participants had at least one abnormal domain score for both MG-ADL and QMG at REGAIN baseline and were, therefore, included in the analysis of total scores. The numbers of patients in the eculizumab/eculizumab and placebo/eculizumab groups, respectively, who had abnormal REGAIN baseline scores and were included in this analysis were: for the MG-ADL domains, 55 and 59 for ocular, 54 and 55 for bulbar, 48 and 45 for respiratory, and 52 and 55 for limb; and for the QMG domains, 56 and 60 for ocular, 31 and 28 for bulbar, 28 and 36 for respiratory, and 56 and 61 for gross motor.

MG-ADL and QMG mean total and mean domain scores were calculated for both the eculizumab and the placebo groups in the REGAIN study and for the eculizumab/eculizumab and placebo/eculizumab groups in the OLE. Two baselines were used for these analyses; the REGAIN baseline (REGAIN day 1) was used to allow for assessment of response to eculizumab from the start of REGAIN, and the open-label baseline (the last available assessment before the first eculizumab dose in the OLE) was used to allow for assessment of response to eculizumab in the placebo/eculizumab group and assessment of maintenance of the response observed during REGAIN in the eculizumab/eculizumab group. Changes from REGAIN baseline to OLE week 130 (156 weeks, in total, for patients who had received eculizumab during REGAIN) and from open-label baseline to OLE week 130 were evaluated. The median duration of eculizumab treatment during the OLE (from open-label baseline to last OLE assessment) was 2.7 years (138.9 weeks; range, 0.1-196.0 weeks).

For the correlation analysis, eculizumab baseline was defined as the first dose of eculizumab received. Each regression line was determined by a simple linear regression model of change in MG-ADL total score from REGAIN baseline to last OLE assessment against change in QMG total score from eculizumab baseline to last OLE assessment, or MG-ADL total score at last OLE assessment as the response variable against QMG total score at last OLE assessment as the predictor variable, for the respective treatment groups. Its 95% confidence band was determined by the pointwise 95% confidence band. Pearson's correlation coefficients (R) by treatment group were determined for MG-ADL and QMG total score changes from eculizumab baseline to last OLE assessment and for MG-ADL and QMG total scores at last OLE assessment; thresholds for moderate and strong correlations were 0.4 and 0.6, respectively.

Repeated-measures analyses for changes in MG-ADL and QMG total and domain scores from open-label baseline were performed. These data are presented as least-squares means and 95% confidence intervals (CIs).

MG-ADL and QMG and QMG Total Scores

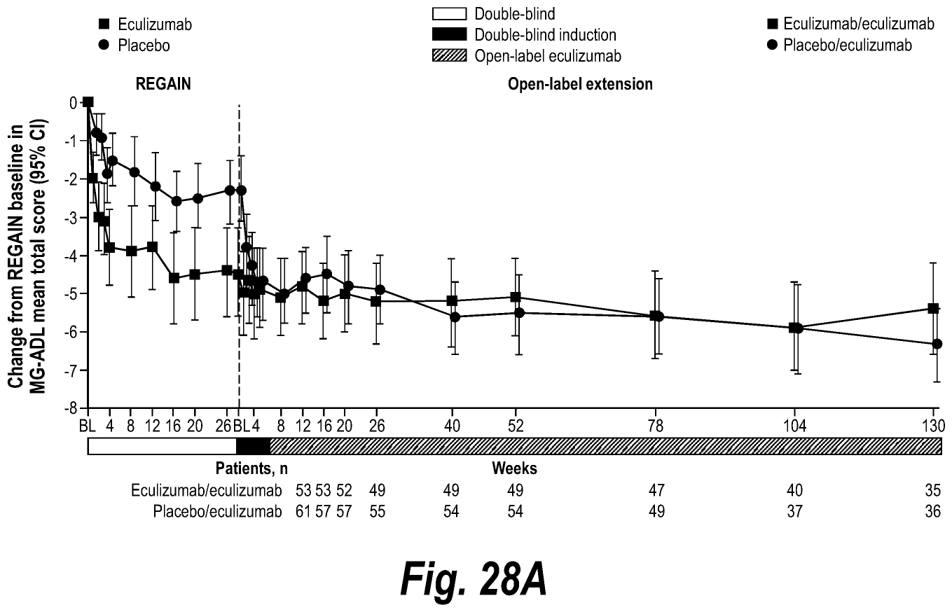

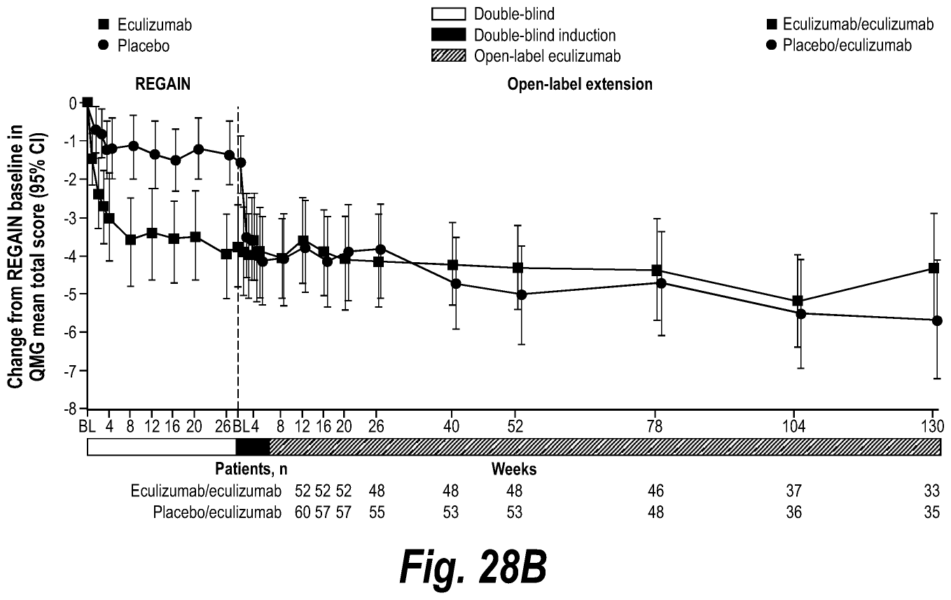

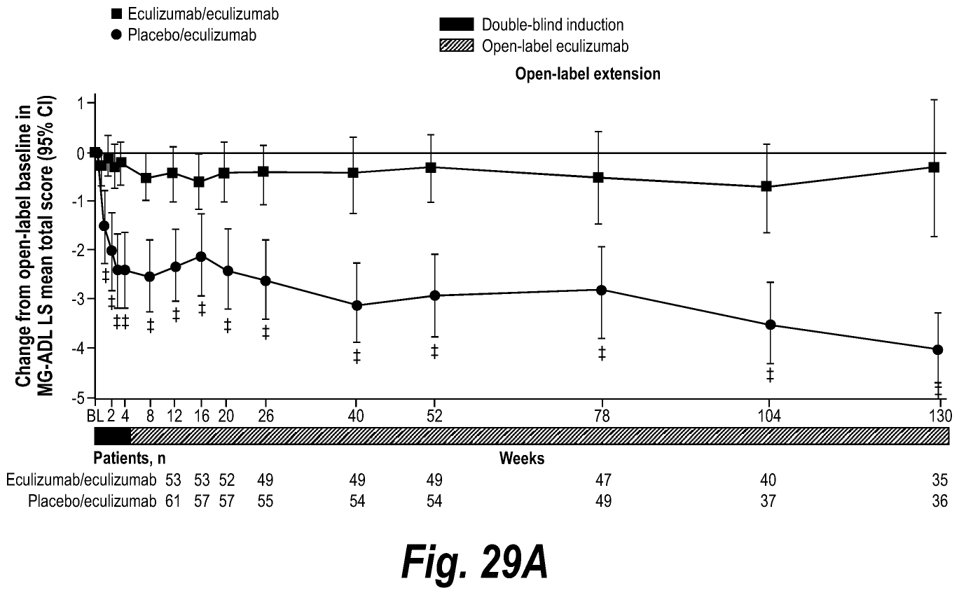

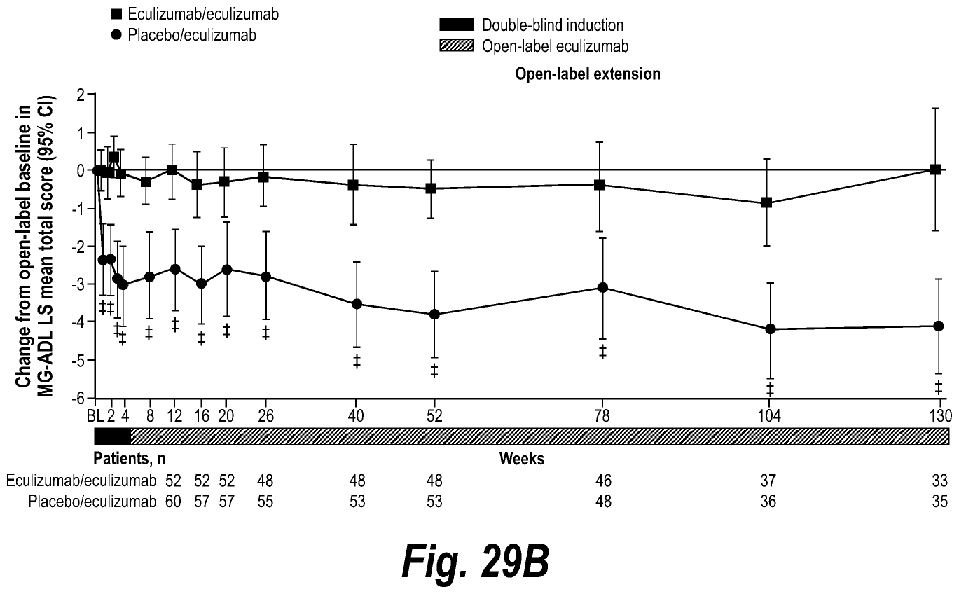

For patients in the eculizumab/eculizumab group, the improvements achieved in both MG-ADL and QMG mean total scores during REGAIN and the interim analysis of OLE data were found to be sustained in this final analysis of the complete OLE data through 130 weeks (FIGS. 28 and 29). Patients in the placebo/eculizumab group experienced rapid improvements in MG-ADL and QMG mean total scores from the start of eculizumab treatment in the OLE during the 4-week blinded induction period (FIGS. 28 and 29). In this group, MG-ADL and QMG mean total scores were significantly improved from open-label baseline (BL) as early as OLE week 1 (p<0.001), and these improvements remained significant for every week through 130 weeks (FIG. 29). Stable scores in the eculizumab/eculizumab group are evidence of maintained improvement achieved during eculizumab treatment in REGAIN in these patients.

Correlation Between MG-ADL and QMG Scores

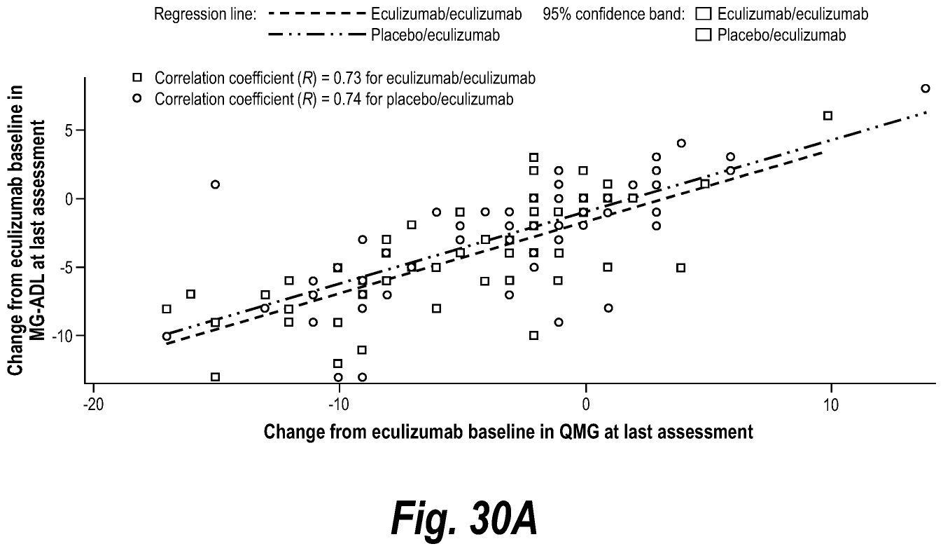

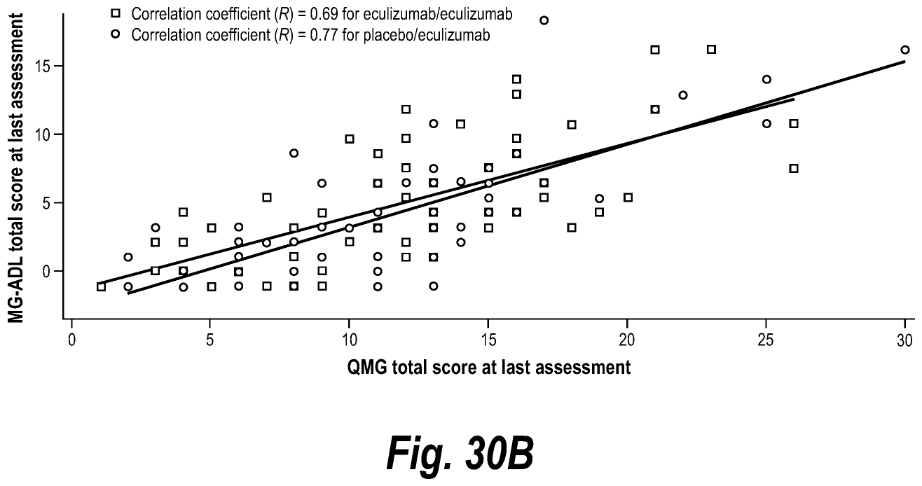

In the eculizumab/eculizumab group, there were strong correlations between changes in MG-ADL and QMG total scores from eculizumab baseline to last OLE assessment (R=0.73; 95% CI, 0.57-0.83; FIG. 30A) and between MG-ADL and QMG total scores at last OLE assessment (R=0.69; 95% Cl, 0.51-0.80; FIG. 30B). In the placebo/eculizumab group, the correlations between MG-ADL and QMG total scores were also strong for changes in total scores from eculizumab baseline to last OLE assessment (R=0.74, 95% CI, 0.59-0.83; FIG. 30A) and for total scores at last OLE assessment (R=0.77; 95% CI, 0.64-0.86; FIG. 30B). For the entire OLE population, the coefficients for the correlations between changes in MG-ADL and QMG total scores from eculizumab baseline to last OLE assessment and between the total scores at last OLE assessment were 0.74 (95% CI, 0.65-0.81) and 0.73 (95% CI, 0.63-0.80), respectively.

The strong correlations between MG-ADL and QMG total scores, both for changes from eculizumab baseline to last OLE assessment and at last OLE assessment, demonstrate that the long-term effect of eculizumab is consistent between the MG-ADL and QMG assessments. This finding further validates use of the MG-ADL in assessing response to treatment in MG and also provides evidence that patient-reported improvements in response to long-term eculizumab treatment are supported by objective physician assessments of patients with refractory AChR+gMG.

MG-ADL Domain Scores

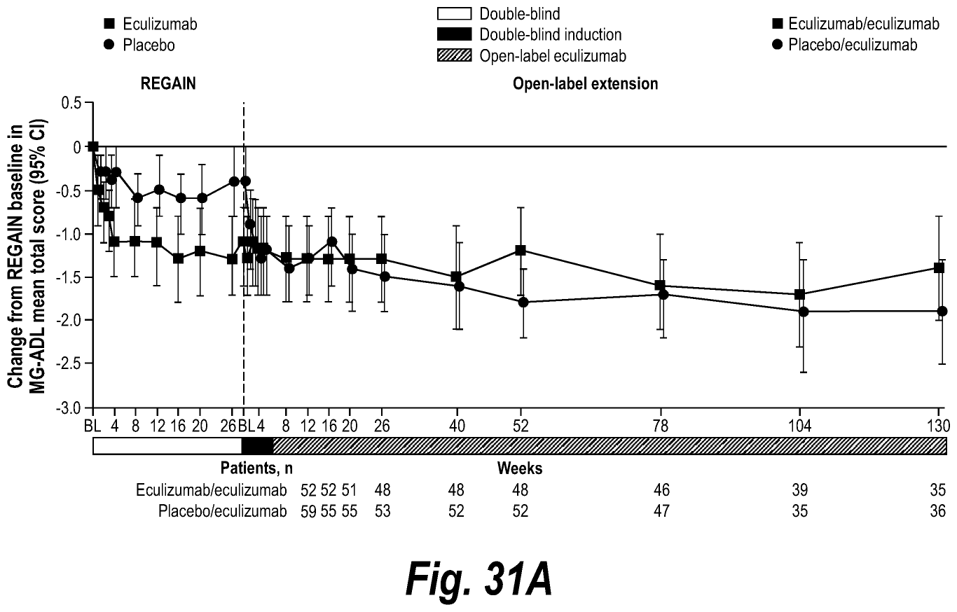

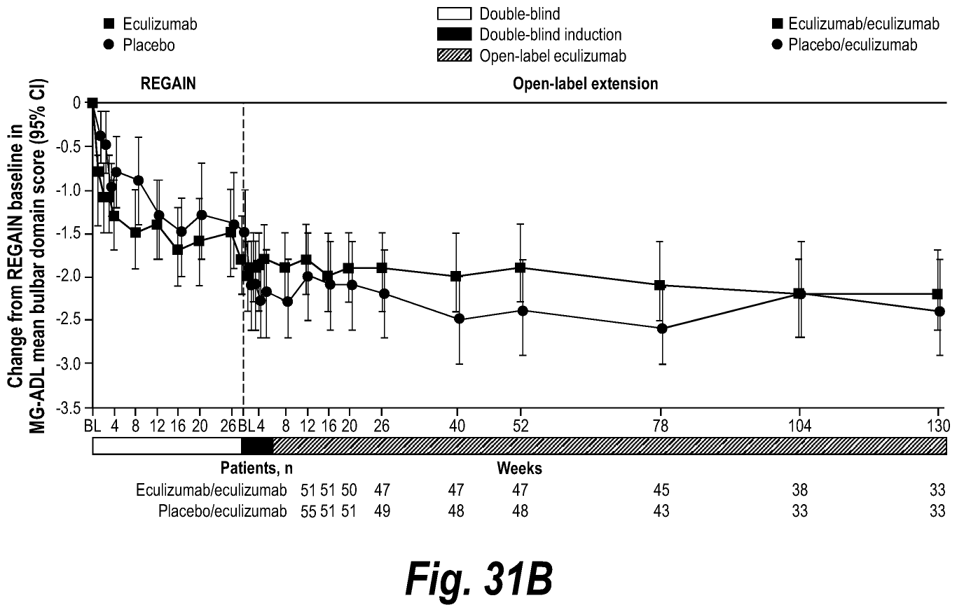

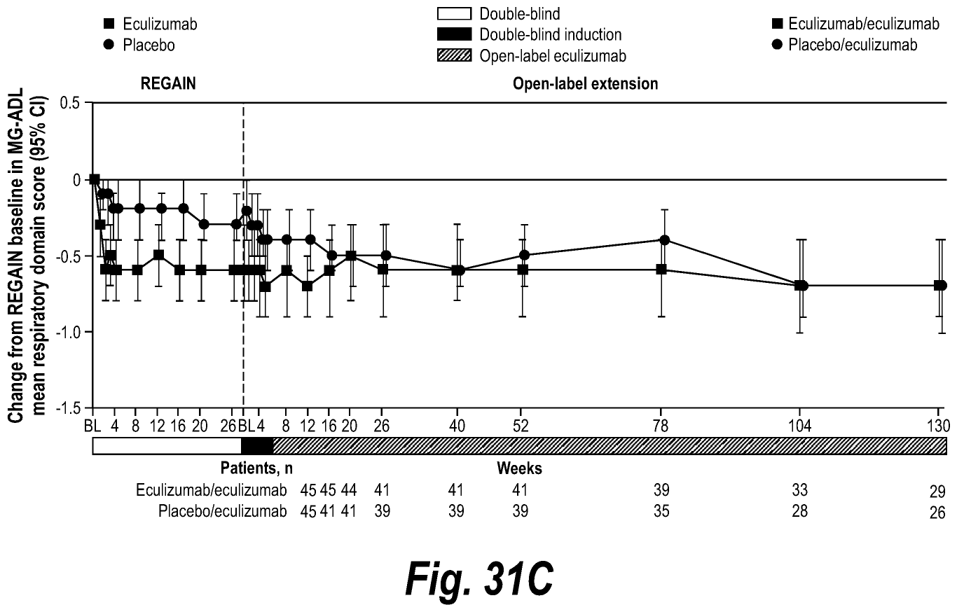

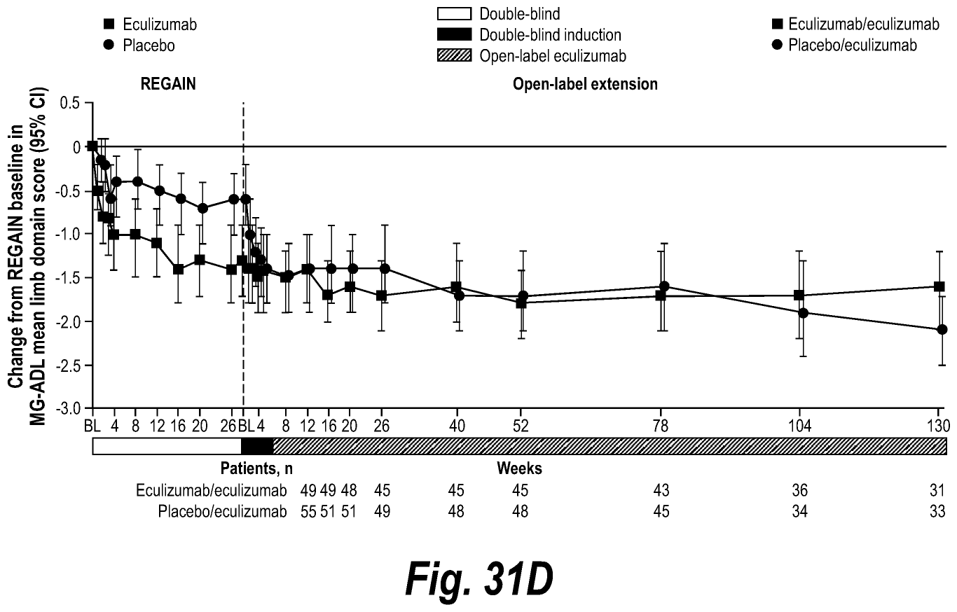

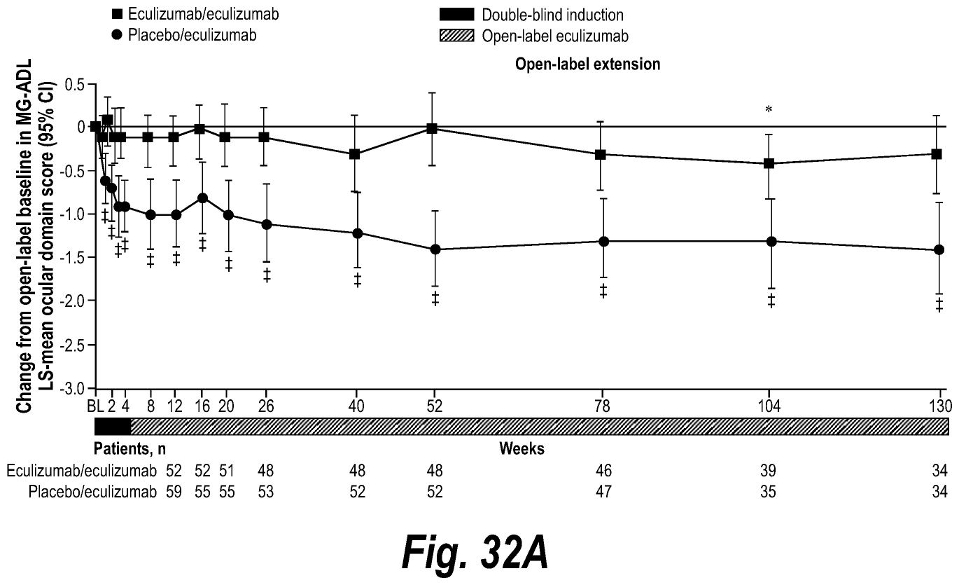

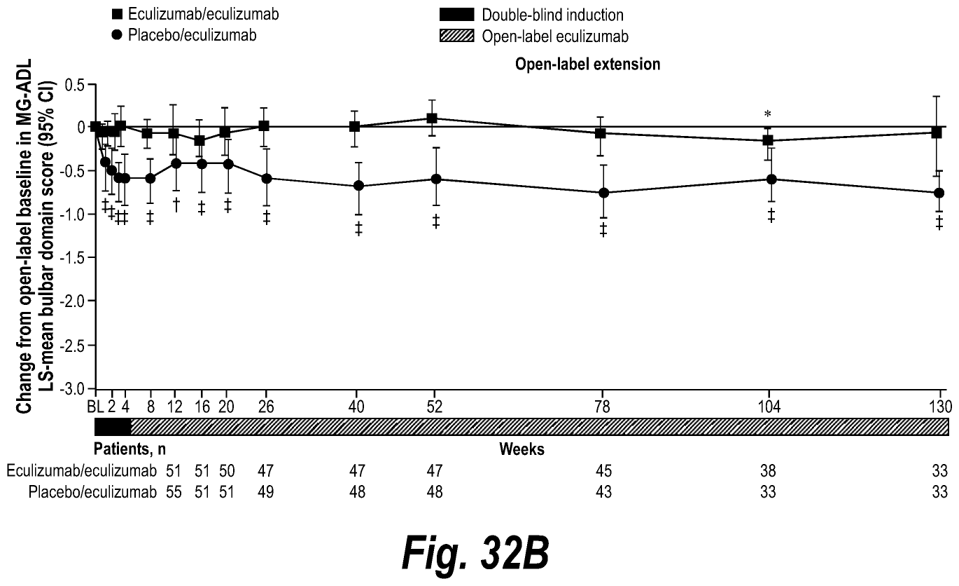

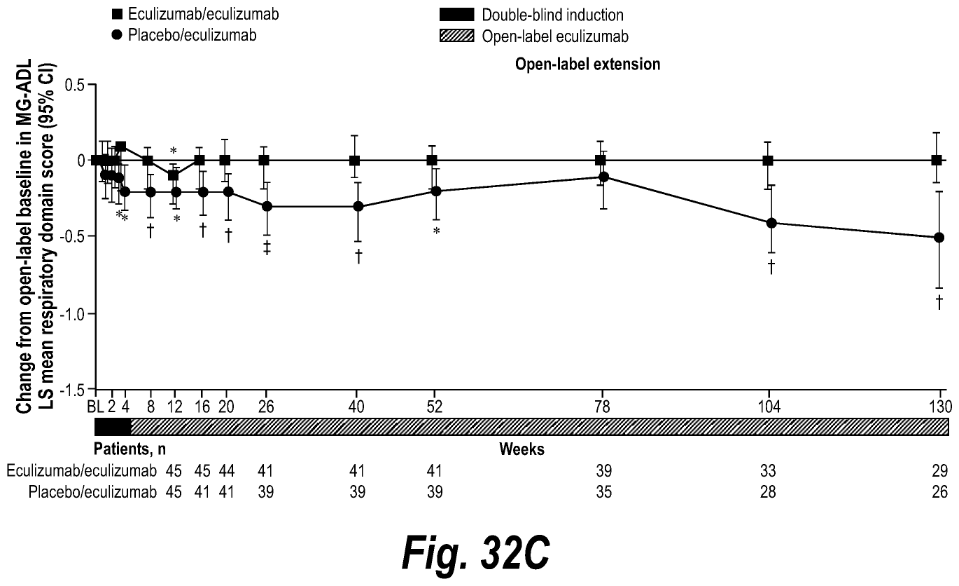

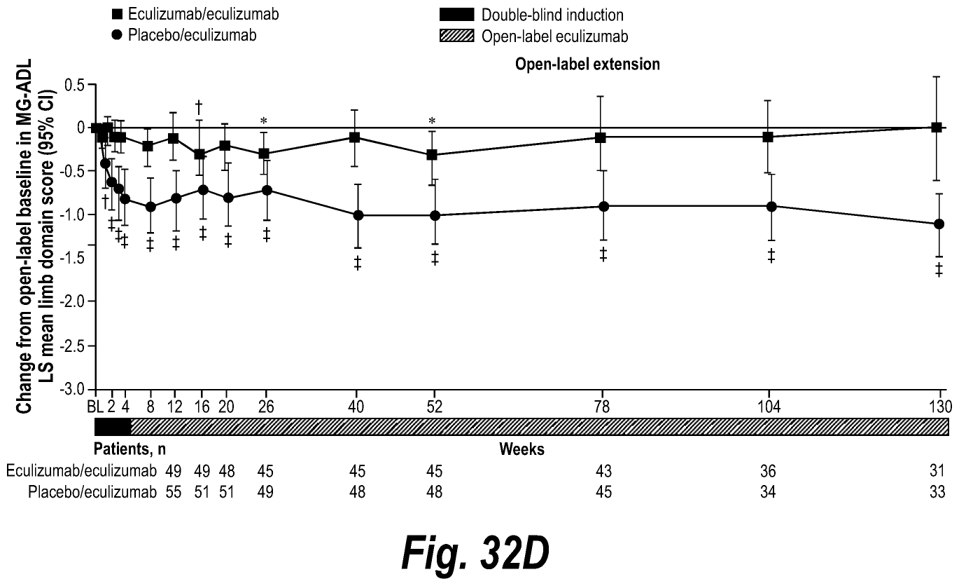

Patients in the eculizumab/eculizumab group experienced rapid improvements across all four MG-ADL domains during REGAIN, and this treatment effect was sustained through 130 weeks of the OLE (FIGS. 31A-D and 32A-D). During REGAIN, peak improvements were observed by week 16 in all domains except the respiratory domain in which it was observed by week 4 (FIGS. 31A-D). Thereafter, the peak improvements achieved in REGAIN were sustained or increased in all domains during the OLE through 130 weeks (FIGS. 32A-D).

In the placebo/eculizumab group, statistically significant improvements in scores from open-label baseline were observed in the ocular, bulbar, and limb domains as early as week 1 and remained significant at each time point through 130 weeks (*p≤0.05, †p≤0.01, ‡p≤0.001; FIGS. 32A, 32B, and 32D). In the respiratory domain, the improvement in score was statistically significant by week 4 and remained significant at each time point through 130 weeks except at week 78 (*p≤0.05, †p≤0.01, ‡p≤0.001; FIG. 32C). Stable scores in the eculizumab/eculizumab group are evidence of maintained improvement achieved during eculizumab treatment in REGAIN in these patients.

QMG Domain Scores

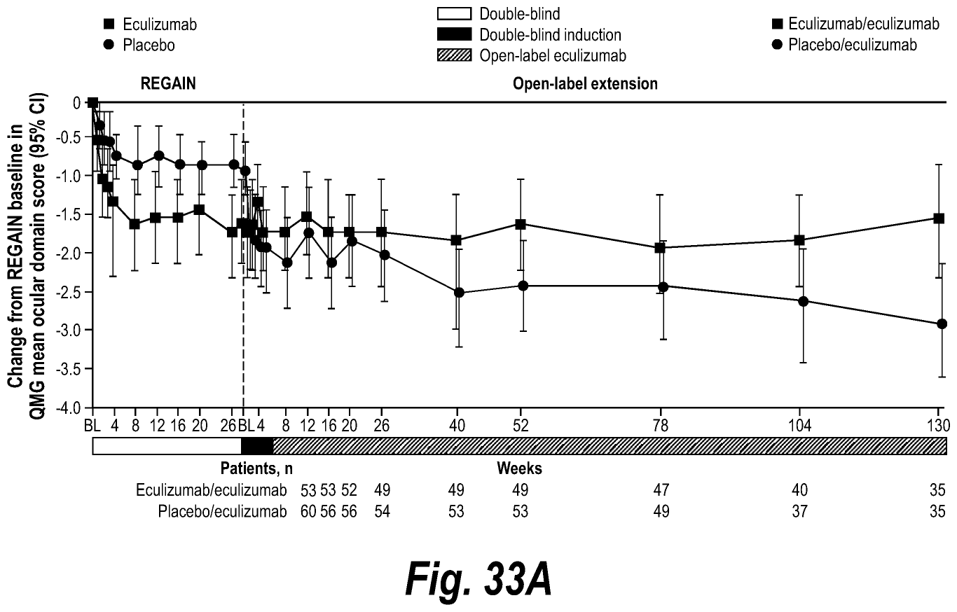

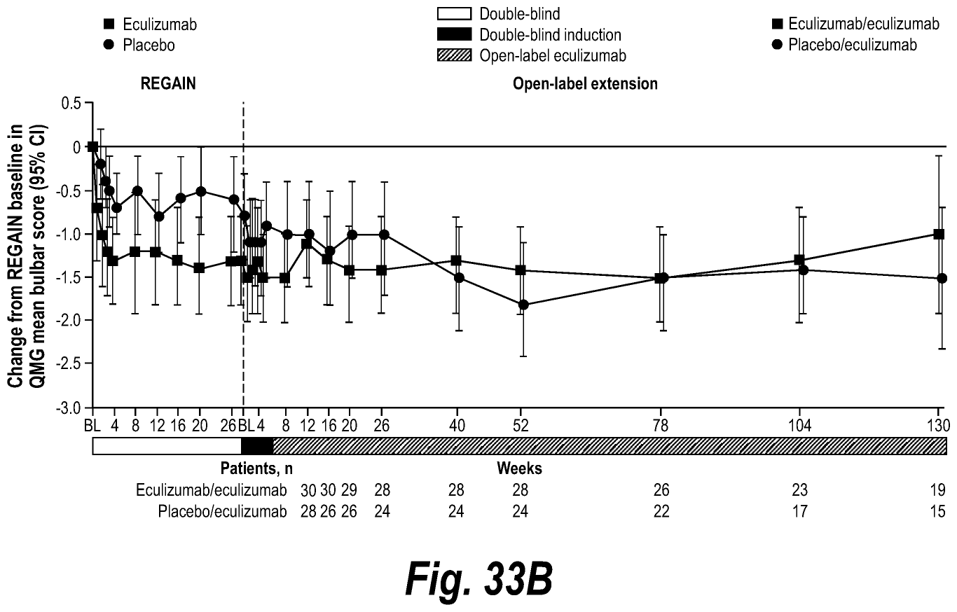

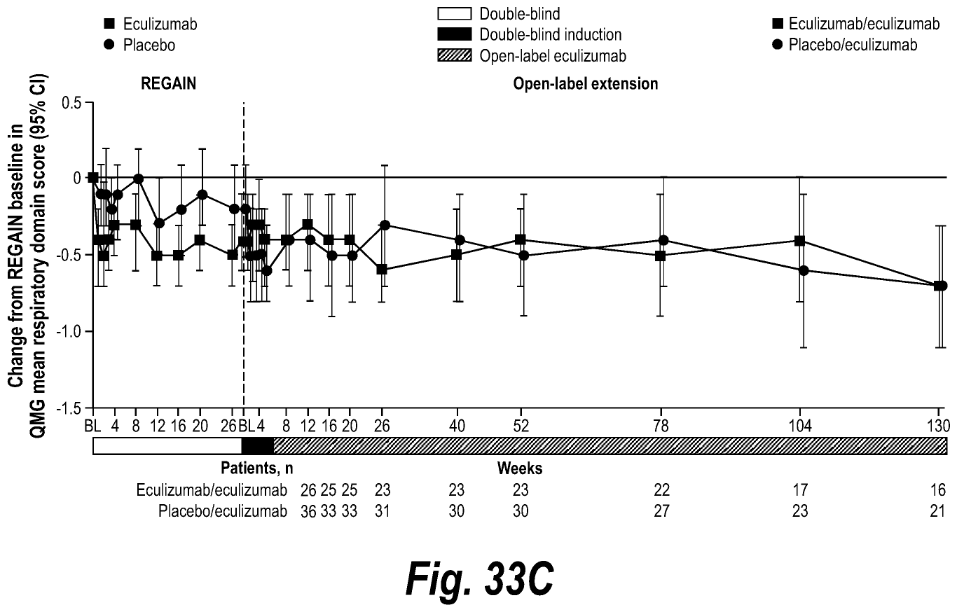

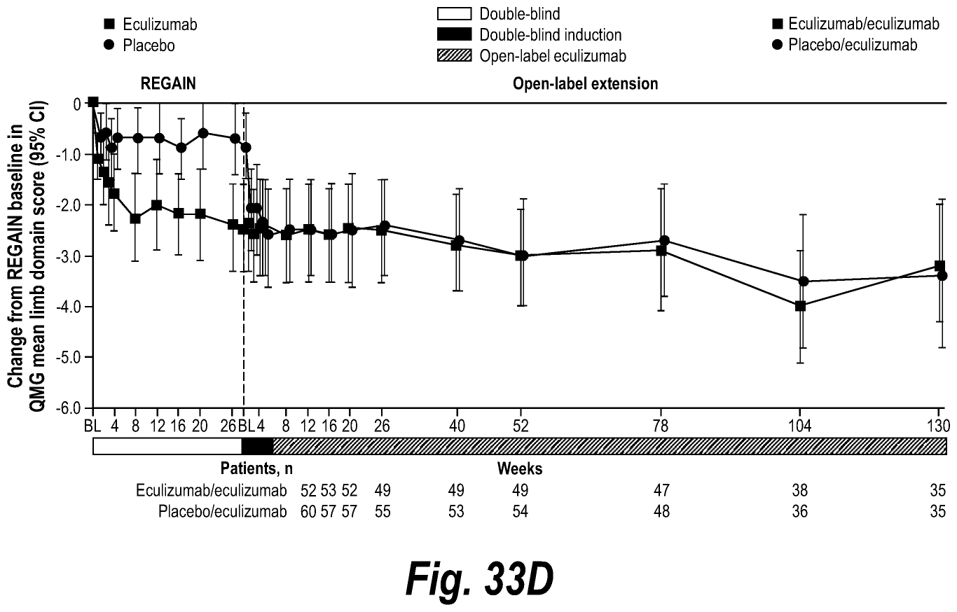

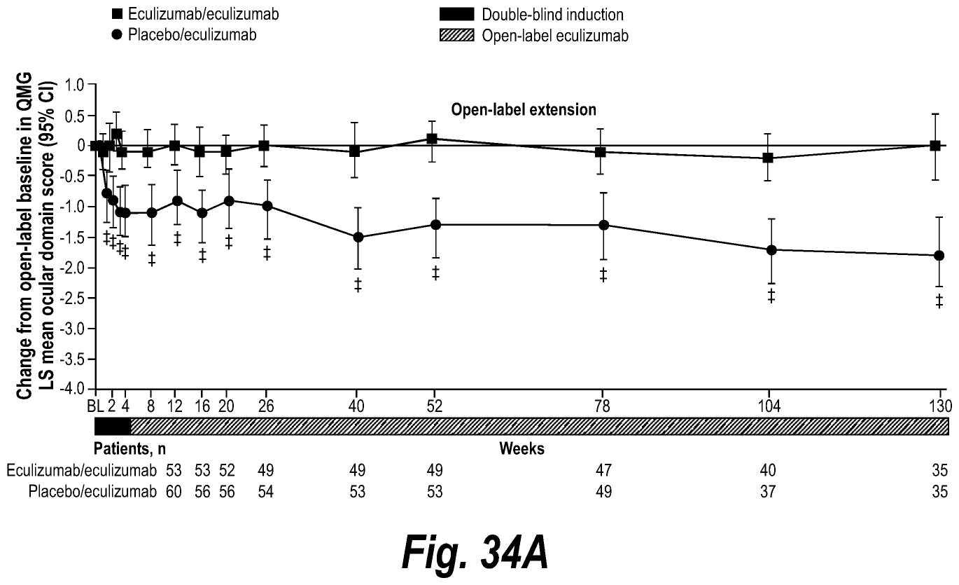

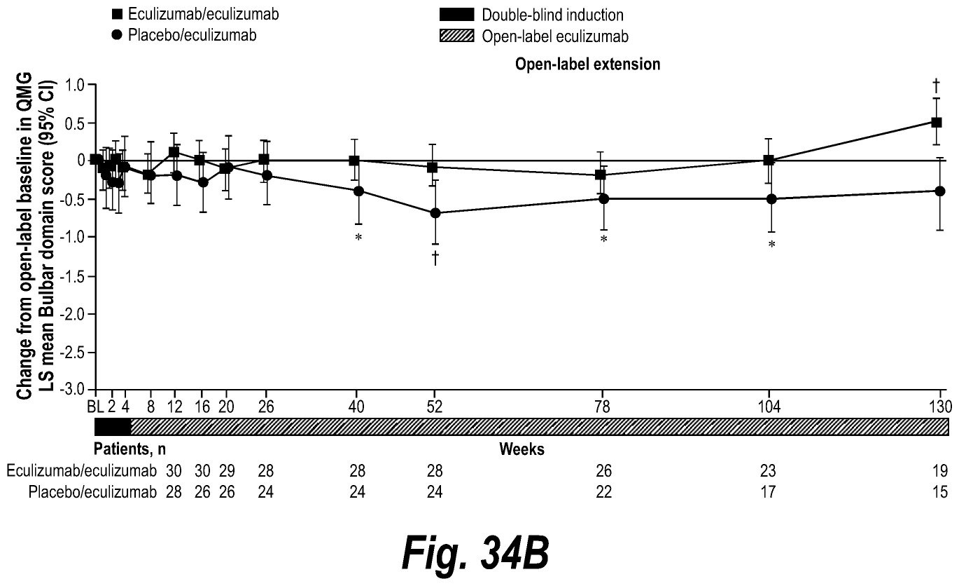

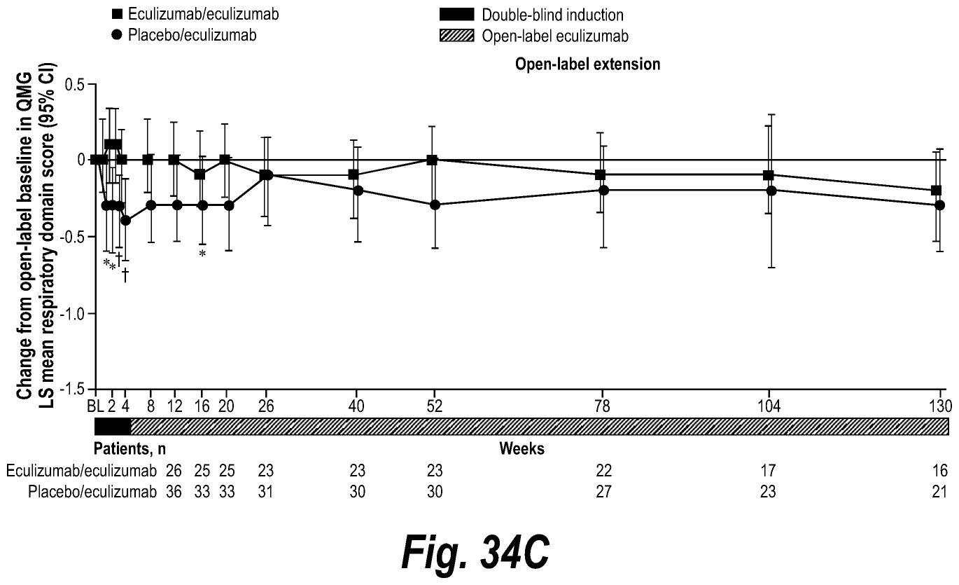

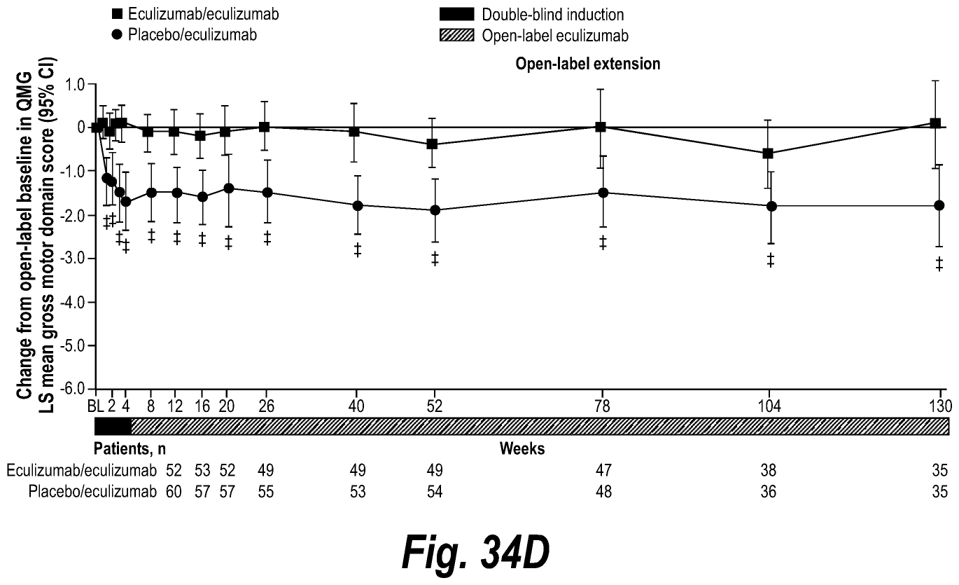

Patients in the eculizumab/eculizumab group experienced rapid improvements across all four QMG domains during REGAIN, and this treatment effect was sustained through 130 weeks of the OLE (FIGS. 33A-D and 34A-D). During REGAIN, peak improvements were observed by week 26 in the ocular and gross motor domains (FIGS. 33A and 33D), week 20 in the bulbar domain (FIG. 33B), and week 12 in the respiratory domain (FIG. 33C). Thereafter, the peak improvements achieved in REGAIN were sustained or increased in all domains during the OLE through 130 weeks, except in the bulbar domain at week 130 (FIGS. 34A-D).

In the placebo/eculizumab group, statistically significant improvements from open-label baseline were observed across all domains by week 1, except for the bulbar domain, which was first significantly improved at week 40 (*p≤0.05, †p≤0.01, ‡p≤0.001; FIGS. 34A-D). In the ocular and gross motor domains, improvements from open-label baseline were significant for every week through 130 weeks (*p≤0.05, †p≤0.01, ‡p≤0.001; FIGS. 34A and 34D). In the bulbar and respiratory domains, improvements from open-label baseline were significant at weeks 40, 52, 78, and 104, and at weeks 1, 2, 3, 4, and 16, respectively (*p≤0.05, †p≤0.01, ‡p≤0.001; FIGS. 34B and 34C). Stable scores in the eculizumab/eculizumab group are evidence of maintained improvement achieved during eculizumab treatment in REGAIN in these patients.

In contrast to other MG therapies that have been reported to have differential effects across muscle groups, eculizumab had benefits that were evident across the MG-ADL domains for all four muscle groups, demonstrating that eculizumab improves patients' breathing and functioning in a wide range of activities of daily living through at least 130 weeks. These patient-reported improvements were supported by objective, physician-reported improvements in muscle strength with eculizumab as evaluated by QMG domain scores across all four muscle groups through at least 130 weeks. It is notable that these improvements were achieved by patients with refractory disease, a population with significant disease burden, and that most participants completed the OLE.

### 4. Other Embodiments

E1. A method of treating refractory generalized myasthenia gravis in a patient in need thereof comprising administering a therapeutically effective amount of an anti-C5 antibody or an antigen binding fragment thereof to the patient;

wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability;

and

wherein the patient is treated for at least 52 weeks and achieves a Myasthenia Gravis Foundation of America (MGFA) post-intervention status of Improved or Minimal Manifestations (MM) after at least 4 weeks of treatment.

E2. The method of embodiment E1, wherein the anti-C5 antibody is eculizumab.
E3. The method of embodiment E2, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.
E4. The method of embodiment E2, further comprising performing plasmapheresis on the patient and administering eculizumab at a dose of between 300 mg and 1200 mg to the patient within 4 hours of completion of plasmapheresis.
E5. The method of embodiment E2, further comprising performing plasmapheresis on the patient and administering eculizumab at a dose of between 600 mg and 900 mg to the patient within 90 minutes of completion of plasmapheresis.
E6. The method of embodiment E2, further comprising performing plasmapheresis on the patient and administering eculizumab at a dose of 600 mg to the patient within 1 hour of completion of plasmapheresis.
E7. The method of embodiment E1, wherein the therapeutically effective amount is based on the weight of the subject.
E8. The method of embodiment E1, wherein the anti-C5 antibody is ravulizumab.
E9. The method of embodiment E8, wherein ravulizumab, or an antigen binding fragment thereof is administered to a patient weighing ≥40 and <60 kg:

(a) once on Day 1 of the administration cycle at a loading dose of 2400 mg; and

(b) on Day 15 of the administration cycle and every eight weeks thereafter at a maintenance dose of 3000 mg.

E10. The method of embodiment E8, wherein ravulizumab, or an antigen binding fragment thereof is administered to a patient weighing ≥60 and <100 kg:

(a) once on Day 1 of the administration cycle at a loading dose of 2700 mg; and

(b) on Day 15 of the administration cycle and every eight weeks thereafter at a maintenance dose of 3300 mg.

E11. The method of embodiment E8, wherein ravulizumab, or antigen binding fragment thereof, is administered to a patient weighing ≥100 kg:

(a) once on Day 1 of the administration cycle at a loading dose of 3000 mg; and

(b) on Day 15 of the administration cycle and every eight weeks thereafter at a maintenance dose of 3600 mg.

E12. The method of embodiment E1, wherein the patient achieves a MGFA post-intervention status of Improved or MM after 4 weeks of treatment.
E13. The method of embodiment E1, wherein the patient achieves a MGFA post-intervention status of Improved or MM after 12 weeks of treatment.
E14. The method of embodiment E1, wherein the patient achieves a MGFA post-intervention status of Improved or MM after 26 weeks of treatment.
E15. The method of embodiment E1, wherein the patient achieves a MGFA post-intervention status of Improved or MM after 52 weeks of treatment.
E16. The method of embodiment E1, wherein the patient achieves a MGFA post-intervention status of Improved or MM after 66 weeks of treatment.
E17. The method of embodiment E1, wherein the patient achieves a MGFA post-intervention status of Improved or MM after 78 weeks of treatment.
E18. The method of embodiment E1, wherein the patient achieves a MGFA post-intervention status of Improved or MM after 104 weeks of treatment.
E19. The method of embodiment E1, wherein the patient achieves a MGFA post-intervention status of Improved or MM after 130 weeks of treatment.
E20. The method of embodiment E1, wherein the patient achieves a MGFA post-intervention status of Improved or MM after 156 weeks of treatment.
E21. The method of any one of embodiments E1-E14, wherein the patient achieves a MGFA post-intervention status of Improved.
E22. The method of any one of embodiments E1-E14, wherein the patient achieves a MGFA post-intervention status of MM.
E23. The method of embodiment E1, wherein the patient experiences a clinically meaningful improvement (reduction) in a measurement of generalized myasthenia gravis severity after 26 weeks of treatment selected from the group consisting of Myasthenia Gravis Activities of Daily Living (MG-ADL) score, quantitative Myasthenia Gravis (QMG), score and Myasthenia Gravis Composite (MGC) score.
E24. The method of embodiment E23, wherein the clinically meaningful improvement the patient experiences is an at least a 3 point reduction in the patient's MG-ADL score after 26 weeks of treatment.
E25. The method of embodiment E23, wherein the clinically meaningful improvement the patient experiences is an at least a 4 point reduction in the patient's QMG score after 26 weeks of treatment.
E26. The method of embodiment E23, wherein the clinically meaningful improvement the patient experiences is an at least a 6 point reduction in the patient's MGC score after 26 weeks of treatment.
E27. The method of embodiment E1, wherein the patient experiences a clinically meaningful improvement (reduction) in quality of life as measured by Myasthenia Gravis Quality of Life (MG-QOL-15) score after 26 weeks of treatment.
E28. The method of embodiment E27, wherein the clinically meaningful improvement the patient experiences is an at least a 6 point reduction in the patient's MG-QOL-15 score after 26 weeks of treatment.
E29. The method of embodiment E1, wherein the patient experiences a clinically meaningful improvement (reduction) in neuro-fatigue as measured by Neuro-QOL Fatigue score after 26 weeks of treatment.
E30. The method of embodiment E29, wherein the clinically meaningful improvement the patient experiences is an at least an 8 point reduction in the patient's Neuro-QOL score after 26 weeks of treatment.
E31. The method of embodiment E1, wherein the patient experiences a clinically meaningful improvement (increase) in health status as measured by EQ-5D health status score after 26 weeks of treatment.
E32. A method of treating refractory generalized myasthenia gravis in a patient in need thereof comprising administering an anti-C5 antibody or an antigen binding fragment thereof to the patient;
wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability;
wherein the patient is treated for at least 52 weeks and achieves a Myasthenia Gravis Foundation of America (MGFA) post-intervention status of Improved or Minimal Manifestations (MM) after at least 4 weeks of treatment; and
wherein the patient has a clinically meaningful improvement (reduction) in at least two measurements of generalized myasthenia gravis severity selected from the group consisting of MG-ADL, QMG, MGC, MG-QOL, and Neuro-QOL.
   E33. The method of embodiment E32, wherein the anti-C5 antibody is eculizumab.
   E34. The method of embodiment E33, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.
   E35. The method of embodiment E32, wherein the therapeutically effective amount is based on the weight of the subject.
   E36. The method of embodiment E32, wherein the anti-C5 antibody is ravulizumab.
   E37. The method of embodiment E36, wherein ravulizumab, or an antigen binding fragment thereof is administered to a patient weighing ≥40 and <60 kg:

(a) once on Day 1 of the administration cycle at a loading dose of 2400 mg; and

(b) on Day 15 of the administration cycle and every eight weeks thereafter at a maintenance dose of 3000 mg.

E38. The method of embodiment E36, wherein ravulizumab, or an antigen binding fragment thereof is administered to a patient weighing ≥60 and <100 kg:

(a) once on Day 1 of the administration cycle at a loading dose of 2700 mg; and

(b) on Day 15 of the administration cycle and every eight weeks thereafter at a maintenance dose of 3300 mg.

E39. The method of embodiment E36, wherein ravulizumab, or antigen binding fragment thereof, is administered to a patient weighing ≥100 kg:

(a) once on Day 1 of the administration cycle at a loading dose of 3000 mg; and

(b) on Day 15 of the administration cycle and every eight weeks thereafter at a maintenance dose of 3600 mg.

E40. A method of treating refractory generalized myasthenia gravis in a patient in need thereof comprising administering an anti-C5 antibody or an antigen binding fragment thereof to the patient;

wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability;

wherein the patient is treated for at least 52 weeks and achieves a Myasthenia Gravis Foundation of America (MGFA) post-intervention status of Improved or Minimal Manifestations (MM) after at least 4 weeks of treatment; and

wherein the patient has a clinically meaningful improvement (reduction) in five measurements of generalized myasthenia gravis severity, wherein the five measurements of generalized myasthenia gravis severity are a reduction in MG-ADL of at least 3 points, a reduction of QMG of at least 4 points, a reduction in MGC of at least 6 points, a reduction in MG-QOL of at least 6 points, and a reduction in Neuro-QOL of at least 8 points.

E41. The method of embodiment E40, wherein the anti-C5 antibody is eculizumab.
E42. The method of embodiment E41 wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.
E43. The method of embodiment E40, wherein the therapeutically effective amount is based on the weight of the subject.
E44. The method of embodiment E40, wherein the anti-C5 antibody is ravulizumab.
E45. The method of embodiment E44, wherein ravulizumab, or an antigen binding fragment thereof is administered to a patient weighing ≥40 and <60 kg:
(a) once on Day 1 of the administration cycle at a loading dose of 2400 mg; and
(b) on Day 15 of the administration cycle and every eight weeks thereafter at a maintenance dose of 3000 mg.
E46. The method of embodiment E44, wherein ravulizumab, or an antigen binding fragment thereof is administered to a patient weighing ≥60 and <100 kg:
(a) once on Day 1 of the administration cycle at a loading dose of 2700 mg; and
(b) on Day 15 of the administration cycle and every eight weeks thereafter at a maintenance dose of 3300 mg.
E47. The method of embodiment E44, wherein ravulizumab, or antigen binding fragment thereof, is administered to a patient weighing ≥100 kg:

(a) once on Day 1 of the administration cycle at a loading dose of 3000 mg; and

(b) on Day 15 of the administration cycle and every eight weeks thereafter at a maintenance dose of 3600 mg.

E48. The method of embodiment E40, wherein the patient has a clinically meaningful improvement (reduction) in five measurements of generalized myasthenia gravis, wherein the five measurements of generalized myasthenia gravis severity are a reduction in MG-ADL of at least 4 points, a reduction of QMG of at least 5 points, a reduction in MGC of at least 10 points, a reduction in MG-QOL of at least 11 points, and a reduction in Neuro-QOL of at least 16 points.
E49. A method of maintaining a Myasthenia Gravis Foundation of America (MGFA) post-intervention status of Improved or Minimal Manifestations (MM) in a patient with refractory generalized myasthenia gravis in need thereof comprising administering a therapeutically effective amount of an anti-C5 antibody or an antigen binding fragment thereof to the patient;

wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; and

wherein the patient had achieved the Improved or MM status.

E50. The method of embodiment E49, wherein the anti-C5 antibody is eculizumab.
E51. The method of embodiment E50, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.
E52. The method of embodiment E50, further comprising performing plasmapheresis on the patient and administering eculizumab at a dose of between 300 mg and 1200 mg to the patient within 4 hours of completion of plasmapheresis.
E53. The method of embodiment E50, further comprising performing plasmapheresis on the patient and administering eculizumab at a dose of between 600 mg and 900 mg to the patient within 90 minutes of completion of plasmapheresis.
E54. The method of embodiment E50, further comprising performing plasmapheresis on the patient and administering eculizumab at a dose of 600 mg to the patient within 1 hour of completion of plasmapheresis.
E55. The method of embodiment E49, wherein the therapeutically effective amount is based on the weight of the subject.
E56. The method of embodiment E49, wherein the anti-C5 antibody is ravulizumab.
E57. The method of embodiment E56, wherein ravulizumab, or an antigen binding fragment thereof is administered to a patient weighing ≥40 and <60 kg:

(a) once on Day 1 of the administration cycle at a loading dose of 2400 mg; and

(b) on Day 15 of the administration cycle and every eight weeks thereafter at a maintenance dose of 3000 mg.

E58. The method of embodiment E56, wherein ravulizumab, or an antigen binding fragment thereof is administered to a patient weighing ≥60 and <100 kg:

(a) once on Day 1 of the administration cycle at a loading dose of 2700 mg; and

(b) on Day 15 of the administration cycle and every eight weeks thereafter at a maintenance dose of 3300 mg.

E59. The method of embodiment E56, wherein ravulizumab, or antigen binding fragment thereof, is administered to a patient weighing ≥100 kg:

(a) once on Day 1 of the administration cycle at a loading dose of 3000 mg; and

(b) on Day 15 of the administration cycle and every eight weeks thereafter at a maintenance dose of 3600 mg.

E60. The method of embodiment E49, wherein the Improved or MM status is maintained for at least 4, 12, 26, 52, 66, 78, 104, 130 or 156 weeks.
E61. The method of embodiment E49, wherein the patient starts the maintenance with the MM status.
E62. The method of embodiment E49, wherein the MM status is maintained for at least 4, 12, 26, 52, 66, 78, 104, 130 or 156 weeks.
E63. The method of any of the preceding embodiments, wherein the anti-C5 antibody or antigen binding fragment thereof is administered by intravenous infusion.
E64. The method of any of the preceding embodiments, wherein the anti-C5 antibody or antigen binding fragment thereof is administered subcutaneously.
E65. The method of any of the preceding embodiments, wherein the eculizumab comprises a heavy chain amino acid sequence according to SEQ ID NO: 10 and a light chain amino acid sequence according to SEQ ID NO: 11.
E66. The method of any of the preceding embodiments, wherein ravulizumab comprises a heavy chain amino acid sequence according to SEQ ID NO: 14 and a light chain amino acid sequence according to SEQ ID NO: 11.
E67. The method of any of the preceding embodiments, wherein the patient has failed treatment over one year or more with two or more ISTs in sequence or in combination.
E68. The method of any of the preceding embodiments, wherein the patient has failed at least one IST and requires chronic plasma exchange or IVIg to control symptoms.
E69. The method of any of the preceding embodiments, wherein the therapeutically effective amount of the anti-C5 antibody or antigen binding fragment thereof is maintained at a concentration of between 50-100 μg/mL in the patient's serum.
E70. The method of any of the preceding embodiments, wherein the patient experiences a reduction in the administration of one or more IST following at least 26 weeks of treatment.
E71. The method of any of the preceding embodiments, wherein the patient experiences a reduction in IST dosing following at least 26 weeks of treatment.
E72. The method of any of the preceding embodiments, wherein the patient experiences a reduction in one or more IST dosing and a discontinuation in one or more IST following at least 26 of treatment.
E73. The method of any of the preceding embodiments, wherein the anti-C5 antibody or an antigen binding fragment thereof is selected from the group consisting of eculizumab, ravulizumab, BNJ421, 7086 antibody, 8110 antibody, 305LO5, SKY59 and REGN3918.
E74. The method of any one of embodiments E1, E32, E40 and E49, wherein a patient switches from receiving one anti-C5 antibody or antigen binding fragment thereof to a different anti-C5 antibody or antigen binding fragment thereof during the course of treatment.
E75. The method of any one of embodiment E1, E32, E40 and E49, wherein different anti-C5 antibodies may be administered during separate treatment periods.
E76. An anti-C5 antibody or an antigen binding fragment thereof for use in treating refractory generalized myasthenia gravis in a patient in need thereof;

wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability;

and

wherein the patient is treated for at least 52 weeks and achieves a Myasthenia Gravis Foundation of America (MGFA) post-intervention status of Improved or Minimal Manifestations (MM) after at least 4 weeks of treatment.

E77. An anti-C5 antibody or an antigen binding fragment thereof for the use according to embodiment E76, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.
E78. An anti-C5 antibody or an antigen binding fragment thereof for the use according to embodiment E76 or embodiment E77, further comprising performing plasmapheresis on the patient and administering eculizumab at a dose of between 300 mg and 1200 mg to the patient within 4 hours of completion of plasmapheresis.
E79. An anti-C5 antibody or an antigen binding fragment thereof for the use according to of embodiment E76 or embodiment E77, further comprising performing plasmapheresis on the patient and administering eculizumab at a dose of between 600 mg and 900 mg to the patient within 90 minutes of completion of plasmapheresis.
E80. An anti-C5 antibody or an antigen binding fragment thereof for the use according to embodiment E76 or embodiment E77, further comprising performing plasmapheresis on the patient and administering eculizumab at a dose of 600 mg to the patient within 1 hour of completion of plasmapheresis.
E81. An anti-C5 antibody or an antigen binding fragment thereof for the use according to any one of embodiments E76 to E80, wherein the therapeutically effective amount is based on the weight of the subject.
E82. An anti-C5 antibody or an antigen binding fragment thereof for the use according to any one of embodiments E76 to E81, wherein the patient achieves a MGFA post-intervention status of Improved or MM after 4 weeks of treatment.
E83. An anti-C5 antibody or an antigen binding fragment thereof for the use according to any one of embodiments E76 to E82, wherein the patient achieves a MGFA post-intervention status of Improved or MM after 12 weeks of treatment.
E84. An anti-C5 antibody or an antigen binding fragment thereof for the use according to any one of embodiments E76 to E83, wherein the patient achieves a MGFA post-intervention status of Improved or MM after 26 weeks of treatment.
E85. An anti-C5 antibody or an antigen binding fragment thereof for the use according to any one of embodiments E76 to E84, wherein the patient achieves a MGFA post-intervention status of Improved or MM after 52 weeks of treatment.
E86. An anti-C5 antibody or an antigen binding fragment thereof for the use according to any one of embodiments E76 to E85, wherein the patient achieves a MGFA post-intervention status of Improved or MM after 66 weeks of treatment.
E87. An anti-C5 antibody or an antigen binding fragment thereof for the use according to any one of embodiments E76 to E86, wherein the patient achieves a MGFA post-intervention status of Improved or MM after 78 weeks of treatment.
E88. An anti-C5 antibody or an antigen binding fragment thereof for the use according to any one of embodiments E76 to E87, wherein the patient achieves a MGFA post-intervention status of Improved or MM after 104 weeks of treatment.
E89. An anti-C5 antibody or an antigen binding fragment thereof for the use according to any one of embodiments E76 to E88, wherein the patient achieves a MGFA post-intervention status of Improved or MM after 130 weeks of treatment.
E90. An anti-C5 antibody or an antigen binding fragment thereof for the use according to any one of embodiments E76 to E89, wherein the patient achieves a MGFA post-intervention status of Improved or MM after 156 weeks of treatment.
E91. An anti-C5 antibody or an antigen binding fragment thereof for the use according to any one of embodiments E76 to E90, wherein the patient achieves a MGFA post-intervention status of Improved.
E92. An anti-C5 antibody or an antigen binding fragment thereof for the use according to any one of embodiments E76-E90, wherein the patient achieves a MGFA post-intervention status of MM.
E93. An anti-C5 antibody or an antigen binding fragment thereof for the use according to any one of embodiments E76 to E92, wherein the patient experiences a clinically meaningful improvement (reduction) in a measurement of generalized myasthenia gravis severity after 26 weeks of treatment selected from the group consisting of Myasthenia Gravis Activities of Daily Living (MG-ADL) score, quantitative Myasthenia Gravis (QMG), score and Myasthenia Gravis Composite (MGC) score.
E94. An anti-C5 antibody or an antigen binding fragment thereof for the use according to embodiment E93, wherein the clinically meaningful improvement the patient experiences is an at least a 3 point reduction in the patient's MG-ADL score after 26 weeks of treatment.
E95. An anti-C5 antibody or an antigen binding fragment thereof for the use according to embodiment E93, wherein the clinically meaningful improvement the patient experiences is an at least a 4 point reduction in the patient's QMG score after 26 weeks of treatment.
E96. An anti-C5 antibody or an antigen binding fragment thereof for the use according to embodiment E93, wherein the clinically meaningful improvement the patient experiences is an at least a 6 point reduction in the patient's MGC score after 26 weeks of treatment.
E97. An anti-C5 antibody or an antigen binding fragment thereof for the use according to any one of embodiments E76 to E96, wherein the patient experiences a clinically meaningful improvement (reduction) in quality of life as measured by Myasthenia Gravis Quality of Life (MG-QOL-15) score after 26 weeks of treatment.
E98. An anti-C5 antibody or an antigen binding fragment thereof for the use according to embodiment E97, wherein the clinically meaningful improvement the patient experiences is an at least a 6 point reduction in the patient's MG-QOL-15 score after 26 weeks of treatment.
E99. An anti-C5 antibody or an antigen binding fragment thereof for the use according to any one of embodiment E76 to E98, wherein the patient experiences a clinically meaningful improvement (reduction) in neuro-fatigue as measured by Neuro-QOL Fatigue score after 26 weeks of treatment.
E100. An anti-C5 antibody or an antigen binding fragment thereof for the use according to embodiment E99, wherein the clinically meaningful improvement the patient experiences is an at least an 8 point reduction in the patient's Neuro-QOL score after 26 weeks of treatment.
E101. An anti-C5 antibody or an antigen binding fragment thereof for the use according to any one of embodiments E76 to E100, wherein the patient experiences a clinically meaningful improvement (increase) in health status as measured by EQ-5D health status score after 26 weeks of treatment.
E102. An anti-C5 antibody or an antigen binding fragment thereof for use in treating refractory generalized myasthenia gravis in a patient in need thereof comprising administering eculizumab to the patient;
wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability;
wherein the patient is treated for at least 52 weeks and achieves a Myasthenia Gravis Foundation of America (MGFA) post-intervention status of Improved or Minimal Manifestations (MM) after at least 4 weeks of treatment; and
wherein the patient has a clinically meaningful improvement (reduction) in at least two measurements of generalized myasthenia gravis severity selected from the group consisting of MG-ADL, QMG, MGC, MG-QOL, and Neuro-QOL.
   E103. An anti-C5 antibody or an antigen binding fragment thereof for the use according to embodiment E102, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.
   E104. An anti-C5 antibody or an antigen binding fragment thereof for the use according to embodiment E102, wherein the therapeutically effective amount is based on the weight of the subject.
   E105. An anti-C5 antibody or an antigen binding fragment thereof for use in treating refractory generalized myasthenia gravis in a patient in need thereof comprising administering eculizumab to the patient;

wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability;

wherein the patient is treated for at least 52 weeks and achieves a Myasthenia Gravis Foundation of America (MGFA) post-intervention status of Improved or Minimal Manifestations (MM) after at least 4 weeks of treatment; and

wherein the patient has a clinically meaningful improvement (reduction) in five measurements of generalized myasthenia gravis severity, wherein the five measurements of generalized myasthenia gravis severity are a reduction in MG-ADL of at least 3 points, a reduction of QMG of at least 4 points, a reduction in MGC of at least 6 points, a reduction in MG-QOL of at least 6 points, and a reduction in Neuro-QOL of at least 8 points.

E106. An anti-C5 antibody or an antigen binding fragment thereof for the use according to embodiment E105 wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.
E107. An anti-C5 antibody or an antigen binding fragment thereof for the use according to embodiment E105, wherein the therapeutically effective amount is based on the weight of the subject.
E108. An anti-C5 antibody or an antigen binding fragment thereof for the use according to any one of embodiments E105 to E107, wherein the patient has a clinically meaningful improvement (reduction) in five measurements of generalized myasthenia gravis, wherein the five measurements of generalized myasthenia gravis severity are a reduction in MG-ADL of at least 4 points, a reduction of QMG of at least 5 points, a reduction in MGC of at least 10 points, a reduction in MG-QOL of at least 11 points, and a reduction in Neuro-QOL of at least 16 points.
E109. An anti-C5 antibody or an antigen binding fragment thereof for use in maintaining a Myasthenia Gravis Foundation of America (MGFA) post-intervention status of Improved or Minimal Manifestations (MM) in a patient with refractory generalized myasthenia gravis in need thereof comprising administering a therapeutically effective amount of eculizumab to the patient;

wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; and

wherein the patient had achieved the Improved or MM status.

E110. An anti-C5 antibody or an antigen binding fragment thereof for the use according to embodiment E109, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.
E111. An anti-C5 antibody or an antigen binding fragment thereof for the use according to embodiment E109 or embodiment E110, further comprising performing plasmapheresis on the patient and administering eculizumab at a dose of between 300 mg and 1200 mg to the patient within 4 hours of completion of plasmapheresis.
E112. An anti-C5 antibody or an antigen binding fragment thereof for the use according to embodiment E109 or embodiment E110, further comprising performing plasmapheresis on the patient and administering eculizumab at a dose of between 600 mg and 900 mg to the patient within 90 minutes of completion of plasmapheresis.
E113. An anti-C5 antibody or an antigen binding fragment thereof for the use according to embodiment E109 or embodiment E110, further comprising performing plasmapheresis on the patient and administering eculizumab at a dose of 600 mg to the patient within 1 hour of completion of plasmapheresis.
E114. An anti-C5 antibody or an antigen binding fragment thereof for the use according to embodiment E109, wherein the therapeutically effective amount is based on the weight of the subject.
E115. An anti-C5 antibody or an antigen binding fragment thereof for the use according to any one of embodiments E109 to E114, wherein the Improved or MM status is maintained for at least 4, 12, 26, 52, 66, 78, 104, 130 or 156 weeks.
E116. An anti-C5 antibody or an antigen binding fragment thereof for the use according to any one of embodiments E109 to E114, wherein the patient starts the maintenance with the MM status.
E117. An anti-C5 antibody or an antigen binding fragment thereof for the use according to any one of embodiments E109 to E114, wherein the MM status is maintained for at least 4, 12, 26, 52, 66, 78, 104, 130 or 156 weeks.
E118. An anti-C5 antibody or an antigen binding fragment thereof for the use according to any one of embodiments E76 to E117, wherein eculizumab is administered by intravenous infusion.
E119. An anti-C5 antibody or an antigen binding fragment thereof for the use according to any one of any one of embodiments E76 to E118, wherein the eculizumab is administered subcutaneously.
E120. An anti-C5 antibody or an antigen binding fragment thereof for the use according to any one of embodiments E76 to E119, wherein the eculizumab comprises a heavy chain amino acid sequence according to SEQ ID NO: 10 and a light chain amino acid sequence according to SEQ ID NO: 11.
E121. An anti-C5 antibody or an antigen binding fragment thereof for the use according to any one of embodiments E76 to E120, wherein the patient has failed treatment over one year or more with two or more ISTs in sequence or in combination.
E122. An anti-C5 antibody or an antigen binding fragment thereof for the use according to any one of embodiments E76 to E120, wherein the patient has failed at least one IST and requires chronic plasma exchange or IVIg to control symptoms.
E123. An anti-C5 antibody or an antigen binding fragment thereof for the use according to any one of embodiments E76 to E122, wherein the therapeutically effective amount of eculizumab is maintained at a concentration of between 50-100 μg/mL in the patient's serum.
E124. An anti-C5 antibody or an antigen binding fragment thereof for the use according to any one of embodiments E76 to E123, wherein the patient experiences a reduction in the administration of one or more IST following at least 26 weeks of treatment.
E125. An anti-C5 antibody or an antigen binding fragment thereof for the use according to any one of embodiments E76 to E124, wherein the patient experiences a reduction in IST dosing following at least 26 weeks of treatment.
E126. An anti-C5 antibody or an antigen binding fragment thereof for the use according to any one of embodiments E76 to E125, wherein the patient experiences a reduction in IST dosing and a discontinuation in one or more IST following at least 26 of treatment.
E127. An anti-C5 antibody or an antigen binding fragment thereof for the use according to any one of embodiments E76 to E126, wherein the patient switches from receiving one anti-C5 antibody or antigen binding fragment thereof to eculizumab during the course of treatment.

### REFERENCES CITED

The following references are hereby incorporated by reference in their entirety:
Conti-Fine et al., “Myasthenia gravis: past, present, and future,” J. Clin. Invest. 116(11): 2843-54 (2006). Howard J. F., “Clinical Overview of MG,” Myasthenia Gravis Foundation of America, Inc. 2006. Phillips, “The epidemiology of myasthenia gravis,” Semin. Neurol. 24(1): 17-20 (2004).
Kim et al., “Treatment of myasthenia gravis based on its immunopathogenesis,” J. Clin. Neurol. 7(4): 173-83 (2011). Vincent et al., “Myasthenia gravis,” Adv. Neurol. 88: 159-88 (2002).
Sahashi et al., “Ultrastructural localization of immune complexes (IgG and C3) at the end-plate in experimental autoimmune myasthenia gravis. J. Neuropathol. Exp. Neurol. 37(2): 212-23 (1978).
Dalakas, “Intravenous immunoglobulin in autoimmune neuromuscular diseases,” JAMA 291(19): 2367-75 (2004).
Engel et al., “Immune complexes (IgG and C3) at the motor end-plate in myasthenia gravis: ultrastructural and light microscopic localization and electrophysiologic correlations,” Mayo Clin. Proc. 52(5): 267-80 (1977).
Nastuk et al., “Changes in serum complement activity in patients with myasthenia gravis,” Proc. Soc. Exp. Biol. Med 105: 177-84 (1960).
Peng et al., “Role of C5 in the development of airway inflammation, airway hyperresponsiveness, and ongoing airway response,” J. Clin. Invest. 115(6): 1590-600 (2005).
Vakeva et al., “Myocardial infarction and apoptosis after myocardial ischemia and reperfusion: role of the terminal complement components and inhibition by anti-C5 therapy,” Circulation 97(22): 2259-67 (1998).
Wang et al., “Complement inhibition with an anti-C5 monoclonal antibody prevents hyperacute rejection in a xenograft heart transplantation model,” Transplantation 68(11): 1643-51 (1999).
Howard et al., “A randomized, double-blind, placebo-controlled phase II study of eculizumab in patients with refractory generalized myasthenia gravis,” Muscle Nerve 48(1): 76-84 (2013).
Muppidi et al., “MG-ADL: still a relevant outcome measure,” Muscle Nerve 44(5): 727-31 (2011).
Benatar et al., “Recommendations for myasthenia gravis clinical trials,” Muscle Nerve 45(6): 909-17 (2012). Burns et al., “The MG Composite: A valid and reliable outcome measure for myasthenia gravis,” Neurology 74(18): 1434-40 (2010).
Burns et al., “The MG-QOL15 for following the health-related quality of life of patients with myasthenia gravis,” Muscle Nerve 43(1): 14-8 (2011).
Cella D., Measuring Quality of Life in Neurological Disorders; Final Report of the Neuro-QOL Study September 2010. 2010. Szende A. and Williams A., “Measuring Self-Reported Population Health: An International Perspective based on EQ-5D, (2004) <http://www.euroqol.org/fileadmin/user_upload/Documenten/PDF/Books/Measuring_Self-Reported_Population_Health_-_An_International_Perspective_based_on_EQ-5D.pdf>. Posner et al., “The Columbia-Suicide Severity Rating Scale: initial validity and internal consistency findings from three multisite studies with adolescents and adults,” Am. J. Psychiatry 168(12): 1266-77 (2011).
Nilsson et al., “Columbia-Suicide Severity Rating Scale Scoring and Data Analysis Guide, (2013)<http://cssrs.columbia.edu/wp-content/uploads/ScoringandDataAnalysisGuide-for-Clinical-Trials.pdf>.
Grob D, Brunner N, Namba T, Pagala M. Lifetime course of myasthenia gravis. Muscle Nerve 2008; 37: 141-149.
Melzer N, Ruck T, Fuhr P, et al. Clinical features, pathogenesis, and treatment of myasthenia gravis: a supplement to the Guidelines of the German Neurological Society. J Neurol 2016; 263: 1473-1494.
Robertson N P, Deans J, Compston D A. Myasthenia gravis: a population based epidemiological study in Cambridgeshire, England. J Neurol Neurosurg Psychiatry 1998; 65: 492-496.
Zieda A, Ravina K, Glazere I, et al. A nationwide epidemiological study of myasthenia gravis in Latvia. Eur J Neurol 2018; 25: 519-526.
Lindstrom J M, Seybold M E, Lennon V A, Whittingham S, Duane D D. Antibody to acetylcholine receptor in myasthenia gravis. Prevalence, clinical correlates, and diagnostic value. Neurology 1976; 26: 1054-1059.
Mantegazza R, Pareyson D, Baggi F, et al. Anti AChR antibody: relevance to diagnosis and clinical aspects of myasthenia gravis. Ital J Neurol Sci 1988; 9: 141-145.
Oh S J, Kim D E, Kuruoglu R, Bradley R J, Dwyer D. Diagnostic sensitivity of the laboratory tests in myasthenia gravis. Muscle Nerve 1992; 15: 720-724.
Somnier F E. Clinical implementation of anti-acetylcholine receptor antibodies. J Neurol Neurosurg Psychiatry 1993; 56: 496-504.
Vincent A, McConville J, Farrugia M E, et al. Antibodies in myasthenia gravis and related disorders. Ann N Y Acad Sci 2003; 998: 324-335.
Vincent A, Newsom-Davis J. Acetylcholine receptor antibody as a diagnostic test for myasthenia gravis: results in 153 validated cases and 2967 diagnostic assays. J Neurol Neurosurg Psychiatry 1985: 48: 1246-1252.
Biesecker G, Gomez C M. Inhibition of acute passive transfer experimental autoimmune myasthenia gravis with Fab antibody to complement C6. J Immunol 1989; 142: 2654-2659.
Christadoss P. C5 gene influences the development of murine myasthenia gravis. J Immunol 1988; 140: 2589-2592.
Conti-Tronconi B, Tzartos S, Lindstrom J. Monoclonal antibodies as probes of acetylcholine receptor structure. 2. Binding to native receptor. Biochemistry 1981; 20: 2181-2191.
Fumagalli G, Engel A G, Lindstrom J. Ultrastructural aspects of acetylcholine receptor turnover at the normal end-plate and in autoimmune myasthenia gravis. J Neuropathol Exp Neurol 1982; 41:567-579.
Karachunski P I, Ostlie N S, Monfardini C, Conti-Fine B M. Absence of IFN-gamma or IL-12 has different effects on experimental myasthenia gravis in C57BL/6 mice. J Immunol 2000; 164: 5236-5244.
Piddlesden S J, Jiang S, Levin J L, Vincent A, Morgan B P. Soluble complement receptor 1 (sCR1) protects against experimental autoimmune myasthenia gravis. J Neuroimmunol 1996; 71: 173-177.
Howard J F Jr, Utsugisawa K, Benatar M, et al. Safety and efficacy of eculizumab in anti-acetylcholine receptor antibody-positive refractory generalised myasthenia gravis (REGAIN): a phase 3, randomised, double-blind, placebo-controlled, multicentre study. Lancet Neurol 2017; 16: 976-986.
Suh J, Goldstein J M, Nowak R J. Clinical characteristics of refractory myasthenia gravis patients. Yale J Biol Med 2013; 86: 255-260.
Silvestri N J, Wolfe G I. Treatment-refractory myasthenia gravis. J Clin Neuromuscul Dis 2014; 15: 167-178.
Sanders D B, Wolfe G I, Benatar M, et al. International consensus guidance for management of myasthenia gravis: executive summary. Neurology 2016; 87: 419-425.
Engel-Nitz N M, Boscoe A, Wolbeck R, Johnson J, Silvestri N J. Burden of illness in patients with treatment refractory myasthenia gravis. Muscle Nerve 2018; 58: 99-105.
Jaretzki A 3rd, Barohn R J, Ernstoff R M, et al. Myasthenia gravis: recommendations for clinical research standards. Task Force of the Medical Scientific Advisory Board of the Myasthenia Gravis Foundation of America. Ann Thorac Surg 2000; 70: 327-334.
Murai H. Japanese clinical guidelines for myasthenia gravis: putting into practice. Clinical and Experimental Neuroimmunology 2015; 6: 21-31.
Rother R P, Rollins S A, Mojcik C F, Brodsky R A, Bell L. Discovery and development of the complement inhibitor eculizumab for the treatment of paroxysmal nocturnal hemoglobinuria. Nat Biotechnol 2007; 25: 1256-1264.
Muppidi S, Utsugisawa K, Benatar M, et al. Long-term safety and efficacy of eculizumab in generalized myasthenia gravis. Muscle Nerve 2019; doi: 10.1002/mus.26447 [Epub ahead of print].
Buzzard K A, Meyer N J, Hardy T A, Riminton D S, Reddel S W (2015) Induction intravenous cyclophosphamide followed by maintenance oral immunosuppression in refractory myasthenia gravis. Muscle Nerve 52:204-210.
Drachman D B, Adams R N, Hu R, Jones R J, Brodsky R A (2008) Rebooting the immune system with high-dose cyclophosphamide for treatment of refractory myasthenia gravis. Ann N Y Acad Sci 1132:305-314.
Nowak R J, Dicapua D B, Zebardast N, Goldstein J M (2011) Response of patients with refractory myasthenia gravis to rituximab: a retrospective study. Ther Adv Neurol Disord 4:259-266.
Wolfe G I, Herbelin L, Nations S P, Foster B, Bryan W W, Barohn R J (1999) Myasthenia gravis activities of daily living profile. Neurology 52:1487-1489.
Burns T M, Conaway M R, Cutter G R, Sanders D B (2008) Less is more, or almost as much: a 15-item quality-of-life instrument for myasthenia gravis. Muscle Nerve 38:957-963.
Burns T M, Grouse C K, Conaway M R, Sanders D B (2010) Construct and concurrent validation of the MG-QOL15 in the practice setting. Muscle Nerve 41:219-226.
Muppidi S, Wolfe G I, Conaway M, Burns T M, MG Composite and MG-QOL15 Study Group (2011) MG-ADL: still a relevant outcome measure. Muscle Nerve 44:727-731.
Hillmen P, Young N S, Schubert J, Brodsky R A, Socie G, Muus P et al (2006) The complement inhibitor eculizumab in paroxysmal nocturnal hemoglobinuria. N Engl J Med 355:1233-1243.
Zuber J, Le Quintrec M, Krid S, Bertoye C, Gueutin V, Lahoche A et al (2012) Eculizumab for atypical hemolytic uremic syndrome recurrence in renal transplantation. Am J Transplant 12:3337-3354.
Zuber J, Fakhouri F, Roumenina L T, Loirat C, Fremeaux-Bacchi V, French Study Group for a HCG (2012) Use of eculizumab for atypical haemolytic uraemic syndrome and C3 glomerulopathies. Nat Rev Nephrol 8:643-657.
Legendre C M, Licht C, Muus P, Greenbaum L A, Babu S, Bedrosian C et al (2013) Terminal complement inhibitor eculizumab in atypical hemolytic-uremic syndrome. N Engl J Med 368:2169-2181.
Licht C, Greenbaum L A, Muus P, Babu S, Bedrosian C L, Cohen D J et al (2015) Efficacy and safety of eculizumab in atypical hemolytic uremic syndrome from 2-year extensions of phase 2 studies. Kidney Int 87:1061-1073.

1. Eculizumab for use in treating refractory generalized myasthenia gravis in a patient in need thereof;
wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability;
and
wherein the patient is treated for at least 52 weeks and achieves a Myasthenia Gravis Foundation of America (MGFA) post-intervention status of Improved or Minimal Manifestations (MM) after at least 4 weeks of treatment.

2. The use according to claim 1, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.

3. The use according to claim 1 or claim 2, further comprising performing plasmapheresis on the patient and administering eculizumab at a dose of between 300 mg and 1200 mg to the patient within 4 hours of completion of plasmapheresis.

4. The use according to claim 1 or claim 2, further comprising performing plasmapheresis on the patient and administering eculizumab at a dose of between 600 mg and 900 mg to the patient within 90 minutes of completion of plasmapheresis.

5. The use according to claim 1 or claim 2, further comprising performing plasmapheresis on the patient and administering eculizumab at a dose of 600 mg to the patient within 1 hour of completion of plasmapheresis.

6. The use according to any one of claims 1 to 5, wherein the therapeutically effective amount is based on the weight of the subject.

7. The use according to any one of claims 1 to 6, wherein the patient achieves a MGFA post-intervention status of Improved or MM after 4 weeks of treatment.

8. The use according to any one of claims 1 to 7, wherein the patient achieves a MGFA post-intervention status of Improved or MM after 12 weeks of treatment.

9. The use according to any one of claims 1 to 8, wherein the patient achieves a MGFA post-intervention status of Improved or MM after 26 weeks of treatment.

10. The use according to any one of claims 1 to 9, wherein the patient achieves a MGFA post-intervention status of Improved or MM after 52 weeks of treatment.

11. The use according to any one of claims 1 to 10, wherein the patient achieves a MGFA post-intervention status of Improved or MM after 66 weeks of treatment.

12. The use according to any one of claims 1 to 11, wherein the patient achieves a MGFA post-intervention status of Improved or MM after 78 weeks of treatment.

13. The use according to any one of claims 1 to 12, wherein the patient achieves a MGFA post-intervention status of Improved or MM after 104 weeks of treatment.

14. The use according to any one of claims 1 to 13, wherein the patient achieves a MGFA post-intervention status of Improved or MM after 130 weeks of treatment.

15. The use according to any one of claims 1 to 14, wherein the patient achieves a MGFA post-intervention status of Improved or MM after 156 weeks of treatment.

16. The use according to any one of claims 1-15, wherein the patient achieves a MGFA post-intervention status of Improved.

17. The use according to any one of claims 1-15, wherein the patient achieves a MGFA post-intervention status of MM.

18. The use according to any one of claims 1 to 17, wherein the patient experiences a clinically meaningful improvement (reduction) in a measurement of generalized myasthenia gravis severity after 26 weeks of treatment selected from the group consisting of Myasthenia Gravis Activities of Daily Living (MG-ADL) score, quantitative Myasthenia Gravis (QMG), score and Myasthenia Gravis Composite (MGC) score.

19. The use according to claim 18, wherein the clinically meaningful improvement the patient experiences is an at least a 3 point reduction in the patient's MG-ADL score after 26 weeks of treatment.

20. The use according to claim 18, wherein the clinically meaningful improvement the patient experiences is an at least a 4 point reduction in the patient's QMG score after 26 weeks of treatment.

21. The use according to claim 18, wherein the clinically meaningful improvement the patient experiences is an at least a 6 point reduction in the patient's MGC score after 26 weeks of treatment.

22. The use according to any one of claims 1 to 21, wherein the patient experiences a clinically meaningful improvement (reduction) in quality of life as measured by Myasthenia Gravis Quality of Life (MG-QOL-15) score after 26 weeks of treatment.

23. The use according to claim 22, wherein the clinically meaningful improvement the patient experiences is an at least a 6 point reduction in the patient's MG-QOL-15 score after 26 weeks of treatment.

24. The use according to any one of claims 1 to 23, wherein the patient experiences a clinically meaningful improvement (reduction) in neuro-fatigue as measured by Neuro-QOL Fatigue score after 26 weeks of treatment.

25. The use according to claim 24, wherein the clinically meaningful improvement the patient experiences is an at least an 8 point reduction in the patient's Neuro-QOL score after 26 weeks of treatment.

26. The use according to any one of claims 1 to 25, wherein the patient experiences a clinically meaningful improvement (increase) in health status as measured by EQ-5D health status score after 26 weeks of treatment.

27. Eculizumab for use in treating refractory generalized myasthenia gravis in a patient in need thereof comprising administering eculizumab to the patient;
wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability;
wherein the patient is treated for at least 52 weeks and achieves a Myasthenia Gravis Foundation of America (MGFA) post-intervention status of Improved or Minimal Manifestations (MM) after at least 4 weeks of treatment; and
wherein the patient has a clinically meaningful improvement (reduction) in at least two measurements of generalized myasthenia gravis severity selected from the group consisting of MG-ADL, QMG, MGC, MG-QOL, and Neuro-QOL.

28. The use according to claim 27, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.

29. The use according to claim 27, wherein the therapeutically effective amount is based on the weight of the subject.

30. Eculizumab for use in treating refractory generalized myasthenia gravis in a patient in need thereof comprising administering eculizumab to the patient;
wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability;
wherein the patient is treated for at least 52 weeks and achieves a Myasthenia Gravis Foundation of America (MGFA) post-intervention status of Improved or Minimal Manifestations (MM) after at least 4 weeks of treatment; and
wherein the patient has a clinically meaningful improvement (reduction) in five measurements of generalized myasthenia gravis severity, wherein the five measurements of generalized myasthenia gravis severity are a reduction in MG-ADL of at least 3 points, a reduction of QMG of at least 4 points, a reduction in MGC of at least 6 points, a reduction in MG-QOL of at least 6 points, and a reduction in Neuro-QOL of at least 8 points.

31. The use according to claim 30 wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.

32. The use according to claim 30, wherein the therapeutically effective amount is based on the weight of the subject.

33. The use according to any one of claims 30 to 32, wherein the patient has a clinically meaningful improvement (reduction) in five measurements of generalized myasthenia gravis, wherein the five measurements of generalized myasthenia gravis severity are a reduction in MG-ADL of at least 4 points, a reduction of QMG of at least 5 points, a reduction in MGC of at least 10 points, a reduction in MG-QOL of at least 11 points, and a reduction in Neuro-QOL of at least 16 points.

34. Eculizumab for use in maintaining a Myasthenia Gravis Foundation of America (MGFA) post-intervention status of Improved or Minimal Manifestations (MM) in a patient with refractory generalized myasthenia gravis in need thereof comprising administering a therapeutically effective amount of eculizumab to the patient;
wherein the patient is positive for auto-antibodies binding to nicotinic acetylcholine receptor (anti-AChR) and shows marked generalized weakness or bulbar signs and symptoms of myasthenia gravis while receiving therapy for myasthenia gravis including anticholinesterase inhibitor therapy and immunosuppressant therapy (IST) and requires chronic plasma exchange or chronic IVIg to maintain clinical stability; and
wherein the patient had achieved the Improved or MM status.

35. The use according to claim 34, wherein eculizumab is administered using a phased dosing schedule with an induction phase comprising administering a 900 mg induction dose of eculizumab on day 1, administering 900 mg doses of eculizumab on days 7, 14, and 21, and administering 1200 mg of eculizumab as a fifth induction dose on day 28, followed by a maintenance phase comprising administering 1200 mg of eculizumab 14 days after the fifth induction dose and administering 1200 mg of eculizumab every 14±2 days thereafter.

36. The use according to claim 34 or claim 35, further comprising performing plasmapheresis on the patient and administering eculizumab at a dose of between 300 mg and 1200 mg to the patient within 4 hours of completion of plasmapheresis.

37. The use according to claim 34 or claim 35, further comprising performing plasmapheresis on the patient and administering eculizumab at a dose of between 600 mg and 900 mg to the patient within 90 minutes of completion of plasmapheresis.

38. The use according to claim 34 or claim 35, further comprising performing plasmapheresis on the patient and administering eculizumab at a dose of 600 mg to the patient within 1 hour of completion of plasmapheresis.

39. The use according to claim 34, wherein the therapeutically effective amount is based on the weight of the subject.

40. The use according to any one of claims 34 to 39, wherein the Improved or MM status is maintained for at least 4, 12, 26, 52, 66, 78, 104, 130 or 156 weeks.

41. The use according to any one of claims 34 to 39, wherein the patient starts the maintenance with the MM status.

42. The use according to any one of claims 34 to 39, wherein the MM status is maintained for at least 4, 12, 26, 52, 66, 78, 104, 130 or 156 weeks.

43. The use according to any one of claims 1 to 42, wherein eculizumab is administered by intravenous infusion.

44. The use according to any one of any one of claims 1 to 43, wherein the eculizumab is administered subcutaneously.

45. The use according to any one of claims 1 to 44, wherein the eculizumab comprises a heavy chain amino acid sequence according to SEQ ID NO: 10 and a light chain amino acid sequence according to SEQ ID NO: 11.

46. The use according to any one of claims 1 to 45, wherein the patient has failed treatment over one year or more with two or more ISTs in sequence or in combination.

47. The use according to any one of claims 1 to 45, wherein the patient has failed at least one IST and requires chronic plasma exchange or IVIg to control symptoms.

48. The use according to any one of claims 1 to 47, wherein the therapeutically effective amount of eculizumab is maintained at a concentration of between 50-100 μg/mL in the patient's serum.

49. The use according to any one of claims 1 to 48, wherein the patient experiences a reduction in the administration of one or more IST following at least 26 weeks of treatment.

50. The use according to any one of claims 1 to 49, wherein the patient experiences a reduction in IST dosing following at least 26 weeks of treatment.

51. The use according to any one of claims 1 to 50, wherein the patient experiences a reduction in IST dosing and a discontinuation in one or more IST following at least 26 of treatment.

52. The use according to any one of claims 1 to 51, wherein the patient switches from receiving one anti-C5 antibody or antigen binding fragment thereof to eculizumab during the course of treatment.


SEQUENCE LISTING (52 sequences)

SEQ ID NO: 1 - protein, 10 aa, Artificial Sequence
  HCDR1
GYIFSNYWIQ

SEQ ID NO: 2 - protein, 17 aa, Artificial Sequence
  HCDR2
EILPGSGSTEYTENFKD

SEQ ID NO: 3 - protein, 13 aa, Artificial Sequence
  HCDR3
YFFGSSPNWYFDV

SEQ ID NO: 4 - protein, 11 aa, Artificial Sequence
  LCDR1
GASENIYGALN

SEQ ID NO: 5 - protein, 7 aa, Artificial Sequence
  LCDR2
GATNLAD

SEQ ID NO: 6 - protein, 9 aa, Artificial Sequence
  LCDR3
QNVLNTPLT

SEQ ID NO: 7 - protein, 122 aa, Artificial Sequence [sequence omitted]

SEQ ID NO: 8 - protein, 107 aa, Artificial Sequence [sequence omitted]

SEQ ID NO: 9 - protein, 326 aa, Artificial Sequence
  Heavy Chain Constant Region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - protein, 448 aa, Artificial Sequence
  Heavy Chain
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 11 - protein, 214 aa, Artificial Sequence [sequence omitted]

SEQ ID NO: 12 - protein, 122 aa, Artificial Sequence [sequence omitted]

SEQ ID NO: 13 - protein, 326 aa, Artificial Sequence
  Heavy Chain Constant Region
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 14 - protein, 448 aa, Artificial Sequence
  Heavy Chain
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVLHEALHSHYTQKSLSLSLGK

SEQ ID NO: 15 - protein, 326 aa, Artificial Sequence
  Heavy Chain Constant Region
ASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 16 - protein, 448 aa, Artificial Sequence
  Heavy Chain
QVQLVQSGAEVKKPGASVKVSCKASGYIFSNYWIQWVRQAPGQGLEWMGEILPGSGSTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVTSSNFGTQTYTCNVDHKPSNTKVDKTVERKCCVECPPCPAPPVAGPSVFLFPPKPKDTLYITREPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 17 - protein, 11 aa, Artificial Sequence
  CDR
GASENIYHALN

SEQ ID NO: 18 - protein, 17 aa, Artificial Sequence
  CDR
EILPGSGHTEYTENFKD

SEQ ID NO: 19 - protein, 10 aa, Artificial Sequence
  CDR
GHIFSNYWIQ

SEQ ID NO: 20 - protein, 448 aa, Artificial Sequence
  Heavy Chain
QVQLVQSGAEVKKPGASVKVSCKASGHIFSNYWIQWVRQAPGQGLEWMGEILPGSGHTEYTENFKDRVTMTRDTSTSTVYMELSSLRSEDTAVYYCARYFFGSSPNWYFDVWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 21 - protein, 5 aa, Artificial Sequence
  HCDR1
SYAIS

SEQ ID NO: 22 - protein, 17 aa, Artificial Sequence
  HCDR2
GIGPFFGTANYAQKFQG

SEQ ID NO: 23 - protein, 7 aa, Artificial Sequence
  HCDR3
DTPYFDY

SEQ ID NO: 24 - protein, 11 aa, Artificial Sequence
  LCDR1
SGDSIPNYYVY

SEQ ID NO: 25 - protein, 7 aa, Artificial Sequence
  LCDR2
DDSNRPS

SEQ ID NO: 26 - protein, 11 aa, Artificial Sequence
  LCDR3
QSFDSSLNAEV

SEQ ID NO: 27 - protein, 116 aa, Artificial Sequence
  Heavy Chain Variable Region
QVQLVQSGAEVKKPGSSVKVSCKASGGTFSSYAISVWRQAPGQGLEWMGGIGPFFGTANYAQKFQGRVTITADESTSTAYMELSSLRSEDTAVYYCARDTPYFDYWGQGTLVTVSS

SEQ ID NO: 28 - protein, 108 aa, Artificial Sequence
  Light Chain Variable Region
DIELTQPPSVSVAPGQTARISCSGDSIPNYYVYWYQQKPGQAPVLVIYDDSNRPSGIPERFSGSNSGNTATLTISGTQAEDEADYYCQSFDSSLNAEVFGGGTKLTVL

SEQ ID NO: 29 - protein, 4 aa, Artificial Sequence
  HCDR1
NYIS

SEQ ID NO: 30 - protein, 17 aa, Artificial Sequence
  HCDR2
IIDPDDSYTEYSPSFQG

SEQ ID NO: 31 - protein, 8 aa, Artificial Sequence
  HCDR3
YEYGGFDI

SEQ ID NO: 32 - protein, 11 aa, Artificial Sequence
  LCDR1
SGDNIGNSYVH

SEQ ID NO: 33 - protein, 7 aa, Artificial Sequence
  LCDR2
KDNDRPS

SEQ ID NO: 34 - protein, 9 aa, Artificial Sequence
  LCDR3
GTYDIESYV

SEQ ID NO: 35 - protein, 116 aa, Artificial Sequence
  Heavy Chain Variable Region
EVQLVQSGAEVKKPGESLKISCKGSGYSFTNYISWVRQMPGKGLEWMGIIDPDDSYTEYSPSFQGQVTISADKSISTAYLQWSSLKASDTAMYYCARYEYGGFDIWGQGTLVTVSS

SEQ ID NO: 36 - protein, 106 aa, Artificial Sequence
  Light Chain Variable Region
SYELTQPPSVSVAPGQTARISCSGDNIGNSYVHWYQQKPGQAPVLVIYKDNDRPSGIPERFSGSNSGNTATLTISGTQAEDEADYYCGTYDIESYVFGGGTKLTVL

SEQ ID NO: 37 - protein, 6 aa, Artificial Sequence
  CDR
SSYYVA

SEQ ID NO: 38 - protein, 17 aa, Artificial Sequence
  CDR
AIYTGSGATYKASWAKG

SEQ ID NO: 39 - protein, 13 aa, Artificial Sequence
  CDR
DGGYDYPTHAMHY

SEQ ID NO: 40 - protein, 11 aa, Artificial Sequence
  CDR
QASQNIGSSLA

SEQ ID NO: 41 - protein, 7 aa, Artificial Sequence
  CDR
GASKTHS

SEQ ID NO: 42 - protein, 12 aa, Artificial Sequence
  CDR
QSTKVGSSYGNH

SEQ ID NO: 43 - protein, 123 aa, Artificial Sequence
  305LO5 Heavy Chain Variable Region
QVQLVESGGGLVQPGGSLRLSCAASGFTSHSSYYVAWVRQAPGKGLEWVGAIYTGSGATYKASWAKGRFTISKDTSKNQVVLTMTNMDPVDTATYYCASDGGYDYPTHAMHYWGQGTLVTVSS

SEQ ID NO: 44 - protein, 110 aa, Artificial Sequence
  305LO5 Light Chain Variable Region
DVVMTQSPSSLSASVGDRVTITCQASQNIGSSLAWYQQKPGQAPRLLIYGASKTHSGVPSRFSGSGSGTDFTLTISSLQPEDVATYYCQSTKVGSSYGNHFGGGTKVEIK

SEQ ID NO: 45 - protein, 451 aa, Artificial Sequence
  SKY59 Heavy Chain
QVQLVESGGGLVQPGRSLRLSCAASGFTVHSSYYMAWVRQAPGKGLEWVGAIFTGSGAEYKAEWAKGRVTISKDTSKNQVVLTMTNMDPVDTATYYCASDAGYDYPTHAMHYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELRRGPKVFLFPPKPKDTLMISRTPEVTCVVVDVSHEDPEVKFNWYVDGVEVHNAKTKPREEQYNSTYRVVSVLTVLHQDWLNGKEYKCKVSNKGLPSSIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHAHYTRKELSLSP

SEQ ID NO: 46 - protein, 217 aa, Artificial Sequence
  SKY59 Light Chain
DIQMTQSPSSLSASVGDRVTITCRASQGISSSLAWYQQKPGKAPKLLIYGASETESGVPSRFSGSGSGTDFTLTISSLQPEDFATYYCQNTKVGSSYGNTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 47 - protein, 120 aa, Artificial Sequence
  REGN3918 Heavy Chain Variable Region
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSS

SEQ ID NO: 48 - protein, 107 aa, Artificial Sequence
  REGN3918 Light Chain Variable Region
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIK

SEQ ID NO: 49 - protein, 447 aa, Artificial Sequence
  REGN3918 Heavy Chain
QVQLQESGPGLVKPSETLSLTCTVSGDSVSSSYWTWIRQPPGKGLEWIGYIYYSGSSNYNPSLKSRATISVDTSKNQFSLKLSSVTAADTAVYYCAREGNVDTTMIFDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 50 - protein, 214 aa, Artificial Sequence
  REGN3918 Light Chain
AIQMTQSPSSLSASVGDRVTITCRASQGIRNDLGWYQQKPGKAPKLLIYAASSLQSGVPSRFAGRGSGTDFTLTISSLQPEDFATYYCLQDFNYPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 123 aa, Artificial Sequence [sequence omitted]

SEQ ID NO: 52 - protein, 110 aa, Artificial Sequence [sequence omitted]